(19) United States
(12) Patent Application Publication
(10) Pub. No.: US 2007/0232607 A1
(43) Pub. Date: Oct. 4, 2007

(54) Quinazoline Derivatives as Erbb Receptor Tyrosine kinases

(76) Inventors: Robert Hugh Bradbury, Macclesfield (GB); Jason Grant Kettle, Macclesfield (GB); James Stewart Scott, Macclesfield (GB); Bernard Christophe Barlaam, Reims (FR)

Correspondence Address: MORGAN LEWIS & BOCKIUS LLP, 1111 PENNSYLVANIA AVENUE NW, WASHINGTON, DC, 20004, US

(21) Appl. No.: 11/628,011

(22) PCT Filed: Jun. 2, 2005
(86) PCT No.: PCT/GB05/02215

§ 371 Date: Nov. 30, 2006

(30) Foreign Application Priority Data
Jun. 4, 2004 (EP) 04291393.9

(51) Int. Cl. A61K 31/517; A61K 31/496; A61K 31/5377; C07D 239/72; C07D 401/00; C07D 413/14

(52) U.S. Cl. 514/234.5; 514/252.17; 514/266.1; 514/266.21; 544/120; 544/122; 544/283; 544/284

(57) ABSTRACT

The invention concerns quinazoline derivatives of the formula (I), wherein each of R¹, R², R³, R⁴, R⁵, R⁶, R⁷, X¹, Q¹, m and n have any of the meanings defined in the description; processes for their preparation, pharmaceutical compositions containing them and their use in the manufacture of a medicament for use as an antiproliferative agent in the prevention or treatment of tumours which are sensitive to inhibition of erbB receptor tyrosine kinases.

The invention concerns certain novel quinazoline derivatives, or pharmaceutically acceptable salts thereof, which possess anti-tumour activity and are accordingly useful in methods of treatment of the human or animal body. The invention also concerns processes for the manufacture of said quinazoline derivatives, pharmaceutical compositions containing them and their use in therapeutic methods, for example in the manufacture of medicaments for use in the prevention or treatment of solid tumour disease in a warm-blooded animal such as man.

Many of the current treatment regimes for diseases resulting from the abnormal regulation of cellular proliferation such as psoriasis and cancer, utilise compounds that inhibit DNA synthesis and cellular proliferation. To date, compounds used in such treatments are generally toxic to cells however their enhanced effects on rapidly dividing cells such as tumour cells can be beneficial. Alternative approaches to these cytotoxic anti-tumour agents are currently being developed, for example selective inhibitors of cell signalling pathways. These types of inhibitors are likely to have the potential to display an enhanced selectivity of action against tumour cells and so are likely to reduce the probability of the therapy possessing unwanted side effects.

Eukaryotic cells are continually responding to many diverse extracellular signals that enable communication between cells within an organism. These signals regulate a wide variety of physical responses in the cell including proliferation, differentiation, apoptosis and motility. The extracellular signals take the form of a diverse variety of soluble factors including growth factors and other autocrine, paracrine and endocrine factors. By binding to specific transmembrane receptors, these ligands integrate the extracellular signal to the intracellular signalling pathways, therefore transducing the signal across the plasma membrane and allowing the individual cell to respond to its extracellular signals. Many of these signal transduction processes utilise the reversible process of the phosphorylation of proteins that are involved in the promotion of these diverse cellular responses. The phosphorylation status of target proteins is regulated by specific kinases and phosphatases that are responsible for the regulation of about one third of all proteins encoded by the mammalian genome. As phosphorylation is such an important regulatory mechanism in the signal transduction process, it is therefore not surprising that aberrations in these intracellular pathways result in abnormal cell growth and differentiation and so promote cellular transformation (reviewed in Cohen et al, Curr Opin Chem Biol, 1999, 3, 459-465).

It has been widely shown that a number of these tyrosine kinases are mutated to constitutively active forms and/or when over-expressed result in the transformation of a variety of human cells. These mutated and over-expressed forms of the kinase are present in a large proportion of human tumours (reviewed in Kolibaba et al, Biochimica et Biophysica Acta, 1997, 133, F217-F248). As tyrosine kinases play fundamental roles in the proliferation and differentiation of a variety of tissues, much focus has centred on these enzymes in the development of novel anti-cancer therapies. This family of enzymes is divided into two groups—receptor and non-receptor tyrosine kinases e.g. EGF Receptors and the SRC family respectively. From the results of a large number of studies including the Human Genome Project, about 90 tyrosine kinase have been identified in the human genome, of this 58 are of the receptor type and 32 are of the non-receptor type. These can be compartmentalised into 20 receptor tyrosine kinase and 10 non-receptor tyrosine kinase sub-families (Robinson et al, Oncogene, 2000, 19, 5548-5557).

The receptor tyrosine kinases are of particular importance in the transmission of mitogenic signals that initiate cellular replication. These large glycoproteins, which span the plasma membrane of the cell, possess an extracellular binding domain for their specific ligands (such as Epidermal Growth Factor (EGF) for the EGF Receptor). Binding of ligand results in the activation of the receptor's kinase enzymatic activity that resides in the intracellular portion of the receptor. This activity phosphorylates key tyrosine amino acids in target proteins, resulting in the transduction of proliferative signals across the plasma membrane of the cell.

It is known that the erbB family of receptor tyrosine kinases, which include EGFR, erbB2, erbB3 and erbB4, are frequently involved in driving the proliferation and survival of tumour cells (reviewed in Olayioye et al., EMBO J., 2000, 19, 3159). One mechanism in which this can be accomplished is by overexpression of the receptor at the protein level, generally as a result of gene amplification. This has been observed in many common human cancers (reviewed in Klapper et al., Adv. Cancer Res., 2000, 77, 25) such as breast cancer (Sainsbury et al., Brit. J. Cancer, 1988, 58, 458; Guerin et al., Oncogene Res., 1988, 3, 21; Slamon et al., Science, 1989, 244, 707; Klijn et al., Breast Cancer Res. Treat., 1994, 29, 73 and reviewed in Salomon et al., Crit. Rev. Oncol. Hematol., 1995, 19, 183), non-small cell lung cancers (NSCLCs) including adenocarcinomas (Cerny et al., Brit. J. Cancer, 1986, 54, 265; Reubi et al., Int. J. Cancer, 1990, 45, 269; Rusch et al., Cancer Research, 1993, 53, 2379; Brabender et al, Clin. Cancer Res., 2001, 7, 1850) as well as other cancers of the lung (Hendler et al., Cancer Cells, 1989, 7, 347; Ohsaki et al., Oncol. Rep., 2000, 7, 603), bladder cancer (Neal et al., Lancet, 1985, 366; Chow et al., Clin. Cancer Res., 2001, 7, 1957, Zhau et al., Mol Carcinog., 3, 254), oesophageal cancer (Mukaida et al., Cancer, 1991, 68, 142), gastrointestinal cancer such as colon, rectal or stomach cancer (Bolen et al., Oncogene Res., 1987, 1, 149; Kapitanovic et al., Gastroenterology, 2000, 112, 1103; Ross et al., Cancer Invest., 2001, 19, 554), cancer of the prostate (Visakorpi et al., Histochem. J., 1992, 24, 481; Kumar et al., 2000, 32, 73; Scher et al., J. Natl. Cancer Inst., 2000, 92, 1866), leukaemia (Konaka et al., Cell, 1984, 37, 1035, Martin-Subero et al., Cancer Genet Cytogenet., 2001, 127, 174), ovarian (Hellstrom et al., Cancer Res., 2001, 61, 2420), head and neck (Shiga et al., Head Neck, 2000, 22, 599) or pancreatic cancer (Ovotny et al., Neoplasma, 2001, 48, 188). As more human tumour tissues are tested for expression of the erbB family of receptor tyrosine kinases it is expected that their widespread prevalence and importance will be further enhanced in the future.

As a consequence of the mis-regulation of one or more of these receptors (in particular erbB2), it is widely believed that many tumours become clinically more aggressive and so correlate with a poorer prognosis for the patient (Brabender et al, Clin. Cancer Res., 2001, 7, 1850; Ross et al, Cancer Investigation, 2001, 19, 554, Yu et al., Bioessays, 2000, 22.7, 673).

In addition to these clinical findings, a wealth of pre-clinical information suggests that the erbB family of receptor tyrosine kinases are involved in cellular transformation. This includes the observations that many tumour cell lines overexpress one or more of the erbB receptors and that EGFR or erbB2 when transfected into non-tumour cells have the ability to transform these cells. This tumourigenic potential has been further verified as transgenic mice that overexpress erbB2 spontaneously develop tumours in the mammary gland. In addition to this, a number of pre-clinical studies have demonstrated that anti-proliferative effects can be induced by knocking out one or more erbB activities by small molecule inhibitors, dominant negatives or inhibitory antibodies (reviewed in Mendelsohn et al., Oncogene, 2000, 19, 6550). Thus it has been recognised that inhibitors of these receptor tyrosine kinases should be of value as a selective inhibitor of the proliferation of mammalian cancer cells (Yaish et al. Science, 1988, 242, 933, Kolibaba et al, Biochimica et Biophysica Acta, 1997, 133, F217-F248; Al-Obeidi et al, 2000, Oncogene, 19, 5690-5701; Mendelsohn et al, 2000, Oncogene, 19, 6550-6565).

In addition to this pre-clinical data, the small molecule EGFR tyrosine kinase inhibitors Iressa (also known as gefitinib and ZD1839) and Tarceva (also known as erlotinib and CP-358,774) have been approved for use in the treatment of advanced non-small cell lung cancer. Furthermore, inhibitory antibodies against EGFR and erbB2 (erbitux (c-225/cetuximab) and herceptin (trastuzumab) respectively) have proven to be beneficial in the clinic for the treatment of selected solid tumours (reviewed in Mendelsohn et al, 2000, Oncogene, 19, 6550-6565).

Recently mutations in the ATP binding pocket of the intracellular catalytic domain of the EGF receptor have been discovered in certain sub-sets of non-small cell lung cancers (NSCLCs). The presence of mutations in the receptor appear to correlate with response to EGFR tyrosine kinase inhibitors such as gefitinib (Lynch et al, N Engl J Med 2004; 350: 2129-2139; Paez et al, Science 2004; 304: 1497-1500), although it is becoming evident that the clinical benefits of compounds such as gefitinib and erlotinib are not likely to be mediated by EGFR mutations alone. It has been demonstrated that ligand stimulation results in a different phosphorylation pattern in mutated receptors compared with that seen in wild-type receptors and it is thought that mutant EGF receptors selectively transduce survival signals on which NSCLCs become dependent. Inhibition of those signals by compounds such as gefitinib may contribute to the efficacy of such drugs (Sordella et al. Science 2004; 305: 1163-1167). Similarly, mutations within the erbB2 kinase domain have recently been discovered in certain primary tumours, such as NSCLC, glioblastoma and gastric and ovarian tumours (Stephens et al., Nature 2004; 431; 525-526). Accordingly the inhibition of the EGF and/or erbB2 tyrosine kinase in both wild-type and mutated receptors is an important target that would be expected to provide an anti-cancer effect.

Amplification and/or activity of members of the erbB type receptor tyrosine kinases have been detected and so have been implicated to play a role in a number of non-malignant proliferative disorders such as psoriasis (Ben-Bassat, Curr. Pharm. Des., 2000, 6, 933; Elder et al., Science, 1989, 243, 811), benign prostatic hyperplasia (BPH) (Kumar et al., Int. Urol. Nephrol., 2000, 32, 73), atherosclerosis and restenosis (Bokemeyer et al., Kidney Int., 2000, 58, 549). It is therefore expected that inhibitors of erbB type receptor tyrosine kinases will be useful in the treatment of these and other non-malignant disorders of excessive cellular proliferation.

International Patent Applications WO 96/09294, WO 96/15118, WO 96/16960, WO 96/30347, WO 96/33977, WO 96/33978, WO 96/33979, WO 96/33980, WO 96/33981, WO 97/03069, WO 97/13771, WO 97/30034, WO 97/30035, WO 97/38983, WO 98/02437, WO 98/02434, WO 98/02438, WO 98/13354, WO 99/35132, WO 99/35146, WO 01/21596, WO 01/55141 and WO 02/18372 disclose that certain quinazoline derivatives which bear an anilino substituent at the 4-position possess receptor tyrosine kinase inhibitory activity.

International Patent Applications WO 97/22596 and WO 98/13354 disclose that certain 4-anilinoquinazoline derivatives that are substituted at the 7-position are VEGF inhibitors or mixed VEGF/EGF receptor tyrosine kinase inhibitors. The anilino group in these applications is substituted with small groups such as halogeno or (1-3C)alkyl.

International Patent Application WO 01/94341 discloses that certain quinazoline derivatives which are substituted at the 5-position are inhibitors of the Src family of non-receptor tyrosine kinases, such as c-Src, c-Yes and c-Fyn. There are no disclosures in WO 01/94341 of 4-anilinoquinazolines wherein the aniline group is substituted in the para position by a substituent containing an aryl or heteroaryl group.

International Patent applications WO 03/040108 and WO 03/040109 disclose that certain 5-substitued quinazoline derivatives are inhibitors of the erbB family of tyrosine kinase inhibitors, particularly EGFR and erbB2 receptor tyrosine kinases. All the quinazoline derivatives in these applications carry a ring containing substituent at the 5-position on the quinazoline ring.

International Patent application WO2004/096226 discloses that certain substituted 4-anilino-quinazoline derivatives are inhibitors of the erbB family of tyrosine kinase inhibitors, particularly EGFR receptor tyrosine kinase. This application does not disclose any quinazoline derivatives in which the anilino group is substituted in the para position by a substituent containing an aryl or heteroaryl group or any quinazoline derivatives that contain a methoxy linked amide substituent at the 5-position on the quinazoline ring.

International Patent application WO2004/106308 discloses that certain substituted 4-anilino-quinazoline derivatives are inhibitors of the erbB family of tyrosine kinase inhibitors, particularly erbB2 receptor tyrosine kinase. None of the quinazoline derivatives disclosed in this application contain a substituent at the 5-position on the quinazoline ring.

International Patent application WO2004/093880 discloses that certain substituted 4-anilino-quinazoline derivatives are inhibitors of the erbB family of tyrosine kinase inhibitors, particularly erbB2 receptor tyrosine kinase. None of the quinazoline derivatives disclosed in this application contain a methoxy linked amide substituent at the 5-position on the quinazoline ring.

None of the prior art discloses 4-anilinoquinazoline derivatives that are substituted at the 5-position by a methoxy linked amide group and which carry an aryl or heteroaryl containing substituent at the para-position on the aniline ring.

We have now found that surprisingly certain 4-anilino-quinazoline derivatives substituted at the 5-position with a substituent containing a methoxy linked amide group possess potent anti-tumour activity. Without wishing to imply that the quinazoline derivatives disclosed in the present invention possess pharmacological activity only by virtue of an effect on a single biological process, it is believed that the quinazoline derivatives provide an anti-tumour effect by way of inhibition of one or more of the erbB family of receptor tyrosine kinases that are involved in the signal transduction steps which lead to the proliferation of tumour cells. In particular, it is believed that the quinazoline derivatives of the present invention provide an anti-tumour effect by way of inhibition of EGFR and/or erbB2 receptor tyrosine kinases.

Generally the quinazoline derivatives of the present invention possess potent inhibitory activity against the erbB receptor tyrosine kinase family, for example by inhibition of EGFR and/or erbB2 and/or erbB4 receptor tyrosine kinases, whilst possessing less potent inhibitory activity against other kinases. Furthermore, generally the quinazoline derivatives of the present invention possess substantially better potency against the erbB2 over that of the EGFR tyrosine kinase, thus potentially providing effective treatment for erbB2 driven tumours. Accordingly, it may be possible to administer a quinazoline derivative according to the present invention at a dose that is sufficient to inhibit erbB2 tyrosine kinase whilst having no significant effect upon EGFR (or other) tyrosine kinases. The selective inhibition provided by the quinazoline derivatives according to the present invention may provide treatments for conditions mediated by erbB2 tyrosine kinase, whilst reducing undesirable side effects that may be associated with the inhibition of other tyrosine kinases. Generally the quinazoline derivatives according to the invention also exhibit favourable DMPK properties, for example high bioavailability, and favourable physical properties such as solubility. Furthermore, many of the quinazoline derivatives according to the present invention are inactive or only weakly active in a hERG assay and/or in the P450 cytochrome inhibition assay.

References to erbB receptors, particularly erbB2, used herein are intended to include both wild-type and mutated receptors unless specifically stated otherwise. The term “mutation” includes, but is not limited to, gene amplification, nucleotide in-frame deletions or substitutions in one or more of the exons that encode receptors such as erbB2.

According to a first aspect of the invention there is provided a quinazoline derivative of the formula I: 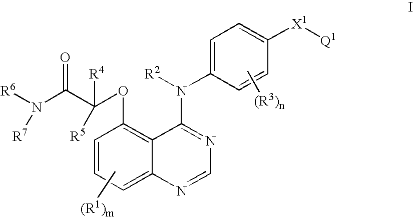
wherein:

m is 0, 1 or 2;

each R¹, which may be the same or different, is selected from hydroxy, (1-6C)alkoxy, (3-7C)cycloalkyl-oxy and (3-7C)cycloalkyl-(1-6C)alkoxy, and wherein any CH₂ or CH₃ group within an R¹ substituent optionally bears on each said CH₂ or CH₃ group one or more substituents independently selected from halogeno, (1-6C)alkyl, hydroxy and (1-6C)alkoxy;

R² is hydrogen or (1-4C)alkyl;

n is 0, 1, 2, 3 or 4;

each R³, which may be the same or different, is selected from halogeno, cyano, (1-4C)alkyl, trifluoromethyl, (1-4C)alkoxy, (2-4C)alkenyl and (2-4C)alkynyl;

X¹ is selected from O, S, SO, SO₂, N(R¹³), CH(OR¹³), CON(R¹³), N(R¹³)CO, SO₂N(R¹³), N(R¹³)SO₂, OC(R¹³)₂, C(R¹³)₂O, SC(R¹³)₂, C(R¹³)₂S, CO, C(R¹³)₂N(R¹³) and N(R¹³)C(R¹³)₂, wherein each R¹³, which may be the same or different, is hydrogen or (1-6C)alkyl;

Q¹ is aryl or heteroaryl,

and wherein Q¹ optionally bears one or more substituents, which may be the same or different, selected from halogeno, cyano, nitro, hydroxy, amino, carboxy, carbamoyl, sulfamoyl, formyl, mercapto, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (3-6C)alkenoyl, (3-6C)alkynoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkylsulfonylamino, N-(1-6C)alkyl-(1-6C)alkylsulfonylamino, and a group of the formula:
—X²—R⁸

wherein X² is a direct bond or is selected from O, CO and N(R⁹), wherein R⁹ is hydrogen or (1-6C)alkyl, and R⁸ is selected from halogeno-(1-6C)alkyl, hydroxy-(1-6C)alkyl, carboxy-(1-6C)alkyl, (1-6C)alkoxy-(1-6C)alkyl, cyano-(1-6C)alkyl, amino-(1-6C)alkyl, N-(1-6C)alkylamino-(1-6C)alkyl, N,N-di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, (1-6C)alkoxycarbonylamino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, (1-6C)alkylthio-(1-6C)alkyl, (1-6C)alkylsulfinyl-(1-6C)alkyl, (1-6C)alkylsulfonyl-(1-6C)alkyl, sulfamoyl-(1-6C)alkyl, N-(1-6C)alkylsulfamoyl-(1-6C)alkyl, N,N-di-(1-6C)alkylsulfamoyl-(1-6C)alkyl, (2-6C)alkanoyl-(1-6C)alkyl, (2-6C)alkanoyloxy-(1-6C)alkyl and (1-6C)alkoxycarbonyl-(1-6C)alkyl,

and wherein any CH₂ or CH₃ group within —X¹-Q¹ optionally bears on each said CH₂ or CH₃ group one or more substituents independently selected from halogeno, (1-6C)alkyl, hydroxy, cyano, amino, (1-4C)alkoxy, (1-4C)alkylamino and di-[(1-4C)alkylamino];

R⁴ and R⁵, which may be the same or different, are selected from hydrogen and (1-6C)alkyl, or

R⁴ and R⁵ together with the carbon atom to which they are attached form a (3-7C)cycloalkyl ring,

and wherein any CH₂ or CH₃ group within any of R⁴ and R⁵ optionally bears on each said CH₂ or CH₃ group one or more substituents independently selected from halogeno, hydroxy, cyano, (1-6C)alkoxy, amino, (2-6C)alkanoyl, (1-6C)alkylamino and di-[(1-6C)alkylamino];

R⁶ and R⁷, which may be the same or different, are selected from hydrogen, (1-6C)alkyl, (2-6C)alkenyl, (2-6C)alkynyl, (3-7C)cycloalkyl, (3-7C)cycloalkyl-(1-6C)alkyl, (3-7C)cycloalkenyl, (3-7C)cycloalkenyl-(1-6C)alkyl, heterocyclyl and heterocyclyl-(1-6C)alkyl, or

R⁶ and R⁷ together with the nitrogen atom to which they are attached form a saturated 4, 5, 6 or 7 membered heterocyclic ring which optionally contains one or more additional heteroatoms independently selected from oxygen, S, SO, SO₂ and NR¹⁰, wherein R¹⁰ is selected from hydrogen, (1-6C)alkyl, (2-6C)alkenyl, (2-6C)alkynyl, (1-6C)alkylsulfonyl, (1-6C)alkylcarbonyl and (1-6C)alkoxycarbonyl;

and wherein any heterocyclyl group within an R⁶ or an R⁷ substituent or any heterocyclic ring formed by R⁶, R⁷ and the nitrogen atom to which they are attached optionally bears one or more substituents, which may be the same or different, selected from halogeno, trifluoromethyl, cyano, nitro, hydroxy, amino, formyl, mercapto, (1-6C)alkyl, (2-6C)alkenyl, (2-6C)alkynyl, hydroxy-(1-6C)alkyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X³—R¹¹

wherein X³ is a direct bond or is selected from O, CO, SO₂ and N(R¹²), wherein R¹² is hydrogen or (1-4C)alkyl, and R¹¹ is selected from halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl,

and wherein any heterocyclyl group within an R⁶ or an R⁷ substituent or any heterocyclic ring formed by R⁶, R⁷ and the nitrogen atom to which they are attached optionally bears 1 or 2 oxo or thioxo substituents;

and wherein any CH₂ or CH₃ group within an R⁶ or an R⁷ substituent, other than a CH₂ group within a heterocyclyl group or heterocyclic ring, optionally bears on each said CH₂ or CH₃ group one or more substituents independently selected from halogeno, (1-6C)alkyl, hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkylsulfonylamino and N-(1-6C)alkyl-(1-6C)alkylsulfonylamino;

or a pharmaceutically acceptable salt thereof.

According to a second aspect of the invention there is provided a quinazoline derivative of the formula I wherein:

m is 0, 1 or 2;

each R¹, which may be the same or different, is selected from hydroxy, (1-6C)alkoxy, (3-7C)cycloalkyl-oxy and (3-7C)cycloalkyl-(1-6C)alkoxy,

and wherein any CH₂ or CH₃ group within an R¹ substituent optionally bears on each said CH₂ or CH₃ group one or more substituents independently selected from halogeno, (1-6C)alkyl, hydroxy and (1-6C)alkoxy;

R² is hydrogen or (1-4C)alkyl;

n is 0, 1, 2, 3 or 4;

each R³, which may be the same or different, is selected from halogeno, (1-4C)alkyl, trifluoromethyl, (1-4C)alkoxy, (2-4C)alkenyl and (2-4C)alkynyl;

X¹ is selected from O, S, SO, SO₂, N(R¹³), CH(OR¹³), CON(R¹³), N(R¹³)CO, SO₂N(R¹³), N(R¹³)SO₂, OC(R¹³)₂, C(R¹³)₂O, SC(R¹³)₂, C(R¹³)₂S, CO, C(R¹³)₂N(R¹³) and N(R¹³)C(R¹³)₂, wherein each R¹³, which may be the same or different, is hydrogen or (1-6C)alkyl;

Q¹ is aryl or heteroaryl,

and wherein Q¹ optionally bears one or more substituents, which may be the same or different, selected from halogeno, cyano, nitro, hydroxy, amino, carboxy, carbamoyl, sulfamoyl, formyl, mercapto, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (3-6C)alkenoyl, (3-6C)alkynoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkylsulfonylamino, N-(1-6C)alkyl-(1-6C)alkylsulfonylamino, and a group of the formula:
—X²—R⁸

wherein X² is a direct bond or is selected from O, CO and N(R⁹), wherein R⁹ is hydrogen or (1-6C)alkyl, and R⁸ is selected from halogeno-(1-6C)alkyl, hydroxy-(1-6C)alkyl, carboxy-(1-6C)alkyl, (1-6C)alkoxy-(1-6C)alkyl, cyano-(1-6C)alkyl, amino-(1-6C)alkyl, N-(1-6C)alkylamino-(1-6C)alkyl, N,N-di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, (1-6C)alkoxycarbonylamino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, (1-6C)alkylthio-(1-6C)alkyl, (1-6C)alkylsulfinyl-(1-6C)alkyl, (1-6C)alkylsulfonyl-(1-6C)alkyl, sulfamoyl-(1-6C)alkyl, N-(1-6C)alkylsulfamoyl-(1-6C)alkyl, N,N-di-(1-6C)alkylsulfamoyl-(1-6C)alkyl, (2-6C)alkanoyl-(1-6C)alkyl, (2-6C)alkanoyloxy-(1-6C)alkyl and (1-6C)alkoxycarbonyl-(1-6C)alkyl,

and wherein any CH₂ or CH₃ group within —X¹-Q¹ optionally bears on each said CH₂ or CH₃ group one or more substituents independently selected from halogeno, (1-6C)alkyl, hydroxy, cyano, amino, (1-4C)alkoxy, (1-4C)alkylamino and di-[(1-4C)alkylamino];

R⁴ and R⁵, which may be the same or different, are selected from hydrogen and (1-6C)alkyl, or

R⁴ and R⁵ together with the carbon atom to which they are attached form a (3-7C)cycloalkyl ring,

and wherein any CH₂ or CH₃ group within any of R⁴ and R⁵ optionally bears on each said CH₂ or CH₃ group one or more substituents independently selected from halogeno, hydroxy, cyano, (1-6C)alkoxy, amino, (2-6C)alkanoyl, (1-6C)alkylamino and di-[(1-6C)alkylamino];

R⁶ and R⁷, which may be the same or different, are selected from hydrogen, (1-6C)alkyl, (2-6C)alkenyl, (2-6C)alkynyl, (3-7C)cycloalkyl, (3-7C)cycloalkyl-(1-6C)alkyl, (3-7C)cycloalkenyl, (3-7C)cycloalkenyl-(1-6C)alkyl, heterocyclyl and heterocyclyl-(1-6C)alkyl, or

R⁶ and R⁷ together with the nitrogen atom to which they are attached form a saturated 5 or 6 membered heterocyclic ring which optionally contains one or more additional heteroatoms independently selected from oxygen and N(R¹⁰), wherein R¹⁰ is selected from hydrogen, (1-6C)alkyl, (2-6C)alkenyl, (2-6C)alkynyl, (1-6C)alkylsulfonyl or (1-6C)alkylcarbonyl,

and wherein any heterocyclyl group within an R⁶ or an R⁷ substituent or any heterocyclic ring formed by R⁶, R⁷ and the nitrogen atom to which they are attached optionally bears one or more substituents, which may be the same or different, selected from halogeno, trifluoromethyl, cyano, nitro, hydroxy, amino, formyl, mercapto, (1-6C)alkyl, (2-6C)alkenyl, (2-6C)alkynyl, hydroxy-(1-6C)alkyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X³—R¹¹

wherein X³ is a direct bond or is selected from O, CO, SO₂ and N(R¹²), wherein R¹² is hydrogen or (1-4C)alkyl, and R¹¹ is selected from halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl,

and wherein any heterocyclyl group within an R⁶ or an R⁷ substituent or any heterocyclic ring formed by R⁶, R⁷ and the nitrogen atom to which they are attached optionally bears 1 or 2 oxo or thioxo substituents;

and wherein any CH₂ or CH₃ group within an R⁶ or an R⁷ substituent, other than a CH₂ group within a heterocyclyl group or a heterocyclic ring, optionally bears on each said CH₂ or CH₃ group one or more substituents independently selected from halogeno, (1-6C)alkyl, hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkylsulfonylamino and N-(1-6C)alkyl-(1-6C)alkylsulfonylamino;

or a pharmaceutically acceptable salt thereof.

According to a third aspect of the invention there is provided a quinazoline derivative of the formula I wherein:

m is 0, 1 or 2;

each R¹, which may be the same or different, is selected from hydroxy, (1-6C)alkoxy, (3-7C)cycloalkyl-oxy and (3-7C)cycloalkyl-(1-6C)alkoxy,

and wherein any CH₂ or CH₃ group within an R¹ substituent optionally bears on each said CH₂ or CH₃ group one or more substituents independently selected from halogeno, (1-6C)alkyl, hydroxy and (1-6C)alkoxy;

R² is hydrogen or (1-4C)alkyl;

n is 0, 1, 2, 3 or 4;

each R³, which may be the same or different, is selected from halogeno, cyano, (1-4C)alkyl, trifluoromethyl, (1-4C)alkoxy, (2-4C)alkenyl and (2-4C)alkynyl;

X¹ is selected from S, SO, SO₂, N(R¹³), CH(OR¹³), CON(R¹³), N(R¹³)CO, SO₂N(R¹³), N(R¹³)SO₂, OC(R¹³)₂, C(R¹³)₂O, SC(R¹³)₂, C(R¹³)₂S, CO, C(R¹³)₂N(R¹³) and N(R¹³)C(R¹³)₂, wherein each R¹³, which may be the same or different, is hydrogen or (1-6C)alkyl;

Q¹ is aryl or heteroaryl,

and wherein Q¹ optionally bears one or more substituents, which may be the same or different, selected from halogeno, cyano, nitro, hydroxy, amino, carboxy, carbamoyl, sulfamoyl, formyl, mercapto, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (3-6C)alkenoyl, (3-6C)alkynoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkylsulfonylamino, N-(1-6C)alkyl-(1-6C)alkylsulfonylamino, and a group of the formula:
—X²—R⁸

wherein X² is a direct bond or is selected from O, CO and N(R⁹), wherein R⁹ is hydrogen or (1-6C)alkyl, and R⁸ is selected from halogeno-(1-6C)alkyl, hydroxy-(1-6C)alkyl, carboxy-(1-6C)alkyl, (1-6C)alkoxy-(1-6C)alkyl, cyano-(1-6C)alkyl, amino-(1-6C)alkyl, N-(1-6C)alkylamino-(1-6C)alkyl, N,N-di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, (1-6C)alkoxycarbonylamino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, (1-6C)alkylthio-(1-6C)alkyl, (1-6C)alkylsulfinyl-(1-6C)alkyl, (1-6C)alkylsulfonyl-(1-6C)alkyl, sulfamoyl-(1-6C)alkyl, N-(1-6C)alkylsulfamoyl-(1-6C)alkyl, N,N-di-(1-6C)alkylsulfamoyl-(1-6C)alkyl, (2-6C)alkanoyl-(1-6C)alkyl, (2-6C)alkanoyloxy-(1-6C)alkyl and (1-6C)alkoxycarbonyl-(1-6C)alkyl,

and wherein any CH₂ or CH₃ group within —X¹-Q¹ optionally bears on each said CH₂ or CH₃ group one or more substituents independently selected from halogeno, (1-6C)alkyl, hydroxy, cyano, amino, (1-4C)alkoxy, (1-4C)alkylamino and di-[(1-4C)alkylamino];

R⁴ and R⁵, which may be the same or different, are selected from hydrogen and (1-6C)alkyl, or

R⁴ and R⁵ together with the carbon atom to which they are attached form a (3-7C)cycloalkyl ring,

and wherein any CH₂ or CH₃ group within any of R⁴ and R⁵ optionally bears on each said CH₂ or CH₃ group one or more substituents independently selected from halogeno, hydroxy, cyano, (1-6C)alkoxy, amino, (2-6C)alkanoyl, (1-6C)alkylamino and di-[(1-6C)alkylamino];

R⁶ and R⁷, which may be the same or different, are selected from hydrogen, (1-6C)alkyl, (2-6C)alkenyl, (2-6C)alkynyl, (3-7C)cycloalkyl, (3-7C)cycloalkyl-(1-6C)alkyl, (3-7C)cycloalkenyl, (3-7C)cycloalkenyl-(1-6C)alkyl, heterocyclyl and heterocyclyl-(1-6C)alkyl, or

R⁶ and R⁷ together with the nitrogen atom to which they are attached form a saturated 4, 5, 6 or 7 membered heterocyclic ring which optionally contains one or more additional heteroatoms independently selected from oxygen, S, SO, SO₂ and NR¹⁰, wherein R¹⁰ is selected from hydrogen, (1-6C)alkyl, (2-6C)alkenyl, (2-6C)alkynyl, (1-6C)alkylsulfonyl, (1-6C)alkylcarbonyl and (1-6C)alkoxycarbonyl;

and wherein any heterocyclyl group within an R⁶ or an R⁷ substituent or any heterocyclic ring formed by R⁶, R⁷ and the nitrogen atom to which they are attached optionally bears one or more substituents, which may be the same or different, selected from halogeno, trifluoromethyl, cyano, nitro, hydroxy, amino, formyl, mercapto, (1-6C)alkyl, (2-6C)alkenyl, (2-6C)alkynyl, hydroxy-(1-6C)alkyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X³—R¹¹

wherein X³ is a direct bond or is selected from O, CO, SO₂ and N(R¹²), wherein R¹² is hydrogen or (1-4C)alkyl, and R¹¹ is selected from halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl,

and wherein any heterocyclyl group within an R⁶ or an R⁷ substituent or any heterocyclic ring formed by R⁶, R⁷ and the nitrogen atom to which they are attached optionally bears 1 or 2 oxo or thioxo substituents;

and wherein any CH₂ or CH₃ group within an R⁶ or an R⁷ substituent, other than a CH₂ group within a heterocyclyl group or heterocyclic ring, optionally bears on each said CH₂ or CH₃ group one or more substituents independently selected from halogeno, (1-6C)alkyl, hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkylsulfonylamino and N-(1-6C)alkyl-(1-6C)alkylsulfonylamino;

or a pharmaceutically acceptable salt thereof.

According to a fourth aspect of the invention there is provided a quinazoline derivative of the formula I wherein:

m is 0, 1 or 2;

each R¹, which may be the same or different, is selected from hydroxy, (1-6C)alkoxy, (3-7C)cycloalkyl-oxy and (3-7C)cycloalkyl-(1-6C)alkoxy,

and wherein any CH₂ or CH₃ group within an R¹ substituent optionally bears on each said CH₂ or CH₃ group one or more substituents independently selected from halogeno, (1-6C)alkyl, hydroxy and (1-6C)alkoxy;

R² is hydrogen or (1-4C)alkyl;

n is 0, 1, 2, 3 or 4;

each R³, which may be the same or different, is selected from halogeno, cyano, (1-4C)alkyl, trifluoromethyl, (1-4C)alkoxy, (2-4C)alkenyl and (2-4C)alkynyl;

X¹ is O;

Q¹ is aryl or heteroaryl,

and wherein Q¹ optionally bears one or more substituents, which may be the same or different, selected from halogeno, cyano, nitro, hydroxy, amino, carboxy, carbamoyl, sulfamoyl, formyl, mercapto, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (3-6C)alkenoyl, (3-6C)alkynoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkylsulfonylamino, N-(1-6C)alkyl-(1-6C)alkylsulfonylamino, and a group of the formula:
—X²—R⁸

wherein X² is a direct bond or is selected from O, CO and N(R⁹), wherein R⁹ is hydrogen or (1-6C)alkyl, and R⁸ is selected from halogeno-(1-6C)alkyl, hydroxy-(1-6C)alkyl, carboxy-(1-6C)alkyl, (1-6C)alkoxy-(1-6C)alkyl, cyano-(1-6C)alkyl, amino-(1-6C)alkyl, N-(1-6C)alkylamino-(1-6C)alkyl, N,N-di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, (1-6C)alkoxycarbonylamino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, (1-6C)alkylthio-(1-6C)alkyl, (1-6C)alkylsulfinyl-(1-6C)alkyl, (1-6C)alkylsulfonyl-(1-6C)alkyl, sulfamoyl-(1-6C)alkyl, N-(1-6C)alkylsulfamoyl-(1-6C)alkyl, N,N-di-(1-6C)alkylsulfamoyl-(1-6C)alkyl, (2-6C)alkanoyl-(1-6C)alkyl, (2-6C)alkanoyloxy-(1-6C)alkyl and (1-6C)alkoxycarbonyl-(1-6C)alkyl,

and wherein any CH₂ or CH₃ group within —X¹-Q¹ optionally bears on each said CH₂ or CH₃ group one or more substituents independently selected from halogeno, (1-6C)alkyl, hydroxy, cyano, amino, (1-4C)alkoxy, (1-4C)alkylamino and di-[(1-4C)alkylamino];

R⁴ and R⁵, which may be the same or different, are selected from hydrogen and (1-6C)alkyl, or

R⁴ and R⁵ together with the carbon atom to which they are attached form a (3-7C)cycloalkyl ring,

and wherein any CH₂ or CH₃ group within any of R⁴ and R⁵ optionally bears on each said CH₂ or CH₃ group one or more substituents independently selected from halogeno, hydroxy, cyano, (1-6C)alkoxy, amino, (2-6C)alkanoyl, (1-6C)alkylamino and di-[(1-6C)alkylamino];

R⁶ and R⁷, which may be the same or different, are selected from hydrogen, (1-6C)alkyl, (2-6C)alkenyl, (2-6C)alkynyl, (3-7C)cycloalkyl, (3-7C)cycloalkyl-(1-6C)alkyl, (3-7C)cycloalkenyl, (3-7C)cycloalkenyl-(1-6C)alkyl, heterocyclyl and heterocyclyl-(1-6C)alkyl, or

R⁶ and R⁷ together with the nitrogen atom to which they are attached form a saturated 4, 5, 6 or 7 membered heterocyclic ring which optionally contains one or more additional heteroatoms independently selected from oxygen, S, SO, SO₂ and NR¹⁰, wherein R¹⁰ is selected from hydrogen, (1-6C)alkyl, (2-6C)alkenyl, (2-6C)alkynyl, (1-6C)alkylsulfonyl, (1-6C)alkylcarbonyl and (1-6C)alkoxycarbonyl;

and wherein any heterocyclyl group within an R⁶ or an R⁷ substituent or any heterocyclic ring formed by R⁶, R⁷ and the nitrogen atom to which they are attached optionally bears one or more substituents, which may be the same or different, selected from halogeno, trifluoromethyl, cyano, nitro, hydroxy, amino, formyl, mercapto, (1-6C)alkyl, (2-6C)alkenyl, (2-6C)alkynyl, hydroxy-(1-6C)alkyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X³—R¹¹

wherein X³ is a direct bond or is selected from O, CO, SO₂ and N(R¹²), wherein R¹² is hydrogen or (1-4C)alkyl, and R¹¹ is selected from halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl,

and wherein any heterocyclyl group within an R⁶ or an R⁷ substituent or any heterocyclic ring formed by R⁶, R⁷ and the nitrogen atom to which they are attached optionally bears 1 or 2 oxo or thioxo substituents;

and wherein any CH₂ or CH₃ group within an R⁶ or an R⁷ substituent, other than a CH₂ group within a heterocyclyl group or heterocyclic ring, optionally bears on each said CH₂ or CH₃ group one or more substituents independently selected from halogeno, (1-6C)alkyl, hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkylsulfonylamino and N-(1-6C)alkyl-(1-6C)alkylsulfonylamino;

or a pharmaceutically acceptable salt thereof.

In this specification the generic term “alkyl” includes both straight-chain and branched-chain alkyl groups such as propyl, isopropyl and tert-butyl. However references to individual alkyl groups such as “propyl” are specific for the straight-chain version only and references to individual branched-chain alkyl groups such as “isopropyl” are specific for the branched-chain version only. An analogous convention applies to other generic terms, for example (1-6C)alkoxy includes methoxy, ethoxy and isopropoxy, (1-6C)alkylamino includes methylamino, ethylamino and isopropylamino and di-[(1-6C)alkyl]amino includes dimethylamino, diethylamino and N-isopropyl-N-methylamino.

It is to be understood that, insofar as certain of the quinazoline derivatives of the formula I defined above may exist in optically active or racemic forms by virtue of one or more asymmetric carbon atoms, the invention includes in its definition any such optically active or racemic form which possesses the above-mentioned activity. In particular, the quinazoline derivatives of the formula I have a chiral centre on the carbon atom to which the groups R⁴ and R⁵ are attached. The present invention encompasses all such stereoisomers having activity as herein defined, for example the (2R) and (2S) isomers (particularly the (2R) isomers). It is further to be understood that in the names of chiral compounds (R,S) denotes any scalemic or racemic mixture while (R) and (S) denote the enantiomers. In the absence of (R,S), (R) or (S) in the name it is to be understood that the name refers to any scalemic or racemic mixture, wherein a scalemic mixture contains R and S enantiomers in any relative proportions and a racemic mixture contains R and S enantiomers in the ratio 50:50. The synthesis of optically active forms may be carried out by standard techniques of organic chemistry well known in the art, for example by synthesis from optically active starting materials or by resolution of a racemic form. Similarly, the above-mentioned activity may be evaluated using the standard laboratory techniques referred to hereinafter.

Suitable values for the generic radicals referred to above include those set out below.

A suitable value for any one of the substituents herein (for example Q¹) when it is aryl is, for example, phenyl or naphthyl, preferably phenyl.

A suitable value for any one of the substituents herein (for example, R¹, R⁶, R⁷ or R⁴ and R⁵ together with the carbon atom to which they are attached) when it is (3-7C)cycloalkyl is, for example, cyclopropyl, cyclobutyl, cyclopentyl, cyclohexyl, cycloheptyl or bicyclo[2.2.1]heptyl. A suitable value for any one of the substituents herein, when it is (3-7C)cycloalkenyl is, for example, cyclobutenyl, cyclopentenyl, cyclohexenyl or cycloheptenyl.

A suitable value for any one of the substituents herein (for example Q¹) when it is heteroaryl is, for example, an aromatic 5 or 6 membered monocyclic ring or an aromatic 9 or 10 membered bicyclic ring with up to five ring heteroatoms selected from oxygen, nitrogen and sulfur, for example furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridinyl, pyridazinyl, pyrimidinyl, pyrazinyl, 1,3,5-triazinyl, 1,3-benzodioxolyl, benzofuranyl, indolyl, benzothienyl, benzoxazolyl, benzimidazolyl, benzothiazolyl, indazolyl, benzofurazanyl, quinolinyl, isoquinolinyl, quinazolinyl, quinoxalinyl, cinnolinyl or naphthyridinyl.

Particular heteroaryl groups include, for example pyridinyl, pyridazinyl, pyrimidinyl, pyrazinyl, thiazolyl, isothiazolyl, oxazolyl, imidazolyl, pyrazolyl and isoxazolyl. Further particular heteroaryl groups include, for example, pyridinyl, pyridazinyl, pyrimidinyl, pyrazinyl, thiazolyl and pyrazolyl.

A suitable value for any one of the substituents herein (for example R⁶, R⁷ or the heterocyclic ring formed by R⁶ and R⁷ together with the nitrogen atom to which they are attached) when it is a heterocyclyl group or a heterocyclylic ring is, for example, a non-aromatic saturated (i.e. ring systems with the maximum degree of saturation) or partially saturated (i.e. ring systems retaining some, but not the full, degree of unsaturation) 3, 4, 5, 6, 7, 8, 9 or 10 membered monocyclic or bicyclic ring with up to five heteroatoms selected from oxygen, nitrogen and sulfur, which, unless specified otherwise, may be carbon or nitrogen linked. Examples of such groups or rings include, for example, oxiranyl, oxetanyl, azetidinyl, dihydrofuranyl, tetrahydrofuranyl, 1,3-dioxolanyl, tetrahydropyranyl, 1,4-dioxanyl, oxepanyl, pyrrolinyl, pyrrolidinyl, morpholinyl, tetrahydro-1,4-thiazinyl, piperidinyl, homopiperidinyl, piperazinyl, homopiperazinyl, dihydropyridinyl, tetrahydropyridinyl, dihydropyrimidinyl, tetrahydropyrimidinyl, tetrahydrothienyl, tetrahydrothiopyranyl, decahydroisoquinolinyl or decahydroquinolinyl, particularly azetidinyl, tetrahydrofuranyl, tetrahydropyranyl, pyrrolidinyl, morpholinyl, 1,4-oxazepanyl, tetrahydro-1,4-thiazinyl, piperidinyl or piperazinyl, more particularly azetidin-1-yl, tetrahydrofuran-3-yl, tetrahydropyran-4-yl, tetrahydrothiopyran-4-yl, pyrrolidin-1-yl, pyrrolidin-2-yl, pyrrolidin-3-yl, morpholin-4-yl, morpholin-2-yl, piperidin-1-yl, piperidin-4-yl, piperidin-3-yl, piperidin-2-yl or piperazin-1-yl. A nitrogen or sulfur atom within a heterocyclyl group may be oxidized to give the corresponding N or S oxide. A suitable value for such a group which bears 1 or 2 oxo or thioxo substituents is, for example, 1,1-dioxotetrahydro-1,4-thiazinyl, 1-oxotetrahydro-1,4-thiazinyl, 1,1-dioxotetrahydrothienyl, 1-oxotetrahydrothienyl, 1,1-dioxotetrahydrothiopyranyl, 1-oxotetrahydrothiopyranyl, 2-oxopyrrolidinyl, 2-thioxopyrrolidinyl, 2-oxoimidazolidinyl, 2-thioxoimidazolidinyl, 2-oxopiperidinyl, 3-oxopiperazinyl, 2,5-dioxopyrrolidinyl, 2,5-dioxoimidazolidinyl or 2,6-dioxopiperidinyl.

Particular examples of heterocyclyl substituent groups include, for example, non-aromatic saturated or partially saturated 3, 4, 5, 6 or 7 membered monocyclic heterocyclyl rings with 1 ring nitrogen or sulfur heteroatom and optionally 1 or 2 additional heteroatoms selected from nitrogen, oxygen and sulfur. Examples of such groups include azetidinyl, oxazepanyl, pyrrolinyl, pyrrolidinyl, morpholinyl, tetrahydro-1,4-thiazinyl, piperidinyl, homopiperidinyl, piperazinyl, homopiperazinyl, dihydropyridinyl, tetrahydropyridinyl, dihydropyrimidinyl, tetrahydropyrimidinyl, tetrahydrothienyl, tetrahydrothiopyranyl or thiomorpholinyl.

Other particular examples of heterocyclyl substituent groups include, for example a 4, 5, 6 or 7 membered monocyclic saturated or partially saturated heterocyclyl ring containing 1 or 2 heteroatoms selected from oxygen, nitrogen and sulfur such as oxetanyl, azetidinyl, dihydrofuranyl, tetrahydrofuranyl, 1,3-dioxolanyl, tetrahydropyranyl, 1,4-dioxanyl, oxepanyl, pyrrolinyl, pyrrolidinyl, morpholinyl, tetrahydro-1,4-thiazinyl, 1,1-dioxotetrahydro-1,4-thiazinyl, piperidinyl, homopiperidinyl, piperazinyl, homopiperazinyl, dihydropyridinyl, tetrahydropyridinyl, dihydropyrimidinyl, tetrahydropyrimidinyl, tetrahydrothienyl or tetrahydrothiopyranyl.

Further particular examples of heterocyclyl substituent groups include, for example 4, 5, 6 or 7 membered saturated or partially saturated monocyclic heterocyclyl rings containing 1 nitrogen atom and optionally 1 additional heteroatom selected from nitrogen and oxygen such as azetidinyl, piperazinyl, pyrrolidinyl, piperidinyl or morpholinyl, particularly azetidin-1-yl, pyrrolidin-1-yl, pyrrolidin-2-yl, piperazin-1-yl, piperidin-4-yl, piperidin-1-yl or morpholin-4-yl.

Other examples of heterocyclyl substituent groups include, for example, non-aromatic saturated or partially saturated 4, 5, 6 or 7 membered monocyclic heterocyclyl rings containing 1 or 2 oxygen atoms such as tetrahydrofuranyl, 1,3-dioxolanyl or tetrahydropyranyl.

A suitable value for a substituent herein when it is heterocyclyl-(1-6C)alkyl is, for example, heterocyclylmethyl, 2-heterocyclylethyl or 3-heterocyclylpropyl. The invention comprises corresponding suitable values for other substituents when, for example, rather than a heterocyclyl-(1-6C)alkyl group, an (3-7C)cycloalkyl-(1-6C)alkyl or (3-7C)cycloalkenyl-(1-6C)alkyl is present.

Suitable values for any of the substituents herein, for example the ‘R’ groups (R¹ to R¹³) or for various groups within a Q¹ or X¹ group include:—

| | |
|---|---|
| for halogeno: | fluoro, chloro, bromo and iodo; |
| for (1-6C)alkyl: | methyl, ethyl, propyl, isopropyl and tert-butyl; |
| for (2-8C)alkenyl: | vinyl, isopropenyl, allyl and but-2-enyl; |
| for (2-8C)alkynyl: | ethynyl, 2-propynyl and but-2-ynyl; |
| for (1-6C)alkoxy: | methoxy, ethoxy, propoxy, isopropoxy and butoxy; |
| for (2-6C)alkenyloxy: | vinyloxy and allyloxy; |
| for (2-6C)alkynyloxy: | ethynyloxy and 2-propynyloxy; |
| for (1-6C)alkylthio: | methylthio, ethylthio and propylthio; |
| for (1-6C)alkylsulfinyl: | methylsulfinyl and ethylsulfinyl; |
| for (1-6C)alkylsulfonyl: | methylsulfonyl and ethylsulfonyl; |
| for (1-6C)alkylamino: | methylamino, ethylamino, propylamino, |
| | isopropylamino and butylamino; |
| for di-[(1-6C)alkyl] amino: | dimethylamino, diethylamino N-ethyl- |
| | N-methylamino and diisopropylamino; |
| for (1-6C)alkylcarbonyl: | methylcarbonyl, ethylcarbonyl, propylcarbonyl and |
| | tert-butylcarbonyl; |
| for (1-6C)alkoxycarbonyl: | methoxycarbonyl, ethoxycarbonyl, propoxycarbonyl |
| | and tert-butoxycarbonyl; |
| for (1-6C)alkoxycarbony-(1-6C)alkyl: | methoxycarbonylmethyl, methoxycarbonylethyl, |
| | methoxycarbonylpropyl, ethoxycarbonylmethyl, |
| | ethoxycarbonylethyl, ethoxycarbonylpropyl, |
| | propoxycarbonylmethyl, propoxycarbonylethyl, |
| | propoxycarbonylpropyl, tert-butylcarbonylmethyl, |
| | tert-butylcarbonylethyl and |
| | tert-butoxycarbonylpropyl; |
| for N-(1-6C)alkylcarbamoyl: | N-methylcarbamoyl, N-ethylcarbamoyl and |
| | N-propylcarbamoyl; |
| for N,N-di-[(1-6C)alkyl]carbamoyl: | N,N-dimethylcarbamoyl, N-ethyl- |
| | N-methylcarbamoyl and N,N-diethylcarbamoyl; |
| for (2-6C)alkanoyl: | acetyl, propionyl, butyryl and isobutyryl; |
| for (3-6C)alkenoyl | acryloyl and but-2-enoly; |
| for (3-6C)alkynoyl: | prop-2-ynoyl; |
| for (2-6C)alkanoyloxy: | acetoxy and propionyloxy; |
| for (2-6C)alkanoylamino: | acetamido and propionamido; |
| for N-(1-6C)alkyl-(2-6C)alkanoylamino: | N-methylacetamido and N-methylpropionamido; |
| for N-(1-6C)alkylsulfamoyl: | N-methylsulfamoyl and N-ethylsulfamoyl; |
| for N,N-di-[(1-6C)alkyl]sulfamoyl: | N,N-dimethylsulfamoyl; |
| for (1-6C)alkylsulfonylamino: | methanesulfonylamino and ethanesulfonylamino; |
| for N-(1-6C)alkyl-(1-6C)alkylsulfonylamino: | N-methylmethanesulfonylamino and |
| | N-methylethanesulfonylamino; |
| for (3-6C)alkenoylamino: | acrylamido, methacrylamido and crotonamido; |
| for N-(1-6C)alkyl-(3-6C)alkenoylamino: | N-methylacrylamido and N-methylcrotonamido; |
| for (3-6C)alkynoylamino: | propiolamido; |
| for N-(1-6C)alkyl-(3-6C)alkynoylamino: | N-methylpropiolamido; |
| for amino-(1-6C)alkyl: | aminomethyl, 2-aminoethyl, 1-aminoethyl and |
| | 3-aminopropyl; |
| for N-(1-6C)alkylamino-(1-6C)alkyl: | methylaminomethyl, ethylaminomethyl, |
| | 1-methylaminoethyl, 2-methylaminoethyl, |
| | 2-ethylaminoethyl and 3-methylaminopropyl; |
| for N,N-di-[(1-6C)alkyl]amino-(1-6C)alkyl: | dimethylaminomethyl, diethylaminomethyl, |
| | 1-dimethylaminoethyl, 2-dimethylaminoethyl and |
| | 3-dimethylaminopropyl; |
| for halogeno-(1-6C)alkyl: | chloromethyl, 2-chloroethyl, 1-chloroethyl and |
| | 3-chloropropyl; |
| for hydroxy-(1-6C)alkyl: | hydroxymethyl, 2-hydroxyethyl, 1-hydroxyethyl and |
| | 3-hydroxypropyl; |
| for (1-6C)alkoxy-(1-6C)alkyl: | methoxymethyl, ethoxymethyl, 1-methoxyethyl, |
| | 2-methoxyethyl, 2-ethoxyethyl and |
| | 3-methoxypropyl; |
| for carboxy-(1-6C)alkyl: | carboxymethyl and 2-carboxyethyl; |
| for cyano-(1-6C)alkyl: | cyanomethyl, 2-cyanoethyl, 1-cyanoethyl and |
| | 3-cyanopropyl; |
| for (1-6C)alkylthio-(1-6C)alkyl: | methylthiomethyl, ethylthiomethyl, |
| | 2-methylthioethyl, 1-methylthioethyl and |
| | 3-methylthiopropyl; |
| for (1-6C)alkylsulfinyl-(1-6C)alkyl: | methylsulfinylmethyl, ethylsulfinylmethyl, |
| | 2-methylsulfinylethyl, 1-methylsulfinylethyl and |
| | 3-methylsulfinylpropyl; |
| for (1-6C)alkylsulfonyl-(1-6C)alkyl: | methylsulfonylmethyl, ethylsulfonylmethyl, |
| | 2-methylsulfonylethyl, 1-methylsulfonylethyl and |
| | 3-methylsulfonylpropyl; |
| for (2-6C)alkanoylamino-(1-6C)alkyl: | acetamidomethyl, propionamidomethyl and |
| | 2-acetamidoethyl; |
| for N-(1-6C)alkyl-(2-6C)alkanoylamino-(1-6C)alkyl: | N-methylacetamidomethyl, 2- |
| | (N-methylacetamido)ethyl and 2- |
| | (N-methylpropionamido)ethyl; |
| for (1-6C)alkoxycarbonylamino-(1-6C)alkyl: | methoxycarbonylaminomethyl, |
| | ethoxycarbonylaminomethyl, |
| | tert-butoxycarbonylaminomethyl and |
| | 2-methoxycarbonylaminoethyl; |
| for (2-6C)alkanoyl-(1-6C)alkyl: | acetylmethyl and 2-acetylethyl; |
| for (2-6C)alkanoyloxy-(1-6C)alkyl: | acetoxymethyl, 2-acetoxyethyl and 2- |
| | propionyloxyethyl; |
| for carbamoyl-(1-6C)alkyl: | carbamoylmethyl, 1-carbamoylethyl, |
| | 2-carbamoylethyl and 3-carbamoylpropyl; |
| for N-(1-6C)alkylcarbamoyl-(1-6C)alkyl: | N-methylcarbamoylmethyl, |
| | N-ethylcarbamoylmethyl, |
| | N-propylcarbamoylmethyl, |
| | 1-(N-methylcarbamoyl)ethyl, |
| | 1-(N-ethylcarbamoyl)ethyl, |
| | 2-(N-methylcarbamoyl)ethyl, |
| | 2-(N-ethylcarbamoyl)ethyl and |
| | 3-(N-methylcarbamoyl)propyl; |
| for N,N-di[(1-6C)alkyl]carbamoyl-(1-6C)alkyl: | N,N-dimethylcarbamoylmethyl, |
| | N,N-diethylcarbamoylmethyl, |
| | 2-(N,N-dimethylcarbamoyl)ethyl, and |
| | 3-(N,N-dimethylcarbamoyl)propyl; |
| for sulfamoyl(1-6C) alkyl: | sulfamoylmethyl, 1-sulfamoylethyl, |
| | 2-sulfamoylethyl and 3-sulfamoylpropyl; |
| for N-(1-6C)alkylsulfamoyl(1-6C)alkyl: | N-methylsulfamoylmethyl, |
| | N-ethylsulfamoylmethyl, N-propylsulfamoylmethyl, |
| | 1-(N-methylsulfamoyl)ethyl, |
| | 2-(N-methylsulfamoyl)ethyl and |
| | 3-(N-methylsulfamoyl)propyl; and |
| for N,N di-(1-6C)alkylsulfamoyl(1-6C)alkyl: | N,N-dimethylsulfamoylmethyl, |
| | N,N-diethylsulfamoylmethyl, N |
| | methyl,N-ethylsulfamoylmethyl, 1-( |
| | N,N-dimethylsulfamoyl)ethyl, |
| | 1-(N,N-diethylsulfamoyl)ethyl, |
| | 2-(N,N-dimethylsulfamoyl)ethyl, |
| | 2-(N,N-diethylsulfamoyl)ethyl and |
| | 3-(N,N-dimethylsulfamoyl)propyl. |

When in this specification reference is made to a (1-4C)alkyl group it is to be understood that such groups refer to alkyl groups containing up to 4 carbon atoms. A skilled person will realise that representative examples of such groups are those listed above under (1-6C)alkyl that contain up to 4 carbon atoms, such as methyl, ethyl, propyl, isopropyl, butyl and tert-butyl. Similarly, reference to a (1-3C)alkyl group refers to alkyl groups containing up to 3 carbon atoms such as methyl, ethyl, propyl and isopropyl. A similar convention is adopted for the other groups listed above such as (1-4C)alkoxy, (2-4C)alkenyl, (2-4C)alkynyl and (2-4C)alkanoyl.

When, as defined hereinbefore, in the group of the formula —X¹-Q¹, X¹ is, for example, a OC(R¹³)₂ linking group, it is the oxygen atom, not the carbon atom, of the OC(R¹³)₂ linking group which is attached to the phenyl ring in the formula I and the carbon atom is attached to the Q¹ group. Similarly when X¹ is a N(R¹³)C(R¹³)₂ linking group, the nitrogen atom of the N(R¹³)C(R¹³)₂ group is attached to the phenyl ring in the formula I and the carbon atom is attached to the Q¹ group. A similar convention is applied to other linking groups used herein.

When reference is made herein to a CH₂ or CH₃ group optionally bearing on each said CH₂ or CH₃ group one or more substituents as defined herein, there are suitably 1 or 2 such substituents present on each said CH₂ group and there are suitably 1, 2 or 3 such substituents present on each said CH₃ group.

Where reference is made herein to any CH₂ or CH₃ group optionally bearing on each said CH₂ or CH₃ group a substituent as defined herein, suitable substituents so formed include, for example, hydroxy-substituted (1-6C)alkyl groups (such as 2-hydroxyethyl and 2-hydroxy-1,1-dimethylethyl), (1-6C)alkylsulfonyl-substituted (1-6C)alkyl groups (such as 2-(methylsulfonyl)ethyl), (1-6C)alkoxy-substituted (1-6C)alkyl groups (such as 2-(methoxy)ethyl) and di-[(1-6C)alkyl]amino-substituted (1-6C)alkyl groups (such as 2-(dimethylamino)ethyl).

Where reference is made herein to, for example, R⁴ and R⁵ together with the carbon atom to which they are attached forming a (3-7C)cycloalkyl ring herein, the ring so formed is a (3-7C)cycloalkylidene group, for example a cyclopropylidene group of the formula: 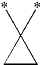
wherein * represent the bonds from the cyclopropylidene group.

Where reference is made herein to R⁶ and R⁷ together with the nitrogen atom to which they are attached forming a saturated 4, 5, 6 or 7 membered heterocyclic ring which optionally contains one or more additional heteroatoms independently selected from oxygen, S, SO, SO₂ or N(R¹⁰) (wherein R¹⁰ is as hereinbefore defined), the ring so formed suitably contains one or two additional heteroatoms and, more suitably contains one additional heteroatom, representative examples of which are listed above. For example, the ring so formed may be selected from azetidin-1-yl, pyrrolidin-1-yl, pyrazolidin-1-yl, piperidin-1-yl, morpholin-4-yl and piperazin-1-yl (particularly azetidin-1-yl, pyrrolidin-1-yl, piperidin-1-yl, morpholin-4-yl and piperazin-1-yl). Any of the heterocyclic rings formed by R⁶ and R⁷ together with the nitrogen atom to which they are attached optionally bears one or more substituents, which may be the same or different, as defined herein and/or optionally bears 1 or 2 oxo or thioxo substituents.

It is to be understood that the quinazoline group in formula I is unsubstituted at the 2-position on the quinazoline ring.

It is to be understood that certain quinazoline derivatives of the formula I may exist in solvated as well as unsolvated forms such as, for example, hydrated forms. It is to be understood that the invention encompasses all such solvated forms which exhibit an inhibitory effect on an erbB receptor tyrosine kinase, such as anti-proliferative activity.

It is also to be understood that certain quinazoline derivatives of the formula I may exhibit polymorphism, and that the invention encompasses all such forms which exhibit an inhibitory effect on an erbB receptor tyrosine kinase, such as anti-proliferative activity.

It is also to be understood that the invention relates to all tautomeric forms of the quinazoline derivatives of the formula I which exhibit an inhibitory effect on an erbB receptor tyrosine kinase, such as anti-proliferative activity.

A suitable pharmaceutically acceptable salt of a quinazoline derivative of the formula I is, for example, an acid-addition salt of a quinazoline derivative of the formula I, for example an acid-addition salt with an inorganic or organic acid. Suitable inorganic acids include, for example, hydrochloric, hydrobromic or sulfuric acid. Suitable organic acids include, for example, trifluoroacetic, citric, fumaric or maleic acid. Another suitable pharmaceutically acceptable salt of a quinazoline derivative of the formula I is for example, a salt of a quinazoline derivative of the formula I which is sufficiently acidic, for example an alkali or alkaline earth metal salt such as a calcium or magnesium salt, or an ammonium salt, or a salt with an organic base such as methylamine, dimethylamine, trimethylamine, piperidine, morpholine or tris-(2-hydroxyethyl)amine.

Particular novel quinazoline derivatives of the invention include, for example, quinazoline derivatives of the formula I, or pharmaceutically acceptable salts thereof, wherein, unless otherwise stated, each of R¹, R², R³, R⁴, R⁵, R⁶, R⁷, Q¹, X¹, m and n has any of the meanings defined hereinbefore or in paragraphs (a) to (eeeeee) hereinafter:—

(a) m is 0 or 1 and R¹, when present, is located at the 7-position on the quinazoline ring in the formula I;

(b) R¹ is selected from hydroxy, (1-6C)alkoxy, hydroxy-(1-6C)alkoxy, (1-6C)alkoxy-(1-6C)alkoxy, (3-7C)cycloalkyl-oxy and (3-7C)cycloalkyl-(1-6C)alkoxy,

and wherein any CH₂ or CH₃ group within an R¹ substituent optionally bears on each said CH₂ or CH₃ group one or more substituents independently selected from fluoro and chloro;

(c) m is 0 or 1 and R¹, when present, is located at the 7-position on the quinazoline ring and is selected from (1-6C)alkoxy, cyclopropyl-(1-4C)alkoxy, cyclobutyl-(1-4C)alkoxy, cyclopentyl-(1-4C)alkoxy and cyclohexyl-(1-6C)alkoxy,

and wherein any CH₂ or CH₃ group within an R¹ substituent optionally bears on each said CH₂ or CH₃ group one or more substituents independently selected from fluoro, chloro, hydroxy, methoxy and ethoxy;

(d) m is 1 and R¹ is located at the 7-position on the quinazoline ring and is (1-4C)alkoxy (for example methoxy or ethoxy),

and wherein any CH₂ or CH₃ group within an R¹ substituent optionally bears on each said CH₂ or CH₃ group one or more substituents independently selected from fluoro, chloro, hydroxy, methoxy and ethoxy;

(e) m is 1 and R¹ is located at the 7-position on the quinazoline ring and is selected from methoxy, ethoxy, propyloxy, isopropyloxy, cyclopropylmethoxy, 2-hydroxyethoxy, 2-fluoroethoxy, 2-methoxyethoxy, 2-ethoxyethoxy, trifluoromethoxy, 2,2-difluoroethoxy and 2,2,2-trifluoroethoxy;

(f) m is 1 and R¹ is located at the 7-position on the quinazoline ring and is methoxy;

(g) m is 0;

(h) R² is hydrogen or methyl;

(i) R² is hydrogen;

(j) n is 0, 1 or 2 (particularly 0 or 1, more particularly 1);

(k) n is 1 or 2 (particularly n is 1);

(l) n is 0, 1 or 2 (particularly 0 or 1) and, when present, at least one R³ is in a meta-position (3-position) relative to the nitrogen of the anilino group in the formula I;

(m) n is 0, 1 or 2 (particularly 0 or 1) and, when present, at least one R³ is in a meta-position (3-position) relative to the nitrogen of the anilino group in the formula I, and R³ is selected from halogeno, cyano, (1-4C)alkyl, (1-4C)alkoxy and (2-4C)alkynyl (particularly halogeno, cyano, (1-4C)alkyl and (1-4C)alkoxy, more particularly halogeno, (1-4C)alkyl and (1-4C)alkoxy);

(n) n is 0, 1 or 2 (particularly 0 or 1) and, when present, at least one R³ is in a meta-position (3-position) relative to the nitrogen of the anilino group in the formula I, and R³ is selected from halogeno, (1-4C)alkyl, (1-4C)alkoxy and (2-4C)alkynyl (particularly halogeno, (1-4C)alkyl and (1-4C)alkoxy);

(o) n is 0, 1 or 2 (particularly 0 or 1) and, when present, at least one R³ is in a meta-position (3-position) relative to the nitrogen of the anilino group in the formula I, and R³ is selected from halogeno (for example fluoro or chloro) and (1-4C)alkyl (for example methyl);

(p) n is 0 or 1 and, when present, R³ is in a meta-position (3-position) relative to the nitrogen of the anilino group in the formula I, and R³ is selected from halogeno (for example fluoro or chloro) and (1-4C)alkyl (for example methyl);

(q) n is 0 or 1 and, when present, R³ is in a meta-position (3-position) relative to the nitrogen of the anilino group in the formula I, and R³ is selected from fluoro, chloro, methyl, methoxy and cyano (particularly fluoro, chloro, methyl and methoxy);

(r) n is 0 or 1 and, when present, R³ is in a meta-position (3-position) relative to the nitrogen of the anilino group in the formula I, and R³ is selected from fluoro, chloro, methyl, methoxy and ethynyl;

(s) n is 0 or 1 and, when present, R³ is in a meta-position (3-position) relative to the nitrogen of the anilino group in the formula I, and R³ is selected from chloro and methyl;

(t) n is 1, R³ is chloro and R³ is in a meta-position (3-position) relative to the nitrogen of the anilino group in the formula I;

(u) n is 1, R³ is methyl and R³ is in a meta-position (3-position) relative to the nitrogen of the anilino group in the formula I;

(v) X¹ is selected from O, S, OC(R¹³)₂, SC(R¹³)₂, SO, SO₂, N(R¹³), CO and N(R¹³)C(R¹³)₂ wherein each R¹³, which may be the same or different, is hydrogen or (1-6C)alkyl;

(w) X¹ is selected from O, S and OC(R¹³)₂ wherein each R¹³, which may be the same or different, is hydrogen or (1-4C)alkyl;

(x) X¹ is selected from S and OC(R¹³)₂ wherein each R¹³, which may be the same or different, is hydrogen or (1-4C)alkyl;

(y) X¹ is selected from O and OC(R¹³)₂ wherein each R¹³, which may be the same or different, is hydrogen or (1-4C)alkyl;

(z) X¹ is selected from O, S and OCH₂;

(aa) X¹ is selected from O and OCH₂;

(bb) X¹ is O;

(cc) X¹ is S;

(dd) X¹ is OCH₂;

(ee) X¹ is OCH₂, n is 0 or 1 and, when present, R³ is selected from halogeno (for example chloro or fluoro), cyano, (1-4C)alkyl (for example methyl) and (1-4C)alkoxy (for example methoxy);

(ff) X¹ is OCH₂, n is 0 or 1 and, when present, R³ is selected from halogeno (for example chloro) and (1-4C)alkyl (for example methyl);

(gg) X¹ is OCH₂, n is 0 or 1 and, when present, R³ is halogeno (for example chloro);

(hh) X¹ is OCH₂, n is 0 or 1 and, when present, R³ is (1-4C)alkyl (for example methyl);

(ii) X¹ is OCH₂, n is 1, R³ is selected from fluoro, chloro, cyano, methyl and methoxy, and R³ is in a meta-position (3-position) relative to the nitrogen of the anilino group in the formula I;

(jj) X¹ is OCH₂, n is 1, R³ is selected from fluoro, chloro and methyl (particularly chloro and methyl), and R³ is in a meta-position (3-position) relative to the nitrogen of the anilino group in the formula I;

(kk) X¹ is O, n is 0 or 1 and, when present, R³ is selected from halogeno (for example chloro or fluoro), cyano, (1-4C)alkyl (for example methyl) and (1-4C)alkoxy (for example methoxy);

(ll) X¹ is O, n is 0 or 1 and, when present, R³ is selected from halogeno (for example chloro) and (1-4C)alkyl (for example methyl);

(mm) X¹ is O, n is 0 or 1 and, when present, R³ is halogeno (for example fluoro or chloro, particularly chloro);

(nn) X¹ is O, n is 0 or 1 and, when present, R³ is (1-4C)alkyl (for example methyl);

(oo) X¹ is O, n is 1, R³ is selected from fluoro, chloro, cyano, methyl and methoxy, and R³ is in a meta-position (3-position) relative to the nitrogen of the anilino group in the formula I;

(pp) X¹ is O, n is 1, R³ is selected from fluoro, chloro and methyl (particularly chloro and methyl), and R³ is in a meta-position (3-position) relative to the nitrogen of the anilino group in the formula I;

(qq) Q¹ is heteroaryl,

and wherein Q¹ optionally bears one or more substituents (for example 1, 2 or 3), which may be the same or different, selected from halogeno, cyano, nitro, hydroxy, amino, carboxy, carbamoyl, sulfamoyl, formyl, mercapto, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (3-6C)alkenoyl, (3-6C)alkynoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkylsulfonylamino, N-(1-6C)alkyl-(1-6C)alkylsulfonylamino, and a group of the formula:
—X²—R⁸

wherein X² is a direct bond or is selected from O, CO and N(R⁹), wherein R⁹ is hydrogen or (1-6C)alkyl, and R⁸ is selected from halogeno-(1-6C)alkyl, hydroxy-(1-6C)alkyl, carboxy-(1-6C)alkyl, (1-6C)alkoxy-(1-6C)alkyl, cyano-(1-6C)alkyl, amino-(1-6C)alkyl, N-(1-6C)alkylamino-(1-6C)alkyl, N,N-di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, (1-6C)alkoxycarbonylamino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, (1-6C)alkylthio-(1-6C)alkyl, (1-6C)alkylsulfinyl-(1-6C)alkyl, (1-6C)alkylsulfonyl-(1-6C)alkyl, sulfamoyl-(1-6C)alkyl, N-(1-6C)alkylsulfamoyl-(1-6C)alkyl, N,N-di-(1-6C)alkylsulfamoyl-(1-6C)alkyl, (2-6C)alkanoyl-(1-6C)alkyl, (2-6C)alkanoyloxy-(1-6C)alkyl and (1-6C)alkoxycarbonyl-(1-6C)alkyl,

and wherein any CH₂ or CH₃ group within —X¹-Q¹ optionally bears on each said CH₂ or CH₃ group one or more (for example 1, 2, or 3) substituents independently selected from halogeno, (1-6C)alkyl, hydroxy, cyano, amino, (1-4C)alkoxy, (1-4C)alkylamino and di-[(1-4C)alkylamino];

(rr) Q¹ is selected from phenyl and a 5 or 6 membered monocyclic heteroaryl ring, which ring contains 1, 2 or 3 heteroatoms independently selected from oxygen, nitrogen and sulfur,

and wherein Q¹ optionally bears one or more substituents (for example 1, 2 or 3), which may be the same or different, as hereinbefore defined in (qq);

(ss) Q¹ is phenyl,

and wherein Q¹ optionally bears one or more substituents (for example 1, 2 or 3), which may be the same or different, as hereinbefore defined in (qq);

(tt) Q¹ is a 5 or 6 membered monocyclic heteroaryl ring, which ring contains 1, 2 or 3 heteroatoms independently selected from oxygen, nitrogen and sulfur,

and wherein Q¹ optionally bears one or more substituents (for example 1, 2 or 3), which may be the same or different, as hereinbefore defined in (qq);

(uu) Q¹ is a 5 or 6 membered monocyclic heteroaryl ring, which ring contains 1 nitrogen heteroatom and optionally 1 additional heteroatom selected from oxygen, nitrogen and sulfur,

and wherein Q¹ optionally bears one or more substituents (for example 1, 2 or 3), which may be the same or different, as hereinbefore defined in (qq);

(vv) Q¹ is selected from phenyl, pyridinyl, pyrimidinyl, pyrazinyl, 1,3-thiazolyl, 1H-imidazolyl, 1H-pyrazolyl, 1,3-oxazolyl and isoxazolyl,

and wherein Q¹ optionally bears one or more substituents (for example 1, 2 or 3), which may be the same or different, as hereinbefore defined in (qq);

(ww) Q¹ is selected from pyridinyl, pyrimidinyl, pyrazinyl, 1,3-thiazolyl, 1H-pyrazolyl and pyridazinyl,

and wherein Q¹ optionally bears one or more substituents (for example 1, 2 or 3), which may be the same or different, as hereinbefore defined in (qq);

(xx) Q¹ is pyridinyl (for example 2-pyridinyl or 3-pyridinyl),

and wherein Q¹ optionally bears one or more substituents (for example 1, 2 or 3), which may be the same or different, as hereinbefore defined in (qq);

(yy) Q¹ is selected from phenyl and a 5 or 6 membered monocyclic heteroaryl ring, which ring contains 1, 2 or 3 heteroatoms independently selected from oxygen, nitrogen and sulfur,

and wherein Q¹ optionally bears one or more substituents (for example 1, 2 or 3), which may be the same or different, selected from halogeno, hydroxy, cyano, carboxy, nitro, amino, (1-4C)alkyl, (1-4C)alkoxy, (2-4C)alkenyl, (2-4C)alkynyl, (1-4C)alkylthio, (1-4C)alkylsulfinyl, (1-4C)alkylsulfonyl, (2-4C)alkanoyl, N-(1-4C)alkylamino, N,N-di-[(1-4C)alkyl]amino, (1-4C)alkoxycarbonyl, carbamoyl, N-(1-4C)alkylcarbamoyl, N,N-di-[(1-4C)alkyl]carbamoyl, (2-4C)alkanoyloxy, (2-4C)alkanoylamino, N-(1-4C)alkyl-(2-4C)alkanoylamino, halogeno-(1-4C)alkyl, hydroxy-(1-4C)alkyl, (1-4C)alkoxy-(1-4C)alkyl, cyano-(1-4C)alkyl, carboxy-(1-4C)alkyl, amino-(1-4C)alkyl, N-(1-4C)alkylamino-(1-4C)alkyl and N,N-di-[(1-4C)alkyl]amino-(1-4C)alkyl;

(zz) Q¹ is a 5 or 6 membered monocyclic heteroaryl ring, which ring contains 1, 2 or 3 heteroatoms independently selected from oxygen, nitrogen and sulfur,

and wherein Q¹ optionally bears one or more substituents (for example 1, 2 or 3), which may be the same or different, as hereinbefore defined in (yy);

(aaa) Q¹ is a 5 or 6 membered monocyclic heteroaryl ring, which ring contains 1 nitrogen heteroatom and optionally 1 additional heteroatom selected from oxygen, nitrogen and sulfur,

and wherein Q¹ optionally bears one or more substituents (for example 1, 2 or 3), which may be the same or different, as hereinbefore defined in (yy);

(bbb) Q¹ is selected from phenyl, pyridinyl, pyrimidinyl, pyrazinyl, 1,3-thiazolyl, 1H-imidazolyl, 1H-pyrazolyl, 1,3-oxazolyl and isoxazolyl,

and wherein Q¹ optionally bears one or more substituents (for example 1, 2 or 3), which may be the same or different, as hereinbefore defined in (yy);

(ccc) Q¹ is selected from pyridinyl, pyrimidinyl, pyrazinyl, 1,3-thiazolyl, 1H-pyrazolyl and pyridazinyl,

and wherein Q¹ optionally bears one or more substituents (for example 1, 2 or 3), which may be the same or different, as hereinbefore defined in (yy);

(ddd) Q¹ is pyridinyl (for example 2-pyridinyl or 3-pyridinyl),

and wherein Q¹ optionally bears one or more substituents (for example 1, 2 or 3), which may be the same or different, as hereinbefore defined in (yy);

(eee) Q¹ is selected from phenyl and a 5 or 6 membered monocyclic heteroaryl ring, which ring contains 1, 2 or 3 heteroatoms independently selected from oxygen, nitrogen and sulfur,

and wherein Q¹ optionally bears one or more substituents (for example 1, 2 or 3), which may be the same or different, selected from fluoro, chloro, bromo, hydroxy, carboxy, cyano, nitro, amino, methyl, ethyl, isopropyl, methoxy, ethoxy, vinyl, allyl, ethynyl, 2-propynyl, methylthio, methylsulfinyl, methylsulfonyl, acetyl, propionyl methylamino, ethylamino, N,N-dimethylamino, N,N-diethylamino, N-methyl-N-ethylamino methoxycarbonyl, ethoxycarbonyl, carbamoyl, N-methylcarbamoyl, N,N-dimethylcarbamoyl, acetoxy, acetamido, fluoromethyl, 2-fluoroethyl, chloromethyl, 2-chloroethyl, hydroxymethyl, 2-hydroxyethyl, methoxymethyl, 2-methoxyethyl, cyanomethyl, 2-cyanoethyl, carboxymethyl, 2-carboxymethyl, aminomethyl, methylaminomethyl, ethylaminomethyl, N,N-dimethylaminomethyl, N,N-diethylaminomethyl, N-methyl-N-ethylaminomethyl, 2-aminoethyl, 2-(methylamino)ethyl, 2-(ethylamino)ethyl, 2-(N,N-dimethylamino)ethyl, 2-(N,N-diethylamino)ethyl, 2-(N-methyl-N-ethylamino)ethyl, carbamoylmethyl, N-methylcarbamoylmethyl and N,N-dimethylcarbamoylmethyl;

(fff) Q¹ is a 5 or 6 membered monocyclic heteroaryl ring, which ring contains 1, 2 or 3 heteroatoms independently selected from oxygen, nitrogen and sulfur,

and wherein Q¹ optionally bears one or more substituents (for example 1, 2 or 3), which may be the same or different, as hereinbefore defined in (eee);

(ggg) Q¹ is a 5 or 6 membered monocyclic heteroaryl ring, which ring contains 1 nitrogen heteroatom and optionally 1 additional heteroatom selected from oxygen, nitrogen and sulfur,

and wherein Q¹ optionally bears one or more substituents (for example 1, 2 or 3), which may be the same or different, as hereinbefore defined in (eee);

(hhh) Q¹ is selected from phenyl, pyridinyl, pyrimidinyl, pyrazinyl, 1,3-thiazolyl, 1H-imidazolyl, 1H-pyrazolyl, 1,3-oxazolyl and isoxazolyl,

and wherein Q¹ optionally bears one or more substituents (for example 1, 2 or 3), which may be the same or different, as hereinbefore defined in (eee);

(iii) Q¹ is selected from pyridinyl, pyrimidinyl, pyrazinyl, 1,3-thiazolyl, 1H-pyrazolyl and pyridazinyl,

and wherein Q¹ optionally bears one or more substituents (for example 1, 2 or 3), which may be the same or different, as hereinbefore defined in (eee);

(jjj) Q¹ is pyridinyl (for example 2-pyridinyl or 3-pyridinyl),

and wherein Q¹ optionally bears one or more substituents (for example 1, 2 or 3), which may be the same or different, as hereinbefore defined in (eee);

(kkk) Q¹ is selected from phenyl and a 5 or 6 membered monocyclic heteroaryl ring, which ring contains 1, 2 or 3 heteroatoms independently selected from oxygen, nitrogen and sulfur,

and wherein Q¹ optionally bears 1, 2, or 3 substituents, which may be the same or different, selected from halogeno (for example fluoro), hydroxy, cyano, (1-4C)alkyl (for example methyl), (1-4C)alkoxy (for example methoxy), halogeno-(1-4C)alkyl (for example fluoromethyl) and hydroxy-(1-4C)alkyl (for example hydroxymethyl);

(lll) Q¹ is selected from phenyl and a 5 or 6 membered monocyclic heteroaryl ring, which ring contains 1, 2 or 3 heteroatoms independently selected from oxygen, nitrogen and sulfur,

and wherein Q¹ optionally bears 1, 2, or 3 substituents, which may be the same or different, selected from halogeno (for example fluoro or chloro), hydroxy, (1-4C)alkyl and (1-4C)alkoxy;

(mmm) Q¹ is a 5 or 6 membered monocyclic heteroaryl ring, which ring contains 1, 2 or 3 heteroatoms independently selected from oxygen, nitrogen and sulfur,

and wherein Q¹ optionally bears one or more substituents (for example 1, 2 or 3), which may be the same or different, as hereinbefore defined in (kkk) or (lll);

(nnn) Q¹ is a 5 or 6 membered monocyclic heteroaryl ring, which ring contains 1 nitrogen heteroatom and optionally 1 additional heteroatom selected from oxygen, nitrogen and sulfur,

and wherein Q¹ optionally bears one or more substituents (for example 1, 2 or 3), which may be the same or different, as hereinbefore defined in (kkk) or (lll);

(ooo) Q¹ is selected from phenyl, pyridinyl, pyrimidinyl, pyrazinyl, 1,3-thiazolyl, 1H-imidazolyl, 1H-pyrazolyl, 1,3-oxazolyl and isoxazolyl,

and wherein Q¹ optionally bears one or more substituents (for example 1, 2 or 3), which may be the same or different, as hereinbefore defined in (kkk) or (lll);

(ppp) Q¹ is selected from pyridinyl, pyrimidinyl, pyrazinyl, 1,3-thiazolyl, 1H-pyrazolyl and pyridazinyl,

and wherein Q¹ optionally bears one or more substituents (for example 1, 2 or 3), which may be the same or different, as hereinbefore defined in (kkk) or (lll);

(qqq) Q¹ is pyridinyl (for example 2-pyridinyl or 3-pyridinyl),

and wherein Q¹ optionally bears one or more substituents (for example 1, 2 or 3), which may be the same or different, as hereinbefore defined in (kkk) or (lll);

(rrr) Q¹ is selected from phenyl and a 5 or 6 membered monocyclic heteroaryl ring, which ring contains 1, 2 or 3 heteroatoms independently selected from oxygen, nitrogen and sulfur,

and wherein Q¹ optionally bears 1, 2, or 3 substituents, which may be the same or different, selected from (1-6C)alkyl (for example (1-3C)alkyl);

(sss) Q¹ is a 5 or 6 membered monocyclic heteroaryl ring, which ring contains 1, 2 or 3 heteroatoms independently selected from oxygen, nitrogen and sulfur,

and wherein Q¹ optionally bears one or more substituents (for example 1 or 2), which may be the same or different, as hereinbefore defined in (rrr);

(ttt) Q¹ is a 5 or 6 membered monocyclic heteroaryl ring, which ring contains 1 nitrogen heteroatom and optionally 1 additional heteroatom selected from oxygen, nitrogen and sulfur,

and wherein Q¹ optionally bears one or more substituents (for example 1 or 2), which may be the same or different, as hereinbefore defined in (rrr);

(uuu) Q¹ is selected from phenyl, pyridinyl, pyrimidinyl, pyrazinyl, 1,3-thiazolyl, 1H-imidazolyl, 1H-pyrazolyl, 1,3-oxazolyl and isoxazolyl,

and wherein Q¹ optionally bears one or more substituents (for example 1 or 2), which may be the same or different, as hereinbefore defined in (rrr);

(vvv) Q¹ is selected from pyridinyl, pyrimidinyl, pyrazinyl, 1,3-thiazolyl, 1H-pyrazolyl and pyridazinyl,

and wherein Q¹ optionally bears one or more substituents (for example 1 or 2), which may be the same or different, as hereinbefore defined in (rrr);

(www) Q¹ is pyridinyl (for example 2-pyridinyl or 3-pyridinyl),

and wherein Q¹ optionally bears one or more substituents (for example 1 or 2), which may be the same or different, as hereinbefore defined in (rrr);

(xxx) Q¹ is selected from 2-pyridinyl, 3-pyridinyl, 4-pyridinyl, 6-methoxypyridin-3-yl, 6-cyanopyridin-3-yl, 6-methylpyridin-3-yl, 6-hydroxymethylpyridin-3-yl, 6-fluoromethylpyridin-3-yl, 6-fluoropyridin-3-yl, pyrazin-2-yl, 1,3-thiazol-2-yl, 1,3-thiazol-5-yl, pyrimidin-5-yl, pyridazin-3-yl and 1-methyl-1H-pyrazol-4-yl;

(yyy) Q¹ is selected from 2-pyridinyl, 3-pyridinyl, 4-pyridinyl, 6-methoxypyridin-3-yl, 6-cyanopyridin-3-yl, 6-methylpyridin-3-yl, 6-fluoromethylpyridin-3-yl, 6-fluoropyridin-3-yl and 6-hydroxymethylpyridinyl;

(zzz) Q¹ is selected from 2-pyridinyl, 3-pyridinyl, 6-fluoromethylpyridin-3-yl and 6-methylpyridin-3-yl;

(aaaa) Q¹ is selected from 2-pyridinyl and 6-methylpyridin-3-yl;

(bbbb) Q¹ is 2-pyridinyl;

(cccc) Q¹ is 6-methylpyridin-3-yl

(dddd) Q¹ is 1,3-thiazolyl (for example 1,3-thiazol-2-yl or 1,3-thiazol-5-yl);

(eeee) Q¹ is pyrimidinyl (for example pyrimidin-5-yl);

(ffff) Q¹ is pyridazinyl (for example pyridazin-3-yl);

(gggg) Q¹ is 1-methyl-1H-pyrazol-4-yl;

(hhhh) Q¹ is selected from phenyl and a 5 or 6 membered monocyclic heteroaryl ring, which ring contains 1, 2 or 3 heteroatoms independently selected from oxygen, nitrogen and sulfur,

and wherein Q¹ optionally bears one or more substituents (for example 1, 2 or 3), which may be the same or different, selected from halogeno (for example fluoro), hydroxy, cyano, (1-4C)alkyl (for example methyl), (1-4C)alkoxy (for example methoxy), halogeno-(1-4C)alkyl (for example fluoromethyl) and hydroxy-(1-4C)alkyl (for example hydroxymethyl),

X¹ is selected from O and OCH₂,

n is 0 or 1, and

R³, when present, is located at the meta-position (3-position) relative to the nitrogen in the anilino group, wherein R³ has any of the values hereinbefore defined (for example R³ is selected from fluoro, chloro, cyano, (1-3C)alkyl (for example methyl) or (1-3C)alkoxy (for example methoxy));

(iiii) Q¹ is selected from phenyl and a 5 or 6 membered monocyclic heteroaryl ring, which ring contains 1, 2 or 3 heteroatoms independently selected from oxygen, nitrogen and sulfur,

and wherein Q¹ optionally bears one or more substituents (for example 1, 2 or 3), which may be the same or different, selected from halogeno (for example fluoro or chloro), hydroxy, (1-4C)alkyl and (1-4C)alkoxy,

X¹ is selected from O and OCH₂,

n is 0 or 1, and

R³, when present, is located at the meta-position (3-position) relative to the nitrogen in the anilino group, wherein R³ has any of the values hereinbefore defined (for example R³ is selected from fluoro, chloro and (1-3C)alkyl (such as methyl));

(jjjj) Q¹ is selected from pyridinyl, pyrimidinyl, pyrazinyl, 1,3-thiazolyl, 1H-pyrazolyl and pyridazinyl,

and wherein Q¹ optionally bears one or more substituents (for example 1 or 2), which may be the same or different, as hereinbefore defined in (hhhh),

X¹ is selected from O and OCH₂,

n is 0 or 1, and

R³, when present, is located at the meta-position (3-position) relative to the nitrogen in the anilino group, wherein R³ has any of the values hereinbefore defined (for example R³ is selected from fluoro, chloro, cyano, (1-3C)alkyl (such as methyl) or (1-3C)alkoxy (such as methoxy));

(kkkk) Q¹ is selected from phenyl, pyridinyl, pyrimidinyl, pyrazinyl, 1,3-thiazolyl, 1H-imidazolyl, 1H-pyrazolyl, 1,3-oxazolyl and isoxazolyl,

and wherein Q¹ optionally bears one or more substituents (for example 1 or 2), which may be the same or different, as hereinbefore defined in (iiii),

X¹ is selected from O and OCH₂,

n is 0 or 1, and

R³, when present, is located at the meta-position (3-position) relative to the nitrogen in the anilino group, wherein R³ has any of the values hereinbefore defined (for example R³ is selected from fluoro, chloro and (1-3C)alkyl (such as methyl));

(llll) Q¹ is pyridinyl (for example 2-pyridinyl or 3-pyridinyl), which optionally bears one or more substituents (for example 1 or 2), which may be the same or different, as hereinbefore defined in (hhhh),

X¹ is selected from O and OCH₂,

n is 0 or 1, and

R³, when present, is located at the meta-position (3-position) relative to the nitrogen in the anilino group, wherein R³ has any of the values hereinbefore defined (for example R³ is selected from fluoro, chloro, cyano, (1-3C)alkyl (for example methyl) or (1-3C)alkoxy (for example methoxy));

(mmmm) Q¹ is pyridinyl (for example 2-pyridinyl or 3-pyridinyl), which optionally bears one or more substituents (for example 1 or 2), which may be the same or different, as hereinbefore defined in (iiii),

X¹ is selected from O and OCH₂,

n is 0 or 1, and

R³, when present, is located at the meta-position (3-position) relative to the nitrogen in the anilino group, wherein R³ has any of the values hereinbefore defined (for example R³ is selected from fluoro, chloro and (1-3C)alkyl (such as methyl));

(nnnn) Q¹ is pyridinyl (for example 2-pyridinyl or 3-pyridinyl), which optionally bears one or more substituents (for example 1 or 2), which may be the same or different, as hereinbefore defined in (hhhh),

X¹ is O,

n is 0 or 1, and

R³, when present, is located at the meta-position (3-position) relative to the nitrogen in the anilino group, wherein R³ has any of the values hereinbefore defined (for example R³ is selected from fluoro, chloro, cyano, (1-3C)alkyl (for example methyl) or (1-3C)alkoxy (for example methoxy));

(oooo) Q¹ is pyridinyl (for example 2-pyridinyl or 3-pyridinyl), which optionally bears one or more substituents (for example 1 or 2), which may be the same or different, as hereinbefore defined in (hhhh),

X¹ is OCH₂,

n is 0 or 1, and

R³, when present, is located at the meta-position (3-position) relative to the nitrogen in the anilino group, wherein R³ has any of the values hereinbefore defined (for example R³ is selected from fluoro, chloro, cyano, (1-3C)alkyl (for example methyl) or (1-3C)alkoxy (for example methoxy));

(pppp) Q¹ is 2-pyridinyl, which optionally bears one or more substituents (for example 1, 2 or 3), which may be the same or different, as hereinbefore defined in (iiii),

X¹ is OCH₂,

n is 0 or 1, and

R³, when present, is located at the meta-position (3-position) relative to the nitrogen in the anilino group, wherein R³ has any of the values hereinbefore defined (for example R³ is selected from fluoro, chloro and (1-3C)alkyl (for example methyl));

(qqqq) Q¹ is 3-pyridinyl, which optionally bears one or more substituents (for example 1, 2 or 3), which may be the same or different, as hereinbefore defined in (iiii),

X¹ is O,

n is 0 or 1, and

R³, when present, is located at the meta-position (3-position) relative to the nitrogen in the anilino group, wherein R³ has any of the values hereinbefore defined (for example R³ is selected from fluoro, chloro and (1-3C)alkyl (for example methyl));

(rrrr) Q¹ is pyridinyl (for example 2-pyridinyl or 3-pyridinyl), which optionally bears one or more substituents (for example 1, 2 or 3), which may be the same or different, as hereinbefore defined in (hhhh) or (iiii), and

X¹ is selected from O and OCH₂;

(ssss) Q¹ is 2-pyridinyl, which optionally bears one or more substituents (for example 1, 2 or 3), which may be the same or different, as hereinbefore defined in (hhhh) or (iiii), and

X¹ is OCH₂;

(tttt) Q¹ is 3-pyridinyl, which optionally bears one or more substituents (for example 1, 2 or 3), which may be the same or different, as hereinbefore defined in (hhhh) or (iiii), and

X¹ is O;

(uuuu) Q¹ is 3-pyridinyl, which optionally bears 1 or 2 substituents, which may be the same or different, selected from (1-4C)alkyl (for example methyl), and

X¹ is O;

(vvvv) R⁴ and R⁵, which may be the same or different, are selected from hydrogen and (1-3C)alkyl,

and wherein any CH₂ or CH₃ group within any of R⁴ and R⁵ optionally bears on each said CH₂ or CH₃ group one or more (for example 1, 2 or 3) substituents independently selected from halogeno, hydroxy, cyano, (1-6C)alkoxy, amino, (2-6C)alkanoyl, (1-6C)alkylamino and di-[(1-6C)alkylamino];

(wwww) R⁴ and R⁵, which may be the same or different, are selected from hydrogen and (1-3C)alkyl,

and wherein any CH₂ or CH₃ group within any of R⁴ and R⁵ optionally bears on each said CH₂ or CH₃ group one or more (for example 1, 2 or 3) substituents independently selected from halogeno, hydroxy, cyano, (1-6C)alkoxy and (2-6C)alkanoyl (particularly hydroxy);

(xxxx) R⁴ and R⁵ are both hydrogen;

(yyyy) R⁴ is hydrogen and R⁵ is (1-6C)alkyl (for example (1-3C)alkyl),

and wherein any CH₂ or CH₃ group within R⁵ optionally bears on each said CH₂ or CH₃ group one or more (for example 1, 2 or 3) substituents independently selected from halogeno, hydroxy, cyano, (1-6C)alkoxy, amino, (2-6C)alkanoyl, (1-6C)alkylamino and di-[(1-6C)alkylamino];

(zzzz) R⁴ is hydrogen and R⁵ is (1-6C)alkyl (for example (1-3C)alkyl),

and wherein any CH₂ or CH₃ group within R⁵ optionally bears on each said CH₂ or CH₃ group one or more (for example 1, 2 or 3) substituents independently selected from halogeno, hydroxy, cyano, (1-6C)alkoxy and (2-6C)alkanoyl (particularly hydroxy);

(aaaaa) R⁴ is hydrogen and R⁵ is (1-3C)alkyl, optionally substituted by hydroxy;

(bbbbb) R⁴ is hydrogen and R⁵ is methyl;

(ccccc) R⁴ is hydrogen and R⁵ is 2-hydroxyethyl;

(ddddd) R⁴ and R⁵ are both (1-6C)alkyl (for example (1-3C)alkyl),

and wherein any CH₂ or CH₃ group within any of R⁴ and R⁵ optionally bears on each said CH₂ or CH₃ group one or more (for example 1, 2 or 3) substituents independently selected from halogeno, hydroxy, cyano, (1-6C)alkoxy, amino, (2-6C)alkanoyl, (1-6C)alkylamino and di-[(1-6C)alkylamino];

(eeeee) R⁴ and R⁵ are both (1-6C)alkyl (for example (1-3C)alkyl),

and wherein any CH₂ or CH₃ group within any of R⁴ and R⁵ optionally bears on each said CH₂ or CH₃ group one or more (for example 1, 2 or 3) substituents independently selected from halogeno, hydroxy, cyano, (1-6C)alkoxy and (2-6C)alkanoyl (particularly hydroxy);

(fffff) R⁴ and R⁵ are both methyl;

(ggggg) R⁴ and R⁵ together with the carbon atom to which they are attached form a (3-7C)cycloalkyl ring (for example a cyclopropyl, cyclobutyl, cyclopentyl or cyclohexyl ring);

(hhhhh) R⁶ and R⁷, which may be the same or different, are selected from hydrogen, (1-6C)alkyl, (2-6C)alkenyl, (2-6C)alkynyl, (3-7C)cycloalkyl, heterocyclyl and heterocyclyl-(1-6C)alkyl, or

R⁶ and R⁷ together with the nitrogen atom to which they are attached form a saturated 4, 5 or 6 membered heterocyclic ring which optionally contains one or more additional heteroatoms independently selected from oxygen, S, SO, SO₂ and N(R¹⁰), wherein R¹⁰ is selected from hydrogen, (1-6C)alkyl, (2-6C)alkenyl, (2-6C)alkynyl, (1-6C)alkylsulfonyl, (1-6C)alkylcarbonyl and (1-6C)alkoxycarbonyl,

and wherein any heterocyclyl group within an R⁶ or an R⁷ substituent or any heterocyclic ring formed by R⁶, R⁷ and the nitrogen atom to which they are attached optionally bears one or more substituents, which may be the same or different, selected from halogeno, trifluoromethyl, cyano, nitro, hydroxy, amino, formyl, mercapto, (1-6C)alkyl, (2-6C)alkenyl, (2-6C)alkynyl, hydroxy-(1-6C)alkyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X³—R¹¹

wherein X³ is a direct bond or is selected from O, CO, SO₂ and N(R¹²), wherein R¹² is hydrogen or (1-4C)alkyl, and R¹¹ is selected from halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl,

and wherein any heterocyclyl group within an R⁶ or an R⁷ substituent or any heterocyclic ring formed by R⁶, R⁷ and the nitrogen atom to which they are attached optionally bears 1 or 2 oxo or thioxo substituents,

and wherein any CH₂ or CH₃ group within an R⁶ or an R⁷ substituent, other than a CH₂ group within a heterocyclyl group or a heterocyclic ring, optionally bears on each said CH₂ or CH₃ group one or more substituents independently selected from halogeno, (1-6C)alkyl, hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkylsulfonylamino and N-(1-6C)alkyl-(1-6C)alkylsulfonylamino;

(iiiii) R⁶ and R⁷, which may be the same or different, are selected from hydrogen, (1-6C)alkyl, (2-6C)alkenyl, (2-6C)alkynyl, (3-7C)cycloalkyl, heterocyclyl and heterocyclyl-(1-6C)alkyl, or

R⁶ and R⁷ together with the nitrogen atom to which they are attached form a saturated 5 or 6 membered heterocyclic ring which optionally contains one or more additional heteroatoms independently selected from oxygen and N(R¹⁰), wherein R¹⁰ is selected from hydrogen, (1-6C)alkyl, (2-6C)alkenyl, (2-6C)alkynyl, (1-6C)alkylsulfonyl and (1-6C)alkylcarbonyl,

and wherein any heterocyclyl group within an R⁶ or an R⁷ substituent or any heterocyclic ring formed by R⁶, R⁷ and the nitrogen atom to which they are attached optionally bears one or more substituents, which may be the same or different, as hereinbefore defined in (hhhhh),

and wherein any heterocyclyl group within an R⁶ or an R⁷ substituent or any heterocyclic ring formed by R⁶, R⁷ and the nitrogen atom to which they are attached optionally bears 1 or 2 oxo or thioxo substituents,

and wherein any CH₂ or CH₃ group within an R⁶ or an R⁷ substituent, other than a CH₂ group within a heterocyclyl group or a heterocyclic ring, optionally bears on each said CH₂ or CH₃ group one or more substituents as hereinbefore defined in (hhhhh);

(jjjj) R⁶ and R⁷, which may be the same or different, are selected from hydrogen, (1-6C)alkyl, (2-6C)alkenyl, (2-6C)alkynyl, (3-7C)cycloalkyl and heterocyclyl-(1-6C)alkyl, or

R⁶ and R⁷ together with the nitrogen atom to which they are attached form a saturated 4, 5 or 6 membered heterocyclic ring which optionally contains one or more additional heteroatoms independently selected from oxygen, S, SO, SO₂ and N(R¹⁰), wherein R¹⁰ is selected from hydrogen, (1-6C)alkyl and (1-6C)alkoxycarbonyl,

and wherein any heterocyclyl group within an R⁶ or an R⁷ substituent or any heterocyclic ring formed by R⁶, R⁷ and the nitrogen atom to which they are attached optionally bears one or more substituents, which may be the same or different, as hereinbefore defined in (hhhhh),

and wherein any heterocyclyl group within an R⁶ or an R⁷ substituent or any heterocyclic ring formed by R⁶, R⁷ and the nitrogen atom to which they are attached optionally bears 1 or 2 oxo or thioxo substituents,

and wherein any CH₂ or CH₃ group within an R⁶ or an R⁷ substituent, other than a CH₂ group within a heterocyclyl group or a heterocyclic ring, optionally bears on each said CH₂ or CH₃ group one or more substituents as hereinbefore defined in (hhhhh);

(kkkkk) R⁶ and R⁷, which may be the same or different, are selected from hydrogen, (1-6C)alkyl, (2-6C)alkenyl, (2-6C)alkynyl, (3-7C)cycloalkyl, heterocyclyl and heterocyclyl-(1-6C)alkyl, wherein when R⁶ and/or R⁷ is a heterocyclyl group it is a 4, 5, 6 or 7 membered monocyclic saturated or partially saturated heterocyclyl group containing 1 or 2 heteroatoms independently selected from oxygen, nitrogen and sulfur, or

R⁶ and R⁷ together with the nitrogen atom to which they are attached form a saturated 4, 5 or 6 membered heterocyclic ring which optionally contains one or more additional heteroatoms independently selected from oxygen, S, SO, SO₂ or N(R¹⁰), wherein R¹⁰ is selected from hydrogen, (1-6C)alkyl, (2-6C)alkenyl, (2-6C)alkynyl, (1-6C)alkylsulfonyl, (1-6C)alkylcarbonyl and (1-6C)alkoxycarbonyl,

and wherein any heterocyclyl group within an R⁶ or an R⁷ substituent or any heterocyclic ring formed by R⁶, R⁷ and the nitrogen atom to which they are attached optionally bears one or more substituents, which may be the same or different, as hereinbefore defined in (hhhhh),

and wherein any heterocyclyl group within an R⁶ or an R⁷ substituent or any heterocyclic ring formed by R⁶, R⁷ and the nitrogen atom to which they are attached optionally bears 1 or 2 oxo or thioxo substituents;

and wherein any CH₂ or CH₃ group within an R⁶ or an R⁷ substituent, other than a CH₂ group within a heterocyclyl group or a heterocyclic ring, optionally bears on each said CH₂ or CH₃ group one or more substituents as hereinbefore defined in (hhhhh);

(lllll) R⁶ and R⁷, which may be the same or different, are selected from hydrogen, (1-6C)alkyl, (2-6C)alkenyl, (2-6C)alkynyl, (3-7C)cycloalkyl, heterocyclyl and heterocyclyl-(1-6C)alkyl, wherein when R⁶ and/or R⁷ is a heterocyclyl group it is a 4, 5, 6 or 7 membered monocyclic saturated or partially saturated heterocyclyl group containing 1 or 2 heteroatoms independently selected from oxygen, nitrogen and sulfur, or

R⁶ and R⁷ together with the nitrogen atom to which they are attached form a saturated 5 or 6 membered heterocyclic ring which optionally contains one or more additional heteroatoms independently selected from oxygen or N(R¹⁰), wherein R¹⁰ is selected from hydrogen, (1-6C)alkyl, (2-6C)alkenyl, (2-6C)alkynyl, (1-6C)alkylsulfonyl and (1-6C)alkylcarbonyl,

and wherein any heterocyclyl group within an R⁶ or an R⁷ substituent or any heterocyclic ring formed by R⁶, R⁷ and the nitrogen atom to which they are attached optionally bears one or more substituents, which may be the same or different, as hereinbefore defined in (hhhhh),

and wherein any heterocyclyl group within an R⁶ or an R⁷ substituent or any heterocyclic ring formed by R⁶, R⁷ and the nitrogen atom to which they are attached optionally bears 1 or 2 oxo or thioxo substituents,

and wherein any CH₂ or CH₃ group within an R⁶ or an R⁷ substituent, other than a CH₂ group within a heterocyclyl group or a heterocyclic ring, optionally bears on each said CH₂ or CH₃ group one or more substituents as hereinbefore defined in (hhhhh);

(mmmmm) R⁶ and R⁷, which may be the same or different, are selected from hydrogen, (1-4C)alkyl, (2-4C)alkenyl, (2-4C)alkynyl, (3-5C)cycloalkyl, heterocyclyl and heterocyclyl-(1-4C)alkyl, wherein when R⁶ and/or R⁷ is a heterocyclyl group it is a 4, 5, 6 or 7 membered monocyclic saturated or partially saturated heterocyclyl group containing 1 or 2 heteroatoms independently selected from oxygen, nitrogen and sulfur, or

R⁶ and R⁷ together with the nitrogen atom to which they are attached form a saturated 4, 5 or 6 membered heterocyclic ring which optionally contains one or more additional heteroatoms independently selected from oxygen, S, SO, SO₂ or N(R¹⁰), wherein R¹⁰ is selected from hydrogen, (1-6C)alkyl and (1-6C)alkoxycarbonyl,

and wherein any heterocyclyl group within an R⁶ or an R⁷ substituent or any heterocyclic ring formed by R⁶, R⁷ and the nitrogen atom to which they are attached optionally bears one or more substituents, which may be the same or different, as hereinbefore defined in (hhhhh),

and wherein any heterocyclyl group within an R⁶ or an R⁷ substituent or any heterocyclic ring formed by R⁶, R⁷ and the nitrogen atom to which they are attached optionally bears 1 or 2 oxo or thioxo substituents,

and wherein any CH₂ or CH₃ group within an R⁶ or an R⁷ substituent, other than a CH₂ group within a heterocyclyl group or a heterocyclic ring, optionally bears on each said CH₂ or CH₃ group one or more substituents as hereinbefore defined in (hhhhh);

(nnnnn) R⁶ and R⁷, which may be the same or different, are selected from hydrogen, (1-4C)alkyl, (2-4C)alkenyl, (2-4C)alkynyl, (3-5C)cycloalkyl, heterocyclyl and heterocyclyl-(1-4C)alkyl, wherein when R⁶ and/or R⁷ is a heterocyclyl group it is a 4, 5, 6 or 7 membered monocyclic saturated or partially saturated heterocyclyl group containing 1 or 2 heteroatoms independently selected from oxygen, nitrogen and sulfur, or

R⁶ and R⁷ together with the nitrogen atom to which they are attached form a saturated 5 or 6 membered heterocyclic ring which optionally contains one or more additional heteroatoms independently selected from oxygen or N(R¹⁰), wherein R¹⁰ is selected from hydrogen and (1-6C)alkyl,

and wherein any heterocyclyl group within an R⁶ or an R⁷ substituent or any heterocyclic ring formed by R⁶, R⁷ and the nitrogen atom to which they are attached optionally bears one or more substituents, which may be the same or different, as hereinbefore defined in (hhhhh),

and wherein any heterocyclyl group within an R⁶ or an R⁷ substituent or any heterocyclic ring formed by R⁶, R⁷ and the nitrogen atom to which they are attached optionally bears 1 or 2 oxo or thioxo substituents,

and wherein any CH₂ or CH₃ group within an R⁶ or an R⁷ substituent, other than a CH₂ group within a heterocyclyl group or a heterocyclic ring, optionally bears on each said CH₂ or CH₃ group one or more substituents as hereinbefore defined in (hhhhh);

(ooooo) R⁶ and R⁷, which may be the same or different, are selected from hydrogen, methyl, ethyl, propyl, isopropyl, butyl, tert-butyl, propenyl, butenyl, propynyl, butynyl, cyclopropyl, cyclobutyl, cyclopentyl, heterocyclyl, heterocyclyl-methyl, heterocyclyl-ethyl and heterocyclyl-propyl, wherein when R⁶ and/or R⁷ is a heterocyclyl group it is a 4, 5, 6 or 7 membered monocyclic saturated or partially saturated heterocyclyl group containing 1 or 2 heteroatoms independently selected from oxygen, nitrogen and sulfur, or

R⁶ and R⁷ together with the nitrogen atom to which they are attached form a heterocyclic ring selected from azetidin-1-yl, pyrrolidin-1-yl, piperidin-1-yl, morpholin-4-yl and piperazin-1-yl,

and wherein when R⁶ and R⁷ together with the nitrogen atom to which they are attached form a heterocyclic ring that is piperazin-1-yl, any nitrogen atom apart from the NR⁶R⁷ nitrogen atom is substituted by R¹⁰, wherein R¹⁰ is selected from hydrogen, (1-4C)alkyl (for example methyl or ethyl) and (1-4C)alkoxycarbonyl (for example tert-butoxycarbonyl),

and wherein any heterocyclyl group within an R⁶ or an R⁷ substituent or any heterocyclic ring formed by R⁶, R⁷ and the nitrogen atom to which they are attached optionally bears one or more substituents, which may be the same or different, as hereinbefore defined in (hhhhh),

and wherein any heterocyclyl group within an R⁶ or an R⁷ substituent or any heterocyclic ring formed by R⁶, R⁷ and the nitrogen atom to which they are attached optionally bears 1 or 2 oxo or thioxo substituents,

and wherein any CH₂ or CH₃ group within an R⁶ or an R⁷ substituent, other than a CH₂ group within a heterocyclyl group or a heterocyclic ring, optionally bears on each said CH₂ or CH₃ group one or more substituents as hereinbefore defined in (hhhhh);

(ppppp) R⁶ and R⁷, which may be the same or different, are selected from hydrogen, methyl, ethyl, propyl, isopropyl, butyl, tert-butyl, propenyl, butenyl, propynyl, butynyl, cyclopropyl, cyclobutyl, cyclopentyl, heterocyclyl, heterocyclyl-methyl, heterocyclyl-ethyl and heterocyclyl-propyl, wherein when R⁶ and/or R⁷ is a heterocyclyl group it is a 4, 5, 6 or 7 membered monocyclic saturated or partially saturated heterocyclyl group containing 1 or 2 heteroatoms independently selected from oxygen, nitrogen and sulfur, or

R⁶ and R⁷ together with the nitrogen atom to which they are attached form a heterocyclic ring selected from pyrrolidin-1-yl, pyrazolidin-1-yl, piperidin-1-yl, morpholin-4-yl and piperazin-1-yl,

and wherein when R⁶ and R⁷ together with the nitrogen atom to which they are attached form a heterocyclic ring selected from pyrazolidin-1-yl and piperazin-1-yl, any nitrogen atom apart from the NR⁶R⁷ nitrogen atom is substituted by R¹⁰, wherein R¹⁰ is selected from hydrogen, (1-4C)alkyl (for example methyl or ethyl) and (1-4C)alkoxycarbonyl (for example tert-butoxycarbonyl),

and wherein any heterocyclyl group within an R⁶ or an R⁷ substituent or any heterocyclic ring formed by R⁶, R⁷ and the nitrogen atom to which they are attached optionally bears one or more substituents, which may be the same or different, as hereinbefore defined in (hhhhh),

and wherein any heterocyclyl group within an R⁶ or an R⁷ substituent or any heterocyclic ring formed by R⁶, R⁷ and the nitrogen atom to which they are attached optionally bears 1 or 2 oxo or thioxo substituents,

and wherein any CH₂ or CH₃ group within an R⁶ or an R⁷ substituent, other than a CH₂ group within a heterocyclyl group or a heterocyclic ring, optionally bears on each said CH₂ or CH₃ group one or more substituents as hereinbefore defined in (hhhhh);

(qqqqq) R⁶ and R⁷, which may be the same or different, are selected from hydrogen, methyl, ethyl, propyl, isopropyl, butyl, isobutyl, tert-butyl, vinyl, isopropenyl, allyl, but-2-enyl, ethynyl, 2-propynyl, butynyl, cyclopropyl, cyclobutyl, cyclopentyl, cyclohexyl, azetidinyl, pyrrolinyl, pyrrolidinyl, piperidinyl, homopiperidinyl, homopiperazinyl, dihydropyridinyl, tetrahydropyridinyl, dihydropyrimidinyl, tetrahydropyrimidinyl, tetrahydrothienyl, tetrahydrothiopyranyl, tetrahydrofuranyl, tetrahydropyranyl, cyclopropylmethyl, cyclobutylmethyl, cyclopentylmethyl, cyclohexylmethyl, 2-cyclopropylethyl, 2-cyclobutylethyl, 2-cyclopentylethyl, 2-cyclohexylethyl, azetidinylmethyl, pyrrolinylmethyl, pyrrolidinylmethyl, morpholinylmethyl, piperidinylmethyl, homopiperidinylmethyl, piperazinylmethyl, homopiperazinylmethyl, dihydropyridinylmethyl, tetrahydropyridinylmethyl, dihydropyrimidinylmethyl, tetrahydropyrimidinylmethyl, tetrahydrothienylmethyl, tetrahydrothiopyranylmethyl, thiomorpholinylmethyl, tetrahydrofuranylmethyl, tetrahydropyranylmethyl, 2-(azetidinyl)ethyl, 2-(pyrrolinyl)ethyl, 2-(pyrrolidinyl)ethyl, 2-(morpholinyl)ethyl, 2-(piperidinyl)ethyl, 2-(homopiperidinyl)ethyl, 2-(piperazinyl)ethyl, 2-(homopiperazinyl)ethyl, 2-(dihydropyridinyl)ethyl, 2-(tetrahydropyridinyl)ethyl, 2-(dihydropyrimidinyl)ethyl, 2-(tetrahydropyrimidinyl)ethyl, 2-(tetrahydrothienyl)ethyl, 2-(tetrahydrothiopyranyl)ethyl, 2-(thiomorpholinyl)ethyl, 2-(tetrahydrofuranyl)ethyl, 2-(tetrahydropyranyl)ethyl, 3-(piperazinyl)propyl and 3-(pyrrolidinyl)propyl, or

R⁶ and R⁷ together with the nitrogen atom to which they are attached form a heterocyclic ring selected from azetidin-1-yl, pyrrolidin-1-yl, pyrazolidin-1-yl, piperidin-1-yl, morpholin-4-yl and piperazin-1-yl,

and wherein when R⁶ and R⁷ together with the nitrogen atom to which they are attached form a heterocyclic ring selected from pyrazolidin-1-yl and piperazin-1-yl, any nitrogen atom apart from the NR⁶R⁷ nitrogen atom is substituted by R¹⁰, wherein R¹⁰ is selected from hydrogen, (1-4C)alkyl (for example methyl or ethyl) and (1-4C)alkoxycarbonyl (for example tert-butoxycarbonyl),

and wherein any heterocyclyl group within an R⁶ or an R⁷ substituent or any heterocyclic ring formed by R⁶, R⁷ and the nitrogen atom to which they are attached optionally bears one or more substituents, which may be the same or different, selected from fluoro, chloro, bromo, oxo, hydroxy, hydroxymethyl, methyl, ethyl, propyl, butyl, isopropyl, isobutyl, trifluoromethyl, vinyl, isopropenyl, allyl, but-2-enyl, ethynyl, 2-propynyl, butynyl, methoxy, ethoxy, propoxy, isopropoxy, trifluoromethoxy, acetyl, propionyl, methoxymethyl, ethoxymethyl, 2-hydroxyethyl, 2-methoxyethyl, butoxycarbonyl and 2-ethoxyethyl,

and wherein any CH₂ or CH₃ group within an R⁶ or an R⁷ substituent, other than a CH₂ group within a heterocyclyl group or a heterocyclic ring, optionally bears on each said CH₂ or CH₃ group one or more substituents independently selected from fluoro, chloro, bromo, methyl, ethyl, propyl, isopropyl, hydroxy, amino, methoxy, ethoxy, methylamino, ethylamino, di-methylamino, di-ethylamino, N-methyl-N-ethylamino, acetylamino, methylsulfonyl, methylthio and ethylsulfonyl;

(rrrrr) R⁶ and R⁷, which may be the same or different, are selected from hydrogen, methyl, ethyl, propyl, isopropyl, tert-butyl, allyl, 2-propynyl, cyclopropyl, cyclobutyl, piperidinyl, 2-(pyrrolidinyl)ethyl, 2-(morpholinyl)ethyl, 3-(piperazinyl)propyl and 3-(pyrrolidinyl)propyl, or

R⁶ and R⁷ together with the nitrogen atom to which they are attached form a heterocyclic ring selected from azetidin-1-yl, pyrrolidin-1-yl, piperidin-1-yl, morpholin-4-yl and piperazin-1-yl,

and wherein when R⁶ and R⁷ together with the nitrogen atom to which they are attached form a heterocyclic ring that is piperazin-1-yl, any nitrogen atom apart from the R⁶R⁷ nitrogen atom is substituted by R¹⁰, wherein R¹⁰ is selected from hydrogen, (1-4C)alkyl (for example methyl or ethyl) and (1-4C)alkoxycarbonyl (for example tert-butoxycarbonyl),

and wherein any heterocyclyl group within an R⁶ or an R⁷ substituent or any heterocyclic ring formed by R⁶, R⁷ and the nitrogen atom to which they are attached optionally bears one or more substituents, which may be the same or different, selected from oxo, hydroxy, hydroxymethyl, methyl, ethyl and butoxycarbonyl,

and wherein any CH₂ or CH₃ group within an R⁶ or an R⁷ substituent, other than a CH₂ group within a heterocyclyl group or a heterocyclic ring, optionally bears on each said CH₂ or CH₃ group one or more substituents independently selected from hydroxy, methoxy, di-methylamino, di-ethylamino, acetylamino, methylsulfonyl and methylthio;

(sssss) R⁶ and R⁷, which may be the same or different, are selected from hydrogen, methyl, ethyl, propyl, isopropyl, butyl, isobutyl, tert-butyl, vinyl, isopropenyl, allyl, but-2-enyl, ethynyl, 2-propynyl, butynyl, cyclopropyl, cyclobutyl, cyclopentyl, cyclohexyl, azetidinyl, pyrrolinyl, pyrrolidinyl, piperidinyl, homopiperidinyl, homopiperazinyl, dihydropyridinyl, tetrahydropyridinyl, dihydropyrimidinyl, tetrahydropyrimidinyl, tetrahydrothienyl, tetrahydrothiopyranyl, tetrahydrofuranyl, tetrahydropyranyl, cyclopropylmethyl, cyclobutylmethyl, cyclopentylmethyl, cyclohexylmethyl, 2-cyclopropylethyl, 2-cyclobutylethyl, 2-cyclopentylethyl, 2-cyclohexylethyl, azetidinylmethyl, pyrrolinylmethyl, pyrrolidinylmethyl, morpholinylmethyl, piperidinylmethyl, homopiperidinylmethyl, piperazinylmethyl, homopiperazinylmethyl, dihydropyridinylmethyl, tetrahydropyridinylmethyl, dihydropyrimidinylmethyl, tetrahydropyrimidinylmethyl, tetrahydrothienylmethyl, tetrahydrothiopyranylmethyl, thiomorpholinylmethyl, tetrahydrofuranylmethyl, tetrahydropyranylmethyl, 2-(azetidinyl)ethyl, 2-(pyrrolinyl)ethyl, 2-(pyrrolidinyl)ethyl, 2-(morpholinyl)ethyl, 2-(piperidinyl)ethyl, 2-(homopiperidinyl)ethyl, 2-(piperazinyl)ethyl, 2-(homopiperazinyl)ethyl, 2-(dihydropyridinyl)ethyl, 2-(tetrahydropyridinyl)ethyl, 2-(dihydropyrimidinyl)ethyl, 2-(tetrahydropyrimidinyl)ethyl, 2-(tetrahydrothienyl)ethyl, 2-(tetrahydrothiopyranyl)ethyl, 2-(thiomorpholinyl)ethyl, 2-(tetrahydrofuranyl)ethyl and 2-(tetrahydropyranyl)ethyl, or

R⁶ and R⁷ together with the nitrogen atom to which they are attached form a heterocyclic ring selected from pyrrolidin-1-yl, pyrazolidin-1-yl, piperidin-1-yl, morpholin-4-yl and piperazin-1-yl,

and wherein when R⁶ and R⁷ together with the nitrogen atom to which they are attached form a heterocyclic ring selected from pyrazolidin-1-yl and piperazin-1-yl, any nitrogen atom apart from the NR⁶R⁷ nitrogen atom is substituted by R¹⁰, wherein R¹⁰ is selected from hydrogen, (1-4C)alkyl (for example methyl or ethyl) and (1-4C)alkoxycarbonyl (for example tert-butoxycarbonyl),

and wherein any heterocyclyl group within an R⁶ or an R⁷ substituent or any heterocyclic ring formed by R⁶, R⁷ and the nitrogen atom to which they are attached optionally bears one or more substituents, which may be the same or different, selected from fluoro, chloro, bromo, oxo, hydroxy, hydroxymethyl, methyl, ethyl, propyl, butyl, isopropyl, isobutyl, trifluoromethyl, vinyl, isopropenyl, allyl, but-2-enyl, ethynyl, 2-propynyl, butynyl, methoxy, ethoxy, propoxy, isopropoxy, trifluoromethoxy, acetyl, propionyl, methoxymethyl, ethoxymethyl, 2-hydroxyethyl, 2-methoxyethyl and 2-ethoxyethyl,

and wherein any CH₂ or CH₃ group within an R⁶ or an R⁷ substituent, other than a CH₂ group within a heterocyclyl group or a heterocyclic ring, optionally bears on each said CH₂ or CH₃ group one or more substituents independently selected from fluoro, chloro, bromo, methyl, ethyl, propyl, isopropyl, hydroxy, amino, methoxy, ethoxy, methylamino, ethylamino, di-methylamino, di-ethylamino, N-methyl-N-ethylamino, methylsulfonyl and ethylsulfonyl;

(ttttt) R⁶ and R⁷, which may be the same or different, are selected from hydrogen, methyl, ethyl, 2-hydroxyethyl, 2-methoxyethyl, 2-hydroxy-1,1-dimethylethyl, propyl, isopropyl, 3-hydroxypropyl, 2-hydroxypropyl, 3-methoxypropyl, 2-methoxypropyl, 2,3-dihydroxypropyl, isopropyl, 2-hydroxy-isopropyl, vinyl, isopropenyl, allyl, but-2-enyl, ethynyl, 2-propynyl, 2-methylsulfonylethyl, 2-(dimethylamino)ethyl, 2-(diethylamino)ethyl, 2-(acetylamino)ethyl, 2-(methylthio)ethyl, cyclopropyl, cyclobutyl, cyclopentyl, cyclohexyl, azetidinyl, pyrrolinyl, pyrrolidinyl, piperidinyl, homopiperidinyl, tetrahydrofuranyl, tetrahydropyranyl, cyclopropylmethyl, cyclobutylmethyl, cyclopentylmethyl, cyclohexylmethyl, 2-cyclopropylethyl, 2-cyclobutylethyl, 2-cyclopentylethyl, 2-cyclohexylethyl, azetidinylmethyl, pyrrolidinylmethyl, piperidinylmethyl, homopiperidinylmethyl, tetrahydrothiopyranylmethyl, tetrahydrofuranylmethyl, tetrahydropyranylmethyl, 2-(azetidinyl)ethyl, 2-(morpholin-4-yl)ethyl, 2-(pyrrolidinyl)ethyl, 2-(piperidinyl)ethyl, 2-(homopiperidinyl)ethyl, 2-(tetrahydrothienyl)ethyl, 2-(tetrahydrothiopyranyl)ethyl, 2-(thiomorpholinyl)ethyl, 2-(tetrahydrofuranyl)ethyl, 2-(tetrahydropyranyl)ethyl, 3-(piperazinyl)propyl and 3-(pyrrolidinyl)propyl, or

R⁶ and R⁷ together with the nitrogen atom to which they are attached form a heterocyclic ring selected from azetidin-1-yl, pyrrolidin-1-yl, piperidin-1-yl, morpholin-4-yl and piperazin-1-yl,

and wherein when R⁶ and R⁷ together with the nitrogen atom to which they are attached form a heterocyclic ring that is piperazin-1-yl, any nitrogen atom apart from the NR⁶R⁷ nitrogen atom is substituted by R¹⁰, wherein R¹⁰ is selected from hydrogen, (1-4C)alkyl (for example methyl or ethyl) and (1-4C)alkoxycarbonyl (for example tert-butoxycarbonyl),

and wherein any heterocyclyl group within an R⁶ or an R⁷ substituent or any heterocyclic ring formed by R⁶, R⁷ and the nitrogen atom to which they are attached optionally bears one or more substituents, which may be the same or different, selected from fluoro, chloro, bromo, oxo, hydroxy, hydroxymethyl, methyl, ethyl, propyl, isopropyl, trifluoromethyl, methoxy, ethoxy, propoxy, isopropoxy and trifluoromethoxy;

and wherein any CH₂ group within a cycloalkyl group within an R⁶ or an R⁷ substituent optionally bears on each CH₂ group 1 or 2 substituents independently selected from hydroxy, methyl, ethyl, methoxy and ethoxy,

and wherein any CH₂ or CH₃ group within an R⁶ or an R⁷ substituent, other than a CH₂ group within a heterocyclyl group or a heterocyclic ring, optionally bears on each said CH₂ or CH₃ group one or more fluoro substituents;

(uuuuu) R⁶ and R⁷, which may be the same or different, are selected from hydrogen, methyl, ethyl, 2-hydroxyethyl, 2-methoxyethyl, 2-hydroxy-1,1-dimethylethyl, propyl, isopropyl, 3-hydroxypropyl, 2-hydroxypropyl, 3-methoxypropyl, 2,3-dihydroxypropyl, isopropyl, 2-hydroxy-isopropyl, allyl, 2-propynyl, 2-methylsulfonylethyl, 2-(dimethylamino)ethyl, 2-(diethylamino)ethyl, 2-(acetylamino)ethyl, 2-(methylthio)ethyl, cyclopropyl, cyclobutyl, piperidinyl, 2-(morpholin-4-yl)ethyl, 2-(pyrrolidinyl)ethyl, 3-(piperazinyl)propyl and 3-(pyrrolidinyl)propyl, or

R⁶ and R⁷ together with the nitrogen atom to which they are attached form a heterocyclic ring selected from azetidin-1-yl, pyrrolidin-1-yl, piperidin-1-yl, morpholin-4-yl and piperazin-1-yl,

and wherein when R⁶ and R⁷ together with the nitrogen atom to which they are attached form a heterocyclic ring that is piperazin-1-yl, any nitrogen atom apart from the NR⁶R⁷ nitrogen atom is substituted by R¹⁰, wherein ¹⁰ is selected from hydrogen, (1-4C)alkyl (for example methyl or ethyl) and (1-4C)alkoxycarbonyl (for example tert-butoxycarbonyl),

and wherein any heterocyclyl group within an R⁶ or an R⁷ substituent or any heterocyclic ring formed by R⁶, R⁷ and the nitrogen atom to which they are attached optionally bears one or more substituents, which may be the same or different, selected from fluoro, chloro, bromo, oxo, hydroxy, hydroxymethyl, methyl, ethyl, propyl, isopropyl, trifluoromethyl, methoxy, ethoxy, propoxy, isopropoxy and trifluoromethoxy,

and wherein any CH₂ group within a cycloalkyl group within an R⁶ or an R⁷ substituent optionally bears on each CH₂ group 1 or 2 substituents independently selected from hydroxy, methyl, ethyl, methoxy and ethoxy,

and wherein any CH₂ or CH₃ group within an R⁶ or an R⁷ substituent, other than a CH₂ group within a heterocyclyl group or a heterocyclic ring, optionally bears on each said CH₂ or CH₃ group one or more fluoro substituents;

(vvvvv) R⁶ and R⁷, which may be the same or different, are selected from hydrogen, methyl, ethyl, 2-hydroxyethyl, 2-methoxyethyl, 2-hydroxy-1,1-dimethylethyl, propyl, isopropyl, 3-hydroxypropyl, 2-hydroxypropyl, 3-methoxypropyl, 2-methoxypropyl, isopropyl, vinyl, isopropenyl, allyl, but-2-enyl, ethynyl, 2-propynyl, 2-methylsulfonylethyl, 2-(dimethylamino)ethyl, cyclopropyl, cyclobutyl, cyclopentyl, cyclohexyl, azetidinyl, pyrrolinyl, pyrrolidinyl, piperidinyl, homopiperidinyl, tetrahydrofuranyl, tetrahydropyranyl, cyclopropylmethyl, cyclobutylmethyl, cyclopentylmethyl, cyclohexylmethyl, 2-cyclopropylethyl, 2-cyclobutylethyl, 2-cyclopentylethyl, 2-cyclohexylethyl, azetidinylmethyl, pyrrolidinylmethyl, piperidinylmethyl, homopiperidinylmethyl, tetrahydrothiopyranylmethyl, tetrahydrofuranylmethyl, tetrahydropyranylmethyl, 2-(azetidinyl)ethyl, 2-(morpholin-4-yl)ethyl, 2-(pyrrolidinyl)ethyl, 2-(piperidinyl)ethyl, 2-(homopiperidinyl)ethyl, 2-(tetrahydrothienyl)ethyl, 2-(tetrahydrothiopyranyl)ethyl, 2-(thiomorpholinyl)ethyl, 2-(tetrahydrofuranyl)ethyl and 2-(tetrahydropyranyl)ethyl, or

R⁶ and R⁷ together with the nitrogen atom to which they are attached form a heterocyclic ring selected from pyrrolidin-1-yl, pyrazolidin-1-yl, piperidin-1-yl, morpholin-4-yl and piperazin-1-yl,

and wherein when R⁶ and R⁷ together with the nitrogen atom to which they are attached form a heterocyclic ring selected from pyrazolidin-1-yl and piperazin-1-yl, any nitrogen atom apart from the NR⁶R⁷ nitrogen atom is substituted by R¹⁰, wherein R¹⁰ is selected from hydrogen, (1-4C)alkyl (for example methyl or ethyl) and (1-4C)alkoxycarbonyl (for example tert-butoxycarbonyl),

and wherein any heterocyclyl group within an R⁶ or an R⁷ substituent or any heterocyclic ring formed by R⁶, R⁷ and the nitrogen atom to which they are attached optionally bears one or more substituents, which may be the same or different, selected from fluoro, chloro, bromo, oxo, hydroxy, hydroxymethyl, methyl, ethyl, propyl, isopropyl, trifluoromethyl, methoxy, ethoxy, propoxy, isopropoxy and trifluoromethoxy,

and wherein any CH₂ group within a cycloalkyl group within an R⁶ or an R⁷ substituent optionally bears on each CH₂ group 1 or 2 substituents independently selected from hydroxy, methyl, ethyl, methoxy and ethoxy,

and wherein any CH₂ or CH₃ group within an R⁶ or an R⁷ substituent, other than a CH₂ group within a heterocyclyl group or a heterocyclic ring, optionally bears on each said CH₂ or CH₃ group one or more fluoro substituents;

(wwwww) R⁶ and R⁷, which may be the same or different, are selected from hydrogen, methyl, ethyl, 2-hydroxyethyl, 2-methoxyethyl, 2-hydroxy-1,1-dimethylethyl, 2-methylsulfonylethyl, 2-(dimethylamino)ethyl, propyl, isopropyl, isopropenyl, 2-propynyl, cyclopropyl, cyclobutyl, 2-(morpholin-4-yl)ethyl and piperidinyl, or

R⁶ and R⁷ together with the nitrogen atom to which they are attached form a heterocyclic ring selected from azetidin-1-yl, pyrrolidin-1-yl, piperidin-1-yl, morpholin-4-yl and piperazin-1-yl,

and wherein when R⁶ and R⁷ together with the nitrogen atom to which they are attached form a heterocyclic ring that is piperazin-1-yl, any nitrogen atom apart from the NR⁶R⁷ nitrogen atom is substituted by R¹⁰, wherein R¹⁰ is selected from hydrogen, (1-4C)alkyl (for example methyl or ethyl) and (1-4C)alkoxycarbonyl (for example tert-butoxycarbonyl),

and wherein any heterocyclyl group within an R⁶ or an R⁷ substituent or any heterocyclic ring formed by R⁶, R⁷ and the nitrogen atom to which they are attached optionally bears one or more substituents, which may be the same or different, selected from fluoro, chloro, bromo, oxo, hydroxy, methyl, hydroxymethyl, ethyl, propyl, isopropyl, trifluoromethyl, methoxy, ethoxy, propoxy, isopropoxy and trifluoromethoxy,

and wherein any CH₂ group within a cycloalkyl group within an R⁶ or an R⁷ substituent optionally bears on each CH₂ group 1 or 2 substituents independently selected from hydroxy methyl, ethyl, methoxy and ethoxy;

(xxxxx) R⁶ and R⁷, which may be the same or different, are selected from hydrogen, methyl, ethyl, 2-hydroxyethyl, 2-methoxyethyl, 2-hydroxy-1,1-dimethylethyl, 2-methylsulfonylethyl, 2-(dimethylamino)ethyl, propyl, isopropyl, isopropenyl, 2-propynyl, cyclopropyl, cyclobutyl, 2-(morpholin-4-yl)ethyl and piperidinyl, or

R⁶ and R⁷ together with the nitrogen atom to which they are attached form a heterocyclic ring selected from pyrrolidin-1-yl, pyrazolidin-1-yl, morpholin-4-yl and piperazin-1-yl,

and wherein when R⁶ and R⁷ together with the nitrogen atom to which they are attached form a heterocyclic ring selected from pyrazolidin-1-yl and piperazin-1-yl, any nitrogen atom apart from the NR⁶R⁷ nitrogen atom is substituted by R¹⁰, wherein R¹⁰ is selected from hydrogen, (1-4C)alkyl (for example methyl or ethyl) and (1-4C)alkoxycarbonyl (for example tert-butoxycarbonyl),

and wherein any heterocyclyl group within an R⁶ or an R⁷ substituent or any heterocyclic ring formed by R⁶, R⁷ and the nitrogen atom to which they are attached optionally bears one or more substituents, which may be the same or different, selected from fluoro, chloro, bromo, oxo, hydroxy, methyl, hydroxymethyl, ethyl, propyl, isopropyl, trifluoromethyl, methoxy, ethoxy, propoxy, isopropoxy and trifluoromethoxy,

and wherein any CH₂ group within a cycloalkyl group within an R⁶ or an R⁷ substituent optionally bears on each CH₂ group 1 or 2 substituents independently selected from hydroxy methyl, ethyl, methoxy and ethoxy;

(yyyyy) R⁶ and R⁷ are both hydrogen;

(zzzzz) R⁶ is hydrogen and R⁷ is selected from (1-6C)alkyl, (2-6C)alkenyl, (2-6C)alkynyl, (3-7C)cycloalkyl and heterocyclyl-(1-6C)alkyl (particularly (1-6C)alkyl).

and wherein any heterocyclyl group within an R⁷ substituent optionally bears one or more substituents, which may be the same or different, as hereinbefore defined in (hhhhh),

and wherein any CH₂ or CH₃ group within an R⁷ substituent optionally bears on each said CH₂ or CH₃ group one or more substituents as hereinbefore defined in (hhhhh);

(aaaaaa) R⁶ is hydrogen and R⁷ is selected from (1-6C)alkyl, (2-6C)alkenyl, (2-6C)alkynyl and (3-7C)cycloalkyl (particularly (1-6C)alkyl),

and wherein any CH₂ or CH₃ group within an R⁷ substituent optionally bears on each said CH₂ or CH₃ group one or more substituents as hereinbefore defined in (iiiii);

(bbbbbb) R⁶ is (1-6C)alkyl and R⁷ is selected from hydrogen, (1-6C)alkyl, (2-6C)alkenyl, (2-6C)alkynyl, (3-7C)cycloalkyl, heterocyclyl and heterocyclyl-(1-6C)alkyl,

and wherein any heterocyclyl group within an R⁷ substituent optionally bears one or more substituents, which may be the same or different, as hereinbefore defined in (hhhhh) or (iiiii),

and wherein any heterocyclyl group within an R⁷ substituent optionally bears 1 or 2 oxo or thioxo substituents,

and wherein any CH₂ or CH₃ group within an R⁶ or an R⁷ substituent, other than a CH₂ group within a heterocyclyl group, optionally bears on each said CH₂ or CH₃ group one or more substituents as hereinbefore defined in (hhhhh) or (iiiii);

(cccccc) R⁶ and R⁷ are selected from (1-4C)alkyl (for example methyl or ethyl),

and wherein any CH₂ or CH₃ group within an R⁶ or an R⁷ substituent optionally bears on each said CH₂ or CH₃ group one or more hydroxy substituents;

(dddddd) R⁶ and R⁷ together with the nitrogen atom to which they are attached form a heterocyclic ring selected from azetidin-1-yl, 3-hydroxy-azetidinyl, morpholin-4-yl, piperazin-1-yl, 4-methyl-piperazin-1-yl, 4-ethyl-piperazin-1-yl, 3-oxo-piperazin-1-yl, 4-butoxycarbonyl-piperazin-1-yl, 4-hydroxy-piperidin-1-yl, 3-hydroxy-piperidin-1-yl, 4-hydroxymethyl-piperidin-1-yl, 3-oxo-piperidin-1-yl, pyrrolidin-1-yl, 3-hydroxy-pyrrolidin-1-yl and 2-hydroxymethyl-pyrrolidin-1-yl; and

(eeeeee) R⁶ and R⁷ together with the nitrogen atom to which they are attached form a heterocyclic ring selected from morpholin-4-yl, piperazin-1-yl, 4-methyl-piperazin-1-yl, pyrrolidin-1-yl, 3-hydroxy-pyrrolidin-1-yl and 2-hydroxymethyl-pyrrolidin-1-yl.

An embodiment of the present invention is a quinazoline derivative of the formula I wherein:

m is 0;

R² is hydrogen;

n is 0 or 1;

each R³, which may be the same or different, is selected from halogeno, cyano, (1-4C)alkyl and (1-4C)alkoxy;

X¹ is selected from O and OC(R¹³)₂, wherein each R¹³, which may be the same or different, is hydrogen or (1-3C)alkyl;

Q¹ is heteroaryl,

and wherein Q¹ optionally bears one or more substituents, which may be the same or different, as hereinbefore defined (for example Q¹ optionally bears 1 or 2 substituents, which may be the same or different, selected from halogeno, cyano, hydroxy, (1-6C)alkyl, (1-6C)alkoxy, and a group of the formula —X²—R⁸ wherein X² is a direct bond and R⁸ is selected from halogeno-(1-4C)alkyl and hydroxy-(1-4C)alkyl);

R⁴ and R⁵, which may be the same or different, are selected from hydrogen and (1-6C)alkyl, and wherein any CH₂ or CH₃ group within any of R⁴ and R⁵ optionally bears on each said CH₂ or CH₃ group one or more substituents independently selected from halogeno, hydroxy, cyano, (1-6C)alkoxy, amino, (2-6C)alkanoyl, (1-6C)alkylamino and di-[(1-6C)alkylamino]; and

R⁶ and R⁷, which may be the same or different, are selected from hydrogen, (1-6C)alkyl, (2-6C)alkenyl, (2-6C)alkynyl, (3-7C)cycloalkyl, (3-7C)cycloalkyl-(1-6C)alkyl, (3-7C)cycloalkenyl, (3-7C)cycloalkenyl-(1-6C)alkyl, heterocyclyl and heterocyclyl-(1-6C)alkyl, or

R⁶ and R⁷ together with the nitrogen atom to which they are attached form a saturated 4, 5, 6 or 7 membered heterocyclic ring which optionally contains one or more additional heteroatoms independently selected from oxygen, S, SO, SO₂ and NR¹⁰, wherein R¹⁰ is selected from hydrogen, (1-6C)alkyl, (2-6C)alkenyl, (2-6C)alkynyl, (1-6C)alkylsulfonyl, (1-6C)alkylcarbonyl and (1-6C)alkoxycarbonyl,

and wherein any heterocyclyl group within an R⁶ or an R⁷ substituent or any heterocyclic ring formed by R⁶, R⁷ and the nitrogen atom to which they are attached optionally bears one or more substituents, which may be the same or different, selected from halogeno, trifluoromethyl, cyano, nitro, hydroxy, amino, formyl, mercapto, (1-6C)alkyl, (2-6C)alkenyl, (2-6C)alkynyl, hydroxy-(1-6C)alkyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X³—R¹¹

wherein X³ is a direct bond or is selected from O, CO, SO₂ and N(R¹²), wherein R¹² is hydrogen or (1-4C)alkyl, and R¹¹ is selected from halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl,

and wherein any heterocyclyl group within an R⁶ or an R⁷ substituent or any heterocyclic ring formed by R⁶, R⁷ and the nitrogen atom to which they are attached optionally bears 1 or 2 oxo or thioxo substituents;

and wherein any CH₂ or CH₃ group within an R⁶ or an R⁷ substituent, other than a CH₂ group within a heterocyclyl group or heterocyclic ring, optionally bears on each said CH₂ or CH₃ group one or more substituents independently selected from halogeno, (1-6C)alkyl, hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkylsulfonylamino and N-(1-6C)alkyl-(1-6C)alkylsulfonylamino;

or a pharmaceutically acceptable salt thereof.

Another embodiment of the present invention is a quinazoline derivative of the formula I wherein:

m is 0;

R² is hydrogen;

n is 0 or 1;

each R³, which may be the same or different, is selected from halogeno, cyano, (1-4C)alkyl and (1-4C)alkoxy;

X¹ is selected from O and OC(R¹³)₂, wherein each R¹³, which may be the same or different, is hydrogen or (1-3C)alkyl;

Q¹ is heteroaryl,

and wherein Q¹ optionally bears one or more substituents, which may be the same or different, as hereinbefore defined (for example Q¹ optionally bears 1 or 2 substituents, which may be the same or different, selected from halogeno, cyano, hydroxy, (1-6C)alkyl, (1-6C)alkoxy, and a group of the formula —X²—R⁸ wherein X² is a direct bond and R⁸ is selected from halogeno-(1-4C)alkyl and hydroxy-(1-4C)alkyl);

R⁴ and R⁵, which may be the same or different, are selected from hydrogen and (1-6C)alkyl, and wherein any CH₂ or CH₃ group within any of R⁴ and R⁵ optionally bears on each said CH₂ or CH₃ group one or more hydroxy substituents; and

R⁶ and R⁷, which may be the same or different, are selected from hydrogen, (1-6C)alkyl, (2-6C)alkenyl, (2-6C)alkynyl, (3-7C)cycloalkyl, heterocyclyl and heterocyclyl-(1-6C)alkyl, or

R⁶ and R⁷ together with the nitrogen atom to which they are attached form a saturated 4, 5, 6 or 7 membered heterocyclic ring which optionally contains one or more additional heteroatoms independently selected from oxygen, S, SO, SO₂ and NR¹⁰, wherein R¹⁰ is selected from hydrogen, (1-6C)alkyl and (1-6C)alkoxycarbonyl,

and wherein any heterocyclyl group within an R⁶ or an R⁷ substituent or any heterocyclic ring formed by R⁶, R⁷ and the nitrogen atom to which they are attached optionally bears one or more substituents, which may be the same or different, selected from halogeno, trifluoromethyl, cyano, nitro, hydroxy, amino, formyl, mercapto, (1-6C)alkyl, (2-6C)alkenyl, (2-6C)alkynyl, hydroxy-(1-6C)alkyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X³—R¹¹

wherein X³ is a direct bond or is selected from O, CO, SO₂ and N(R¹²), wherein R¹² is hydrogen or (1-4C)alkyl, and R¹¹ is selected from halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl,

and wherein any heterocyclyl group within an R⁶ or an R⁷ substituent or any heterocyclic ring formed by R⁶, R⁷ and the nitrogen atom to which they are attached optionally bears 1 or 2 oxo or thioxo substituents,

and wherein any CH₂ or CH₃ group within an R⁶ or an R⁷ substituent, other than a CH₂ group within a heterocyclyl group or heterocyclic ring, optionally bears on each said CH₂ or CH₃ group one or more substituents independently selected from halogeno, (1-6C)alkyl, hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkylsulfonylamino and N-(1-6C)alkyl-(1-6C)alkylsulfonylamino;

or a pharmaceutically acceptable salt thereof.

Another embodiment of the present invention is a quinazoline derivative of the formula I wherein:

m is 0;

R² is hydrogen;

n is 0 or 1 (particularly 1);

each R³, which may be the same or different, is selected from halogeno (such as chloro or fluoro), (1-4C)alkyl (such as methyl) and (1-4C)alkoxy (such as methoxy);

X¹ is selected from O and OC(R¹³)₂, wherein each R¹³ is hydrogen;

Q¹ is pyridinyl (such as pyridin-2-yl or pyridin-3-yl),

and wherein Q¹ optionally bears one substituent selected from (1-4C)alkyl (such as methyl) and a group of the formula:
—X²—R⁸

wherein X² is a direct bond and R⁸ is halogeno-(1-4C)alkyl (such as fluoromethyl);

R⁴ is hydrogen;

R⁵ is (1-4C)alkyl, wherein any CH₂ or CH₃ group within R⁵ optionally bears on each said CH₂ or CH₃ group one or more hydroxy substituent;

R⁶ and R⁷, which may be the same or different, are selected from hydrogen, (1-4C)alkyl and (3-6C)cycloalkyl, or

R⁶ and R⁷ together with the nitrogen atom to which they are attached form a saturated 4, 5 or 6 membered heterocyclic ring which optionally contains one additional oxygen heteroatom,

and wherein any heterocyclic ring formed by R⁶, R⁷ and the nitrogen atom to which they are attached optionally bears one substituent selected from hydroxy and from a group of the formula:
—X³—R¹¹

wherein X³ is a direct bond and R¹¹ is hydroxy-(1-4C)alkyl,

and wherein any heterocyclic ring formed by R⁶, R⁷ and the nitrogen atom to which they are attached optionally bears 1 oxo substituent;

and wherein any CH₂ or CH₃ group within an R⁶ or an R⁷ substituent, other than a CH₂ group within a heterocyclyl group or heterocyclic ring, optionally bears on each said CH₂ or CH₃ group one hydroxy substituent;

or a pharmaceutically acceptable salt thereof.

Another embodiment of the present invention is a quinazoline derivative of the formula I wherein:

m is 0;

R² is hydrogen;

n is 1;

R³ is selected from (1-4C)alkyl (such as methyl) and (1-4C)alkoxy (such as methoxy) (particularly R³ is (1-4C)alkyl);

X¹ is O;

Q¹ is pyridinyl (such as pyridin-2-yl or pyridin-3-yl), and wherein Q¹ bears one substituent selected from (1-4C)alkyl (such as methyl) and a group of the formula:
—X²—R⁸

wherein X² is a direct bond and R⁸ is halogeno-(1-4C)alkyl (such as fluoromethyl) (Q¹ particularly bears one (1-4C)alkyl substituent);

R⁴ is hydrogen;

R⁵ is (1-4C)alkyl;

R⁶ and R⁷, which may be the same or different, are selected from hydrogen and (1-4C)alkyl,

and wherein any CH₂ or CH₃ group within an R⁶ or an R⁷ substituent, optionally bears on each said CH₂ or CH₃ group one hydroxy substituent;

or a pharmaceutically acceptable salt thereof.

Another embodiment of the present invention is a quinazoline derivative of the formula I wherein:

m is 0;

R² is hydrogen;

n is 0 or 1;

each R³, which may be the same or different, is selected from halogeno (such as chloro or fluoro), cyano, (1-4C)alkyl (such as methyl) and (1-4C)alkoxy (such as methoxy);

X¹ is selected from O and OC(R¹³)₂, wherein each R¹³ is hydrogen;

Q¹ is pyridinyl (such as pyridin-2-yl or pyridin-3-yl),

and wherein Q¹ optionally bear one substituent selected from cyano and (1-4C)alkyl;

R⁴ and R⁵, which may be the same or different, are selected from hydrogen and (1-4C)alkyl;

R⁶ and R⁷, which may be the same or different, are selected from hydrogen, (1-4C)alkyl and (3-6C)cycloalkyl, or

R⁶ and R⁷ together with the nitrogen atom to which they are attached form a saturated 4, 5 or 6 membered heterocyclic ring which optionally contains one additional heteroatom independently selected from oxygen and NR¹⁰, wherein R¹⁰ is (1-4C)alkyl;

and wherein any heterocyclic ring formed by R⁶, R⁷ and the nitrogen atom to which they are attached optionally bears one substituent, selected from hydroxy and from a group of the formula:
—X³—R¹¹

wherein X³ is a direct bond and R¹¹ is hydroxy-(1-4C)alkyl,

and wherein any heterocyclic ring formed by R⁶, R⁷ and the nitrogen atom to which they are attached optionally bears 1 oxo substituent;

and wherein any CH₂ or CH₃ group within an R⁶ or an R⁷ substituent, other than a CH₂ group within a heterocyclyl group or heterocyclic ring, optionally bears on each said CH₂ or CH₃ group one or 2 substituents independently selected from hydroxy, (1-4C)alkoxy and (1-4C)alkylsulfonyl;

or a pharmaceutically acceptable salt thereof.

A particular embodiment of the quinazoline derivatives of the formula I is a quinazoline derivative of the formula Ia: 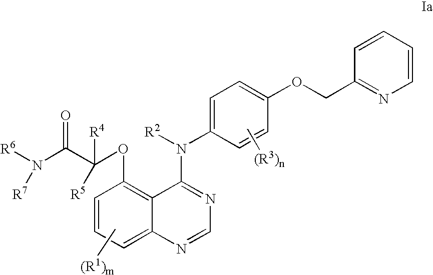
wherein:

m is 0, 1 or 2;

each R¹, which may be the same or different, is selected from hydroxy, (1-6C)alkoxy, (3-7C)cycloalkyl-oxy and (3-7C)cycloalkyl-(1-6C)alkoxy,

and wherein any CH₂ or CH₃ group within an R¹ substituent optionally bears on each said CH₂ or CH₃ group one or more substituents independently selected from halogeno, (1-6C)alkyl, hydroxy and (1-6C)alkoxy;

R² is hydrogen or (1-4C)alkyl;

n is 0, 1, 2, 3 or 4;

each R³, which may be the same or different, is selected from halogeno, cyano, (1-4C)alkyl, trifluoromethyl, (1-4C)alkoxy, (2-4C)alkenyl and (2-4C)alkynyl;

R⁴ and R⁵, which may be the same or different, are selected from hydrogen and (1-6C)alkyl, or

R⁴ and R⁵ together with the carbon atom to which they are attached form a (3-7C)cycloalkyl ring,

and wherein any CH₂ or CH₃ group within any of R⁴ and R⁵ optionally bears on each said CH₂ or CH₃ group one or more substituents independently selected from halogeno, hydroxy, cyano, (1-6C)alkoxy, amino, (2-6C)alkanoyl, (1-6C)alkylamino and di-[(1-6C)alkylamino];

R⁶ and R⁷, which may be the same or different, are selected from hydrogen, (1-6C)alkyl, (2-6C)alkenyl, (2-6C)alkynyl, (3-7C)cycloalkyl, (3-7C)cycloalkyl-(1-6C)alkyl, (3-7C)cycloalkenyl, (3-7C)cycloalkenyl-(1-6C)alkyl, heterocyclyl and heterocyclyl-(1-6C)alkyl, or

R⁶ and R⁷ together with the nitrogen atom to which they are attached form a saturated 4, 5, 6 or 7 membered heterocyclic ring which optionally contains one or more additional heteroatoms independently selected from oxygen, S, SO, SO₂ and NR¹⁰, wherein R¹⁰ is selected from hydrogen, (1-6C)alkyl, (2-6C)alkenyl, (2-6C)alkynyl, (1-6C)alkylsulfonyl, (1-6C)alkylcarbonyl and (1-6C)alkoxycarbonyl,

and wherein any heterocyclyl group within an R⁶ or an R⁷ substituent or any heterocyclic ring formed by R⁶, R⁷ and the nitrogen atom to which they are attached optionally bears one or more substituents, which may be the same or different, selected from halogeno, trifluoromethyl, cyano, nitro, hydroxy, amino, formyl, mercapto, (1-6C)alkyl, (2-6C)alkenyl, (2-6C)alkynyl, hydroxy-(1-6C)alkyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X³—R¹¹

wherein X³ is a direct bond or is selected from O, CO, SO₂ and N(R¹²), wherein R¹² is hydrogen or (1-4C)alkyl, and R¹¹ is selected from halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl,

and wherein any heterocyclyl group within an R⁶ or an R⁷ substituent or any heterocyclic ring formed by R⁶, R⁷ and the nitrogen atom to which they are attached optionally bears 1 or 2 oxo or thioxo substituents,

and wherein any CH₂ or CH₃ group within an R⁶ or an R⁷ substituent, other than a CH₂ group within a heterocyclyl group or heterocyclic ring, optionally bears on each said CH₂ or CH₃ group one or more substituents independently selected from halogeno, (1-6C)alkyl, hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkylsulfonylamino and N-(1-6C)alkyl-(1-6C)alkylsulfonylamino;

or a pharmaceutically acceptable salt thereof.

Another particular embodiment is a quinazoline derivative of the formula Ia wherein:

m is 0, 1 or 2;

each R¹, which may be the same or different, is selected from hydroxy, (1-6C)alkoxy, (3-7C)cycloalkyl-oxy and (3-7C)cycloalkyl-(1-6C)alkoxy,

and wherein any CH₂ or CH₃ group within an R¹ substituent optionally bears on each said CH₂ or CH₃ group one or more substituents independently selected from halogeno, (1-6C)alkyl, hydroxy and (1-6C)alkoxy,

R² is hydrogen or (1-4C)alkyl;

n is 0, 1, 2, 3 or 4;

each R³, which may be the same or different, is selected from halogeno, (1-4C)alkyl, trifluoromethyl, (1-4C)alkoxy, (2-4C)alkenyl and (2-4C)alkynyl;

R⁴ and R⁵, which may be the same or different, are selected from hydrogen and (1-6C)alkyl, or

R⁴ and R⁵ together with the carbon atom to which they are attached form a (3-7C)cycloalkyl ring,

and wherein any CH₂ or CH₃ group within any of R⁴ and R⁵ optionally bears on each said CH₂ or CH₃ group one or more substituents independently selected from halogeno, hydroxy, cyano, (1-6C)alkoxy, amino, (2-6C)alkanoyl, (1-6C)alkylamino and di-[(1-6C)alkylamino];

R⁶ and R⁷, which may be the same or different, are selected from hydrogen, (1-6C)alkyl, (2-6C)alkenyl, (2-6C)alkynyl, (3-7C)cycloalkyl, (3-7C)cycloalkyl-(1-6C)alkyl, (3-7C)cycloalkenyl, (3-7C)cycloalkenyl-(1-6C)alkyl, heterocyclyl and heterocyclyl-(1-6C)alkyl, or

R⁶ and R⁷ together with the nitrogen atom to which they are attached form a saturated 5 or 6 membered heterocyclic ring which optionally contains one or more additional heteroatoms independently selected from oxygen and NR¹⁰, wherein R¹⁰ is selected from hydrogen, (1-6C)alkyl, (2-6C)alkenyl, (2-6C)alkynyl, (1-6C)alkylsulfonyl and (1-6C)alkylcarbonyl,

and wherein any heterocyclyl group within an R⁶ or an R⁷ substituent or any heterocyclic ring formed by R⁶, R⁷ and the nitrogen atom to which they are attached optionally bears one or more substituents, which may be the same or different, selected from halogeno, trifluoromethyl, cyano, nitro, hydroxy, amino, formyl, mercapto, (1-6C)alkyl, (2-6C)alkenyl, (2-6C)alkynyl, hydroxy-(1-6C)alkyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X³—R¹¹

wherein X³ is a direct bond or is selected from O, CO, SO₂ and N(R¹²), wherein R¹² is hydrogen or (1-4C)alkyl, and R¹¹ is halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl,

and wherein any heterocyclyl group within an R⁶ or an R⁷ substituent or any heterocyclic ring formed by R⁶, R⁷ and the nitrogen atom to which they are attached optionally bears 1 or 2 oxo or thioxo substituents,

and wherein any CH₂ or CH₃ group within an R⁶ or an R⁷ substituent, other than a CH₂ group within a heterocyclyl group or a heterocyclic ring, optionally bears on each said CH₂ or CH₃ group one or more substituents independently selected from halogeno, (1-6C)alkyl, hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkylsulfonylamino and N-(1-6C)alkyl-(1-6C)alkylsulfonylamino;

or a pharmaceutically acceptable salt thereof.

Particularly, in the quinazoline derivatives of the formula Ia, n is 0, 1 or 2 (more particularly 0 or 1, even more particularly 1) and, when present, at least one R³ is in a meta-position (3-position) relative to the nitrogen of the anilino group in the formula Ia.

In one aspect of the quinazoline derivatives of the formula Ia, R³ may be selected from halogeno, (1-4C)alkyl, (1-4C)alkoxy and (2-4C)alkynyl, for example R³ may be selected from chloro and methyl.

In another aspect of the quinazoline derivatives of the formula Ia, R³ may be selected from halogeno, cyano, (1-4C)alkyl and (1-4C)alkoxy, for example R³ may be selected from chloro, fluoro, cyano, methyl and methoxy (particularly chloro and methyl).

Particularly, in the quinazoline derivatives of the formula Ia, m is 0 or 1 (for example m is 0) and R¹, when present, is located at the 7-position on the quinazoline ring in the formula Ia. When m is 1, R¹ is suitably located at the 7-position on the quinazoline ring and is selected from methoxy, ethoxy, propyloxy, isopropyloxy, cyclopropylmethoxy, 2-hydroxyethoxy, 2-fluoroethoxy, 2-methoxyethoxy, 2-ethoxyethoxy, trifluoromethoxy, 2,2-difluoroethoxy and 2,2,2-trifluoroethoxy (particularly methoxy).

Particularly, in the quinazoline derivatives of the formula Ia, R² is selected from hydrogen and methyl (more particularly hydrogen).

Particularly, in the quinazoline derivatives of the formula Ia, R⁴ and R⁵, which may be the same or different, are selected from hydrogen and (1-3C)alkyl, wherein any CH₂ or CH₃ group within any of R⁴ and R⁵ optionally bears on each said CH₂ or CH₃ group one or more (for example 1, 2 or 3) substituents independently selected from halogeno, hydroxy, cyano, (1-6C)alkoxy, amino, (2-6C)alkanoyl, (1-6C)alkylamino and di-[(1-6C)alkylamino] (particularly hydroxy).

More particularly, in the quinazoline derivatives of the formula Ia, (i) R⁴ and R⁵ are both hydrogen, (ii) R⁴ is hydrogen and R⁵ is (1-3C)alkyl, optionally substituted by hydroxy, or (iii) R⁴ and R⁵ are both methyl.

In one aspect of the quinazoline derivatives of the formula Ia, Q¹ may be selected from 2-pyridinyl, 3-pyridinyl, 4-pyridinyl, 6-methoxypyridin-3-yl, 6-cyanopyridin-3-yl, 6-methylpyridin-3-yl, 6-hydroxymethylpyridin-3-yl, 6-fluoromethylpyridin-3-yl, 6-fluoropyridin-3-yl, pyrazin-2-yl, 1,3-thiazol-2-yl, 1,3-thiazol-5-yl, pyrimidin-5-yl, pyridazin-3-yl and 1-methyl-1H-pyrazol-4-yl.

Another particular embodiment of the quinazoline derivatives of the formula I is a quinazoline derivative of the formula Ib: 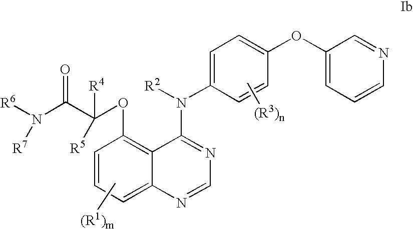
wherein:

m is 0, 1 or 2;

each R¹, which may be the same or different, is selected from hydroxy, (1-6C)alkoxy, (3-7C)cycloalkyl-oxy and (3-7C)cycloalkyl-(1-6C)alkoxy,

and wherein any CH₂ or CH₃ group within an R¹ substituent optionally bears on each said CH₂ or CH₃ group one or more substituents independently selected from halogeno, (1-6C)alkyl, hydroxy and (1-6C)alkoxy;

R² is hydrogen or (1-4C)alkyl;

n is 0, 1, 2, 3 or 4;

each R³, which may be the same or different, is selected from halogeno, cyano, (1-4C)alkyl, trifluoromethyl, (1-4C)alkoxy, (2-4C)alkenyl and (2-4C)alkynyl;

R⁴ and R⁵, which may be the same or different, are selected from hydrogen and (1-6C)alkyl, or

R⁴ and R⁵ together with the carbon atom to which they are attached form a (3-7C)cycloalkyl ring,

and wherein any CH₂ or CH₃ group within any of R⁴ and R⁵ optionally bears on each said CH₂ or CH₃ group one or more substituents independently selected from halogeno, hydroxy, cyano, (1-6C)alkoxy, amino, (2-6C)alkanoyl, (1-6C)alkylamino and di-[(1-6C)alkylamino];

R⁶ and R⁷, which may be the same or different, are selected from hydrogen, (1-6C)alkyl, (2-6C)alkenyl, (2-6C)alkynyl, (3-7C)cycloalkyl, (3-7C)cycloalkyl-(1-6C)alkyl, (3-7C)cycloalkenyl, (3-7C)cycloalkenyl-(1-6C)alkyl, heterocyclyl and heterocyclyl-(1-6C)alkyl, or

R⁶ and R⁷ together with the nitrogen atom to which they are attached form a saturated 4, 5, 6 or 7 membered heterocyclic ring which optionally contains one or more additional heteroatoms independently selected from oxygen, S, SO, SO₂ and NR¹⁰, wherein R¹⁰ is selected from hydrogen, (1-6C)alkyl, (2-6C)alkenyl, (2-6C)alkynyl, (1-6C)alkylsulfonyl, (1-6C)alkylcarbonyl and (1-6C)alkoxycarbonyl,

and wherein any heterocyclyl group within an R⁶ or an R⁷ substituent or any heterocyclic ring formed by R⁶, R⁷ and the nitrogen atom to which they are attached optionally bears one or more substituents, which may be the same or different, selected from halogeno, trifluoromethyl, cyano, nitro, hydroxy, amino, formyl, mercapto, (1-6C)alkyl, (2-6C)alkenyl, (2-6C)alkynyl, hydroxy-(1-6C)alkyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X³—R¹¹

wherein X³ is a direct bond or is selected from O, CO, SO₂ and N(R¹²), wherein R¹² is hydrogen or (1-4C)alkyl, and R¹¹ is selected from halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl,

and wherein any heterocyclyl group within an R⁶ or an R⁷ substituent or any heterocyclic ring formed by R⁶, R⁷ and the nitrogen atom to which they are attached optionally bears 1 or 2 oxo or thioxo substituents,

and wherein any CH₂ or CH₃ group within an R⁶ or an R⁷ substituent, other than a CH₂ group within a heterocyclyl group or heterocyclic ring, optionally bears on each said CH₂ or CH₃ group one or more substituents independently selected from halogeno, (1-6C)alkyl, hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkylsulfonylamino and N-(1-6C)alkyl-(1-6C)alkylsulfonylamino;

or a pharmaceutically acceptable salt thereof.

Another particular embodiment is a quinazoline derivative of the formula Ib wherein:

m is 0, 1 or 2;

each R¹, which may be the same or different, is selected from hydroxy, (1-6C)alkoxy, (3-7C)cycloalkyl-oxy and (3-7C)cycloalkyl-(1-6C)alkoxy,

and wherein any CH₂ or CH₃ group within an R¹ substituent optionally bears on each said CH₂ or CH₃ group one or more substituents independently selected from halogeno, (1-6C)alkyl, hydroxy and (1-6C)alkoxy,

R² is hydrogen or (1-4C)alkyl;

n is 0, 1, 2, 3 or 4;

each R³, which may be the same or different, is selected from halogeno, (1-4C)alkyl, trifluoromethyl, (1-4C)alkoxy, (2-4C)alkenyl and (2-4C)alkynyl;

R⁴ and R⁵, which may be the same or different, are selected from hydrogen and (1-6C)alkyl, or

R⁴ and R⁵ together with the carbon atom to which they are attached form a (3-7C)cycloalkyl ring,

and wherein any CH₂ or CH₃ group within any of R⁴ and R⁵ optionally bears on each said CH₂ or CH₃ group one or more substituents independently selected from halogeno, hydroxy, cyano, (1-6C)alkoxy, amino, (2-6C)alkanoyl, (1-6C)alkylamino and di-[(1-6C)alkylamino],

R⁶ and R⁷, which may be the same or different, are selected from hydrogen, (1-6C)alkyl, (2-6C)alkenyl, (2-6C)alkynyl, (3-7C)cycloalkyl, (3-7C)cycloalkyl-(1-6C)alkyl, (3-7C)cycloalkenyl, (3-7C)cycloalkenyl-(1-6C)alkyl, heterocyclyl and heterocyclyl-(1-6C)alkyl, or

R⁶ and R⁷ together with the nitrogen atom to which they are attached form a saturated 5 or 6 membered heterocyclic ring which optionally contains one or more additional heteroatoms independently selected from oxygen and NR¹⁰, wherein R¹⁰ is selected from hydrogen, (1-6C)alkyl, (2-6C)alkenyl, (2-6C)alkynyl, (1-6C)alkylsulfonyl and (1-6C)alkylcarbonyl,

and wherein any heterocyclyl group within an R⁶ or an R⁷ substituent or any heterocyclic ring formed by R⁶, R⁷ and the nitrogen atom to which they are attached optionally bears one or more substituents, which may be the same or different, selected from halogeno, trifluoromethyl, cyano, nitro, hydroxy, amino, formyl, mercapto, (1-6C)alkyl, (2-6C)alkenyl, (2-6C)alkynyl, hydroxy-(1-6C)alkyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X³—R¹¹

wherein X³ is a direct bond or is selected from O, CO, SO₂ and N(R¹²), wherein R¹² is hydrogen or (1-4C)alkyl, and R¹¹ is halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl,

and wherein any heterocyclyl group within an R⁶ or an R⁷ substituent or any heterocyclic ring formed by R⁶, R⁷ and the nitrogen atom to which they are attached optionally bears 1 or 2 oxo or thioxo substituents,

and wherein any CH₂ or CH₃ group within an R⁶ or an R⁷ substituent, other than a CH₂ group within a heterocyclyl group or a heterocyclic ring, optionally bears on each said CH₂ or CH₃ group one or more substituents independently selected from halogeno, (1-6C)alkyl, hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkylsulfonylamino and N-(1-6C)alkyl-(1-6C)alkylsulfonylamino;

or a pharmaceutically acceptable salt thereof.

Particularly, in the quinazoline derivatives of the formula Ib, n is 0, 1 or 2 (more particularly 0 or 1, even more particularly 1) and, when present, at least one R³ is in a meta-position (3-position) relative to the nitrogen of the anilino group in the formula Ib.

In one aspect of the quinazoline derivatives of the formula Ib, R³ may be selected from halogeno, (1-4C)alkyl, (1-4C)alkoxy and (2-4C)alkynyl, for example R³ may be selected from chloro and methyl (particularly methyl).

In another aspect of the c quinazoline derivatives of the formula Ib, R³ may be selected from halogeno, cyano, (1-4C)alkyl and (1-4C)alkoxy, for example R³ may be selected from chloro, fluoro, cyano, methyl and methoxy (particularly chloro and methyl).

Particularly, in the quinazoline derivatives of the formula Ib, m is 0 or 1 (for example m is 0) and R¹, when present, is located at the 7-position on the quinazoline ring in the formula Ib. When m is 1, R¹ is suitably located at the 7-position on the quinazoline ring and is selected from methoxy, ethoxy, propyloxy, isopropyloxy, cyclopropylmethoxy, 2-hydroxyethoxy, 2-fluoroethoxy, 2-methoxyethoxy, 2-ethoxyethoxy, trifluoromethoxy, 2,2-difluoroethoxy and 2,2,2-trifluoroethoxy (particularly methoxy).

Particularly, in the quinazoline derivatives of the formula Ib, R² is selected from hydrogen and methyl (more particularly hydrogen).

Particularly, in the quinazoline derivatives of the formula Ib, R⁴ and R⁵, which may be the same or different, are selected from hydrogen and (1-3C)alkyl, wherein any CH₂ or CH₃ group within any of R⁴ and R⁵ optionally bears on each said CH₂ or CH₃ group one or more (for example 1, 2 or 3) substituents independently selected from halogeno, hydroxy, cyano, (1-6C)alkoxy, amino, (2-6C)alkanoyl, (1-6C)alkylamino and di-[(1-6C)alkylamino].

More particularly, in the quinazoline derivatives of the formula Ib, (i) R⁴ and R⁵ are both hydrogen, (ii) R⁴ is hydrogen and R⁵ is (1-3C)alkyl, optionally substituted by hydroxy, or (iii) R⁴ and R⁵ are both methyl.

In one aspect of the quinazoline derivatives of the formula Ib, Q¹ may be selected from 2-pyridinyl, 3-pyridinyl, 4-pyridinyl, 6-methoxypyridin-3-yl, 6-cyanopyridin-3-yl, 6-methylpyridin-3-yl, 6-hydroxymethylpyridin-3-yl, 6-fluoromethylpyridin-3-yl, 6-fluoropyridin-3-yl, pyrazin-2-yl, 1,3-thiazol-2-yl, 1,3-thiazol-5-yl, pyrimidin-5-yl, pyridazin-3-yl and 1-methyl-1H-pyrazol-4-yl.

Another particular embodiment of the quinazoline derivatives of the formula I is a quinazoline derivative of the formula Ic: 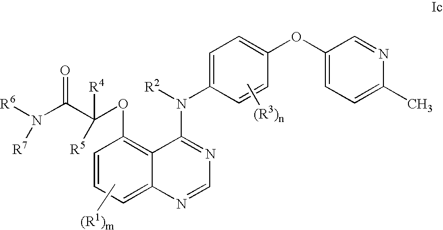
wherein:

m is 0, 1 or 2;

each R¹, which may be the same or different, is selected from hydroxy, (1-6C)alkoxy, (3-7C)cycloalkyl-oxy and (3-7C)cycloalkyl-(1-6C)alkoxy,

and wherein any CH₂ or CH₃ group within an R¹ substituent optionally bears on each said CH₂ or CH₃ group one or more substituents independently selected from halogeno, (1-6C)alkyl, hydroxy and (1-6C)alkoxy;

R² is hydrogen or (1-4C)alkyl;

n is 0, 1, 2, 3 or 4;

each R³, which may be the same or different, is selected from halogeno, cyano, (1-4C)alkyl, trifluoromethyl, (1-4C)alkoxy, (2-4C)alkenyl and (2-4C)alkynyl;

R⁴ and R⁵, which may be the same or different, are selected from hydrogen and (1-6C)alkyl, or

R⁴ and R⁵ together with the carbon atom to which they are attached form a (3-7C)cycloalkyl ring,

and wherein any CH₂ or CH₃ group within any of R⁴ and R⁵ optionally bears on each said CH₂ or CH₃ group one or more substituents independently selected from halogeno, hydroxy, cyano, (1-6C)alkoxy, amino, (2-6C)alkanoyl, (1-6C)alkylamino and di-[(1-6C)alkylamino];

R⁶ and R⁷, which may be the same or different, are selected from hydrogen, (1-6C)alkyl, (2-6C)alkenyl, (2-6C)alkynyl, (3-7C)cycloalkyl, (3-7C)cycloalkyl-(1-6C)alkyl, (3-7C)cycloalkenyl, (3-7C)cycloalkenyl-(1-6C)alkyl, heterocyclyl and heterocyclyl-(1-6C)alkyl, or

R⁶ and R⁷ together with the nitrogen atom to which they are attached form a saturated 4, 5, 6 or 7 membered heterocyclic ring which optionally contains one or more additional heteroatoms independently selected from oxygen, S, SO, SO₂ and NR¹⁰, wherein R¹⁰ is selected from hydrogen, (1-6C)alkyl, (2-6C)alkenyl, (2-6C)alkynyl, (1-6C)alkylsulfonyl, (1-6C)alkylcarbonyl and (1-6C)alkoxycarbonyl,

and wherein any heterocyclyl group within an R⁶ or an R⁷ substituent or any heterocyclic ring formed by R⁶, R⁷ and the nitrogen atom to which they are attached optionally bears one or more substituents, which may be the same or different, selected from halogeno, trifluoromethyl, cyano, nitro, hydroxy, amino, formyl, mercapto, (1-6C)alkyl, (2-6C)alkenyl, (2-6C)alkynyl, hydroxy-(1-6C)alkyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X³—R¹¹

wherein X³ is a direct bond or is selected from O, CO, SO₂ and N(R¹²), wherein R¹² is hydrogen or (1-4C)alkyl, and R¹¹ is selected from halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl,

and wherein any heterocyclyl group within an R⁶ or an R⁷ substituent or any heterocyclic ring formed by R⁶, R⁷ and the nitrogen atom to which they are attached optionally bears 1 or 2 oxo or thioxo substituents,

and wherein any CH₂ or CH₃ group within an R⁶ or an R⁷ substituent, other than a CH₂ group within a heterocyclyl group or heterocyclic ring, optionally bears on each said CH₂ or CH₃ group one or more substituents independently selected from halogeno, (1-6C)alkyl, hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkylsulfonylamino and N-(1-6C)alkyl-(1-6C)alkylsulfonylamino;

or a pharmaceutically acceptable salt thereof.

Another particular embodiment is a quinazoline derivative of the formula Ic wherein:

m is 0, 1 or 2;

each R¹, which may be the same or different, is selected from hydroxy, (1-6C)alkoxy, (3-7C)cycloalkyl-oxy and (3-7C)cycloalkyl-(1-6C)alkoxy,

and wherein any CH₂ or CH₃ group within an R¹ substituent optionally bears on each said CH₂ or CH₃ group one or more substituents independently selected from halogeno, (1-6C)alkyl, hydroxy and (1-6C)alkoxy,

R² is hydrogen or (1-4C)alkyl;

n is 0, 1, 2, 3 or 4;

each R³, which may be the same or different, is selected from halogeno, (1-4C)alkyl, trifluoromethyl, (1-4C)alkoxy, (2-4C)alkenyl and (2-4C)alkynyl;

R⁴ and R⁵, which may be the same or different, are selected from hydrogen and (1-6C)alkyl, or

R⁴ and R⁵ together with the carbon atom to which they are attached form a (3-7C)cycloalkyl ring,

and wherein any CH₂ or CH₃ group within any of R⁴ and R⁵ optionally bears on each said CH₂ or CH₃ group one or more substituents independently selected from halogeno, hydroxy, cyano, (1-6C)alkoxy, amino, (2-6C)alkanoyl, (1-6C)alkylamino and di-[(1-6C)alkylamino],

R⁶ and R⁷, which may be the same or different, are selected from hydrogen, (1-6C)alkyl, (2-6C)alkenyl, (2-6C)alkynyl, (3-7C)cycloalkyl, (3-7C)cycloalkyl-(1-6C)alkyl, (3-7C)cycloalkenyl, (3-7C)cycloalkenyl-(1-6C)alkyl, heterocyclyl and heterocyclyl-(1-6C)alkyl, or

R⁶ and R⁷ together with the nitrogen atom to which they are attached form a saturated 5 or 6 membered heterocyclic ring which optionally contains one or more additional heteroatoms independently selected from oxygen and NR¹⁰, wherein R¹⁰ is selected from hydrogen, (1-6C)alkyl, (2-6C)alkenyl, (2-6C)alkynyl, (1-6C)alkylsulfonyl and (1-6C)alkylcarbonyl,

and wherein any heterocyclyl group within an R⁶ or an R⁷ substituent or any heterocyclic ring formed by R⁶, R⁷ and the nitrogen atom to which they are attached optionally bears one or more substituents, which may be the same or different, selected from halogeno, trifluoromethyl, cyano, nitro, hydroxy, amino, formyl, mercapto, (1-6C)alkyl, (2-6C)alkenyl, (2-6C)alkynyl, hydroxy-(1-6C)alkyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X³—R¹¹

wherein X³ is a direct bond or is selected from O, CO, SO₂ and N(R¹²), wherein R¹² is hydrogen or (1-4C)alkyl, and R¹¹ is halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl,

and wherein any heterocyclyl group within an R⁶ or an R⁷ substituent or any heterocyclic ring formed by R⁶, R⁷ and the nitrogen atom to which they are attached optionally bears 1 or 2 oxo or thioxo substituents;

and wherein any CH₂ or CH₃ group within an R⁶ or an R⁷ substituent, other than a CH₂ group within a heterocyclyl group or a heterocyclic ring, optionally bears on each said CH₂ or CH₃ group one or more substituents independently selected from halogeno, (1-6C)alkyl, hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkylsulfonylamino and N-(1-6C)alkyl-(1-6C)alkylsulfonylamino;

or a pharmaceutically acceptable salt thereof.

Particularly, in the quinazoline derivatives of the formula Ic, n is 0, 1 or 2 (more particularly 0 or 1, even more particularly 1) and, when present, at least one R³ is in a meta-position (3-position) relative to the nitrogen of the anilino group in the formula Ic.

In one aspect of the quinazoline derivatives of the formula Ic, R³ may be selected from halogeno, (1-4C)alkyl, (1-4C)alkoxy and (2-4C)alkynyl, for example R³ may be selected from chloro and methyl (particularly methyl).

In another aspect of the quinazoline derivatives of the formula Ic, R³ may be selected from halogeno, cyano, (1-4C)alkyl and (1-4C)alkoxy, for example R³ may be selected from chloro, fluoro, cyano, methyl and methoxy (particularly chloro and methyl).

Particularly, in the quinazoline derivatives of the formula Ic, m is 0 or 1 (for example m is 0) and R¹, when present, is located at the 7-position on the quinazoline ring in the formula Ic. When m is 1, R¹ is suitably located at the 7-position on the quinazoline ring and is selected from methoxy, ethoxy, propyloxy, isopropyloxy, cyclopropylmethoxy, 2-hydroxyethoxy, 2-fluoroethoxy, 2-methoxyethoxy, 2-ethoxyethoxy, trifluoromethoxy, 2,2-difluoroethoxy and 2,2,2-trifluoroethoxy (particularly methoxy).

Particularly, in the quinazoline derivatives of the formula Ic, R² is selected from hydrogen and methyl (more particularly hydrogen).

Particularly, in the quinazoline derivatives of the formula Ic, R⁴ and R⁵, which may be the same or different, are selected from hydrogen and (1-3C)alkyl, wherein any CH₂ or CH₃ group within any of R⁴ and R⁵ optionally bears on each said CH₂ or CH₃ group one or more (for example 1, 2 or 3) substituents independently selected from halogeno, hydroxy, cyano, (1-6C)alkoxy, amino, (2-6C)alkanoyl, (1-6C)alkylamino and di-[(1-6C)alkylamino].

More particularly, in the quinazoline derivatives of the formula Ic, (i) R⁴ and R⁵ are both hydrogen, (ii) R⁴ is hydrogen and R⁵ is (1-3C)alkyl, optionally substituted by hydroxy, or (iii) R⁴ and R⁵ are both methyl.

In one aspect of the quinazoline derivatives of the formula Ic, Q¹ may be selected from 2-pyridinyl, 3-pyridinyl, 4-pyridinyl, 6-methoxypyridin-3-yl, 6-cyanopyridin-3-yl, 6-methylpyridin-3-yl, 6-hydroxymethylpyridin-3-yl, 6-fluoromethylpyridin-3-yl, 6-fluoropyridin-3-yl, pyrazin-2-yl, 1,3-thiazol-2-yl, 1,3-thiazol-5-yl, pyrimidin-5-yl, pyridazin-3-yl and 1-methyl-1H-pyrazol-4-yl.

A particular quinazoline derivative of the invention is, for example, any one or more of the quinazoline derivatives of the formula I selected from:
2-[(4-{[3-chloro-4-(pyridin-2-ylmethoxy)phenyl]amino}quinazolin-5-yl)oxy]acetamide;
2-{4-[3-chloro-4-(pyridin-2-ylmethoxy)-phenylamino]-quinazolin-5-yloxy}-N-(2-methanesulfonyl-ethyl)-acetamide;
2-{4-[3-chloro-4-(pyridin-2-ylmethoxy)-phenylamino]-quinazolin-5-yloxy}-N-cyclopropyl-acetamide;
2-{4-[3-chloro-4-(pyridin-2-ylmethoxy)-phenylamino]-quinazolin-5-yloxy}-N-cyclobutyl-acetamide;
2-{4-[3-chloro-4-(pyridin-2-ylmethoxy)-phenylamino]-quinazolin-5-yloxy}-N-(2-methoxy-ethyl)-acetamide;
2-{4-[3-chloro-4-(pyridin-2-ylmethoxy)-phenylamino]-quinazolin-5-yloxy}-N-ethyl-acetamide;
N-allyl-2-{4-[3-chloro-4-(pyridin-2-ylmethoxy)-phenylamino]-quinazolin-5-yloxy}-acetamide;
2-{4-[3-chloro-4-(pyridin-2-ylmethoxy)-phenylamino]-quinazolin-5-yloxy}-N-ethyl-N-methyl-acetamide;
2-[(4-{[3-chloro-4-(pyridin-2-ylmethoxy)phenyl]amino}quinazolin-5-yl)oxy]-N-(2-morpholin-4-ylethyl)acetamide;
2-{4-[3-chloro-4-(pyridin-2-ylmethoxy)-phenylamino]-quinazolin-5-yloxy}-N-methyl-N-prop-2-ynyl-acetamide;
2-[(4-{[3-chloro-4-(pyridin-2-ylmethoxy)phenyl]amino}quinazolin-5-yl)oxy]-N-(2-hydroxyethyl)-N-methylacetamide;
2-{4-[3-chloro-4-(pyridin-2-ylmethoxy)-phenylamino]-quinazolin-5-yloxy}-N-(2-methanesulfonyl-ethyl)-N-methyl-acetamide;
2-{4-[3-chloro-4-(pyridin-2-ylmethoxy)-phenylamino]-quinazolin-5-yloxy}-N-methyl-N-(1-methyl-piperidin-4-yl)-acetamide;
2-{4-[3-chloro-4-(pyridin-2-ylmethoxy)-phenylamino]-quinazolin-5-yloxy}-N-isopropyl-N-methyl-acetamide;
2-{4-[3-chloro-4-(pyridin-2-ylmethoxy)-phenylamino]-quinazolin-5-yloxy}-N-(2-dimethylamino-ethyl)-N-methyl-acetamide;
N-[3-chloro-4-(pyridin-2-ylmethoxy)phenyl]-5-(2-morpholin-4-yl-2-oxoethoxy)quinazolin-4-amine;
N-[3-chloro-4-(pyridin-2-ylmethoxy)phenyl]-5-(2-oxo-2-piperazin-1-ylethoxy)quinazolin-4-amine;
N-[3-chloro-4-pyridin-2-ylmethoxy)phenyl]-5-[2-(4-methylpiperazin-1-yl)-2-oxoethoxy]quinazolin-4-amine;
(2R)-2-[(4-{[3-chloro-4-(pyridin-2-ylmethoxy)phenyl]amino}quinazolin-5-yl)oxy]propanamide;
(2R)-2-[(4-{[3-chloro-4-(pyridin-2-ylmethoxy)phenyl]amino}quinazolin-5-yl)oxy]-N-methylpropanamide;
(2R)-2-[(4-{[3-chloro-4-(pyridin-2-ylmethoxy)phenyl]amino}quinazolin-5-yl)oxy]-N,N-dimethylpropanamide;
(2R)-2-[(4-{[3-chloro-4-(pyridin-2-ylmethoxy)phenyl]amino}quinazolin-5-yl)oxy]-N-(2-hydroxyethyl)-N-methylpropanamide;
N-[3-chloro-4-(pyridin-2-ylmethoxy)phenyl]-5-[(1R)-1-methyl-2-oxo-2-pyrrolidin-1-ylethoxy]quinazolin-4-amine;
(3R)-1-{(2R)-2-[(4-{[3-chloro-4-(pyridin-2-ylmethoxy)phenyl]amino}quinazolin-5-yl)oxy]propanoyl}pyrrolidin-3-ol;
((2S)-1-{(2R)-2-[(4-{[3-chloro-4-(pyridin-2-ylmethoxy)phenyl]amino}quinazolin-5-yl)oxy]propanoyl}pyrrolidin-2-yl)methanol;
((2R)-1-{(2R)-2-[(4-{[3-chloro-4-(pyridin-2-ylmethoxy)phenyl]amino}quinazolin-5-yl)oxy]propanoyl}pyrrolidin-2-yl)methanol;
N-[3-chloro-4-(pyridin-2-ylmethoxy)phenyl]-5-[(1R)-1-methyl-2-morpholin-4-yl-2-oxoethoxy]quinazolin-4-amine;
(2S)-2-[(4-{[3-chloro-4-(pyridin-2-ylmethoxy)phenyl]amino}quinazolin-5-yl)oxy]-propanamide;
(2S)-2-[(4-{[3-chloro-4-(pyridin-2-ylmethoxy)phenyl]amino}quinazolin-5-yl)oxy]-N-methylpropanamide;
(2S)-2-[(4-{[3-chloro-4-(pyridin-2-ylmethoxy)phenyl]amino}quinazolin-5-yl)oxy]-N,N-dimethylpropanamide;
(2S)-2-[(4-{[3-chloro-4-(pyridin-2-ylmethoxy)phenyl]amino}quinazolin-5-yl)oxy]-N-(2-hydroxyethyl)-N-methylpropanamide;
(3R)-1-{(2S)-2-[(4-{[3-chloro-4-(pyridin-2-ylmethoxy)phenyl]amino}quinazolin-5-yl)oxy]propanoyl}pyrrolidin-3-ol;
(3S)-1-{(2S)-2-[(4-{[3-chloro-4-(pyridin-2-ylmethoxy)phenyl]amino}quinazolin-5-yl)oxy]propanoyl}pyrrolidin-3-ol;
((2S)-1-{(2S)-2-[(4-{[3-chloro-4-(pyridin-2-ylmethoxy)phenyl]amino}quinazolin-5-yl)oxy]propanoyl}pyrrolidin-2-yl)methanol;
(2R)-2-[(4-{[3-chloro-4-(pyridin-2-ylmethoxy)phenyl]amino}quinazolin-5-yl)oxy]-4-hydroxy-N-methylbutanamide;
(2R)-2-[(4-{[3-chloro-4-(pyridin-2-ylmethoxy)phenyl]amino}quinazolin-5-yl)oxy]-4-hydroxy-N-(2-hydroxy-1,1-dimethylethyl)butanamide;
(2R)-2-[(4-{[3-chloro-4-(pyridin-2-ylmethoxy)phenyl]amino}quinazolin-5-yl)oxy]-4-hydroxy-N,N-dimethylbutanamide;
(2R)-2-[(4-{[3-chloro-4-(pyridin-2-ylmethoxy)phenyl]amino}quinazolin-5-yl)oxy]-4-hydroxy-N-(2-hydroxyethyl)-N-methylbutanamide;
(3R)-3-[(4-{[3-chloro-4-(pyridin-2-ylmethoxy)phenyl]amino}quinazolin-5-yl)oxy]-4-morpholin-4-yl-4-oxobutan-1-ol;
(3R)-3-[(4-{[3-chloro-4-(pyridin-2-ylmethoxy)phenyl]amino}quinazolin-5-yl)oxy]-4-oxo-4-pyrrolidin-1-ylbutan-1-ol;
(3R)-3-[(4-{[3-chloro-4-(pyridin-2-ylmethoxy)phenyl]amino}quinazolin-5-yl)oxy]-4-(4-methylpiperazin-1-yl)-4-oxobutan-1-ol;
2-[(4-{[3-chloro-4-(pyridin-2-ylmethoxy)phenyl]amino}quinazolin-5-yl)oxy]-2-methylpropanamide;
2-[(4-{[3-chloro-4-(pyridin-2-ylmethoxy)phenyl]amino}quinazolin-5-yl)oxy]-N,2-dimethylpropanamide;
2-[(4-{[3-chloro-4-(pyridin-2-ylmethoxy)phenyl]amino}quinazolin-5-yl)oxy]-N-(2-hydroxy-1,1-dimethylethyl)-2-methylpropanamide;
2-[(4-{[3-chloro-4-(pyridin-2-ylmethoxy)phenyl]amino}quinazolin-5-yl)oxy]-N-(2-hydroxyethyl)-2-methylpropanamide;
2-[(4-{[3-chloro-4-(pyridin-2-ylmethoxy)phenyl]amino}quinazolin-5-yl)oxy]-N,N-bis(2-hydroxyethyl)-2-methylpropanamide;
2-[(4-{[3-chloro-4-(pyridin-2-ylmethoxy)phenyl]amino}quinazolin-5-yl)oxy]-N-(2-hydroxyethyl)-N,2-dimethylpropanamide;
(3R)-1-{2-[(4-{[3-chloro-4-(pyridin-2-ylmethoxy)phenyl]amino}quinazolin-5-yl)oxy]-2-methylpropanoyl}pyrrolidin-3-ol;
N-(2-hydroxyethyl)-2-methyl-2-[(4-{[3-methyl-4-(pyridin-2-ylmethoxy)phenyl]amino}quinazolin-5-yl)oxy]propanamide;
N,2-dimethyl-2-[(4-{[3-methyl-4-(pyridin-2-ylmethoxy)phenyl]amino}quinazolin-5-yl)oxy]propanamide;
2-{[4-({3-methyl-4-[(6-methylpyridin-3-yl)oxy]phenyl}amino)quinazolin-5-yl]oxy}acetamide;
N-(2-hydroxyethyl)-2-{[4-({3-methyl-4-[(6-methylpyridin-3-yl)oxy]phenyl}amino)quinazolin-5-yl]oxy}acetamide;
N-methyl-2-{[4-({3-methyl-4-[(6-methylpyridin-3-yl)oxy]phenyl}amino)quinazolin-5-yl]oxy}acetamide;
N-(2-hydroxyethyl)-N-methyl-2-{[4-({3-methyl-4-[(6-methylpyridin-3-yl)oxy]phenyl}amino)quinazolin-5-yl]oxy}acetamide;
N-{3-methyl-4-[(6-methylpyridin-3-yl)oxy]phenyl}-5-(2-oxo-2-pyrrolidin-1-ylethoxy)quinazolin-4-amine;
N-{3-methyl-4-[(6-methylpyridin-3-yl)oxy]phenyl}-5-(2-oxo-2-piperazin-1-ylethoxy)quinazolin-4-amine;
N-{3-methyl-4-[(6-methylpyridin-3-yl)oxy]phenyl}-5-[2-(4-methylpiperazin-1-yl)-2-oxoethoxy]quinazolin-4-amine;
(2S)-2-{[4-({3-methyl-4-[(6-methylpyridin-3-yl)oxy]phenyl}amino)quinazolin-5-yl]oxy}propanamide;
(2R)-2-{[4-({3-methyl-4-[(6-methylpyridin-3-yl)oxy]phenyl}amino)quinazolin-5-yl]oxy}propanamide;
(2R)—N-(2-hydroxyethyl)-N-methyl-2-{[4-({3-methyl-4-[(6-methylpyridin-3-yl)oxy]phenyl}amino)quinazolin-5-yl]oxy}propanamide;
2-methyl-2-{[4-({3-methyl-4-[(6-methylpyridin-3-yl)oxy]phenyl}amino)quinazolin-5-yl]oxy}propanamide;
N,2-dimethyl-2-{[4-({3-methyl-4-[(6-methylpyridin-3-yl)oxy]phenyl}amino)quinazolin-5-yl]oxy}propanamide;
(3R)-1-{(2S)-2-[(4-{[3-chloro-4-(pyridin-2-ylmethoxy)phenyl]amino}quinazolin-5-yl)oxy]propanoyl}pyrrolidin-3-ol;
(3S)-1-{(2S)-2-[(4-{[3-chloro-4-(pyridin-2-ylmethoxy)phenyl]amino}quinazolin-5-yl)oxy]propanoyl}pyrrolidin-3-ol;
(3R)-1-{(2R)-2-[(4-{[3-methyl-4-(pyridin-2-ylmethoxy)phenyl]amino}quinazolin-5-yl)oxy]propanoyl}pyrrolidin-3-ol;
(2R)—N-methyl-2-[(4-{[3-methyl-4-(pyridin-2-ylmethoxy)phenyl]amino}quinazolin-5-yl)oxy]propanamide;
(2R)—N-(2-hydroxyethyl)-N-methyl-2-[(4-{[3-methyl-4-(pyridin-2-ylmethoxy)phenyl]amino}quinazolin-5-yl)oxy]propanamide;
5-[(1R)-1-methyl-2-oxo-2-pyrrolidin-1-ylethoxy]-N-[3-methyl-4-(pyridin-2-ylmethoxy)phenyl]quinazolin-4-amine;
2-methyl-2-[(4-{[3-methyl-4-(pyridin-2-ylmethoxy)phenyl]amino}quinazolin-5-yl)oxy]propanamide;
N-(2-hydroxyethyl)-2-methyl-2-{[4-({3-methyl-4-[(6-methylpyridin-3-yl)oxy]phenyl}amino)quinazolin-5-yl]oxy}propanamide;
N-(2-hydroxyethyl)-N,2-dimethyl-2-{[4-({3-methyl-4-[(6-methylpyridin-3-yl)oxy]phenyl}amino)quinazolin-5-yl]oxy}propanamide;
(2S)—N-methyl-2-{[4-({3-methyl-4-[(6-methylpyridin-3-yl)oxy]phenyl}amino)quinazolin-5-yl]oxy}propanamide;
(2S)—N-(2-hydroxyethyl)-2-{[4-({3-methyl-4-[(6-methylpyridin-3-yl)oxy]phenyl}amino)quinazolin-5-yl]oxy}propanamide;
(2S)—N-(2-hydroxyethyl)-N-methyl-2-{[4-({3-methyl-4-[(6-methylpyridin-3-yl)oxy]phenyl}amino)quinazolin-5-yl]oxy}propanamide;
N-{3-methyl-4-[(6-methylpyridin-3-yl)oxy]phenyl}-5-[(1S)-1-methyl-2-morpholin-4-yl-2-oxoethoxy]quinazolin-4-amine;
(3S)-1-((2S)-2-{[4-({3-methyl-4-[(6-methylpyridin-3-yl)oxy]phenyl}amino)quinazolin-5-yl]oxy}propanoyl)pyrrolidin-3-ol;
(3S)-1-((2R)-2-{[4-({3-methyl-4-[(6-methylpyridin-3-yl)oxy]phenyl}amino)quinazolin-5-yl]oxy}propanoyl)pyrrolidin-3-ol;
(3R)-1-((2R)-2-{[4-({3-methyl-4-[(6-methylpyridin-3-yl)oxy]phenyl}amino)quinazolin-5-yl]oxy}propanoyl)pyrrolidin-3-ol;
(2R)—N-methyl-2-{[4-({3-methyl-4-[(6-methylpyridin-3-yl)oxy]phenyl}amino)quinazolin-5-yl]oxy}propanamide;
(2R)—N-(2-hydroxyethyl)-2-{[4-({3-methyl-4-[(6-methylpyridin-3-yl)oxy]phenyl}amino)quinazolin-5-yl]oxy}propanamide;
(2R)—N,N-dimethyl-2-{[4-({3-methyl-4-[(6-methylpyridin-3-yl)oxy]phenyl}amino)quinazolin-5-yl]oxy}propanamide;
(2R)—N-isopropyl-2-{[4-({3-methyl-4-[(6-methylpyridin-3-yl)oxy]phenyl}amino)quinazolin-5-yl]oxy}propanamide;
(2R)—N-ethyl-2-{[4-({3-methyl-4-[(6-methylpyridin-3-yl)oxy]phenyl}amino)quinazolin-5-yl]oxy}propanamide;
(2R)—N-[2-(diethylamino)ethyl]-2-{[4-({3-methyl-4-[(6-methylpyridin-3-yl)oxy]phenyl}amino)quinazolin-5-yl]oxy}propanamide;
(2R)—N-[2-(dimethylamino)ethyl]-2-{[4-({3-methyl-4-[(6-methylpyridin-3-yl)oxy]phenyl}amino)quinazolin-5-yl]oxy}propanamide;
(2R)—N-cyclopropyl-2-{[4-({3-methyl-4-[(6-methylpyridin-3-yl)oxy]phenyl}amino)quinazolin-5-yl]oxy}propanamide;
(2R)—N-(3-hydroxypropyl)-2-{[4-({3-methyl-4-[(6-methylpyridin-3-yl)oxy]phenyl}amino)quinazolin-5-yl]oxy}propanamide;
(2R)—N-(2-methoxyethyl)-2-{[4-({3-methyl-4-[(6-methylpyridin-3-yl)oxy]phenyl}amino)quinazolin-5-yl]oxy}propanamide;
(2R)-2-{[4-({3-methyl-4-[(6-methylpyridin-3-yl)oxy]phenyl}amino)quinazolin-5-yl]oxy}-N-(2-morpholin-4-ylethyl)propanamide;
(2R)-2-{[4-({3-methyl-4-[(6-methylpyridin-3-yl)oxy]phenyl}amino)quinazolin-5-yl]oxy}-N-(2-pyrrolidin-1-ylethyl)propanamide;
(2R)—N-[2-(acetylamino)ethyl]-2-{[4-({3-methyl-4-[(6-methylpyridin-3-yl)oxy]phenyl}amino)quinazolin-5-yl]oxy}propanamide;
(2R)-2-{[4-({3-methyl-4-[(6-methylpyridin-3-yl)oxy]phenyl}amino)quinazolin-5-yl]oxy}-N-[3-(4-methylpiperazin-1-yl)propyl]propanamide;
(2R)-2-{[4-({3-methyl-4-[(6-methylpyridin-3-yl)oxy]phenyl}amino)quinazolin-5-yl]oxy}-N-[3-(2-oxopyrrolidin-1-yl)propyl]propanamide;
(2R)-2-{[4-({3-methyl-4-[(6-methylpyridin-3-yl)oxy]phenyl}amino)quinazolin-5-yl]oxy}-N-[2-(methylthio)ethyl]propanamide;
(2R)—N-(3-methoxypropyl)-2-{[4-({3-methyl-4-[(6-methylpyridin-3-yl)oxy]phenyl}amino)quinazolin-5-yl]oxy}propanamide;
(2R)—N-cyclobutyl-2-{[4-({3-methyl-4-[(6-methylpyridin-3-yl)oxy]phenyl}amino)quinazolin-5-yl]oxy}propanamide;
(2R)—N-[(2R)-2-hydroxypropyl]-2-{[4-({3-methyl-4-[(6-methylpyridin-3-yl)oxy]phenyl}amino)quinazolin-5-yl]oxy}propanamide;
(2R)—N-[(2S)-2-hydroxypropyl]-2-{[4-({3-methyl-4-[(6-methylpyridin-3-yl)oxy]phenyl}amino)quinazolin-5-yl]oxy}propanamide;
(2R)—N-[(2S)-2,3-dihydroxypropyl]-2-{[4-({3-methyl-4-[(6-methylpyridin-3-yl)oxy]phenyl}amino)quinazolin-5-yl]oxy}propanamide;
(2R)—N-[(1R)-2-hydroxy-1-methylethyl]-2-{[4-({3-methyl-4-[(6-methylpyridin-3-yl)oxy]phenyl}amino)quinazolin-5-yl]oxy}propanamide;
(2R)—N-[(18)-2-hydroxy-1-methylethyl]-2-{[4-({3-methyl-4-[(6-methylpyridin-3-yl)oxy]phenyl}amino)quinazolin-5-yl]oxy}propanamide;
N-{3-methyl-4-[(6-methylpyridin-3-yl)oxy]phenyl}-5-[(1R)-1-methyl-2-morpholin-4-yl-2-oxoethoxy]quinazolin-4-amine;
(2R)—N-[2-(dimethylamino)ethyl]-N-methyl-2-{[4-({3-methyl-4-[(6-methylpyridin-3-yl)oxy]phenyl}amino)quinazolin-5-yl]oxy}propanamide;
5-[(1R)-1-methyl-2-(4-methylpiperazin-1-yl)-2-oxoethoxy]-N-{3-methyl-4-[(6-methylpyridin-3-yl)oxy]phenyl}quinazolin-4-amine;
[(2R)-1-((2R)-2-{[4-({3-methyl-4-[(6-methylpyridin-3-yl)oxy]phenyl}amino)quinazolin-5-yl]oxy}propanoyl)pyrrolidin-2-yl]methanol;
[(2S)-1-((2R)-2-{[4-({3-methyl-4-[(6-methylpyridin-3-yl)oxy]phenyl}amino)quinazolin-5-yl]oxy}propanoyl)pyrrolidin-2-yl]methanol;
1-((2R)-2-{[4-({3-methyl-4-[(6-methylpyridin-3-yl)oxy]phenyl}amino)quinazolin-5-yl]oxy}propanoyl)piperidin-4-ol;
(2R)—N,N-bis(2-hydroxyethyl)-2-{[4-({3-methyl-4-[(6-methylpyridin-3-yl)oxy]phenyl}amino)quinazolin-5-yl]oxy}propanamide;
(2R)—N-ethyl-N-(2-hydroxyethyl)-2-{[4-({3-methyl-4-[(6-methylpyridin-3-yl)oxy]phenyl}amino)quinazolin-5-yl]oxy}propanamide;
(2R)—N,N-bis(2-methoxyethyl)-2-{[4-({3-methyl-4-[(6-methylpyridin-3-yl)oxy]phenyl}amino)quinazolin-5-yl]oxy}propanamide;
5-[(1R)-2-(4-ethylpiperazin-1-yl)-1-methyl-2-oxoethoxy]-N-{3-methyl-4-[(6-methylpyridin-3-yl)oxy]phenyl}quinazolin-4-amine;
(3R)-1-((2R)-2-{[4-({3-methyl-4-[(6-methylpyridin-3-yl)oxy]phenyl}amino)quinazolin-5-yl]oxy}propanoyl)piperidin-3-ol;
(3S)-1-((2R)-2-{[4-({3-methyl-4-[(6-methylpyridin-3-yl)oxy]phenyl}amino)quinazolin-5-yl]oxy}propanoyl)piperidin-3-ol;
4-((2R)-2-{[4-({3-methyl-4-[(6-methylpyridin-3-yl)oxy]phenyl}amino)quinazolin-5-yl]oxy}propanoyl)piperazin-2-one;
[1-((2R)-2-{[4-({3-methyl-4-[(6-methylpyridin-3-yl)oxy]phenyl}amino)quinazolin-5-yl]oxy}propanoyl)piperidin-4-yl]methanol;
tert-butyl 4-((2R)-2-{[4-({3-methyl-4-[(6-methylpyridin-3-yl)oxy]phenyl}amino)quinazolin-5-yl]oxy}propanoyl)piperazine-1-carboxylate;
N-{3-methyl-4-[(6-methylpyridin-3-yl)oxy]phenyl}-5-[(1R)-1-methyl-2-oxo-2-piperazin-1-ylethoxy]quinazolin-4-amine;
5-[(1R)-2-azetidin-1-yl-1-methyl-2-oxoethoxy]-N-{3-methyl-4-[(6-methylpyridin-3-yl)oxy]phenyl}quinazolin-4-amine;
1-((2R)-2-{[4-({3-methyl-4-[(6-methylpyridin-3-yl)oxy]phenyl}amino)quinazolin-5-yl]oxy}propanoyl)azetidin-3-ol;
(2R)—N-(2-methoxyethyl)-N-methyl-2-{[4-({3-methyl-4-[(6-methylpyridin-3-yl)oxy]phenyl}amino)quinazolin-5-yl]oxy}propanamide;
(2R)—N,N-diethyl-2-{[4-({3-methyl-4-[(6-methylpyridin-3-yl)oxy]phenyl}amino)quinazolin-5-yl]oxy}propanamide;
N-{3-methyl-4-[(6-methylpyridin-3-yl)oxy]phenyl}-5-[(1R)-1-methyl-2-oxo-2-pyrrolidin-1-ylethoxy]quinazolin-4-amine;
(2R)—N-(3-hydroxypropyl)-N-methyl-2-{[4-({3-methyl-4-[(6-methylpyridin-3-yl)oxy]phenyl}amino)quinazolin-5-yl]oxy}propanamide;
N-[3-fluoro-4-(pyridin-3-yloxy)phenyl]-5-[(1R)-1-methyl-2-morpholin-4-yl-2-oxoethoxy]quinazolin-4-amine;
N-{3-chloro-4-[(6-methylpyridin-3-yl)oxy]phenyl}-5-[(1R)-1-methyl-2-morpholin-4-yl-2-oxoethoxy]quinazolin-4-amine;
N-[3-chloro-4-(pyridin-3-yloxy)phenyl]-5-[(1R)-1-methyl-2-morpholin-4-yl-2-oxoethoxy]quinazolin-4-amine;
5-[(1R)-1-methyl-2-morpholin-4-yl-2-oxoethoxy]-N-{4-[(6-methylpyridin-3-yl)oxy]phenyl}quinazolin-4-amine;
5-[(1R)-1-methyl-2-morpholin-4-yl-2-oxoethoxy]-N-[4-(pyridin-3-yloxy)phenyl]-quinazolin-4-amine;
N-{3-methoxy-4-[(6-methylpyridin-3-yl)oxy]phenyl}-5-[(1R)-1-methyl-2-morpholin-4-yl-2-oxoethoxy]quinazolin-4-amine;
N-[3-methoxy-4-(pyridin-3-yloxy)phenyl]-5-[(1R)-1-methyl-2-morpholin-4-yl-2-oxoethoxy]quinazolin-4-amine;
N-{3-fluoro-4-[(6-methylpyridin-3-yl)oxy]phenyl}-5-[(1R)-1-methyl-2-morpholin-4-yl-2-oxoethoxy]quinazolin-4-amine;
N-{3-cyano-4-[(6-methylpyridin-3-yl)oxy]phenyl}-5-[(1R)-1-methyl-2-morpholin-4-yl-2-oxoethoxy]quinazolin-4-amine;
N-[3-cyano-4-(pyridin-3-yloxy)phenyl]-5-[(1R)-1-methyl-2-morpholin-4-yl-2-oxoethoxy]quinazolin-4-amine;
5-[(1R)-1-methyl-2-morpholin-4-yl-2-oxoethoxy]-N-[3-methyl-4-(pyridin-2-yloxy)phenyl]quinazolin-4-amine;
5-[(1R)-1-methyl-2-morpholin-4-yl-2-oxoethoxy]-N-[3-methyl-4-(pyridin-3-yloxy)phenyl]quinazolin-4-amine;
5-[(1R)-1-methyl-2-morpholin-4-yl-2-oxoethoxy]-N-[3-methyl-4-(pyridin-4-yloxy)phenyl]quinazolin-4-amine;
5-[(1R)-1-methyl-2-morpholin-4-yl-2-oxoethoxy]-N-[3-methyl-4-(pyrazin-2-yloxy)phenyl]quinazolin-4-amine;
5-[(1R)-1-methyl-2-morpholin-4-yl-2-oxoethoxy]-N-[3-methyl-4-(1,3-thiazol-2-yloxy)phenyl]quinazolin-4-amine;
N-{4-[(6-methoxypyridin-3-yl)oxy]-3-methylphenyl}-5-[(1R)-1-methyl-2-morpholin-4-yl-2-oxoethoxy]quinazolin-4-amine;
5-[(1R)-1-methyl-2-morpholin-4-yl-2-oxoethoxy]-N-[3-methyl-4-(1,3-thiazol-5-yloxy)phenyl]quinazolin-4-amine;
5-[(1R)-1-methyl-2-morpholin-4-yl-2-oxoethoxy]-N-[3-methyl-4-(pyrimidin-5-yloxy)phenyl]quinazolin-4-amine;
5-[2-methyl-4-({5-[(1R)-1-methyl-2-morpholin-4-yl-2-oxoethoxy]quinazolin-4-yl}amino)phenoxy]pyridine-2-carbonitrile;
5-[(1R)-1-methyl-2-morpholin-4-yl-2-oxoethoxy]-N-[3-methyl-4-(pyridazin-3-yloxy)phenyl]quinazolin-4-amine;
(2R)—N-(2-hydroxyethyl)-2-{[4-({3-methoxy-4-[(6-methylpyridin-3-yl)oxy]phenyl}amino)quinazolin-5-yl]oxy}-N-methylpropanamide;
(2R)-2-{[4-({3-methoxy-4-[(6-methylpyridin-3-yl)oxy]phenyl}amino)quinazolin-5-yl]oxy}-N,N-dimethylpropanamide;
(2R)—N-ethyl-2-{[4-({3-methoxy-4-[(6-methylpyridin-3-yl)oxy]phenyl}amino)quinazolin-5-yl]oxy}propanamide;
(2R)—N-(2-hydroxyethyl)-2-{[4-({3-methoxy-4-[(6-methylpyridin-3-yl)oxy]phenyl}amino)quinazolin-5-yl]oxy}propanamide;
4-((2R)-2-{[4-({3-methoxy-4-[(6-methylpyridin-3-yl)oxy]phenyl}amino)quinazolin-5-yl]oxy}propanoyl)piperazin-2-one;
(2R)—N-(2-methoxyethyl)-2-{[4-({3-methoxy-4-[(6-methylpyridin-3-yl)oxy]phenyl}amino)quinazolin-5-yl]oxy}-N-methylpropanamide;
(3R)-1-((2R)-2-{[4-({3-methoxy-4-[(6-methylpyridin-3-yl)oxy]phenyl}amino)quinazolin-5-yl]oxy}propanoyl)piperidin-3-ol;
N-{3-methoxy-4-[(6-methylpyridin-3-yl)oxy]phenyl}-5-[(1R)-1-methyl-2-oxo-2-piperazin-1-ylethoxy]quinazolin-4-amine;
(2R)—N,N-dimethyl-2-[(4-{[3-methyl-4-(pyridin-2-yloxy)phenyl]amino}quinazolin-5-yl)oxy]propanamide;
(2R)—N-ethyl-2-[(4-{[3-methyl-4-(pyridin-2-yloxy)phenyl]amino}quinazolin-5-yl)oxy]propanamide;
(2R)—N-(2-hydroxyethyl)-2-[(4-{[3-methyl-4-pyridin-2-yloxy)phenyl]amino}quinazolin-5-yl)oxy]propanamide;
(2R)—N-(2-hydroxyethyl)-N-methyl-2-[(4-{[3-methyl-4-(pyridin-2-yloxy)phenyl]amino}quinazolin-5-yl)oxy]propanamide;
4-{(2R)-2-[(4-{[3-methyl-4-(pyridin-2-yloxy)phenyl]amino}quinazolin-5-yl)oxy]propanoyl}piperazin-2-one;
(2R)—N-(2-methoxyethyl)-N-methyl-2-[(4-{[3-methyl-4-(pyridin-2-yloxy)phenyl]amino}quinazolin-5-yl)oxy]propanamide;
(3R)-1-{(2R)-2-[(4-{[3-methyl-4-(pyridin-2-yloxy)phenyl]amino}quinazolin-5-yl)oxy]propanoyl}piperidin-3-ol;
5-[(1R)-1-methyl-2-oxo-2-piperazin-1-ylethoxy]-N-[3-methyl-4-(pyridin-2-yloxy)phenyl]quinazolin-4-amine;
5-[(1R)-1-methyl-2-morpholin-4-yl-2-oxoethoxy]-N-[3-methyl-4-(pyridin-2-ylmethoxy)phenyl]quinazolin-4-amine;
{5-[2-methyl-4-({5-[(1R)-1-methyl-2-morpholin-4-yl-2-oxoethoxy]quinazolin-4-yl}amino)phenoxy]pyridin-2-yl}methanol;
N-{4-[(6-fluoropyridin-3-yl)oxy]-3-methylphenyl}-5-[(1R)-1-methyl-2-morpholin-4-yl-2-oxoethoxy]quinazolin-4-amine;
N-[3-chloro-4-(pyridin-2-yloxy)phenyl]-5-[(1R)-1-methyl-2-morpholin-4-yl-2-oxoethoxy]quinazolin-4-amine;
(2R)-2-[(4-{[3-chloro-4-(pyridin-2-yloxy)phenyl]amino}quinazolin-5-yl)oxy]-N-(2-hydroxyethyl)-N-methylpropanamide;
(2R)-2-[(4-{[3-chloro-4-(pyridin-2-yloxy)phenyl]amino}quinazolin-5-yl)oxy]-N,N-dimethylpropanamide;
(2R)-2-[(4-{[3-chloro-4-(pyridin-2-yloxy)phenyl]amino}quinazolin-5-yl)oxy]-N-(2-hydroxyethyl)propanamide;
(2R)-2-[(4-{[3-chloro-4-(pyridin-2-yloxy)phenyl]amino}quinazolin-5-yl)oxy]-N-ethyl-N-(2-hydroxyethyl)propanamide;
(2R)-2-[(4-{[3-chloro-4-(pyridin-2-yloxy)phenyl]amino}quinazolin-5-yl)oxy]-N-(2-methoxyethyl)-N-methylpropanamide;
4-{(2R)-2-[(4-{[3-chloro-4-(pyridin-2-yloxy)phenyl]amino}quinazolin-5-yl)oxy]propanoyl}piperazin-2-one;
N-[3-chloro-4-(pyridin-2-yloxy)phenyl]-5-[(1R)-1-methyl-2-oxo-2-piperazin-1-ylethoxy]quinazolin-4-amine;
1-{(2R)-2-[(4-{[3-chloro-4-(pyridin-2-yloxy)phenyl]amino}quinazolin-5-yl)oxy]propanoyl}piperidin-3-ol;
N-{3-methyl-4-[(1-methyl-1H-pyrazol-4-yl)oxy]phenyl}-5-[(1R)-1-methyl-2-morpholin-4-yl-2-oxoethoxy]quinazolin-4-amine;
N-{3-chloro-4-[(1-methyl-1H-pyrazol-4-yl)oxy]phenyl}-5-[(1R)-1-methyl-2-morpholin-4-yl-2-oxoethoxy]quinazolin-4-amine;
N-(4-{[6-(fluoromethyl)pyridin-3-yl]oxy}-3-methylphenyl)-5-[(1R)-1-methyl-2-morpholin-4-yl-2-oxoethoxy]quinazolin-4-amine;
N-[3-chloro-4-(1,3-thiazol-2-yloxy)phenyl]-5-[(1R)-1-methyl-2-morpholin-4-yl-2-oxoethoxy]quinazolin-4-amine;
(2S)—N,N-dimethyl-2-{[4-({3-methyl-4-[(6-methylpyridin-3-yl)oxy]phenyl}amino)quinazolin-5-yl]oxy}propanamide;
(2R)-2-{[4-({3-chloro-4-[(6-methylpyridin-3-yl)oxy]phenyl}amino)quinazolin-5-yl]oxy}-N-(2-hydroxyethyl)-N-methylpropanamide;
(2R)-2-{[4-({3-chloro-4-[(6-methylpyridin-3-yl)oxy]phenyl}amino)quinazolin-5-yl]oxy}-N,N-dimethylpropanamide;
N-{3-chloro-4-[(6-fluoropyridin-3-yl)oxy]phenyl}-5-[(1R)-1-methyl-2-morpholin-4-yl-2-oxoethoxy]quinazolin-4-amine;
N-[3-chloro-4-(pyrazin-2-yloxy)phenyl]-5-[(1R)-1-methyl-2-morpholin-4-yl-2-oxoethoxy]quinazolin-4-amine; and
N-[3-chloro-4-(1,3-thiazol-5-yloxy)phenyl]-5-[(1R)-1-methyl-2-morpholin-4-yl-2-oxoethoxy]quinazolin-4-amine;
   or a pharmaceutically acceptable salt thereof.

A quinazoline derivative of the formula I, or a pharmaceutically acceptable salt thereof, may be prepared by any process known to be applicable to the preparation of chemically-related compounds. Suitable processes include, for example, those illustrated in International Patent Applications WO 96/15118, WO 01/94341, WO 03/040108 and WO 03/040109. Such processes, when used to prepare a quinazoline derivative of the formula I are provided as a further feature of the invention and are illustrated by the following representative process variants in which, unless otherwise stated, R¹, R², R³, R⁴, R⁵, R⁶, R⁷, X¹, Q¹, m and n have any of the meanings defined hereinbefore. Necessary starting materials may be obtained by standard procedures of organic chemistry. The preparation of such starting materials is described in conjunction with the following representative process variants and within the accompanying Examples. Alternatively necessary starting materials are obtainable by analogous procedures to those illustrated which are within the ordinary skill of an organic chemist.
Process (a) The reaction of a quinazoline of the formula II:

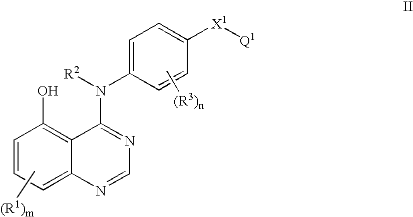

wherein R¹, R², R³, X¹, Q¹, m and n have any of the meanings defined hereinbefore except that any functional group is protected if necessary, with an amide of the formula III:

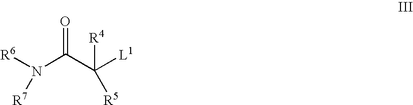

wherein R⁴, R⁵, R⁶ and R⁷ have any of the meanings defined hereinbefore except that any functional group is protected if necessary and L¹ is a suitable displaceable group, such as halogeno (for example chloro or bromo), a sulfonyloxy group (for example a methylsulfonyloxy or a toluene-4-sulfonyloxy group) or a hydroxy group;

or

Process (b) The coupling, conveniently in the presence of a suitable base, of a quinazoline of the formula IV (or a suitable salt thereof, for example an alkali earth metal salt or an alkali metal salt, such as a sodium or a potassium salt, thereof):

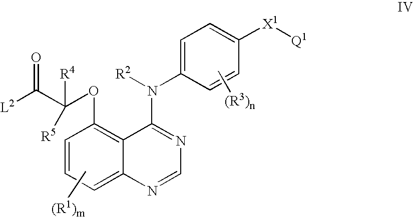

wherein R¹, R², R³, R⁴, R⁵, X¹, Q¹, m and n have any of the meanings defined hereinbefore except that any functional group is protected if necessary, and L² is a suitable displaceable group, for example (C1-C3)alkoxy (such as methoxy or ethoxy) or L² is hydroxy, which hydroxy group is conveniently combined with a suitable coupling agent to produce a displaceable group, with an amine of the formula V:

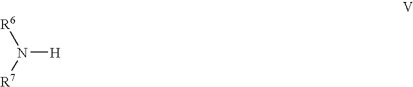

wherein R⁶ and R⁷ have any of the meanings defined hereinbefore except that any functional group is protected if necessary;

or

Process (c) For quinazoline derivatives of the formula I wherein at least one of R⁴ and R⁵ is 2-hydroxyethyl, the reaction of a quinazoline of the formula VI:

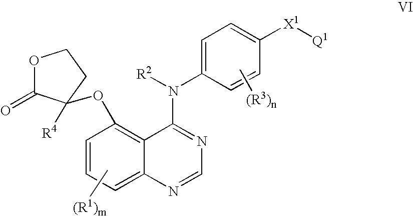

wherein R¹, R², R³, R⁴, X¹, Q¹, m and n have any of the meanings defined hereinbefore except that any functional group is protected if necessary, with an amine of the formula V as defined above;

or

Process (d) The reaction of a quinazoline of the formula VII:

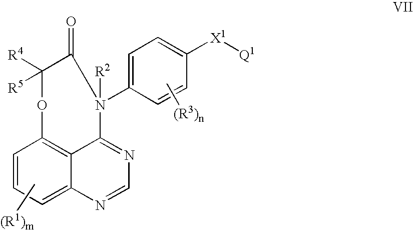

wherein R¹, R², R³, R⁴, R⁵, X¹, Q¹, m and n have any of the meanings defined hereinbefore except that any functional group is protected if necessary, with an amine of the formula V as defined above;

or

Process (e) The reaction of a quinazolin-4(3H)-one of the formula VIII:

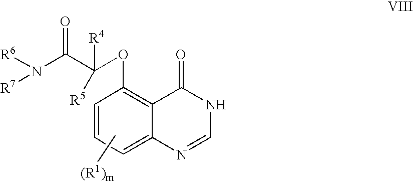

wherein R¹, R⁴, R⁵, R⁶, R⁷ and m have any of the meanings defined hereinbefore except that any functional group is protected if necessary, with a suitable activating group and an amine of the formula IX:

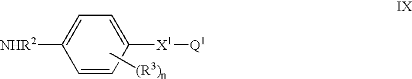

wherein R², R³, X¹, Q¹ and n have any of the meanings defined hereinbefore except that any functional group is protected if necessary;

or

Process (f) When X¹ is O, S, OC(R¹³)₂ or SC(R¹³)₂, the reaction of a quinazoline of the formula X:

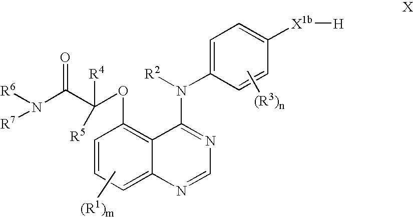

wherein R¹, R², R³, R⁴, R⁵, R⁶, R⁷, n and m have any of the meanings defined hereinbefore except that any functional group is protected if necessary and X^{1b} is O or S, with a compound of the formula Q¹-[C(R¹³)₂]ᵣ-L³ wherein r is 0 or 1, L³ is a suitable displaceable group such as halogeno (for example chloro or fluoro) and R¹³ and Q¹ have any of the meanings defined hereinbefore except that any functional group is protected if necessary. For example, when r is 0, Q¹ may suitably be selected from 2-pyrimidinyl, 2-pyrazinyl or 2-pyridinyl;

or

Process (g) The reaction of a quinazoline of the formula XI:

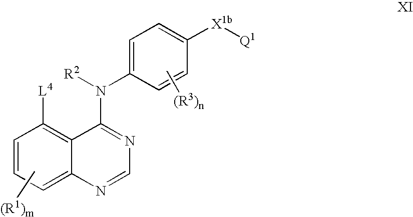

wherein L⁴ is a suitable displaceable group such as halogeno (for example fluoro) and R¹, R², R³, X¹, Q¹, n and m have any of the meanings defined hereinbefore except that any functional group is protected if necessary with a compound of the formula XII:

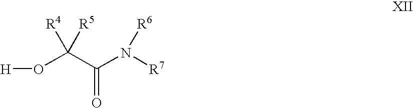

wherein R⁴, R⁵, R⁶ and R⁷ have any of the meanings defined hereinbefore except that any functional group is protected if necessary;

and thereafter, if necessary:

(i) converting a quinazoline derivative of the formula I into another quinazoline derivative of the formula I;

(ii) removing any protecting group that is present (by conventional means);

(iii) forming a pharmaceutically acceptable salt.

Specific conditions for the above reactions are as follows:

Process (a)

When L¹ is, for example, halogeno or a sulfonyloxy group, the reaction of process (a) is conveniently carried out in the presence of a suitable base. A suitable base is, for example, an alkali or alkaline earth metal carbonate, such as sodium carbonate, potassium carbonate, caesium carbonate or calcium carbonate. The reaction is, optionally, carried out in the presence of a source of iodide such as sodium iodide or potassium iodide or in the presence of a suitable alkali metal hydride such as sodium hydride or potassium hydride.

The reaction is conveniently carried out in the presence of a suitable inert solvent or diluent, for example an ester such as ethyl acetate, a halogenated solvent such as methylene chloride, chloroform or carbon tetrachloride, an ether such as tetrahydrofuran or 1,4-dioxan, an aromatic solvent such as toluene, an alcohol such as methanol or ethanol, or a dipolar aprotic solvent such as N,N-dimethylformamide, N,N-dimethylacetamide, N-methylpyrrolidin-2-one or dimethylsulfoxide. The reaction is conveniently carried out at a temperature in the range, for example, from 0 to 120° C., conveniently at or near ambient temperature and/or at about 50° C.

When L¹ is hydroxy, the reaction of process (a) is conveniently carried out under suitable Mitsunobu conditions. Suitable Mitsunobu conditions include, for example, reaction in the presence of a suitable tertiary phosphine and a di-alkylazodicarboxylate in an organic solvent such as THF, or suitably dichloromethane and in the temperature range 0° C. to 60° C., but conveniently at ambient temperature. A suitable tertiary phosphine includes for example tri-n-butylphosphine or suitably tri-phenylphosphine. A suitable di-alkylazodicarboxylate includes for example diethyl azodicarboxylate (DEAD) or suitably di-tert-butyl azodicarboxylate (DTAD). Details of Mitsunobu reactions are contained in Tet. Letts., 31, 699, (1990); The Mitsunobu Reaction, D. L. Hughes, Organic Reactions, 1992, Vol. 42, 335-656 and Progress in the Mitsunobu Reaction, D. L. Hughes, Organic Preparations and Procedures International, 1996, Vol. 28, 127-164.

Process (b)

When L² is hydroxy, the reaction of process (b) is conveniently carried out in the presence of a suitable coupling agent. A suitable coupling agent is, for example, a suitable peptide coupling agent, such as O-(7-azabenzotriazol-1-yl)-N,N,N′,N′-tetramethyluronium hexafluoro-phosphate (HATU) or a carbodiimide such as dicyclohexylcarbodiimide or 1-(3-dimethylaminopropyl)-3-ethylcarbodiimide hydrochloride (EDCI). The reaction of process (b) is optionally carried out in the presence of a suitable catalyst such as dimethylaminopyridine, 4-pyrrolidinopyridine, 2-hydroxypyridine N-oxide (HOPO) or 1-hydroxybenzotriazole (HOBT).

When L² is hydroxy, the reaction of process (b) may conveniently be carried out in the presence of a suitable base. A suitable base is, for example, an organic amine base such as pyridine, 2,6-lutidine, collidine, 4-dimethylaminopyridine, triethylamine, di-isopropylethylamine, N-methylmorpholine or diazabicyclo[5.4.0]undec-7-ene, or an alkali or alkaline earth metal carbonate, such as sodium carbonate, potassium carbonate, caesium carbonate or calcium carbonate.

The reaction of process (b) is conveniently carried out in the presence of a suitable inert solvent or diluent, for example an ester such as ethyl acetate, a halogenated solvent such as methylene chloride, chloroform or carbon tetrachloride, an ether such as tetrahydrofuran or 1,4-dioxan, an aromatic solvent such as toluene, an alcohol such as methanol or ethanol, or a dipolar aprotic solvent such as N,N-dimethylformamide, N,N-dimethylacetamide, N-methylpyrrolidin-2-one or dimethylsulfoxide. The reaction is conveniently carried out at a temperature in the range, for example, from 0 to 120° C. When L² is hydroxy, the reaction may conveniently be carried out at or near ambient temperature. When L² is (C₁-C₃)alkoxy, the reaction may conveniently be carried out at or near about 60° C.

Conveniently, this reaction may also be performed by heating the reactants in a sealed vessel using a suitable heating apparatus such as a microwave heater.

Process (c)

The reaction of process (c) is conveniently carried out in the presence of a suitable inert solvent or diluent, for example an ester such as ethyl acetate, a halogenated solvent such as methylene chloride, chloroform or carbon tetrachloride, an ether such as tetrahydrofuran or 1,4-dioxan, an aromatic solvent such as toluene, an alcohol such as ethanol, or a dipolar aprotic solvent such as N,N-dimethylformamide, N,N-dimethylacetamide, N-methylpyrrolidin-2-one or dimethylsulfoxide. The reaction is conveniently carried out at a temperature in the range, for example, from 0 to 120° C., conveniently at or near ambient temperature.

Process (d)

The reaction of process (d) is conveniently carried out in the presence of a suitable inert solvent or diluent, for example an ester such as ethyl acetate, a halogenated solvent such as methylene chloride, chloroform or carbon tetrachloride, an ether such as tetrahydrofuran or 1,4-dioxan, an aromatic solvent such as toluene, an alcohol such as ethanol, or a dipolar aprotic solvent such as N,N-dimethylformamide, N,N-dimethylacetamide, N-methylpyrrolidin-2-one or dimethylsulfoxide. The reaction is conveniently carried out at a temperature in the range, for example, from 0 to 120° C., conveniently at or near ambient temperature.

Process (e)

In process (e), the quinazolin-4(3H)-one of the formula VIII is conveniently reacted with a suitable activating agent, so as to replace the oxo group at the 4-position on the quinazolin-4(3H)-one ring by a suitable displaceable group, for example halogeno (for such as chloro) and to form a quinazoline (hereinafter referred to as the “activated quinazoline”) for reaction with the amine of the formula IX. The activated quinazoline so formed may conveniently be used in situ without further purification.

The reaction of the quinazolin-4(3H)-one of the formula VIII with a suitable activating agent is conveniently carried out using conventional methods. For example, the quinazolin-4(3H)-one of the formula VIII may be reacted with a suitable halogenating agent such as thionyl chloride, phosphoryl chloride or a mixture of carbon tetrachloride and triphenylphosphine.

The reaction of the activated quinazoline with the amine of the formula IX is conveniently carried out in the presence of an acid, for example in the presence of a catalytic amount of an acid. Suitable acids include, for example hydrogen chloride gas (conveniently dissolved in a suitable inert solvent such as diethyl ether or dioxane) or hydrochloric acid.

Alternatively, when the activated quinazoline contains a halogeno group (for example chloro) at the 4-position on the quinazoline ring, the reaction with the amine of the formula IX may be carried out in the absence of an acid or a base. In this reaction displacement of the halogeno leaving group results in the formation of the acid (H-halogeno) in-situ and the autocatalysis of the reaction.

Alternatively, the reaction of the activated quinazoline with the amine of the formula IX may be carried out in the presence of a suitable base. A suitable base is, for example, lithium diisopropyl amine (LDA) or sodium bis(trimethylsilyl)amide (NaHMDS).

The above reactions are conveniently carried out in the presence of a suitable inert solvent or diluent, for example an alcohol or ester such as methanol, ethanol, isopropanol or ethyl acetate, a halogenated solvent such as methylene chloride, chloroform or carbon tetrachloride, an ether such as tetrahydrofuran, diethyl ether or 1,4-dioxan, an aromatic solvent such as toluene, or a dipolar aprotic solvent such as N,N-dimethylformamide, N,N-dimethylacetamide, N-methylpyrrolidin-2-one or dimethylsulfoxide.

When conducted in the presence or absence of an acid, the above reactions are conveniently carried out at a temperature in the range, for example, 0 to 250° C., conveniently in the range 40 to 80° C. or, preferably, at or near the reflux temperature of the solvent when used. When conducted in the presence of a base, the above reactions are conveniently carried out at a temperature in the range, for example, −78 to 30° C.

Process (f)

Process (f) may conveniently be carried out using analogous conditions to those used in step (i) of Reaction Scheme 2 as discussed below.

Process (g)

Process (g) may conveniently be carried out in the presence of a suitable base. A suitable base is, for example, an alkali metal hydride, such as sodium hydride.

The reaction is conveniently carried out in the presence of a suitable inert solvent or diluent, for example an ether such as tetrahydrofuran or 1,4-dioxan, an aromatic solvent such as toluene, or a dipolar aprotic solvent such as N,N-dimethylformamide, N,N-dimethylacetamide, N-methylpyrrolidin-2-one or dimethylsulfoxide. The reaction is conveniently carried out at a temperature in the range, for example, from 0 to 120° C.

Starting Materials for Process (a)

The quinazoline of the formula II may be obtained by conventional procedures, for example as illustrated in Reaction Scheme 1:

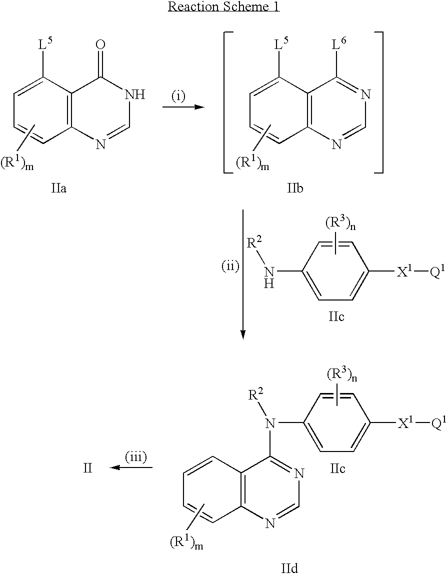

wherein L⁵ and L⁶ are suitable displaceable groups, provided that L⁶ is more labile than L⁵, and R¹, R², R³, X¹, Q¹, m and n have any of the meanings defined hereinbefore except that any functional group is protected if necessary.

A suitable displaceable group L⁵ is for example halogeno or a sulfonyloxy group, for example fluoro, chloro, methylsulfonyloxy or toluene-4-sulfonyloxy group, particularly fluoro. A suitable displaceable group L⁶ is, for example, halogeno (such as fluoro or chloro), alkoxy, aryloxy, mercapto, alkylthio, arylthio, alkylsulfinyl, arylsulfinyl, alkylsulfonyl, arylsulfonyl, alkylsulfonyloxy or arylsulfonyloxy group, for example a chloro, bromo, methoxy, phenoxy, pentafluorophenoxy, methylthio, methanesulfonyl, methanesulfonyloxy or toluene-4-sulfonyloxy group. Preferably L⁵ and L⁶ are both halogeno, for example L⁵ is fluoro and L⁶ is chloro.

Alternatively, as would be appreciated by a person skilled in the art, the quinazoline of the formula IId may conveniently be prepared by reaction of the quinazoline of the formula IIb with an appropriate 4-aminophenol compound, followed by alkylation of the phenol by conventional procedures.

Notes for Reaction Scheme 1:

Step (i)

As the skilled person would appreciate, the conversion of a quinazolone of the formula IIa to a quinazoline of the formula IIb may be conducted using conventional methods, for example by reacting the compound of the formula Ia with a suitable activating agent. For example, when m is 0, L⁵ is fluoro and L⁶ is halogeno (for example chloro), 5-fluoro-quinazolin-4(3H)-one may be reacted with a suitable halogenating agent such as thionyl chloride, phosphoryl chloride or a mixture of carbon tetrachloride and triphenylphosphine.

Step (ii)

The reaction of step (ii) may conveniently be carried out using analogous conditions to those used in process (e) as discussed above.

Step (iii)

The conversion of a quinazoline of the formula IId to a quinazoline of the formula II may be carried out by reaction with a suitably protected oxygen nucleophile, followed by removal of the protecting group by conventional means. For example, the conversion may conveniently be carried out by reaction with N-acetylethanolamine in the presence of a suitable base. A suitable base is, for example, a strong non-nucleophilic base such as an alkali metal hydride (for example sodium hydride) or an alkali metal amide (for example lithium di-isopropylamide (LDA)). The reaction is conveniently carried out in the presence of a suitable inert solvent or diluent, for example an ether such as tetrahydrofuran or 1,4-dioxane, an aromatic solvent such as toluene, or a dipolar aprotic solvent such as N,N-dimethylformamide, N,N-dimethylacetamide, N-methylpyrrolidin-2-one or dimethylsulfoxide. The reaction is conveniently carried out at a temperature in the range, for example, from 10 to 250° C., preferably in the range from 100 to 150° C.

The conversion may alternatively be carried out by reaction with a suitable alkali metal alkoxide (for example sodium methoxide), followed by a conventional demethylation reaction. Any suitable demethylation reaction conditions may be used. For example, the demethylation step may be carried out by reaction with pyridinium hydrochloride at a temperature in the range from 50 to 180° C., by reaction with boron tribromide at a temperature in the range from −78 to 30° C. or by reaction with a suitable thiolate, such as sodium thiophenolate at a temperature in the range from 50 to 200° C.

Starting Materials for Reaction Scheme 1

The compounds of the formula IIa are commercially available or may be prepared using conventional methods. For example, the 5-fluoro-quinazolin-4(3H)-one starting material is commercially available or can be prepared using conventional methods, for example as described in J. Org. Chem. 1952, 17, 164-176.

Compounds of the formula IIc are commercially available compounds or they are known in the literature, or they can be prepared by standard processes known in the art. For example, the compound of the formula IIc wherein R² is hydrogen and X¹ is O, S, SO, SO₂, N(R¹³), OC(R¹³)₂, SC(R¹³)₂ or N(R¹³)C(R¹³)₂, wherein R¹³ is as hereinbefore defined (particularly wherein X¹ is O or S), may be prepared in accordance with Reaction Scheme 2:

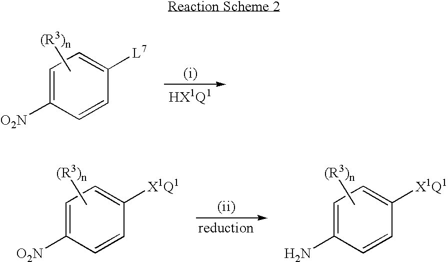

wherein L⁷ is a suitable displaceable group, for example halogeno (such as fluoro or chloro) and Q¹, X¹, R³ and n are as hereinbefore defined, except any functional group is protected if necessary.

Notes for Reaction Scheme 2

Step (i)

The reaction in step (i) is conveniently carried out in the presence of a suitable base and in the presence of a suitable inert diluent or solvent. Suitable bases include, for example, an organic amine base such as, for example, pyridine, 2,6-lutidine, collidine, 4-dimethylaminopyridine, triethylamine, di-isopropylethylamine, N-methylmorpholine or diazabicyclo[5.4.0]undec-7-ene, or, for example, an alkali or alkaline earth metal carbonate, for example sodium carbonate, potassium carbonate, caesium carbonate, calcium carbonate, or, for example, an alkali metal hydride, for example sodium hydride. A particular base when X¹ is O or S is, for example, an alkali or alkaline earth metal carbonate, such as potassium carbonate. A particular base when X¹ is O, S or OCH₂ is, for example, an alkali metal hydride, such as sodium hydride.

The reaction is conveniently carried out in the presence of a suitable inert solvent or diluent, for example a halogenated solvent such as methylene chloride, chloroform or carbon tetrachloride, an ether such as tetrahydrofuran or 1,4-dioxane, an aromatic solvent such as toluene, or a dipolar aprotic solvent such as N,N-dimethylformamide, N,N-dimethylacetamide, N-methylpyrrolidin-2-one or dimethylsulfoxide. The reaction is conveniently carried out at a temperature in the range of, for example, from 25 to 100° C., conveniently at or near ambient temperature.

The compounds of the formula HX¹Q¹ are commercially available, or they are known in the literature, or can be prepared using well-known processes in the art. For example compounds of the formula Q¹CH₂OH may be prepared using known methods, for example by reduction of the corresponding ester of the formula Q¹COOR′, wherein R′ is, for example (1-6C)alkyl or benzyl, with a suitable reducing agent, for example lithium aluminium hydride.

Step (ii)

The reduction of the nitro group in step (ii) may be carried out under standard conditions, for example by catalytic hydrogenation over a platinum/carbon, palladium/carbon or nickel catalyst, treatment with a metal such as iron, titanium (III) chloride, tin (II) chloride or indium, or treatment with another suitable reducing agent such as sodium dithionite.

Compounds of the formula IIc wherein R² is hydrogen and X¹ is OC(R¹³)₂, SC(R¹³)₂ or N(R¹³)C(R¹³)₂ (particularly OC(R¹³)₂ wherein R¹³ is hydrogen) may, for example, be prepared in accordance with Reaction Scheme 3:

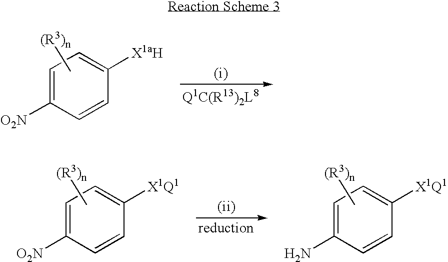

wherein L⁸ is a suitable leaving group for example a halogeno or a sulfonyloxy group, such as a fluoro, chloro, methylsulfonyloxy or toluene-4-sulfonyloxy group, X^{1a} is O, S or N(R¹³), X¹ is OC(R¹³)₂, SC(R¹³)₂ or N(R¹³)C(R¹³)₂ and R³, R¹³, Q¹ and n are as hereinbefore defined except any functional group is protected if necessary.

Notes for Reaction Scheme 3

Step (i): Analogous conditions to those used in step (i) of Reaction Scheme 2.

Step (ii) Analogous conditions to those used in step (ii) of Reaction Scheme 2.

Other suitable methods for preparing compounds of the formula IIc are disclosed in for example WO 03/040108 and as illustrated by the examples herein.

Compounds of the formula IIc wherein X¹ is OC(R¹³)₂ may also be prepared by coupling the appropriate starting nitro phenol in Reaction Scheme 3 (i.e. wherein X^{1a}H is OH) with a compound of the formula Q¹C(R¹³)₂OH, conveniently in the presence of a suitable dehydrating agent. A suitable dehydrating agent is, for example, a carbodiimide reagent such as dicyclohexylcarbodiimide or 1-(3-dimethylaminopropyl)-3-ethylcarbodiimide or a mixture of an azo compound such as diethyl or di-tert-butyl azodicarboxylate and a phosphine such as triphenylphosphine. The reaction is conveniently carried out in the presence of a suitable inert solvent or diluent, for example a halogenated solvent such as methylene chloride, chloroform or carbon tetrachloride and at a temperature in the range, for example, 0 to 150° C., preferably at or near ambient temperature.

The amides of the formula III are commercially available, or they are known in the literature, or can be prepared using well-known processes in the art.

Starting Materials for Process (b)

The quinazoline of the formula IV may be obtained by conventional procedures. For example quinazoline compounds of the formula IV wherein L² is (1-3C)alkoxy may be prepared by reaction of a compound of the formula II as defined above or a compound of the formula IId as defined above with a compound of the formula IVa:

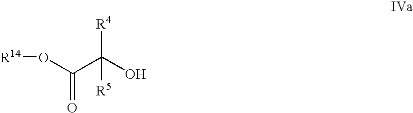

wherein R¹⁴ is a (1-3C)alkyl group and R⁴ and R⁵ have any of the meanings defined hereinbefore except that any functional group is protected if necessary.

The reaction of a compound of the formula II with a compound of the formula IVa may conveniently be carried out under suitable Mitsunobu conditions as described above.

The reaction of a compound of the formula IId with a compound of the formula IVa is conveniently be carried out in the presence of a suitable base. A suitable base would be an alkali metal alkoxide, for example sodium methoxide or sodium ethoxide.

Quinazoline compounds of the formula IV wherein L² is hydroxy (or a suitable salt thereof) may be prepared by reaction of a compound of the formula IV wherein L² is (1-3C)alkoxy with a suitable alkali metal hydroxide, for example sodium hydroxide at room temperature. This reaction is conveniently carried out in the presence of a suitable inert solvent or diluent, for example an ether such as tetrahydrofuran or 1,4-dioxane or an alcohol such as methanol.

Quinazoline compounds of the formula IV wherein L² is hydroxy (or a suitable salt thereof) may alternatively be prepared by reaction of a compound of the formula II with a suitable halogenated (for example chlorinated) alcohol under suitable chlorotone reaction conditions, as appreciated by a person skilled in the art and, for example, described in Reference Example 27 of WO 03/077847.

The compounds of the formulae IVa and V are commercially available, or they are known in the literature, or can be prepared using well-known processes in the art.

Starting Materials for Process (c)

The compounds of the formula VI can be prepared using well-known processes in the art. For example, the compounds of the formula VI can be prepared by reaction of a compound of the formula II as discussed above with a compound of the formula VIa: 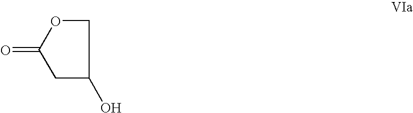
for example under suitable Mitsunobu conditions, as discussed above.

The compounds of the formula V and VIa are commercially available, or they are known in the literature, or can be prepared using well-known processes in the art.

Starting Materials for Process (d)

The compounds of the formula V are discussed above.

The compounds of the formula VII may be prepared from compounds of the formula IV wherein L² is hydroxy by an internal coupling reaction using a suitable coupling agent and a suitable base as described above (for example HATU and di-isopropylethylamine) under the reaction conditions discussed above for process (b).

Starting Materials for Process (e)

The compounds of the formula VIII may be prepared using well-known processes in the art. Compounds of the formula VIII may, for example, be prepared by reaction of an appropriate quinazolin-4(3H)-one compound VIIIa:

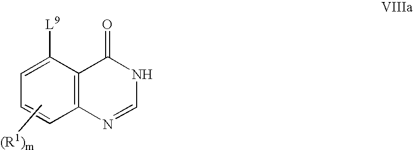

wherein L⁹ is a suitable displaceable group and R¹ and m have any of the meanings defined hereinbefore except that any functional group is protected if necessary, with a compound of the formula III as defined above. A suitable displaceable group L⁹ is for example halogeno or a sulfonyloxy group, for example fluoro, chloro, methylsulfonyloxy or toluene-4-sulfonyloxy group, particularly fluoro.

The reaction of a compound of the formula VIIIa with a compound of the formula III is conveniently carried out using analogous conditions to those used in step (iii) of Reaction Scheme 1 as described above.

Alternatively, the group L⁹ may represent hydroxy and the reaction of a compound of the formula VIIIa with a compound of the formula III is conveniently carried out under the conditions described above for process (a).

The compounds of the formula IX are commercially available, or they are known in the literature, or can be prepared using well-known processes in the art.

Starting Materials for Process (f)

Quinazolines of the formula X may be prepared using processes as discussed above.

The compounds of the formula Q¹-[C(R¹³)₂]ᵣ-L³ are commercially available, or they are known in the literature, or can be prepared using well-known processes in the art.

Starting Materials for Process (g)

Quinazolines of the formula XI may be prepared using processes as discussed above, for example as discussed in Reaction Scheme 1.

The compounds of the formula XII are commercially available, or they are known in the literature, or can be prepared using well-known processes in the art.

The quinazoline derivative of the formula I may be obtained from the above processes in the form of the free base or alternatively it may be obtained in the form of a salt, such as an acid addition salt. When it is desired to obtain the free base from a salt of the quinazoline derivative of the formula I, the salt may be treated with a suitable base, for example, an alkali or alkaline earth metal carbonate or hydroxide, for example sodium carbonate, potassium carbonate, calcium carbonate, sodium hydroxide or potassium hydroxide, or by treatment with ammonia for example using a methanolic ammonia solution such as 7N ammonia in methanol.

The protecting groups used in the processes above may in general be chosen from any of the groups described in the literature or known to the skilled chemist as appropriate for the protection of the group in question and may be introduced by conventional methods. Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Specific examples of protecting groups are given below for the sake of convenience, in which “lower”, as in, for example, lower alkyl, signifies that the group to which it is applied preferably has 1 to 4 carbon atoms. It will be understood that these examples are not exhaustive. Where specific examples of methods for the removal of protecting groups are given below these are similarly not exhaustive. The use of protecting groups and methods of deprotection not specifically mentioned are, of course, within the scope of the invention.

A carboxy protecting group may be the residue of an ester-forming aliphatic or arylaliphatic alcohol or of an ester-forming silanol (the said alcohol or silanol preferably containing 1 to 20 carbon atoms). Examples of carboxy protecting groups include straight or branched chain (1-12C)alkyl groups (for example isopropyl, and tert-butyl); lower alkoxy-lower alkyl groups (for example methoxymethyl, ethoxymethyl and isobutoxymethyl); lower acyloxy-lower alkyl groups, (for example acetoxymethyl, propionyloxymethyl, butyryloxymethyl and pivaloyloxymethyl); lower alkoxycarbonyloxy-lower alkyl groups (for example 1-methoxycarbonyloxyethyl and 1-ethoxycarbonyloxyethyl); aryl-lower alkyl groups (for example benzyl, 4-methoxybenzyl, 2-nitrobenzyl, 4-nitrobenzyl, benzhydryl and phthalidyl); tri(lower alkyl)silyl groups (for example trimethylsilyl and tert-butyldimethylsilyl); tri(lower alkyl)silyl-lower alkyl groups (for example trimethylsilylethyl); and (2-6C)alkenyl groups (for example allyl). Methods particularly appropriate for the removal of carboxyl protecting groups include for example acid-, base-, metal- or enzymically-catalysed cleavage.

Examples of hydroxy protecting groups include lower alkyl groups (for example tert-butyl), lower alkenyl groups (for example allyl); lower alkanoyl groups (for example acetyl); lower alkoxycarbonyl groups (for example tert-butoxycarbonyl); lower alkenyloxycarbonyl groups (for example allyloxycarbonyl); aryl-lower alkoxycarbonyl groups (for example benzyloxycarbonyl, 4-methoxybenzyloxycarbonyl, 2-nitrobenzyloxycarbonyl and 4-nitrobenzyloxycarbonyl); tri(lower alkyl)silyl (for example trimethylsilyl and tert-butyldimethylsilyl) and aryl-lower alkyl (for example benzyl) groups.

Examples of amino protecting groups include formyl, aryl-lower alkyl groups (for example benzyl and substituted benzyl, 4-methoxybenzyl, 2-nitrobenzyl and 2,4-dimethoxybenzyl, and triphenylmethyl); lower alkenyl groups (for example allyl); di-4-anisylmethyl and furylmethyl groups; lower alkoxycarbonyl (for example tert-butoxycarbonyl); lower alkenyloxycarbonyl (for example allyloxycarbonyl); aryl-lower alkoxycarbonyl groups (for example benzyloxycarbonyl, 4-methoxybenzyloxycarbonyl, 2-nitrobenzyloxycarbonyl and 4-nitrobenzyloxycarbonyl); lower alkanoyloxyalkyl groups (for example pivaloyloxymethyl); trialkylsilyl (for example trimethylsilyl and tert-butyldimethylsilyl); alkylidene (for example methylidene) and benzylidene and substituted benzylidene groups.

Methods appropriate for removal of hydroxy and amino protecting groups include, for example, acid-, base-, metal- or enzymically-catalysed hydrolysis for groups such as 2-nitrobenzyloxycarbonyl and allyl, hydrogenation for groups such as benzyl and photolytically for groups such as 2-nitrobenzyloxycarbonyl. For example a tert butoxycarbonyl protecting group may be removed from an amino group by an acid catalysed hydrolysis using trifluoroacetic acid.

The reader is referred to Advanced Organic Chemistry, 4th Edition, by J. March, published by John Wiley & Sons 1992, for general guidance on reaction conditions and reagents and to Protective Groups in Organic Synthesis, 2^{nd} Edition, by T. Green et al., also published by John Wiley & Son, for general guidance on protecting groups.

It will be appreciated that certain of the various ring substituents in the quinazoline derivatives of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group.

When a pharmaceutically acceptable salt of a quinazoline derivative of the formula I is required, for example an acid-addition salt, it may be obtained by, for example, reaction of said quinazoline derivative with a suitable acid using a conventional procedure.

As mentioned hereinbefore some of the quinazoline derivatives according to the present invention may contain one or more chiral centers and may therefore exist as stereoisomers (for example when R⁴ is alkyl and R⁵ is hydrogen). Stereoisomers may be separated using conventional techniques, e.g. chromatography or fractional crystallisation. The enantiomers may be isolated by separation of a racemate for example by fractional crystallisation, resolution or HPLC. The diastereoisomers may be isolated by separation by virtue of the different physical properties of the diastereoisomers, for example, by fractional crystallisation, HPLC or flash chromatography. Alternatively particular stereoisomers may be made by chiral synthesis from chiral starting materials under conditions which will not cause racemisation or epimerisation, or by derivatisation, with a chiral reagent. When a specific stereoisomer is isolated it is suitably isolated substantially free for other stereoisomers, for example containing less than 20%, particularly less than 10% and more particularly less than 5% by weight of other stereoisomers.

In the section above relating to the preparation of the quinazoline derivatives of the formula I, the expression “inert solvent” refers to a solvent which does not react with the starting materials, reagents, intermediates or products in a manner which adversely affects the yield of the desired product.

Persons skilled in the art will appreciate that, in order to obtain quinazoline derivatives of the invention in an alternative and in some occasions, more convenient manner, the individual process steps mentioned hereinbefore may be performed in different order, and/or the individual reactions may be performed at different stage in the overall route (i.e. chemical transformations may be performed upon different intermediates to those associated hereinbefore with a particular reaction).

Certain intermediates used in the processes described above are novel and form a further feature of the present invention. Accordingly there is provided a compound of the formula IV as hereinbefore defined, or a salt thereof. There is further provided a compound of the formula VI as hereinbefore defined, or a salt thereof. There is further provided a compound of the formula VII as hereinbefore defined, or a salt thereof. There is still further provided a compound of the formula VIII as hereinbefore defined, or a salt thereof and there is further provided a compound of the formula X as hereinbefore defined, or a salt thereof.

The intermediate may be in the form of a salt of the intermediate. Such salts need not be a pharmaceutically acceptable salt. For example it may be useful to prepare an intermediate in the form of a pharmaceutically non-acceptable salt if for example, such salts are useful in the manufacture of a compound of the formula I.

A particular compound of the invention is, for example, any one or more of the compounds of the formula IV selected from:
ethyl[(4-{[3-chloro-4-(pyridin-2-ylmethoxy)phenyl]amino}quinazolin-5-yl)oxy]acetate;
[(4-{[3-chloro-4-(pyridin-2-ylmethoxy)phenyl]amino}quinazolin-5-yl)oxy]acetic acid;
methyl (2R)-2-[(4-{[3-chloro-4-(pyridin-2-ylmethoxy)phenyl]amino}quinazolin-5-yl)oxy]propanoate;
(2R)-2-[(4-{[3-chloro-4-(pyridin-2-ylmethoxy)phenyl]amino}quinazolin-5-yl)oxy]propanoic acid;
methyl (2S)-2-[(4-{[3-chloro-4-(pyridin-2-ylmethoxy)phenyl]amino}quinazolin-5-yl)oxy]propanoate;
(2S)-2-[(4-{[3-chloro-4-(pyridin-2-ylmethoxy)phenyl]amino}quinazolin-5-yl)oxy]propanoic acid;
2-[(4-{[3-chloro-4-(pyridin-2-ylmethoxy)phenyl]amino}quinazolin-5-yl)oxy]-2-methylpropanoic acid;
2-[(4-{[3-methyl-4-(pyridin-2-ylmethoxy)phenyl]amino}quinazolin-5-yl)oxy]-2-methylpropanoic acid;
methyl 4-({3-methyl-4-[(6-methylpyridin-3-yl)oxy]phenyl}amino)quinazolin-5-yl]oxy}acetate;
4-({3-methyl-4-[(6-methylpyridin-3-yl)oxy]phenyl}amino)quinazolin-5-yl]oxy}acetic acid;
methyl (2S)-2-{[4-({3-methyl-4-[(6-methylpyridin-3-yl)oxy]phenyl}amino)quinazolin-5-yl]oxy}propanoate;
(2S)-2-{[4-({3-methyl-4-[(6-methylpyridin-3-yl)oxy]phenyl}amino)quinazolin-5-yl]oxy}propanoic acid;
methyl (2R)-2-{[4-({3-methyl-4-[(6-methylpyridin-3-yl)oxy]phenyl}amino)quinazolin-5-yl]oxy}propanoate;
(2R)-2-{[4-({3-methyl-4-[(6-methylpyridin-3-yl)oxy]phenyl}amino)quinazolin-5-yl]oxy}propanoic acid;
2-methyl-2-{[4-({3-methyl-4-[(6-methylpyridin-3-yl)oxy]phenyl}amino)quinazolin-5-yl]oxy}propanoic acid;
methyl (2R)-2-[(4-{[3-methyl-4-(pyridin-2-ylmethoxy)phenyl]amino}quinazolin-5-yl)oxy]propanoate;
methyl (2R)-2-{[4-({3-methoxy-4-[(6-methylpyridin-3-yl)oxy]phenyl}amino)quinazolin-5-yl]oxy}propanoate;
(2R)-2-{[4-({3-methoxy-4-[(6-methylpyridin-3-yl)oxy]phenyl}amino)quinazolin-5-yl]oxy}propanoic acid;
methyl (2R)-2-[(4-{[3-methyl-4-(pyridin-2-yloxy)phenyl]amino}quinazolin-5-yl)oxy]propanoate;
(2R)-2-[(4-{[3-methyl-4-(pyridin-2-yloxy)phenyl]amino}quinazolin-5-yl)oxy]propanoic acid;
methyl (2R)-2-[(4-{[3-chloro-4-(pyridin-2-yloxy)phenyl]amino}quinazolin-5-yl)oxy]propanoate;
(2R)-2-[(4-{[3-chloro-4-(pyridin-2-yloxy)phenyl]amino}quinazolin-5-yl)oxy]propanoic acid;
methyl (2R)-2-[(4-{[3-chloro-4-(pyridin-2-yloxy)phenyl]amino}quinazolin-5-yl)oxy]propanoate; and
methyl (2R)-2-{[4-({3-chloro-4-[(6-methylpyridin-3-yl)oxy]phenyl}amino)quinazolin-5-yl]oxy}propanoate;
   or a salt thereof.

Another particular compound of the invention is, for example, any one or more of the compounds of the formula VII selected from:
4-[3-chloro-4-(pyridin-2-ylmethoxy)phenyl]-6,6-dimethyl-4H-[1,4]oxazepino[5,6,7-de]quinazolin-5(6H)-one;
4-[3-methyl-4-(pyridin-2-ylmethoxy)phenyl]-6,6-dimethyl-4H-[1,4]oxazepino[5,6,7-de]quinazolin-5(6H)-one; and
6,6-dimethyl-4-{3-methyl-4-[(6-methylpyridin-3-yl)oxy]phenyl}-4H-[1,4]oxazepino[5,6,7-de]quinazolin-5(6H)-one;
   or a salt thereof.

Another particular compound of the invention is, for example, a compound of the formula VIII selected from:
5-[(1R)-1-methyl-2-morpholin-4-yl-2-oxoethoxy]quinazolin-4(3H)-one;
   or a salt thereof.
   Biological Assays

The inhibitory activities of compounds were assessed in non-cell based protein tyrosine kinase assays as well as in cell based proliferation assays before their in vivo activity was assessed in Xenograft studies.

a) Protein Tyrosine Kinase Phosphorylation Assays

This test measures the ability of a test compound to inhibit the phosphorylation of a tyrosine containing polypeptide substrate by an erb receptor tyrosine kinase enzyme.

Recombinant intracellular fragments of EGFR, erbB2 and erbB4 (accession numbers X00588, X03363 and L07868 respectively) were cloned and expressed in the baculovirus/Sf21 system. Lysates were prepared from these cells by treatment with ice-cold lysis buffer (20 mM N-2-hydroxyethylpiperizine-N′-2-ethanesulfonic acid (HEPES) pH7.5, 150 mM NaCl, 10% glycerol, 1% Triton X-100, 1.5 mM MgCl₂, 1 mM ethylene glycol-bis(β-aminoethyl ether) N′,N′,N′,N′-tetraacetic acid (EGTA), plus protease inhibitors and then cleared by centrifugation.

Constitutive kinase activity of these recombinant proteins was determined by their ability to phosphorylate a synthetic peptide (made up of a random co-polymer of Glutamic Acid, Alanine and Tyrosine in the ratio of 6:3:1). Specifically, Maxisorb™ 96-well immunoplates were coated with synthetic peptide (0.2 μg of peptide in a 100 μl phosphate buffered saline (PBS) solution and incubated at 4° C. overnight). Plates were washed in 50 mM HEPES pH 7.4 at room temperature to remove any excess unbound synthetic peptide. EGFR or erbB2 activities were assessed by incubation in peptide coated plates for 20 minutes at room temperature in 50 mM HEPES pH 7.4 at room temperature, adenosine trisphosphate (ATP) at Km concentration for the respective enzyme, 10 mM MnCl₂, 0.05 mM Na₃VO₄, 0.1 mM DL-dithiothreitol (DTT), 0.05% Triton X-100 with test compound in DMSO (final concentration of 2.5%). Reactions were terminated by the removal of the liquid components of the assay followed by washing of the plates with PBS-T (phosphate buffered saline with 0.05% Tween 20).

The immobilised phospho-peptide product of the reaction was detected by immunological methods. Firstly, plates were incubated for 90 minutes at room temperature with anti-phosphotyrosine primary antibodies that were raised in the mouse (4G10 from Upstate Biotechnology). Following extensive washing, plates were treated with Horseradish Peroxidase (HRP) conjugated sheep anti-mouse secondary antibody (NXA931 from Amersham) for 60 minutes at room temperature. After further washing, HRP activity in each well of the plate was measured colorimetrically using 22′-Azino-di-[3-ethylbenzthiazoline sulfonate (6)] diammonium salt crystals (ABTS™ from Roche) as a substrate.

Quantification of colour development and thus enzyme activity was achieved by the measurement of absorbance at 405 nm on a Molecular Devices ThermoMax microplate reader. Kinase inhibition for a given compound was expressed as an IC₅₀ value. This was determined by calculation of the concentration of compound that was required to give 50% inhibition of phosphorylation in this assay. The range of phosphorylation was calculated from the positive (vehicle plus ATP) and negative (vehicle minus ATP) control values.

b) EGFR Driven KB Cell Proliferation Assay

This assay measures the ability of a test compound to inhibit the proliferation of human tumour cell line, KB obtained from the American Type Culture Collection (ATCC)).

KB cells were cultured in Dulbecco's modified Eagle's medium (DMEM) containing 10% foetal calf serum, 2 mM glutamine and non-essential amino acids at 37° C. in a 7.5% CO₂ air incubator. Cells were harvested from the stock flasks using Trypsin/ethylaminediaminetetraacetic acid (EDTA). Cell density was measured using a haemocytometer and viability was calculated using trypan blue solution before being seeded at a density of 1.25×10³ cells per well of a 96 well plate in DMEM containing 2.5% charcoal stripped serum, 1 mM glutamine and non-essential amino acids at 37° C. in 7.5% CO₂ and allowed to settle for 4 hours.

Following adhesion to the plate, the cells are treated with or without EGF (final concentration of 1 ng/ml) and with or without compound at a range of concentrations in dimethylsulfoxide (DMSO) (0.1% final) before incubation for 4 days. Following the incubation period, cell numbers were determined by addition of 50 μl of 3-(4,5-Dimethylthiazol-2-yl)-2,5-diphenyltetrazolium bromide (MTT) (stock 5 mg/ml) for 2 hours. MTT solution was then tipped off, the plate gently tapped dry and the cells dissolved upon the addition of 100 μl of DMSO.

Absorbance of the solubilised cells was read at 540 nm using a Molecular Devices ThermoMax microplate reader. Inhibition of proliferation was expressed as an IC₅₀ value. This was determined by calculation of the concentration of compound that was required to give 50% inhibition of proliferation. The range of proliferation was calculated from the positive (vehicle plus EGF) and negative (vehicle minus EGF) control values.

c) Cellular EGFR Phosphorylation Assay

This assay measures the ability of a test compound to inhibit the phosphorylation of EGFR in KB cells (human naso-pharangeal carcinoma obtained from the American Type Culture Collection (ATCC).

KB cells were cultured in Dulbecco's modified Eagle's medium (DMEM) containing 10% foetal calf serum, 2 mM glutamine and non-essential amino acids at 37° C. in a 7.5% CO₂ air incubator. Cells were harvested from the stock flasks using Trypsin/ethylaminediaminetetraacetic acid (EDTA). Cell density was measured using a haemocytometer and viability was calculated using trypan blue solution before being seeded at a density of 2×10⁵ cells per well of a 6 well plate in DMEM containing 2.5% charcoal stripped serum, 2 mM glutamine and non-essential amino acids at 37° C. in 7.5% CO₂ and allowed to settle for 72 hours.

Following the 72 hour incubation period, the stripped serum containing media was then replaced with serum-free media (DMEM containing 2 mM glutamine and non-essential amino acids) and incubated at 37° C. in 7.5% CO₂ for 72 hours. Following this incubation period, the cells were treated with or without compound at a range of concentrations in dimethylsulfoxide (DMSO) (0.1% final) in serum free DMEM. Following incubation for 1.5 hours at 37° C. in 7.5% CO₂, the cells were treated with EGF (final concentration of 1 μg/ml) and incubated at 37° C. in 7.5% CO₂ for 3 minutes. The media was then removed and the cells washed twice in ice cold Phosphate Buffered Saline before lysis of the cells with 1 ml of ice cold lysis buffer containing 120 mM NaCl₂, 25 mM HEPES, pH 7.6, 5 mM B-Glycerophosphate, 2.5 mM MgCl₂, 1 mM EGTA, 0.2 mM EDTA, 1 mM Na₃VO₄, 1% Triton X-100, 100 mM NaF, 1 mM DTT, 1 mM PMSF, 10 μg/ml Leupeptin and 10 μg/ml Benzamidine. The lysates were centrifuged in a microfuge at 13000 rpm for 15 minutes and the supernatants taken before analysis by sandwich Elisa.

Nunc Maxisorb F96 Immunoplates were coated with EGFR capture antibody (sc-120, Santa Cruz Biotechnology, Inc.) by incubation at a concentration of 0.16 μg/ml in 100 μl of 50 mM carbonate/bicarbonate buffer, pH 9.6. The plates were incubated at 4° C. overnight with a gentle shaking action. Following overnight incubation, the plates were washed extensively with PBS containing 0.05% Tween before blocking with Superblock (Pierce). 100 μl of lysate was then added to each well and incubated overnight at 4° C. before extensive washing with PBS containing 0.05% Tween.

The immobilised EGFR was then probed with an anti-phosphotyrosine HRP conjugated antibody (4G10, Upstate Biotechnology Inc.) at a dilution of 1 in 800 in PBS containing 0.05% Tween plus 0.5% Bovine Serum Albumen. After further washing, HRP activity in each well of the plate was measured colorimetrically using Tetra Methyl Benzidine (TMB) from Bushranger (Roche Applied Sciences) in phosphate-citrate-perborate buffer containing 10% DMSO as a substrate. This reaction was stopped by the addition of 100 ul of 1M H₂SO₄ after 12 minutes and quantified by measurement of the absorbance at 450 nm using a Molecular Devices ThermoMax microplate reader.

Inhibition of EGFR phosphorylation for a given compound was expressed as an IC₅₀ value. This was determined by calculation of the concentration of compound that was required to give 50% inhibition of phosphorylation in this assay. The range of phosphorylation was calculated from the positive (vehicle plus EGF) and negative (vehicle minus EGF) control values.

d) Clone 24 Phospho-erbB2 Cell Assay

This immunofluorescence end point assay measures the ability of a test compound to inhibit the phosphorylation of erbB2 in a MCF7 (breast carcinoma) derived cell line which was generated by transfecting MCF7 cells with the full length erbB2 gene using standard methods to give a cell line that overexpresses full length wild type erbB2 protein (hereinafter ‘Clone 24’ cells).

Clone 24 cells were cultured in Growth Medium (phenol red free Dulbecco's modified Eagle's medium (DMEM) containing 10% foetal bovine serum, 2 mM glutamine and 1.2 mg/ml G418) in a 7.5% CO₂ air incubator at 37° C. Cells were harvested from T75 stock flasks by washing once in PBS (phosphate buffered saline, pH7.4, Gibco No. 10010-015) and harvested using 2 mls of Trypsin (1.25 mg/ml)/ethylaminediaminetetraacetic acid (EDTA) (0.8 mg/ml) solution. The cells were resuspended in Growth Medium. Cell density was measured using a haemocytometer and viability was calculated using Trypan Blue solution before being further diluted in Growth Medium and seeded at a density of 1×10⁴ cells per well (in 100 ul) into clear bottomed 96 well plates (Packard, No. 6005182).

3 days later, Growth Medium was removed from the wells and replaced with 100 ul Assay Medium (phenol red free DMEM, 2 mM glutamine, 1.2 mg/ml G418) either with or without erbB inhibitor compound. Plates were returned to the incubator for 4 hours and then 20 μl of 20% formaldehyde solution in PBS was added to each well and the plate was left at room temperature for 30 minutes. This fixative solution was removed with a multichannel pipette, 100 μl of PBS was added to each well and then removed with a multichannel pipette and then 50 μl PBS was added to each well. Plates were then sealed and stored for up to 2 weeks at 4° C.

Immunostaining was performed at room temperature. Cells were washed once with 200 μl PBS/Tween 20 (made by adding 1 sachet of PBS/Tween dry powder (Sigma, No. P3563) to 1 L of double distilled H₂O) using a plate washer, then 100 μl of 0.5% Triton X-100/PBS was added to each well to permeabalise the cells. After 10 minutes, the plates were washed with 200 μl PBS/Tween 20 and then 100 μl Blocking Solution (5% Marvel dried simmed milk (Nestle) in PBS) was added per well and plates were incubated for 15 minutes. Following removal of the Blocking Solution with a plate washer, 30 μl of rabbit polyclonal anti-phospho ErbB2 IgG antibody (epitope phospho-Tyr 1248, SantaCruz, No. SC-12352-R), diluted 1:250 in Blocking Solution, was added to each well and incubated for 2 hours. Then this primary antibody solution was removed from the wells using a plate washer followed by two 200 μl PBS/Tween 20 washes using a plate washer. 100 μl of Blocking Solution was added per well and plates were incubated for 10 minutes. Then 30 μl of Alexa-Fluor 488 goat anti-rabbit IgG secondary antibody (Molecular Probes, No. A-11008), diluted 1:750 in Blocking Solution, was added to each well. From now onwards, wherever possible, plates were protected from light exposure, at this stage by sealing with black backing tape. The plates were incubated for 45 minutes and then the secondary antibody solution was removed from the wells followed by three 200 ul PBS/Tween 20 washes using a plate washer. Then 50 μl of PBS was added to each well and plates were resealed with black backing tape and stored at 4° C. before analysis. Plates were analysed within six hours of completing the immunostaining.

The Fluorescence signal is each well was measured using an Acumen Explorer Instrument (Acumen Bioscience Ltd.), a plate reader that can be used to rapidly quantitate features of images generated by laser-scanning. The instrument was set to measure the number of fluorescent objects above a pre-set threshold value and this provided a measure of the phosphorylation status of erbB2 protein. Fluorescence dose response data obtained with each compound was exported into a suitable software package (such as Origin) to perform curve fitting analysis. Inhibition of erbB2 phosphorylation was expressed as an IC₅₀ value. This was determined by calculation of the concentration of compound that was required to give 50% inhibition of erbB2 phosphorylation signal.

e) In Vivo BT-474C Xenograft Assay

This assay measures the ability of a test compound to inhibit the growth of a specific variant of the BT-474 tumour cell line grown as a xenograft in Female Swiss athymic mice (Alderley Park, nu/nu genotype) (Baselga, J. et al. (1998) Cancer Research, 58, 2825-2831).

The BT-474 tumour cell line (human mammary carcinoma) was obtained from Dr Baselga (at Laboratorio Recerca Oncologica, Paseo Vall D'Hebron 119-129, Barcelona 08035, Spain). This cell line was subcloned and a certain population (hereinafter referred to as “BT-474C”) was obtained.

Female Swiss athymic (nu/nu genotype) mice were bred and maintained in Alderley Park in negative pressure Isolators (PFI Systems Ltd.). Mice were housed in a barrier facility with 12 hr light/dark cycles and provided with sterilised food and water ad libitum. All procedures were performed on mice of at least 8 weeks of age. BT-474C tumour cell xenografts were established in the hind flank of donor mice by sub-cutaneous injections of 1×10⁷ freshly cultured cells in 100 μl of serum free media with 50% Matrigel per animal. Animals were supplemented with oestradiol benzoate (Mesalin, Intravet UK 0.2 mg/ml), 100 μg/animal injected sub-cutaneously on the day before cell implant, with subsequent weekly boosts of 50 μg/animal. On day 14 post-implant, mice were randomised into groups of 10 prior to the treatment with compound or vehicle control that was administered once daily at 0.1 ml/10 g body weight. Tumour volume was assessed twice weekly by bilateral Vernier calliper measurement, using the formula (length×width)×√(length×width)×(π/6), where length was the longest diameter across the tumour, and width was the corresponding perpendicular. Growth inhibition from start of treatment was calculated by comparison of the mean changes in tumour volume for the control and treated groups, and statistical significance between the two groups was evaluated using a Students t test.

f) hERG-Encoded Potassium Channel Inhibition Assay

This assay determines the ability of a test compound to inhibit the tail current flowing through the human ether-a-go-go-related-gene (hERG)-encoded potassium channel.

Human embryonic kidney (HEK) cells expressing the hERG-encoded channel were grown in Minimum Essential Medium Eagle (EMEM; Sigma-Aldrich catalogue number M2279), supplemented with 10% Foetal Calf Serum (Labtech International; product number 4-101-500), 10% Ml serum-free supplement (Egg Technologies; product number 70916) and 0.4 mg/ml Geneticin G418 (Sigma-Aldrich; catalogue number G7034). One or two days before each experiment, the cells were detached from the tissue culture flasks with Accutase (TCS Biologicals) using standard tissue culture methods. They were then put onto glass coverslips resting in wells of a 12 well plate and covered with 2 ml of the growing media.

For each cell recorded, a glass coverslip containing the cells was placed at the bottom of a Perspex chamber containing bath solution (see below) at room temperature (˜20° C.). This chamber was fixed to the stage of an inverted, phase-contrast microscope. Immediately after placing the coverslip in the chamber, bath solution was perfused into the chamber from a gravity-fed reservoir for 2 minutes at a rate of ˜2 ml/min. After this time, perfusion was stopped.

A patch pipette made from borosilicate glass tubing (GC120F, Harvard Apparatus) using a P-97 micropipette puller (Sutter Instrument Co.) was filled with pipette solution (see hereinafter). The pipette was connected to the headstage of the patch clamp amplifier (Axopatch 200B, Axon Instruments) via a silver/silver chloride wire. The headstage ground was connected to the earth electrode. This consisted of a silver/silver chloride wire embedded in 3% agar made up with 0.85% sodium chloride.

The cell was recorded in the whole cell configuration of the patch clamp technique. Following “break-in”, which was done at a holding potential of −80 mV (set by the amplifier), and appropriate adjustment of series resistance and capacitance controls, electrophysiology software (Clampex, Axon Instruments) was used to set a holding potential (−80 mV) and to deliver a voltage protocol. This protocol was applied every 15 seconds and consisted of a 1 s step to +40 mV followed by a 1 s step to −50 mV. The current response to each imposed voltage protocol was low pass filtered by the amplifier at 1 kHz. The filtered signal was then acquired, on line, by digitising this analogue signal from the amplifier with an analogue to digital converter. The digitised signal was then captured on a computer running Clampex software (Axon Instruments). During the holding potential and the step to +40 mV the current was sampled at 1 kHz. The sampling rate was then set to 5 kHz for the remainder of the voltage protocol.

The compositions, pH and osmolarity of the bath and pipette solution are tabulated below.

| Salt | Pipette (mM) | Bath (mM) |
|---|---|---|
| NaCl | — | 137 |
| KCl | 130 | 4 |
| MgCl₂ | 1 | 1 |
| CaCl₂ | — | 1.8 |
| HEPES | 10 | 10 |
| glucose | — | 10 |
| Na₂ATP | 5 | — |
| EGTA | 5 | — |

| Parameter | Pipette | Bath |
|---|---|---|
| pH | 7.18-7.22 | 7.40 |
| pH adjustment with | 1M KOH | 1M NaOH |
| Osmolarity (mOsm) | 275-285 | 285-295 |

The amplitude of the hERG-encoded potassium channel tail current following the step from +40 mV to −50 mV was recorded on-line by Clampex software (Axon Instruments). Following stabilisation of the tail current amplitude, bath solution containing the vehicle for the test substance was applied to the cell. Providing the vehicle application had no significant effect on tail current amplitude, a cumulative concentration effect curve to the compound was then constructed.

The effect of each concentration of test compound was quantified by expressing the tail current amplitude in the presence of a given concentration of test compound as a percentage of that in the presence of vehicle.

Test compound potency (IC₅₀) was determined by fitting the percentage inhibition values making up the concentration-effect to a four parameter Hill equation using a standard data-fitting package. If the level of inhibition seen at the highest test concentration did not exceed 50%, no potency value was produced and a percentage inhibition value at that concentration was quoted.

Although the pharmacological properties of the quinazoline derivatives of the formula I vary with structural change as expected, in general activity possessed by quinazoline derivatives of the formula I, may be demonstrated at the following concentrations or doses in one or more of the above tests (a), (b), (c) and (d):—

Test (a):—IC₅₀ in the range, for example, 0.001-5 μM;

Test (b):—IC₅₀ in the range, for example, 0.001-5 μM;

Test (c):—IC₅₀ in the range, for example, 0.001-5 μM;

Test (d):—IC₅₀ in the range, for example, 0.001-5 μM;

Test (e):—activity in the range, for example, 1-200 mg/kg/day;

No physiologically unacceptable toxicity was observed in Test (e) at the effective dose for quinazoline derivatives tested of the present invention. Accordingly no untoward toxicological effects are expected when a quinazoline derivative of the formula I, or a pharmaceutically acceptable salt thereof, as defined hereinbefore is administered at the dosage ranges defined hereinafter.

By way of example, Table A illustrates the activity of representative quinazoline derivatives according to the invention. Column 2 of Table A shows IC₅₀ data from Test (a) for the inhibition of EGFR tyrosine kinase protein phosphorylation; column 3 shows IC₅₀ data from Test (a) for the inhibition of erbB2 tyrosine kinase protein phosphorylation; and column 4 shows IC₅₀ data for inhibition of phosphorylation of erbB2 in a MCF7 derived cell line in Test (d) described above:

TABLE A

| | | | IC₅₀ (μM) |
|---|---|---|---|
| | IC₅₀ (μM) Test (a): | IC₅₀ (μM) Test (a): | Test (e): |
| | Inhibition of EGFR | Inhibition of erbB2 | Inhibition of |
| | tyrosine kinase | tyrosine kinase | erbB2 tyrosine |
| Example | protein | protein | kinase protein |
| Number | phosphorylation | phosphorylation | phosphorylation |
| 21 | 0.072 | 0.002 | 0.001 |
| 34 | 0.135 | 0.002 | 0.001 |
| 42 | 24.789 | 0.012 | 0.002 |
| 52 | 1.473 | 0.002 | 0.019 |

According to a further aspect of the invention there is provided a pharmaceutical composition which comprises a quinazoline derivative of the formula I, or a pharmaceutically acceptable salt thereof, as defined hereinbefore in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a quinazoline derivative of the formula I will naturally vary according to the nature and severity of the conditions, the age and sex of the animal or patient and the route of administration, according to well known principles of medicine.

In using a quinazoline derivative of the formula I for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration is however preferred, particularly in tablet form. Typically, unit dosage forms will contain about 0.5 mg to 0.5 g of a quinazoline derivative of this invention.

We have found that the quinazoline derivatives of the present invention possess anti-proliferative properties such as anti-cancer properties that are believed to arise from their erbB, particularly EGFR and more particularly erbB2 receptor tyrosine kinase inhibitory activity. Furthermore, certain of the quinazoline derivatives according to the present invention possess substantially better potency against the erbB2 receptor tyrosine kinase, than against other tyrosine kinases enzymes, such as EGFR tyrosine kinase. Such quinazoline derivatives possess sufficient potency against the erbB2 receptor tyrosine kinase that they may be used in an amount sufficient to inhibit erbB2 receptor tyrosine kinase whilst demonstrating little, or significantly lower, activity against other tyrosine kinases such as EGFR. Such quinazoline derivatives are likely to be useful for the selective inhibition of erbB2 receptor tyrosine kinase and are likely to be useful for the effective treatment of, for example erbB2 driven tumours.

Accordingly, the quinazoline derivatives of the present invention are expected to be useful in the treatment of diseases or medical conditions mediated alone or in part by erbB, particularly erbB2, receptor tyrosine kinases, i.e. the quinazoline derivatives may be used to produce an erbB, particularly an erbB2, receptor tyrosine kinase inhibitory effect in a warm-blooded animal in need of such treatment. Thus the quinazoline derivatives of the present invention provide a method for the treatment of malignant cells characterised by inhibition of the erbB, particularly erbB2, receptor tyrosine kinase. Particularly the quinazoline derivatives of the invention may be used to produce an anti-proliferative and/or pro-apoptotic and/or anti-invasive effect mediated alone or in part by the inhibition of erbB, particularly erbB2, receptor tyrosine kinases. Particularly, the quinazoline derivatives of the present invention are expected to be useful in the prevention or treatment of those tumours that are sensitive to inhibition of an erbB, particularly the erbB2, receptor tyrosine kinase that are involved in the signal transduction steps which drive proliferation and survival of these tumour cells. Accordingly the quinazoline derivatives of the present invention are expected to be useful in the treatment and/or prevention of a number of hyperproliferative disorders by providing an anti-proliferative effect. These disorders include, for example psoriasis, benign prostatic hyperplasia (BPH), atherosclerosis and restenosis and, in particular, erb-B, more particularly erbB2, receptor tyrosine kinase driven tumours. Such benign or malignant tumours may affect any tissue and include non-solid tumours such as leukaemia, multiple myeloma or lymphoma, and also solid tumours, for example bile duct, bone, bladder, brain/CNS, breast, colorectal, cervical, endometrial, gastric, head and neck, hepatic, lung, muscle, neuronal, oesophageal, ovarian, pancreatic, pleural/peritoneal membranes, prostate, renal, skin, testicular, thyroid, uterine and vulval tumours.

According to this aspect of the invention there is provided a quinazoline derivative of the formula I, or a pharmaceutically acceptable salt thereof, for use as a medicament.

Thus according to this aspect of the invention there is provided the use of a quinazoline derivative of the formula I, or a pharmaceutically acceptable salt thereof, as defined hereinbefore in the manufacture of a medicament for use in the production of an anti-proliferative effect in a warm-blooded animal such as man.

According to a further feature of this aspect of the invention there is provided a method for producing an anti-proliferative effect in a warm-blooded animal, such as man, in need of such treatment which comprises administering to said animal an effective amount of a quinazoline derivative of the formula I, or a pharmaceutically acceptable salt thereof, as hereinbefore defined.

According to a further aspect of the invention there is provided a quinazoline derivative of the formula I, or a pharmaceutically acceptable salt thereof, for use in the production of an anti-proliferative effect in a warm-blooded animal such as man.

According to a further aspect of the invention there is provided the use of a quinazoline derivative of the formula I, or a pharmaceutically acceptable salt thereof, as defined hereinbefore in the manufacture of a medicament for use in the production of an anti-proliferative effect which effect is produced alone or in part by inhibiting erbB2 receptor tyrosine kinase in a warm-blooded animal such as man.

According to a further feature of this aspect of the invention there is provided a method for producing an anti-proliferative effect which effect is produced alone or in part by inhibiting erbB2 receptor tyrosine kinase in a warm-blooded animal, such as man, in need of such treatment which comprises administering to said animal an effective amount of a quinazoline derivative of the formula I, or a pharmaceutically acceptable salt thereof, as hereinbefore defined.

According to a further aspect of the invention there is provided a quinazoline derivative of the formula I, or a pharmaceutically acceptable salt thereof, for use in the production of an anti-proliferative effect which effect is produced alone or in part by inhibiting erbB2 receptor tyrosine kinase in a warm-blooded animal such as man.

According to a further aspect of the present invention there is provided the use of a quinazoline derivative of the formula I, or a pharmaceutically acceptable salt thereof as defined hereinbefore in the manufacture of a medicament for use in the treatment of a disease or medical condition (for example a cancer as mentioned herein) mediated alone or in part by erbB, particularly erbB2, receptor tyrosine kinase.

According to a further feature of this aspect of the invention there is provided a method for treating a disease or medical condition (for example a cancer as mentioned herein) mediated alone or in part by erbB, particularly erbB2, receptor tyrosine kinase in a warm-blooded animal, such as man, in need of such treatment, which comprises administering to said animal an effective amount of a quinazoline derivative of the formula I, or a pharmaceutically acceptable salt thereof, as defined hereinbefore.

According to a further aspect of the invention there is provided a quinazoline derivative of the formula I, or a pharmaceutically acceptable salt thereof, for use in the treatment of a disease or medical condition (for example a cancer as mentioned herein) mediated alone or in part by erbB, particularly erbB2, receptor tyrosine kinase.

According to a further aspect of the invention there is provided the use of a quinazoline derivative of the formula I, or a pharmaceutically acceptable salt thereof, as defined hereinbefore in the manufacture of a medicament for use in the prevention or treatment of those tumours which are sensitive to inhibition of one or more erbB receptor tyrosine kinases, such as EGFR and/or erbB2 and/or erbB4 (especially erbB2) receptor tyrosine kinase, that are involved in the signal transduction steps which lead to the proliferation of tumour cells.

According to a further feature of this aspect of the invention there is provided a method for the prevention or treatment of those tumours which are sensitive to inhibition of one or more erbB receptor tyrosine kinases, such as EGFR and/or erbB2 and/or erbB4 (especially erbB2) receptor tyrosine kinase, that are involved in the signal transduction steps which lead to the proliferation and/or survival of tumour cells in a warm-blooded animal, such as man, in need of such treatment, which comprises administering to said animal an effective amount of a quinazoline derivative of the formula I, or a pharmaceutically acceptable salt thereof, as defined hereinbefore.

According to a further aspect of the invention there is provided a quinazoline derivative of the formula I, or a pharmaceutically acceptable salt thereof, for use in the prevention or treatment of those tumours which are sensitive to inhibition of one or more erbB receptor tyrosine kinases, such as EGFR and/or erbB2 and/or erbB4 (especially erbB2) receptor tyrosine kinase, that are involved in the signal transduction steps which lead to the proliferation and/or survival of tumour cells.

According to a further aspect of the invention there is provided the use of a quinazoline derivative of the formula I, or a pharmaceutically acceptable salt thereof, as defined hereinbefore in the manufacture of a medicament for use in providing an EGFR and/or erbB2 and/or erbB4 (especially erbB2) receptor tyrosine kinase inhibitory effect.

According to a further feature of this aspect of the invention there is provided a method for providing an EGFR and/or erbB2 and/or erbB4 (especially erbB2) receptor tyrosine kinase inhibitory effect in a warm-blooded animal, such as man, in need of such treatment, which comprises administering to said animal an effective amount of a quinazoline derivative of the formula I, or a pharmaceutically acceptable salt thereof, as defined hereinbefore.

According to a further aspect of the invention there is provided a quinazoline derivative of the formula I, or a pharmaceutically acceptable salt thereof, for use in providing an EGFR and/or erbB2 and/or erbB4 (especially erbB2) receptor tyrosine kinase inhibitory effect.

According to a further aspect of the invention there is provided the use of a quinazoline derivative of the formula I, or a pharmaceutically acceptable salt thereof as defined hereinbefore in the manufacture of a medicament for use in providing a selective erbB2 kinase inhibitory effect.

According to a further feature of this aspect of the invention there is provided a method for providing a selective erbB2 kinase inhibitory effect in a warm-blooded animal, such as man, in need of such treatment, which comprises administering to said animal an effective amount of a quinazoline derivative of the formula I, or a pharmaceutically acceptable salt thereof, as defined hereinbefore.

According to a further aspect of the invention there is provided a quinazoline derivative of the formula I, or a pharmaceutically acceptable salt thereof, for use in providing a selective erbB2 kinase inhibitory effect.

By “a selective erbB2 kinase inhibitory effect” is meant that the quinazoline derivative of the formula I is more potent against erbB2 receptor tyrosine kinase than it is against other kinases. In particular some of the quinazoline derivatives according to the invention are more potent against erbB2 receptor kinase than they are against other tyrosine kinases such as other erb-B receptor tyrosine kinases, particularly EGFR tyrosine kinase. For example a selective erbB2 kinase inhibitor according to the invention is at least 5 times, preferably at least 10 times, more preferably at least 100 times more potent against erbB2 receptor tyrosine kinase than it is against EGFR tyrosine kinase, as determined from the relative IC₅₀ values in suitable assays (for example by comparing the IC₅₀ value from the Clone 24 phospho-erbB2 cell assay (assay d) described above which measures the inhibition of erbB2 phosphorylation in cells with the IC₅₀ from the KB cellular EGFR phosphorylation assay (assay c) described above which measures the inhibition of EGFR phosphorylation in cells for a given test compound as described above).

According to a further aspect of the present invention there is provided the use of a quinazoline derivative of the formula I, or a pharmaceutically acceptable salt thereof, as defined hereinbefore in the manufacture of a medicament for use in the treatment of a cancer, for example a cancer selected from leukaemia, multiple myeloma, lymphoma, bile duct, bone, bladder, brain/CNS, breast, colorectal, cervical, endometrial, gastric, head and neck, hepatic, lung, muscle, neuronal, oesophageal, ovarian, pancreatic, pleural/peritoneal membranes, prostate, renal, skin, testicular, thyroid, uterine and vulval cancer.

According to a further feature of this aspect of the invention there is provided a method for treating a cancer, for example a cancer selected from selected from leukaemia, multiple myeloma, lymphoma, bile duct, bone, bladder, brain/CNS, breast, colorectal, cervical, endometrial, gastric, head and neck, hepatic, lung, muscle, neuronal, oesophageal, ovarian, pancreatic, pleural/peritoneal membranes, prostate, renal, skin, testicular, thyroid, uterine and vulval cancer in a warm-blooded animal, such as man, in need of such treatment, which comprises administering to said animal an effective amount of a quinazoline derivative of the formula I, or a pharmaceutically acceptable salt thereof, as defined hereinbefore.

According to a further aspect of the invention there is provided a quinazoline derivative of the formula I, or a pharmaceutically acceptable salt thereof, for use in the treatment of a cancer, for example a cancer selected from leukaemia, multiple myeloma, lymphoma, bile duct, bone, bladder, brain/CNS, breast, colorectal, cervical, endometrial, gastric, head and neck, hepatic, lung, muscle, neuronal, oesophageal, ovarian, pancreatic, pleural/peritoneal membranes, prostate, renal, skin, testicular, thyroid, uterine and vulval cancer.

As mentioned above the size of the dose required for the therapeutic or prophlyactic treatment of a particular disease will necessarily be varied depending upon, amongst other things, the host treated, the route of administration and the severity of the illness being treated.

The quinazoline derivatives of the invention may be administered in the form of a pro-drug, by which we mean a compound that is broken down in a warm-blooded animal, such as man, to release a quinazoline derivative of the invention. A pro-drug may be used to alter the physical properties and/or the pharmacokinetic properties of a quinazoline derivative of the invention. A pro-drug can be formed when the quinazoline derivative of the invention contains a suitable group or substituent to which a property-modifying group can be attached.

Accordingly, the present invention includes those quinazoline derivatives of the formula I as defined hereinbefore when made available by organic synthesis and when made available within the human or animal body by way of cleavage of a pro-drug thereof. Accordingly, the present invention includes those quinazoline derivatives of the formula I that are produced by organic synthetic means and also such quinazoline derivatives that are produced in the human or animal body by way of metabolism of a precursor compound, that is a quinazoline derivative of the formula I may be a synthetically-produced quinazoline derivative or a metabolically-produced quinazoline derivative.

A suitable pharmaceutically-acceptable pro-drug of a quinazoline derivative of the formula I is one that is based on reasonable medical judgement as being suitable for administration to the human or animal body without undesirable pharmacological activities and without undue toxicity.

Various forms of pro-drug have been described, for example in the following documents:—

a) Methods in Enzmmology, Vol. 42, p. 309 to 396, edited by K. Widder, et al. (Academic Press, 1985);

b) Design of Pro-drugs, edited by H. Bundgaard, (Elsevier, 1985);

c) A Textbook of Drug Design and Development, edited by Krogsgaard-Larsen and H. Bundgaard, Chapter 5 “Design and Application of Pro-drugs”, edited by H. Bundgaard, p. 113 to 191 (1991);

d) H. Bundgaard, Advanced Drug Delivery Reviews, 8, 1 to 38 (1992); and

e) H. Bundgaard, et al., Journal of Pharmaceutical Sciences, 77, 285 (1988).

The anti-proliferative treatment defined hereinbefore may be applied as a sole therapy or may involve, in addition to the quinazoline derivative of the invention, conventional surgery or radiotherapy or chemotherapy. Such chemotherapy may include one or more of the following categories of anti-tumour agents:—

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulfan and nitrosoureas); antimetabolites (for example antifolates such as fluoropyrimidines like 5-fluorouracil and tegafur, raltitrexed, methotrexate, cytosine arabinoside and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);

(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;

(iii) agents which inhibit cancer cell invasion (for example metalloproteinase inhibitors like marimastat and inhibitors of urokinase plasminogen activator receptor function);

(iv) inhibitors of growth factor function, for example such inhibitors include growth factor antibodies, growth factor receptor antibodies (for example the anti-erbB2 antibody trastuzumab [Herceptin™] and the anti-erbB1 antibody cetuximab [C225]), farnesyl transferase inhibitors, tyrosine kinase inhibitors and serine/threonine kinase inhibitors, for example other inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;

(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);

(vi) vascular damaging agents such as Combretastatin A4 and compounds disclosed in International Patent Applications WO 99/02166, WO 00/40529, WO 00/41669, WO 01/92224, WO 02/04434 and WO 02/08213;

(vii) antisense therapies, for example those which are directed to the targets listed above, such as ISIS 2503, an anti-ras antisense;

(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and

(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment. Such combination products employ the quinazoline derivatives of this invention within the dosage range described hereinbefore and the other pharmaceutically-active agent within its approved dosage range.

According to this aspect of the invention there is provided a pharmaceutical product comprising a quinazoline derivative of the formula I, or a pharmaceutically acceptable salt thereof, as defined hereinbefore and an additional anti-tumour agent as defined hereinbefore for the conjoint treatment of cancer.

Although the quinazoline derivatives of the formula I are primarily of value as therapeutic agents for use in warm-blooded animals (including man), they are also useful whenever it is required to inhibit the effects of the erbB receptor tyrosine protein kinases. Thus, they are useful as pharmacological standards for use in the development of new biological tests and in the search for new pharmacological agents.

The invention will now be illustrated by the following non-limiting examples in which, unless stated otherwise:

(i) temperatures are given in degrees Celsius (° C.); operations were carried out at room or ambient temperature, that is, at a temperature in the range of 18 to 25° C.;

(ii) organic solutions were dried over anhydrous magnesium sulfate; evaporation of solvent was carried out using a rotary evaporator under reduced pressure (600-4000 Pascals; 4.5-30 mmHg) with a bath temperature of up to 80° C.;

(iii) chromatography means flash chromatography on silica gel; thin layer chromatography (TLC) was carried out on silica gel plates;

(iv) in general, the course of reactions was followed by TLC and/or analytical LC-MS, and reaction times are given for illustration only;

(v) final products had satisfactory proton nuclear magnetic resonance (NMR) spectra and/or mass spectral data;

(vi) yields are given for illustration only and are not necessarily those which can be obtained by diligent process development; preparations were repeated if more material was required;

(vii) when given, NMR data is in the form of delta values for major diagnostic protons, given in parts per million (ppm) relative to tetramethylsilane (TMS) as an internal standard, determined at 300 MHz using perdeuterio dimethyl sulfoxide (DMSO-d₆) as solvent unless otherwise indicated; the following abbreviations have been used: s, singlet; d, doublet; t, triplet; q, quartet; m, multiplet; b, broad;

(viii) chemical symbols have their usual meanings; SI units and symbols are used;

(ix) solvent ratios are given in volume:volume (v/v) terms; and

(x) mass spectra were run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported; and unless otherwise stated, the mass ion quoted is (MH)⁺ which refers to the protonated mass ion; reference to M⁺ is to the mass ion generated by loss of an electron; and reference to M-H⁺ is to the mass ion generated by loss of a proton;

(xi) unless stated otherwise compounds containing an asymmetrically substituted carbon and/or sulfur atom have not been resolved;

(xii) where a synthesis is described as being analogous to that described in a previous example the amounts used are the millimolar ratio equivalents to those used in the previous example;

(xiii) all microwave reactions were carried out in a CEM Discover™ microwave synthesis or CEM Marrs microwave synthesisor;

(xiv) preparative high performance liquid chromatography (HPLC) was performed on a Gilson instrument using the following conditions:

Column: 21 mm×10 cm Hichrom RPB
Solvent A: Water+0.1% trifluoroacetic acid,
Solvent B: Acetonitrile+0.1% trifluoroacetic acid
Flow rate: 18 ml/min
Run time: 15 minutes with a 10 minute gradient from 5-95% B
Wavelength: 254 nm, bandwidth 10 nm
Injection volume 2.0-4.0 ml;
   (xv) analytical HPLC was performed on a LC/MS Waters 2790/ZMD Micromass system using the following conditions (so as to measure retention times (t_{R}):
Waters Symmetry column: C18, 3.5 μM, 4.6×50 mm
Detection: UV 254 nM and MS
Elution: flow rate 2.5 ml/min, linear gradient from 95% water and 5% methanol containing 5% formic acid to 40% water, 55% acetonitrile and 5% methanol containing 5% formic acid over 3 minutes, then linear gradiant to 95% acetonitrile and 5% methanol containing 5% formic acid over 1 minute;
   (xvi) the following abbreviations have been used:
   HATU O-(7-azabenzotriazol-1-yl)-N,N,N′,N′-tetramethyluronium hexafluoro-phosphate;
   THF tetrahydrofuran;
   DMF N,N-dimethylformamide;
   DMA N,N-dimethylacetamide;
   DCM dichloromethane;
   DMSO dimethylsulfoxide;
   IPA isopropyl alcohol;
   ether diethyl ether;
   DIPEA di-isopropylethylamine;
   TFA trifluoroacetic acid
   DEAD diethyl azodicarboxylate;
   DTAD di-tert-butyl azodicarboxylate; and
   EDCI 1-(3-dimethylaminopropyl)-3-ethylcarbodiimide hydrochloride.

### EXAMPLE 1

### 2-[(4-{[3-Chloro-4-(pyridin-2-ylmethoxy)phenyl]amino}quinazolin-5-yl)oxy]acetamide

To a suspension of 4-{[3-chloro-4-(pyridin-2-ylmethoxy)phenyl]amino}quinazolin-5-ol (200 mg, 0.52 mmol) in DMA (15 ml) was added potassium carbonate (359 mg, 2.60 mmol) and 2-bromoacetamide (80 mg, 0.58 mmol). The reaction was sonicated for 5 minutes in an ultrasonic cleaning bath and then stirred for 16 hours at room temperature. The solvent was removed in vacuo, water was then added to the residue and the resultant precipitate was filtered and washed with water. The solid was crystallised from ethyl acetate to give the title compound as an off-white solid (30 mg, 13%); NMR spectrum: 4.86 (s, 2H), 5.31 (s, 2H), 6.97 (d, 1H), 7.26 (d, 1H), 7.39 (m, 2H), 7.60 (d, 1H), 7.62 (s, 1H), 7.76 (t, 1H), 7.83 (s, 1H), 7.90 (td, 1H), 8.04 (dd, 1H), 8.34 (d, 1H), 8.57 (s, 1H), 8.62 (d, 1H), 10.96 (s, 1H); Mass spectrum: MH⁺ 436.

The 4-{[3-chloro-4-(pyridin-2-ylmethoxy)phenyl]amino}quinazolin-5-ol used as starting material was obtained as follows:

DMF (0.2 ml) was added to a suspension of 5-fluoro-3,4-dihydro-3H-quinazolin-4-one (1.64 g) in thionyl chloride (10 ml) and the mixture was stirred and heated at 80° C. for 6 hours. Volatile material was removed by evaporation and the residue was azeotroped with toluene (20 ml). The resulting solid was added portionwise to a vigorously stirred mixture of saturated sodium bicarbonate (50 ml), crushed ice (50 g) and DCM (50 ml) such that the temperature was kept below 5° C. The organic phase was separated, dried and concentrated to give 4-chloro-5-fluoroquinazoline as a solid (1.82 g, 99%), which was used without purification; NMR spectrum: (CDCl₃) 7.35-7.45 (m, 1H), 7.85-7.95 (m, 2H), 9.0 (s, 1H).

4-Chloro-5-fluoroquinazoline (6.75 g) was added to a stirred solution of 3-chloro-4-(2-pyridylmethoxy)aniline (obtained as described in Example 15 of WO 96/15118, 9.27 g) in IPA (200 ml), and the solution was stirred and heated under reflux for 8 hours. The solution was allowed to cool to ambient temperature overnight and the precipitated solid was filtered off, washed with acetone and dried. The solid was added to 50% aqueous methanol (400 ml) and the mixture was heated on a steam bath until all of the solid had dissolved. The solution was basified by careful addition of aqueous ammonia (0.880), and the mixture was concentrated to remove methanol. Water (300 ml) was added and the mixture was extracted with DCM (600 ml). The extract washed with water and saturated brine and dried. The solvent was removed by evaporation to give a solid, which was re-crystallised from a mixture of ethyl acetate, tetrahydrofuran and isohexane to give N-[3-chloro-4-(pyridin-2-ylmethoxy)phenyl]-5-fluoroquinazolin-4-amine as beige crystals (6.75 g, 48%); NMR spectrum: 5.3 (s, 2H), 7.2-7.3 (d, 1H), 7.35-7.5 (m, 2H), 7.5-7.65 (m, 3H), 7.8-7.95 (m, 3H), 8.55 (s, 1H), 8.55-8.6 (d, 1H), 9.1-9.2 (b s, 1H); Mass spectrum: MH⁺ 381.4.

N-Acetylethanolamine (24.3 ml, 0.264 mol) was added slowly to a suspension of sodium hydride (60% dispersion in mineral oil, 25.28 g, 0.632 mmol) in dry DMA (400 ml). Upon complete addition, the mixture was stirred for 30 minutes. N-[3-Chloro-4-(pyridin-2-ylmethoxy)phenyl]-5-fluoroquinazolin-4-amine (40 g, 0.105 mol) was added in one portion and the mixture was heated at 120° C. for 18 hours. Saturated ammonium chloride (15 ml) was added to the cooled reaction mixture and stirred for 10 minutes. The DMA was removed in vacuo, water (1000 ml) was added to the residue and stirred for 1 hour. The resultant precipitate was filtered and air-dried. The solid washed with diethyl ether (2×200 ml). This was then stirred in hot ethyl acetate (300 ml) and the cool mixture was filtered to give 4-{[3-chloro-4-(pyridin-2-ylmethoxy)phenyl]amino}quinazolin-5-ol as a tan solid (31.1 g, 78%); NMR spectrum: 5.28 (s, 2H), 6.63-6.81 (m, 2H), 7.22 (d, 1H), 7.32-7.39 (m, 1H), 7.39-7.52 (m, 2H), 7.57 (d, 1H), 7.87 (t, 1H), 7.97 (s, 1H), 8.33 (s, 1H), 8.58 (d, 1H); Mass spectrum: MH⁺ 379.

### EXAMPLE 2

### 2-{4-[3-Chloro-4-(pyridin-2-ylmethoxy)-phenylamino]-quinazolin-5-yloxy}-N-(2-methanesulfonyl-ethyl)-acetamide

2-Methanesulfonyl-ethylamine (48 mg, 0.40 mmol) and DIPEA (140 μl, 0.80 mmol) were added to a warmed solution of [(4-{[3-chloro-4-(pyridin-2-ylmethoxy)phenyl]amino}quinazolin-5-yl)oxy]acetic acid sodium salt (150 mg, 0.34 mmol) in DMF (3 ml). HATU (149 mg, 0.4 mmol) was added and the resulting yellow solution was stirred at 65° C. for 18 hours. The solvent was removed in vacuo, and water (5 ml) added. The suspension was sonicated before filtering the solid. This washed well with water and dried in vacuo to give the title compound as a yellow solid (146 mg, 89%); NMR spectrum: 3.00 (s, 3H), 3.30 (m, 2H), 3.60 (m, 2H), 4.90 (s, 1.5H), 5.00 (s, 0.5H), 5.30 (s, 2H), 7.00 (d, 0.75H), 7.10 (d, 0.25H), 7.30 (m, 1H), 7.35 (m, 2H), 7.60 (d, 1H), 7.70 (m, 1H), 7.90 (m, 2H), 8.20 (m, 0.25H), 8.30 (m, 0.75H), 8.50-8.70 (m, 3H), 10.80 (s, 1H); Mass spectrum: MH⁺ 542.

The [(4-{[3-chloro-4-(pyridin-2-ylmethoxy)phenyl]amino}quinazolin-5-yl)oxy]acetic acid sodium salt used as starting material was obtained as follows:

Sodium ethoxide (4.5 g, 66.2 mmol) was added to a suspension of N-[3-chloro-4-(pyridin-2-ylmethoxy)phenyl]-5-fluoroquinazolin-4-amine (obtained as described in Example 1, preparation of starting materials, 5.0 g, 13.2 mmol) in ethyl glycolate (75 ml) and the reaction heated at reflux for 16 hours. The reaction was then cooled, and the resulting solid precipitate filtered and washed with methanol to give ethyl[(4-{[3-chloro-4-(pyridin-2-ylmethoxy)phenyl]amino}quinazolin-5-yl)oxy]acetate as a white powder (4.92 g, 81%); NMR spectrum: 1.27 (t, 3H), 4.30 (q, 2H), 5.07 (s, 2H), 5.29 (s, 2H), 7.10 (d, 1H), 7.29 (d, 1H), 7.36 (m, 2H), 7.57 (d, 1H), 7.72 (t, 1H), 7.80 (dd, 1H), 8.08 (dt, 1H), 8.24 (d, 1H), 8.53 (s, 1H), 8.59 (d, 1H), 10.44 (bs, 1H); Mass spectrum: MH⁺ 465.

3M Sodium hydroxide solution (35 ml, 105 mmol) was added to a stirred solution of ethyl[(4-{[3-chloro-4-(pyridin-2-ylmethoxy)phenyl]amino}quinazolin-5-yl)oxy]acetate (4.92 g, 10.6 mmol) in THF (125 ml) and methanol (125 ml). After 30 minutes a dense white solid was precipitated which was filtered, washed with water, then methanol and dried in vacuo to give [(4-{[3-chloro-4-(pyridin-2-ylmethoxy)phenyl]amino}quinazolin-5-yl)oxy]acetic acid sodium salt as a white solid (2.35 g, 51%); NMR spectrum: 4.90 (m, 2H), 5.26 (s, 2H), 7.10 (m, 1H), 7.25 (m, 1H), 7.33 (m, 2H), 7.55 (m, 1H), 7.70 (m, 1H), 7.83 (m, 1H), 7.94 (m, 1H), 8.25 (m, 1H), 8.57 (m, 2H), 10.82 (bs, 1H); Mass spectrum: MH⁺ 437.

### EXAMPLE 3

### 2-{4-[3-Chloro-4-(pyridin-2-ylmethoxy)-phenylamino]-quinazolin-5-yloxy}-N-cyclopropyl-acetamide

The procedure described in Example 2 was repeated using [(4-{[3-chloro-4-(pyridin-2-ylmethoxy)phenyl]amino}quinazolin-5-yl)oxy]acetic acid sodium salt (obtained as described in Example 2, preparation of starting materials, 100 mg, 0.23 mmol), HATU (308 mg, 0.81 mmol), DIPEA (120 μl, 0.69 mmol) and cyclopropylamine (92 mg, 1.61 mmol) to give the title compound as a solid (3 mg, 2%); Mass spectrum: MH⁺ 477.

### EXAMPLE 4

### 2-{4-[3-Chloro-4-(pyridin-2-ylmethoxy)-phenylamino]-quinazolin-5-yloxy}-N-cyclobutyl-acetamide

The procedure described in Example 2 was repeated using [(4-{[3-chloro-4-(pyridin-2-ylmethoxy)phenyl]amino}quinazolin-5-yl)oxy]acetic acid sodium salt (obtained as described in Example 2, preparation of starting materials, 100 mg, 0.23 mmol), HATU (308 mg, 0.81 mmol), DIPEA (120 μl, 0.69 mmol) and cyclobutylamine (114 mg, 1.61 mmol) to give the title compound as a solid (10 mg, 6%); NMR spectrum: 1.60 (m, 2H), 1.90 (m, 2H), 2.20 (m, 2H), 4.25 (m, 1H), 4.80 (s, 2H), 5.20 (s, 2H), 6.90 (d, 1H), 7.20 (d, 1H), 7.25 (dd, 1H), 7.30 (d, 1H), 7.50 (d, 1H), 7.7 (dd, 1H), 7.80-7.90 (m, 2H), 8.15 (d, 1H), 8.20 (bs, 1H), 8.45 (s, 1H), 8.50 (d, 1H), 10.50 (s, 1H); Mass spectrum: MH⁺ 491.

### EXAMPLE 5

### 2-{4-[3-Chloro-4-(pyridin-2-ylmethoxy)-phenylamino]-quinazolin-5-yloxy}-N-(2-methoxy-ethyl)-acetamide

The procedure described in Example 2 was repeated using [(4-{[3-chloro-4-(pyridin-2-ylmethoxy)phenyl]amino}quinazolin-5-yl)oxy]acetic acid sodium salt (obtained as described in Example 2, preparation of starting materials, 100 mg, 0.23 mmol), HATU (308 mg, 0.81 mmol), DIPEA (120 μl, 0.69 mmol) and 2-methoxy-ethylamine (121 mg, 1.61 mmol) to give the title compound as a solid (19 mg, 12%); NMR spectrum: 3.40 (s, 3H), 3.50 (m, 2H), 3.60 (m, 2H), 5.00 (s, 2H), 5.40 (s, 2H), 7.10 (d, 1H), 7.35 (d, 1H), 7.45 (m, 1H), 7.55 (d, 1H), 7.70 (d, 1H), 7.85 (t, 1H), 7.90-8.05 (m, 2H), 8.20 (bs, 1H), 8.35 (m, 1H), 8.65 (s, 1H), 8.70 (d, 1H), 10.75 (s, 1H); Mass spectrum: MH⁺ 495.

### EXAMPLE 6

### 2-{4-[3-Chloro-4-(pyridin-2-ylmethoxy)-phenylamino]-quinazolin-5-yloxy}-N-ethyl-acetamide

The procedure described in Example 2 was repeated using [(4-{[3-chloro-4-(pyridin-2-ylmethoxy)phenyl]amino}quinazolin-5-yl)oxy]acetic acid sodium salt (obtained as described in Example 2, preparation of starting materials, 100 mg, 0.23 mmol), HATU (308 mg, 0.81 mmol), DIPEA (120 μl, 0.69 mmol) and ethylamine (72 mg, 1.61 mmol) to give the title compound as a solid (6 mg, 4%); Mass spectrum: MH⁺ 465.

### EXAMPLE 7

### N-Allyl-2-{4-[3-chloro-4-(pyridin-2-ylmethoxy)-phenylamino]-quinazolin-5-yloxy}-acetamide

The procedure described in Example 2 was repeated using [(4-{[3-chloro-4-(pyridin-2-ylmethoxy)phenyl]amino}quinazolin-5-yl)oxy]acetic acid sodium salt (obtained as described in Example 2, preparation of starting materials, 100 mg, 0.23 mmol), HATU (308 mg, 0.81 mmol), DIPEA (120 μl, 0.69 mmol) and allylamine (92 mg, 1.61 mmol) to give the title compound as a solid (7 mg, 5%); NMR spectrum: 3.80 (m, 2H), 4.80 (s, 2H), 5.00-5.15 (m, 2H), 5.20 (s, 2H), 5.80 (m, 1H), 6.90 (d, 1H), 7.10-7.20 (d, 2H), 7.25 (m, 1H), 7.30 (d, 1H), 7.50 (d, 1H), 7.60 (t, 1H), 7.75-8.00 (m, 2H), 8.20 (m, 1H), 8.45 (s, 1H), 8.55 (d, 1H), 10.50 (s, 1H); Mass spectrum: MH⁺ 477.

### EXAMPLE 8

### 2-{4-[3-Chloro-4-(pyridin-2-ylmethoxy)-phenylamino]-quinazolin-5-yloxy}-N-ethyl-N-methyl-acetamide

The procedure described in Example 2 was repeated using [(4-{[3-chloro-4-(pyridin-2-ylmethoxy)phenyl]amino}quinazolin-5-yl)oxy]acetic acid sodium salt (obtained as described in Example 2, preparation of starting materials, 100 mg, 0.23 mmol), HATU (308 mg, 0.81 mmol), DIPEA (120 μl, 0.69 mmol) and ethyl-methyl-amine (93 mg, 1.61 mmol) to give the title compound as a solid (11 mg, 7%); NMR spectrum: 1.00 (t, 3H), 2.90 (s, 3H), 3.40 (m, 2H), 5.00 (s, 2H), 5.15 (s, 2H), 7.10 (d, 1H), 7.15 (d, 1H), 7.20-7.30 (m, 2H), 7.45 (d, 1H), 7.60 (t, 1H), 7.75 (t, 1H), 7.90 (m, 1H), 8.20 (s, 1H), 8.40 (s, 1H), 8.50 (d, 1H), 10.90 (s, 1H); Mass spectrum: MH⁺ 479.

### EXAMPLE 9

### 2-[(4-{[3-Chloro-4-(pyridin-2-ylmethoxy)phenyl]amino}quinazolin-5-yl)oxy]-N-(2-morpholin-4-ylethyl)acetamide

A mixture of 4-{[3-chloro-4-(pyridin-2-ylmethoxy)phenyl]amino}quinazolin-5-ol (obtained as described in Example 1, preparation of starting materials, 245 mg, 0.65 mmol), 2-chloro-N-(2-morpholin-4-ylethyl)acetamide (147 mg, 0.71 mmol), potassium carbonate (268 mg, 1.94 mmol) and potassium iodide (107 mg, 0.65 mmol) in DMA (2.5 ml) was stirred at room temperature for 36 hours and then at 50° C. for 6 hours. After evaporation of the solvents in vacuo, the residue was purified on an HPLC column (C18, 5 microns, 19 mm diameter, 100 mm length) of a preparative HPLC-MS system eluting with a mixture of water and acetonitrile containing 2 g/l of ammonium carbonate (gradient). Further purification by chromatography on silica gel eluting with 5%-7% 7N ammonia/methanol in DCM gave the title compound as a pale solid (98 mg, 27%); NMR spectrum: (400 MHz; DMSO-d6+CF₃CO₂D) 3.16 (m, 2H), 3.30 (m, 2H), 3.68-3.53 (m, 6H), 3.98 (m, 2H), 5.06 (s, 2H), 5.56 (s, 2H), 7.32 (d, 1H), 7.43 (d, 1H), 7.49 (d, 1H), 7.82 (m, 2H), 7.98 (d, 1H), 8.08 (m, 2H), 8.39 (m, 1H), 8.88 (d, 1H), 8.99 (s, 1H); Mass spectrum: MH⁺ 549.

The 2-chloro-N-(2-morpholin-4-ylethyl)acetamide used as starting material was made as follows:

Chloroacetyl chloride (5.7 ml, 71.8 mmol) was added dropwise to an ice-cooled solution of 4-(2-aminoethyl)morpholine (8.5 g, 65.3 mmol) and triethylamine (10 ml, 71.8 mmol) in DCM (120 ml). The mixture was stirred at room temperature for 90 minutes, washed with water and dried over MgSO₄. After evaporation of the solvents in vacuo, the residue was purified by chromatography on silica gel eluting with 3% MeOH in DCM to give 2-chloro-N-(2-morpholin-4-ylethyl)acetamide as a solid (4.4 g, 33%); Mass spectrum: MH⁺ 207.

### EXAMPLE 10

### 2-{4-[3-Chloro-4-(pyridin-2-ylmethoxy)-phenylamino]-quinazolin-5-yloxy}-N-methyl-N-prop-2-ynyl-acetamide

The procedure described in Example 2 was repeated using [(4-{[3-chloro-4-(pyridin-2-ylmethoxy)phenyl]amino}quinazolin-5-yl)oxy]acetic acid sodium salt (obtained as described in Example 2, preparation of starting materials, 100 mg, 0.23 mmol), HATU (308 mg, 0.81 mmol), DIPEA (120 μl, 0.69 mmol) and methyl-prop-2-ynyl-amine (109 mg, 1.61 mmol) to give the title compound as a solid (54 mg, 35%); NMR spectrum: 2.60 (m, 1H), 3.00 (s, 3H), 4.20 (s, 2H), 5.10 (s, 2H), 5.25 (s, 2H), 7.10 (d, 1H), 7.15 (d, 1H), 7.25 (m, 1H), 7.35 (d, 1H), 7.50 (d, 1H), 7.65 (t, 1H), 7.80 (t, 1H), 7.90 (d, 1H), 8.25 (d, 1H), 8.45 (s, 1H), 8.50 (d, 1H), 10.80 (s, 1H); Mass spectrum: MH⁺ 489.

### EXAMPLE 11

### 2-[(4-{[3-Chloro-4-(pyridin-2-ylmethoxy)phenyl]amino}quinazolin-5-yl)oxy]-N-(2-hydroxyethyl)-N-methylacetamide

HATU (0.2 g, 0.53 mmol) was added to a solution of [(4-{[3-chloro-4-(pyridin-2-ylmethoxy)phenyl]amino}quinazolin-5-yl)oxy]acetic acid sodium salt (obtained as described in Example 2, preparation of starting materials, 0.15 g, 0.33 mmol), 2-(methylamino)ethanol (0.039 g, 0.52 mmol) and DIPEA (0.18 ml, 1.03 mmol) in DMF (10 ml), and the solution stirred overnight. The reaction was concentrated in vacuo and the residue triturated with water to give a white solid. The solid was isolated by filtration and triturated with ether to give the title compound as a white solid (0.11 g, 65%); NMR spectrum: 3.29 (s, 3H), 3.46 (m, 2H), 3.60 (m, 2H), 4.71 and 4.95 (1H, broad t, split), 5.12 and 5.20 (s, 2H, split), 5.29 (s, 2H), 7.18 (m, 1H), 7.27 (d, 1H), 7.35 (d, 2H), 7.58 (d, 1H), 7.73 (t, 1H), 7.87 (t, 1H), 7.98 (dt, 1H), 8.38 (s, 1H), 8.54 (s, 1H), 8.58 (d, 1H), 11.14 (bs, 1H); Mass spectrum: MH⁺ 494.

### EXAMPLE 12

### 2-{4-[3-Chloro-4-(pyridin-2-ylmethoxy)-phenylamino]-quinazolin-5-yloxy}-N-(2-methanesulfonyl-ethyl)-N-methyl-acetamide

The procedure described in Example 2 was repeated using [(4-{[3-chloro-4-(pyridin-2-ylmethoxy)phenyl]amino}quinazolin-5-yl)oxy]acetic acid sodium salt (obtained as described in Example 2, preparation of starting materials, 150 mg, 0.34 mmol), HATU (462 mg, 1.22 mmol), DIPEA (180 μl, 1.03 mmol) and (2-methanesulfonyl-ethyl)-methyl-amine (54 mg, 0.40 mmol) to give the title compound as a solid (149 mg, 84%); NMR spectrum: 2.90-3.10 (m, 6H), 3.40-3.60 (m, 2H), 3.80 (m, 2H), 5.20 (s, 1.3H), 5.30 (s, 2.7H), 7.20-7.40 (m, 4H), 7.60 (d, 1H), 7.80-8.00 (m, 3H), 8.30 (m, 1H), 8.60 (d, 1H), 8.65 (s, 1H); Mass spectrum: MH⁺ 556.

### EXAMPLE 13

### 2-{4-[3-Chloro-4-(pyridin-2-ylmethoxy)-phenylamino]-quinazolin-5-yloxy}-N-methyl-N-(1-methyl-piperidin-4-yl)-acetamide

The procedure described in Example 2 was repeated using [(4-{[3-chloro-4-(pyridin-2-ylmethoxy)phenyl]amino}quinazolin-5-yl)oxy]acetic acid sodium salt (obtained as described in Example 2, preparation of starting materials, 100 mg, 0.23 mmol), HATU (308 mg, 0.81 mmol), DIPEA (120 μl, 0.69 mmol) and methyl-(1-methyl-piperidin-4-yl)-amine (206 mg, 1.61 mmol) to give the title compound as a solid (22 mg, 13%); NMR spectrum: 1.50 (m, 2H), 1.75 (m, 2H), 2.00 (m, 2H), 2.10 (s, 3H), 3.70 (m, 2H), 3.80-3.90 (m, 4H), 5.00 (s, 2H), 5.15 (s, 2H), 7.10 (m, 2H), 7.25 (m, 2H), 7.45 (d, 1H), 7.60 (t, 1H), 7.75 (t, 1H), 7.85 (m, 1H), 8.20 (s, 1H), 8.40 (s, 1H), 8.50 (d, 1H), 11.00 (s, 1H); Mass spectrum: MH⁺ 546.

### EXAMPLE 14

### 2-{4-[3-Chloro-4-(pyridin-2-ylmethoxy)-phenylamino]-quinazolin-5-yloxy}-N-isopropyl-N-methyl-acetamide

The procedure described in Example 2 was repeated using [(4-{[3-chloro-4-(pyridin-2-ylmethoxy)phenyl]amino}quinazolin-5-yl)oxy]acetic acid sodium salt (obtained as described in Example 2, preparation of starting materials, 100 mg, 0.23 mmol), HATU (308 mg, 0.81 mmol), DIPEA (120 μl, 0.69 mmol) and isopropyl-methyl-amine (116 mg, 1.61 mmol) to give the title compound as a solid (15 mg, 16%); NMR spectrum: 1.20 (d, 6H), 2.80 (s, 3H), 2.90 (m, 1H), 5.10 (s, 2H), 5.25 (s, 2H), 7.10 (d, 1H), 7.20 (d, 1H), 7.25-7.35 (m, 2H), 7.50 (d, 1H), 7.65 (t, 1H), 7.75 (t, 1H), 7.90 (m, 1H), 8.25 (m, 1H), 8.45 (s, 1H), 8.55 (d, 1H), 10.75 (s, 1H); Mass spectrum: MH⁺ 493.

### EXAMPLE 15

### 2-{4-[3-Chloro-4-(pyridin-2-ylmethoxy)-phenylamino]-quinazolin-5-yloxy}-N-(2-dimethylamino-ethyl)-N-methyl-acetamide

The procedure described in Example 2 was repeated using [(4-{[3-chloro-4-(pyridin-2-ylmethoxy)phenyl]amino}quinazolin-5-yl)oxy]acetic acid sodium salt (obtained as described in Example 2, preparation of starting materials, 100 mg, 0.23 mmol), HATU (308 mg, 0.81 mmol), DIPEA (120 μl, 0.69 mmol) and N,N,N′-trimethyl-ethane-1,2-diamine (164 mg, 1.61 mmol) to give the title compound as a solid (14 mg, 8%); Mass spectrum: MH⁺ 522.

### EXAMPLE 16

### N-[3-Chloro-4-(pyridin-2-ylmethoxy)phenyl]-5-(2-morpholin-4-yl-2-oxoethoxy)quinazolin-4-amine

A mixture of [(4-{[3-chloro-4-(pyridin-2-ylmethoxy)phenyl]amino}quinazolin-5-yl)oxy]acetic acid sodium salt (obtained as described in Example 2, preparation of starting materials, 197 mg, 0.43 mmol), di-isopropylethylamine (0.22 ml, 1.3 mmol), morpholine (56 μl, 0.64 mmol) and HATU (195 mg, 0.51 mmol) in DMA (2 ml) was stirred at room temperature for 18 hours. After evaporation of the solvents in vacuo, the residue was purified by chromatography on silica gel eluting with 3%-5% 7N ammonia-methanol in DCM to give the title compound as a white solid (46 mg, 22%); NMR spectrum: (400 MHz) 3.67-3.51 (m, 8H), 5.18 (s, 2H), 5.29 (s, 2H), 7.22 (d, 1H), 7.28 (d, 1H), 7.37 (m, 2H), 7.59 (d, 1H), 7.76 (m, 1H), 7.88 (m, 1H), 7.99 (m, 1H), 8.40 (s, 1H), 8.56 (s, 1H), 8.60 (d, 1H); Mass spectrum: MH⁺ 506.

### EXAMPLE 17

### N-[3-Chloro-4-(pyridin-2-ylmethoxy)phenyl]-5-(2-oxo-2-piperazin-1-ylethoxy)quinazolin-4-amine

The procedure described in Example 9 was repeated with 4-{[3-chloro-4-(pyridin-2-ylmethoxy)phenyl]amino}quinazolin-5-ol (obtained as described in Example 1, preparation of starting materials, 245 mg, 0.65 mmol) and tert-butyl 4-(chloroacetyl)piperazine-1-carboxylate (prepared according to the method described by Shuttleworth S. J. et al, Bioorg. Med. Chem. Lett., 2000, 10, 2501, 204 mg, 0.78 mmol) except that, at the end of the reaction after evaporation of the solvents in vacuo, the residue was stirred with TFA (5 ml) for 1 hour. After evaporation of the solvents, the residue was dissolved in 7N ammonia-methanol and the solvents removed in vacuo. The residue was purified on silica gel eluting with 10% 7N ammonia-methanol in DCM to give the title compound as a white solid (223 mg, 68%); NMR spectrum: (400 MHz) 2.71 (m, 2H), 2.75 (m, 2H), 3.40 (m, 2H), 3.51 (m, 2H), 5.14 (s, 2H), 5.29 (s, 2H), 7.22 (d, 1H), 7.28 (d, 1H), 7.36 (m, 2H), 7.59 (d, 1H), 7.76 (m, 1H), 7.88 (m, 1H), 7.99 (m, 1H), 8.41 (s, 1H), 8.55 (s, 1H), 8.59 (d, 1H); Mass spectrum: MH⁺ 505.

### EXAMPLE 18

### N-[3-Chloro-4-(pyridin-2-ylmethoxy)phenyl]-5-[2-(4-methylpiperazin-1-yl)-2-oxoethoxy]quinazolin-4-amine

A mixture of N-[3-chloro-4-(pyridin-2-ylmethoxy)phenyl]-5-(2-oxo-2-piperazin-1-ylethoxy)quinazolin-4-amine (obtained as described in Example 17, 230 mg, 0.45 mmol), 37% aqueous formaldehyde (40 μl, 0.45 mmol) and formic acid (17 μl, 0.45 mmol) in DMSO (1.2 ml) was irradiated in a Personal Chemistry EMRYS™ Optimizer EXP microwave synthesisor at 180° C. for 3 minutes. After cooling, the resulting solid was filtered, washed with the minimum of DMSO, then with water (×2) and dried over P₂O₅ under high vacuum to give the title compound as a white solid (133 mg, 56%); NMR spectrum: (400 MHz) 2.21 (s, 3H), 2.34 (m, 2H), 2.39 (m, 2H), 3.49 (m, 2H), 3.59 (m, 2H), 5.15 (s, 2H), 5.29 (s, 2H), 7.22 (d, 1H), 7.28 (d, 1H), 7.36 (m, 2H), 7.59 (d, 1H), 7.74 (m, 1H), 7.88 (m, 1H), 7.99 (m, 1H), 8.40 (s, 1H), 8.55 (s, 1H), 8.59 (d, 1H); Mass spectrum: MH⁺ 519.

### EXAMPLE 19

### (2R)-2-[(4-{[3-Chloro-4-(pyridin-2-ylmethoxy)phenyl]amino}quinazolin-5-yl)oxy]propanamide

Methyl (2R)-2-[(4-{[3-chloro-4-(pyridin-2-ylmethoxy)phenyl]amino}quinazolin-5-yl)oxy]propanoate (200 mg, 0.432 mmol) was dissolved in a mixture of aqueous 880 ammonia (0.6 ml) in ethanol (2 ml) and the solution heated in a microwave synthesisor (CEM) at 150° C. for 15 minutes. The solution was added to water (5 ml) and extracted into dichloromethane (2×10 ml). The combined extracts were dried by passing through a phase separating column, and then loaded onto a prepacked silica column (20 g) and eluted with 10% methanol in ethyl acetate. The relevant fractions were combined to give the title compound as a solid (35 mg, 19%); NMR spectrum: (373K) 1.65 (d, 3H), 5.10-5.15 (q, 1H), 5.25 (s, 2H), 7.08-7.13 (d, 1H), 7.23-7.28 (d, 1H), 7.30-7.40 (m, 2H), 7.55-7.60 (d, 1H), 7.65-7.75 (t, 1H), 7.80-7.90 (m, 2H), 8.20 (d, 1H), 8.50 (s, 1H), 8.55-8.65 (d, 1H), 10.85-11.0 (bs, 1H); Mass spectrum: MH⁺ 450.

The methyl (2R)-2-[(4-{[3-chloro-4-(pyridin-2-ylmethoxy)phenyl]amino}quinazolin-5-yl)oxy]propanoate used as starting material was obtained as follows:

4-{[3-Chloro-4-(pyridin-2-ylmethoxy)phenyl]amino}quinazolin-5-ol (obtained as described in Example 1, preparation of starting materials, 1.5 g, 3.97 mmol), methyl (2S)-2-hydroxypropanoate (0.57 ml, 5.96 mmol) and triphenylphosphine (1.56 g, 5.96 mmol) were suspended in DCM (30 ml). DTAD (1.37 g, 5.96 mmol) was added in one portion and the mixture was stirred vigorously for 3 hours. The mixture was filtered to remove a fine precipitate and the filtrate was concentrated to approximately 15 ml. This was loaded onto a silica column and eluted with 0-10% MeOH in ethyl acetate. The required fractions were combined and concentrated to give a glassy solid. This was triturated with Et₂O to give methyl (2R)-2-[(4-{[3-chloro-4-(pyridin-2-ylmethoxy)phenyl]amino}quinazolin-5-yl)oxy]propanoate as an off-white solid (1.26 g, 69%); NMR spectrum: 1.69 (d, 3H), 3.79 (s, 3H), 5.29 (s, 2H), 5.51 (q, 1H), 7.17 (d, 1H), 7.28 (d, 1H), 7.33-7.40 (m, 2H), 7.57 (d, 1H), 7.67-7.75 (m, 2H), 7.88 (t, 1H), 8.22 (d, 1H), 8.54 (s, 1H), 8.59 (dd, 1H), 10.42 (s, 1H); Mass spectrum: MH⁺ 465.

### EXAMPLE 20

### (2R)-2-[(4-{[3-Chloro-4-(pyridin-2-ylmethoxy)phenyl]amino}quinazolin-5-yl)oxy]-N-methylpropanamide

Methyl (2R)-2-[(4-{[3-chloro-4-(pyridin-2-ylmethoxy)phenyl]amino}quinazolin-5-yl)oxy]propanoate (obtained as described in Example 19, preparation of starting materials, 100 mg, 0.216 mmol) was dissolved in dry THF (2 ml) to which was added 2M methylamine in THF (1 ml). The mixture was heated to 120° C. in a microwave synthesisor (CEM) for 10 minutes. More 2M methylamine in THF (1 ml) was added and heated at 120° C. for 20 minutes. More 2M methylamine in THF (0.5 ml) was added and heated at 120° C. for 40 minutes. More 2M methylamine in THF (0.5 ml) was added and heated at 120° C. for 20 minutes (this was done so that the reaction would take place without a pressure build-up). The reaction mixture was concentrated and the resultant residue was stirred in Et₂O (15 ml) for 2 hours. The precipitate was filtered to give the title compound as a yellow solid (70 mg, 70%); NMR spectrum: 1.62 (d, 3H), 2.68 (d, 3H), 5.15 (q, 1H), 5.29 (s, 2H), 7.00 (d, 1H), 7.28 (d, 1H), 7.32-7.40 (m, 2H), 7.58 (d, 1H), 7.68-7.81 (m, 2H), 7.83-7.91 (m, 1H), 8.27-8.38 (m, 2H), 8.54 (s, 1H), 8.59 (d, 1H), 10.63 (s, 1H); Mass spectrum: MH⁺ 464.

### EXAMPLE 21

### (2R)-2-[(4-{[3-Chloro-4-(pyridin-2-ylmethoxy)phenyl]amino}quinazolin-5-yl)oxy]-N,N-dimethylpropanamide

To a solution of (2R)-2-[(4-{[3-chloro-4-(pyridin-2-ylmethoxy)phenyl]amino}quinazolin-5-yl)oxy]propanoic acid (112 mg, 0.25 mmol) in dimethylacetamide (2 ml) was added N,N-diisopropylethylamine (0.2 ml) and HATU, (115 mg, 0.30 mmol), and the solution was stirred and heated at 70° C. for 90 minutes. More HATU (50 mg) was added and the solution heated at 70° C. for a further 60 minutes. A 2M solution of dimethylamine in 1,4 dioxane (2 ml, 4 mmol) was added and the solution heated in a microwave synthesisor (CEM) at 140° C. for 40 minutes. The solution was added to water (10 ml) and extracted into dichloromethane (2×10 ml). The combined extracts were dried by passing through a phase separating column, and then loaded onto a pre-packed silica column (20 g) and eluted with 1% 880 NH₃/10% methanol in dichloromethane. The relevant fractions were combined to give the title compound as a solid (110 mg, 92%); NMR spectrum: (373K) 1.60 (d, 3H), 2.80-3.25 (bs, 6H), 5.25 (s, 2H), 5.75-5.85 (q, 1H), 7.20-7.45 (m, 4H), 7.55-7.60 (d, 1H), 7.75-7.90 (m, 3H), 8.20 (d, 1H), 8.60 (d, 1H), 8.65 (s, 1H), 11.40-11.50 (s, 1H); Mass spectrum: MH⁺ 478.

The (2R)-2-[(4-{[3-chloro-4-(pyridin-2-ylmethoxy)phenyl]amino}quinazolin-5-yl)oxy]propanoic acid used as starting material was obtained as follows:

To a solution of methyl (2R)-2-[(4-{[3-chloro-4-(pyridin-2-ylmethoxy)phenyl]amino}quinazolin-5-yl)oxy]propanoate (obtained as described in Example 19, preparation of starting materials, 0.7 g, 1.5 mmol) in THF (10 ml) and methanol (10 ml) was added a solution of 2M aqueous sodium hydroxide (2 ml). The reaction was stirred at ambient temperature for 3 hours. The solution was evaporated in vacuo and the solid suspended in water (30 ml), acidified by addition of glacial acetic acid to pH=4 and stirred vigorously for an hour. The solid was filtered, washed with water, acetone and ether to give (2R)-2-[(4-{[3-chloro-4-(pyridin-2-ylmethoxy)phenyl]amino}quinazolin-5-yl)oxy]propanoic acid as a yellow solid (0.62 g, 95%); NMR spectrum: 1.60-1.75 (d, 3H), 5.20-5.30 (s, 2H), 5.30-5.40 (q, 1H), 7.10-7.20 (d, 1H), 7.20-7.30 (d, 1H), 7.30-7.40 (m, 2H), 7.50-7.60 (d, 1H), 7.70-7.80 (t, 1H), 7.80-7.95 (m, 2H), 8.2 (d, 1H), 8.50 (s, 1H), 8.50-8.60 (d, 1H), 10.66-10.76 (s, 1H); Mass spectrum: MH⁺ 451.

### EXAMPLE 22

### (2R)-2-[(4-{[3-Chloro-4-(pyridin-2-ylmethoxy)phenyl]amino}quinazolin-5-yl)oxy]-N-(2-hydroxyethyl)-N-methylpropanamide

The procedure described in Example 19 was repeated using methyl (2R)-2-[(4-{[3-chloro-4-(pyridin-2-ylmethoxy)phenyl]amino}quinazolin-5-yl)oxy]propanoate (obtained as described in Example 19, preparation of starting materials, 200 mg, 0.432 mmol) and N-methylethanolamine (2 ml) to give the title compound as a gum (75 mg, 34%); NMR spectrum: (373K) 1.65 (d, 3H), 2.90-3.20 (m, 4H), 3.50-3.70 (bs, 3H), 4.20-4.70 (bs, 1H), 5.25 (s, 2H), 5.70-5.85 (bs, 1H), 7.20-7.25 (m, 2H), 7.25-7.40 (m, 2H), 7.55-7.60 (d, 1H), 7.65-7.75 (t, 1H), 7.80-7.95 (m, 2H), 8.28 (d, 1H), 8.60 (s, 1H), 8.65-8.70 (d, 1H), 10.85-10.95 (s, 1H); Mass spectrum: MH⁺ 508.

### EXAMPLE 23

### N-[3-Chloro-4-(pyridin-2-ylmethoxy)phenyl]-5-[(1R)-1-methyl-2-oxo-2-pyrrolidin-1-ylethoxy]quinazolin-4-amine

The procedure described in Example 19 was repeated using methyl (2R)-2-[(4-{[3-chloro-4-(pyridin-2-ylmethoxy)phenyl]amino}quinazolin-5-yl)oxy]propanoate (obtained as described in Example 19, preparation of starting materials, 200 mg, 0.432 mmol) and pyrrolidine (2 ml) to give the title compound as a gum (80 mg, 37%); NMR spectrum: (373K) 1.65 (d, 3H), 1.70-2.15 (m, 4H), 3.40-3.55 (m, 3H), 3.60-3.80 (bs, 1H), 5.25 (s, 2H), 5.50-5.60 (q, 1H), 7.15-7.25 (m, 2H), 7.25-7.40 (m, 2H), 7.55-7.60 (d, 1H), 7.65-7.75 (t, 1H), 7.80-7.95 (m, 2H), 8.28 (d, 1H), 8.50 (s, 1H), 8.55-8.60 (d, 1H), 10.85 (s, 1H); Mass spectrum: MH⁺ 504.

### EXAMPLE 24

### (3R)-1-{(2R)-2-[(4-{[3-Chloro-4-(pyridin-2-ylmethoxy)phenyl]amino}quinazolin-5-yl)oxy]propanoyl}pyrrolidin-3-ol

Methyl (2R)-2-[(4-{[3-chloro-4-(pyridin-2-ylmethoxy)phenyl]amino}quinazolin-5-yl)oxy]propanoate (obtained as described in Example 19, preparation of starting materials, 200 mg, 0.432 mmol) was dissolved in (R)-(+)-3-pyrrolidinol (2 ml) and the solution heated in a microwave synthesisor (CEM) at 150° C. for 15 minutes. The solution was cooled and water added and the precipitated solid filtered off and washed with water and dried to give the title compound as a solid (54 mg, 24%); NMR spectrum: (373K) 1.62 (d, 3H), 1.80-2.05 (m, 2H), 3.30-3.80 (m, 4H), 4.25-4.60 (m, 2H), 5.25 (s, 2H), 5.50-5.60 (q, 1H), 7.16-7.20 (d, 1H), 7.20-7.25 (d, 1H) 7.30-7.35 (m, 1H), 7.35-7.40 (d, 1H), 7.55-7.60 (d, 1H), 7.65-7.72 (t, 1H), 7.80-7.90 (m, 2H), 8.23 (d, 1H), 8.50 (s, 1H), 8.55-8.60 (d, 1H), 10.70-10.80 (s, 1H); Mass spectrum: MH⁺ 520.

### EXAMPLE 25

### ((2S)-1-{(2R)-2-[(4-{[3-Chloro-4-(pyridin-2-ylmethoxy)phenyl]amino}quinazolin-5-yl)oxy]propanoyl}pyrrolidin-2-yl)methanol

The procedure described in Example 19 was repeated using methyl (2R)-2-[(4-{[3-chloro-4-(pyridin-2-ylmethoxy)phenyl]amino}quinazolin-5-yl)oxy]propanoate (obtained as described in Example 19, preparation of starting materials, 200 mg, 0.432 mmol) and (S)-(−)-2-(hydroxymethyl)-pyrrolidine (1.0 ml) to give the title compound as a solid (110 mg, 47%); NMR spectrum: (373K) 1.62 (d, 3H), 1.80-2.10 (m, 4H), 3.45-3.80 (m, 4H), 4.05-4.25 (bs, 1H), 4.25-4.60 (bs, 1H), 5.25 (s, 2H), 5.40-5.65 (bs, 1H), 7.15-7.30 (m, 2H), 7.30-7.45 (m, 2H), 7.60-7.65 (d, 1H), 7.65-7.75 (t, 1H), 7.80-7.90 (m, 2H), 8.25 (d, 1H), 8.50 (s, 1H), 8.55-8.60 (d, 1H), 10.65-10.82 (bs, 1H); Mass spectrum: MH⁺ 534.

### EXAMPLE 26

### ((2R)-1-{(2R)-2-[(4-{[3-Chloro-4-(pyridin-2-ylmethoxy)phenyl]amino}quinazolin-5-yl)oxy]propanoyl}pyrrolidin-2-yl)methanol

The procedure described in Example 19 was repeated using methyl (2R)-2-[(4-{[3-chloro-4-(pyridin-2-ylmethoxy)phenyl]amino}quinazolin-5-yl)oxy]propanoate (obtained as described in Example 19, preparation of starting materials, 200 mg, 0.432 mmol) and (R)-(−)-2-(hydroxymethyl)-pyrrolidine (1.0 ml) to give the title compound as a solid (43 mg, 19%); NMR spectrum: (373K) 1.62 (d, 3H), 1.80-2.10 (m, 4H), 3.40-3.75 (m, 4H), 4.00-4.25 (bs, 1H), 4.25-4.40 (bs, 1H), 5.30 (s, 2H), 5.45-5.65 (bs, 1H), 7.10-7.25 (m, 2H), 7.30-7.45 (m, 2H), 7.55-7.60 (d, 1H), 7.70-7.75 (t, 1H), 7.85-7.90 (m, 2H), 8.25 (d, 1H), 8.50 (s, 1H), 8.60-8.65 (d, 1H), 10.65-10.82 (bs, 1H); Mass spectrum: MH⁺ 534.

### EXAMPLE 27

### N-[3-Chloro-4-(pyridin-2-ylmethoxy)phenyl]-5-[(1R)-1-methyl-2-morpholin-4-yl-2-oxoethoxy]quinazolin-4-amine

To a solution of (2R)-2-[(4-{[3-chloro-4-(pyridin-2-ylmethoxy)phenyl]amino}quinazolin-5-yl)oxy]propanoic acid (obtained as described in Example 21, preparation of starting materials, 112 mg, 0.25 mmol) in dimethylacetamide (2 ml) was added N,N-diisopropylethylamine (0.2 ml) and HATU, (115 mg, 0.30 mmol), and the solution was stirred and heated at 70° C. for 90 minutes. More HATU (50 mg) was added and the solution heated at 70° C. for a further 60 minutes. Morpholine (0.8 ml) was added and the solution heated in a microwave synthesisor (CEM) at 140° C. for 40 minutes. The solution was added to water (10 ml) and extracted into dichloromethane (2×10 ml). The combined extracts were dried by passing through a phase separating column, and then loaded onto a pre-packed silica column (20 g) and eluted with 1% 880 NH₃/10% methanol in dichloromethane. The relevant fractions were combined to give the title compound as a solid (11 mg, 9%); NMR spectrum: (373K) 1.60 (d, 3H), 3.55-3.70 (m, 8H), 5.25 (s, 2H), 5.75-5.85 (q, 1H), 7.20-7.30 (m, 2H), 7.30-7.40 (m, 2H), 7.55-7.60 (d, 1H), 7.65-7.75 (t, 1H), 7.80-7.92 (m, 2H), 8.25 (d, 1H), 8.50 (s, 1H), 8.60 (d, 1H), 10.90 (s, 1H); Mass spectrum: MH⁺ 520.

### EXAMPLE 28

### (2S)-2-[(4-{[3-Chloro-4-(pyridin-2-ylmethoxy)phenyl]amino}quinazolin-5-yl)oxy]-propanamide

The procedure described in Example 21 was repeated using (2S)-2-[(4-{[3-chloro-4-(pyridin-2-ylmethoxy)phenyl]amino}quinazolin-5-yl)oxy]propanoic acid (224 mg, 0.50 mmol) and ammonia (0.5 M solution in tetrahydrofuran, 4 ml, 2 mmol) to give the title compound as a solid (20 mg, 9%); NMR spectrum: (373K) 1.65 (d, 3H), 5.10-5.15 (q, 1H), 5.25 (s, 2H), 7.08-7.13 (d, 1H), 7.23-7.28 (d, 1H), 7.30-7.40 (m, 2H), 7.55-7.60 (d, 1H), 7.65-7.75 (t, 1H), 7.80-7.90 (m, 2H), 8.20 (d, 1H), 8.50 (s, 1H), 8.55-8.65 (d, 1H), 10.85-11.0 (bs, 1H); Mass spectrum: MH⁺ 450.

The (2S)-2-[(4-{[3-chloro-4-(pyridin-2-ylmethoxy)phenyl]amino}quinazolin-5-yl)oxy]propanoic acid used as starting material was obtained as follows:

The procedure described in Example 19 was repeated using 4-{[3-chloro-4-(pyridin-2-ylmethoxy)phenyl]amino}quinazolin-5-ol (obtained as described in Example 1, preparation of starting materials, 1.5 g, 3.97 mmol) and methyl (2R)-2-hydroxypropanoate (624 mg, 6.0 mmol) to give the methyl (2S)-2-[(4-{[3-chloro-4-(pyridin-2-ylmethoxy)phenyl]amino}quinazolin-5-yl)oxy]propanoate as a solid (2.4 g, 70%); NMR spectrum: 1.69 (d, 3H), 3.79 (s, 3H), 5.29 (s, 2H), 5.51 (q, 1H), 7.17 (d, 1H), 7.28 (d, 1H), 7.33-7.40 (m, 2H), 7.57 (d, 1H), 7.67-7.75 (m, 2H), 7.88 (t, 1H), 8.22 (d, 1H), 8.54 (s, 1H), 8.59 (dd, 1H), 10.42 (s, 1H); Mass spectrum: MH⁺ 465.

The procedure described in Example 21, preparation of starting materials was repeated but starting with methyl (2S)-2-[(4-{[3-chloro-4-(pyridin-2-ylmethoxy)phenyl]amino}quinazolin-5-yl)oxy]propanoate (2.1 g, 4.5 mmol) to give the title compound as a yellow solid (1.96 g, 95%); NMR spectrum: 1.60-1.75 (d, 3H), 5.20-5.30 (s, 2H), 5.30-5.40 (q, 1H), 7.10-7.20 (d, 1H), 7.20-7.30 (d, 1H), 7.30-7.40 (m, 2H), 7.50-7.60 (d, 1H), 7.70-7.80 (t, 1H), 7.80-7.95 (m, 2H), 8.2 (d, 1H), 8.50 (s, 1H), 8.50-8.60 (d, 1H), 10.66-10.76 (s, 1H); Mass spectrum: MH⁺ 451.

### EXAMPLE 29

### (2S)-2-[(4-{[3-Chloro-4-(pyridin-2-ylmethoxy)phenyl]amino}quinazolin-5-yl)oxy]-N-methylpropanamide

The procedure described in Example 21 was repeated using (2S)-2-[(4-{[3-chloro-4-(pyridin-2-ylmethoxy)phenyl]amino}quinazolin-5-yl)oxy]propanoic acid (obtained as described in Example 28, preparation of starting materials, 224 mg, 0.50 mmol) and a 2.0 M solution of methylamine in tetrahydrofuran (2 ml, 4 mmol) to give the title compound as a solid (144 mg, 62%); NMR spectrum: 1.62 (d, 3H), 2.68 (d, 3H), 5.15 (q, 1H), 5.29 (s, 2H), 7.00 (d, 1H), 7.28 (d, 1H), 7.32-7.40 (m, 2H), 7.58 (d, 1H), 7.68-7.81 (m, 2H), 7.83-7.91 (m, 1H), 8.27-8.38 (m, 2H), 8.54 (s, 1H), 8.59 (d, 1H), 10.63 (s, 1H); Mass spectrum: MH⁺ 464.

### EXAMPLE 30

### (2S)-2-[(4-{[3-Chloro-4-(pyridin-2-ylmethoxy)phenyl]amino}quinazolin-5-yl)oxy]-N,N-dimethylpropanamide

The procedure described in Example 21 was repeated using (2S)-2-[(4-{[3-chloro-4-(pyridin-2-ylmethoxy)phenyl]amino}quinazolin-5-yl)oxy]propanoic acid (obtained as described in Example 28, preparation of starting materials, 224 mg, 0.25 mmol) and 2.0 M solution of dimethylamine in tetrahydrofuran (2 ml, 4 mmol) to give the title compound as a solid (71 mg, 30%); NMR spectrum: (373K) 1.60 (d, 3H), 2.80-3.25 (bs, 6H), 5.25 (s, 2H), 5.75-5.85 (q, 1H), 7.20-7.45 (m, 4H), 7.55-7.60 (d, 1H), 7.75-7.90 (m, 3H), 8.20 (d, 1H), 8.60 (d, 1H), 8.65 (s, 1H), 11.40-11.50 (s, 1H); Mass spectrum: MH⁺ 478.

### EXAMPLE 31

### (2S)-2-[(4-{[3-Chloro-4-(pyridin-2-ylmethoxy)phenyl]amino}quinazolin-5-yl)oxy]-N-(2-hydroxyethyl)-N-methylpropanamide

The procedure described in Example 19 was repeated using methyl (2S)-2-[(4-{[3-chloro-4-(pyridin-2-ylmethoxy)phenyl]amino}quinazolin-5-yl)oxy]propanoate (obtained as described in Example 28, preparation of starting materials) and N-methylethanolamine (2 ml) to give the title compound as a gum (140 mg, 64%); NMR spectrum: (373K); 1.65 (d, 3H), 2.90-3.20 (m, 4H), 3.50-3.70 (bs, 3H), 4.20-4.70 (bs, 1H), 5.25 (s, 2H), 5.70-5.85 (bs, 1H), 7.20-7.25 (m, 2H), 7.25-7.40 (m, 2H), 7.55-7.60 (d, 1H), 7.65-7.75 (t, 1H), 7.80-7.95 (m, 2H), 8.28 (d, 1H), 8.60 (s, 1H), 8.65-8.70 (d, 1H), 10.85-10.95 (s, 1H); Mass spectrum: MH⁺ 508.

### EXAMPLE 32

### (3R)-1-{(2S)-2-[(4-{[3-Chloro-4-(pyridin-2-ylmethoxy)phenyl]amino}quinazolin-5-yl)oxy]propanoyl}pyrrolidin-3-ol

The procedure described in Example 21 was repeated using (2S)-2-[(4-{[3-chloro-4-(pyridin-2-ylmethoxy)phenyl]amino}quinazolin-5-yl)oxy]propanoic acid (obtained as described in Example 28, preparation of starting materials, 224 mg, 0.25 mmol) and (R)-(+)-3-pyrrolidinol (1.0 ml) in tetrahydrofuran (1.0 ml) to give the title compound as a solid (55 mg, 21%); NMR spectrum: (373K) 1.65 (d, 3H), 1.70-2.15 (bm, 2H), 3.30-3.90 (bm, 4H), 4.40-4.90 (bm, 2H), 5.30 (s, 2H), 5.20-5.70 (bq, 1H), 7.20-7.30 (m, 2H), 7.30-7.45 (m, 2H), 7.60 (d, 1H), 7.70-7.80 (t, 1H), 7.80-7.95 (m, 2H), 8.30 (d, 1H), 8.50 (s, 1H), 8.60 (s, 1H), 10.85-10.95 (d, 1H); Mass spectrum: MH⁺ 520.

### EXAMPLE 33

### (3S)-1-{(2S)-2-[(4-{[3-Chloro-4-(pyridin-2-ylmethoxy)phenyl]amino}quinazolin-5-yl)oxy]propanoyl}pyrrolidin-3-ol

The procedure described in Example 19 was repeated using methyl (2S)-2-[(4-{[3-chloro-4-(pyridin-2-ylmethoxy)phenyl]amino}quinazolin-5-yl)oxy]propanoate (obtained as described in Example 28, preparation of starting materials) and (S)-(−)-3-pyrrolidinol (1 ml) to give the title compound as a gum (60 mg, 26%); NMR spectrum: (373K) 1.62 (d, 3H), 1.80-2.05 (m, 2H), 3.30-3.80 (m, 4H), 4.25-4.60 (m, 2H), 5.25 (s, 2H), 5.50-5.60 (q, 1H), 7.16-7.20 (d, 1H), 7.20-7.25 (d, 1H) 7.30-7.35 (m, 1H), 7.35-7.40 (d, 1H), 7.55-7.60 (d, 1H), 7.65-7.72 (t, 1H), 7.80-7.90 (m, 2H), 8.23 (d, 1H), 8.50 (s, 1H), 8.55-8.60 (d, 1H), 10.70-10.80 (s, 1H); Mass spectrum: MH⁺ 520.

### EXAMPLE 34

### ((2S)-1-{(2S)-2-[(4-{[3-Chloro-4-(pyridin-2-ylmethoxy)phenyl]amino}quinazolin-5-yl)oxy]propanoyl}pyrrolidin-2-yl)methanol

The procedure described in Example 19 was repeated using methyl (2S)-2-[(4-{[3-chloro-4-(pyridin-2-ylmethoxy)phenyl]amino}quinazolin-5-yl)oxy]propanoate (obtained as described in Example 28, preparation of starting materials) and (S)-(−)-2-(hydroxymethyl)-pyrrolidine (1 ml) to give the title compound as a gum (60 mg, 26%); NMR spectrum: (373K) 1.62 (d, 3H), 1.80-2.10 (m, 4H), 3.40-3.75 (m, 4H), 4.00-4.25 (bs, 1H), 4.25-4.40 (bs, 1H), 5.30 (s, 2H), 5.45-5.65 (bs, 1H), 7.10-7.25 (m, 2H), 7.30-7.45 (m, 2H), 7.55-7.60 (d, 1H), 7.70-7.75 (t, 1H), 7.85-7.90 (m, 2H), 8.25 (d, 1H), 8.50 (s, 1H), 8.60-8.65 (d, 1H), 10.65-10.82 (bs, 1H); Mass spectrum: MH⁺ 534.

### EXAMPLE 35

### (2R)-2-[(4-{[3-Chloro-4-(pyridin-2-ylmethoxy)phenyl]amino}quinazolin-5-yl)oxy]-4-hydroxy-N-methylbutanamide

4-{[3-Chloro-4-(pyridin-2-ylmethoxy)phenyl]amino}quinazolin-5-ol (obtained as described in Example 1, preparation of starting materials, 150 mg, 0.40 mmol), (S)-(−)-α-hydroxy-γ-butyrolactone (47 μl, 0.60 mmol) and triphenylphosphine (157 mg, 0.60 mmol) were stirred in DCM (10 ml) to which was added DTAD (138 mg, 0.60 mmol). The mixture was stirred for 2 hours at ambient temperature. Triphenylphosphine (157 mg, 0.60 mmol) and DTAD (138 mg, 0.60 mmol) were added and the reaction was stirred for a further 1 hour. 2M methylamine in THF (2 ml) was added and the reaction was stirred at ambient temperature for 64 hours. The reaction mixture was concentrated and the residue was separated between water (10 ml) and DCM (15 ml). The DCM was loaded onto a silica column and eluted with 2.5 to 5% (20:1 MeOH/conc. NH_{3(aq)}) in DCM. The required fractions were combined to give the title compound as a solid (40 mg, 20%); NMR spectrum: 2.06-2.22 (m, 2H), 2.64 (d, 3H), 3.55-3.67 (m, 2H), 4.83 (t, 1H), 5.06-5.15 (m, 1H), 5.29 (s, 2H), 6.99 (d, 1H), 7.27 (d, 1H), 7.32-7.40 (m, 2H), 7.58 (d, 1H), 7.63-7.76 (m, 2H), 7.82-7.92 (m, 1H), 8.21 (d, 1H), 8.36 (d, 1H), 8.52 (s, 1H), 8.59 (d, 1H), 10.45 (s, 1H); Mass spectrum: MH⁺ 494.

### EXAMPLE 36

### (2R)-2-[(4-{[3-Chloro-4-(pyridin-2-ylmethoxy)phenyl]amino}quinazolin-5-yl)oxy]-4-hydroxy-N-(2-hydroxy-1,1-dimethylethyl)butanamide

The procedure described in Example 35 was repeated using 4-{[3-chloro-4-(pyridin-2-ylmethoxy)phenyl]amino}quinazolin-5-ol (obtained as described in Example 1, preparation of starting materials, 150 mg, 0.40 mmol), (S)-(−)-α-hydroxy-γ-butyrolactone (47 μl, 0.60 mmol) and 2-(methylamino)ethanol (192 μl, 2.0 mmol) to give the title compound as a solid (135 mg, 61%); NMR spectrum: (140° C.) 1.27 (s, 6H), 2.20 (q, 2H), 3.38-3.47 (m, 2H), 3.65-3.73 (m, 2H), 4.23 (bs, 2H), 5.20 (t, 1H), 5.27 (s, 2H), 7.08 (d, 1H), 7.22 (d, 1H), 7.23-7.33 (m, 2H), 7.37 (d, 1H), 7.58 (d, 1H), 7.67 (t, 1H), 7.75 (d, 1H), 7.83 (t, 1H), 8.13 (d, 1H), 8.50 (s, 1H), 8.57 (d, 1H), 10.42 (s, 1H); Mass spectrum: MH⁺ 552.

### EXAMPLE 37

### (2R)-2-[(4-{[3-Chloro-4-(pyridin-2-ylmethoxy)phenyl]amino}quinazolin-5-yl)oxy]-4-hydroxy-N,N-dimethylbutanamide

The procedure described in Example 35 was repeated using 4-{[3-chloro-4-(pyridin-2-ylmethoxy)phenyl]amino}quinazolin-5-ol (obtained as described in Example 1, preparation of starting materials, 150 mg, 0.40 mmol), (S)-(−)-α-hydroxy-γ-butyrolactone (47 μl, 0.60 mmol) and 2M dimethylamine in THF (1.0 ml, 2.0 mmol) to give the title compound as a solid (111 mg, 52%); NMR spectrum: (140° C.); 2.16-2.30 (m, 2H), 3.07 (s, 6H), 3.72 (t, 2H), 4.28 (bs, 1H), 5.28 (s, 2H), 5.80 (t, 1H), 7.20-7.27 (m, 2H), 7.33 (dd, 1H), 7.40 (d, 1H), 7.60 (d, 1H), 7.70 (t, 1H), 7.77-7.87 (m, 2H), 8.20 (s, 1H), 8.53 (s, 1H), 8.60 (d, 1H), 10.70 (s, 1H); Mass spectrum: MH⁺ 508.

### EXAMPLE 38

### (2R)-2-[(4-{[3-Chloro-4-(pyridin-2-ylmethoxy)phenyl]amino}quinazolin-5-yl)oxy]-4-hydroxy-N-(2-hydroxyethyl)-N-methylbutanamide

The procedure described in Example 35 was repeated using 4-{[3-chloro-4-(pyridin-2-ylmethoxy)phenyl]amino}quinazolin-5-ol (obtained as described in Example 1, preparation of starting materials, 150 mg, 0.40 mmol), (S)-(−)-α-hydroxy-γ-butyrolactone (47 μl, 0.60 mmol) and N-methylethanolamine (162 μl, 2.0 mmol) to give the title compound as a solid (90 mg, 42%); NMR spectrum: (140° C.) 2.13-2.28 (m, 2H), 3.10 (s, 3H), 3.37-3.48 (m, 1H), 3.63 (s, 3H), 3.70 (t, 2H), 4.25 (bs, 2H), 5.28 (s, 2H), 5.83 (t, 1H), 7.25 (d, 2H), 7.33 (dd, 1H), 7.38 (d, 1H), 7.62 (d, 1H), 7.68 (t, 1H), 7.69-7.88 (m, 2H), 8.20 (d, 1H), 8.52 (s, 1H), 8.60 (d, 1H), 10.73 (s, 1H); Mass spectrum: MH⁺ 538.

### EXAMPLE 39

### (3R)-3-[(4-{[3-Chloro-4-(pyridin-2-ylmethoxy)phenyl]amino}quinazolin-5-yl)oxy]-4-morpholin-4-yl-4-oxobutan-1-ol

The procedure described in Example 35 was repeated using 4-{[3-chloro-4-(pyridin-2-ylmethoxy)phenyl]amino}quinazolin-5-ol (obtained as described in Example 1, preparation of starting materials, 150 mg, 0.40 mmol), (S)-(−)-α-hydroxy-γ-butyrolactone (47 μl, 0.60 mmol) and morpholine (175 μl, 2.0 mmol) to give the title compound as a solid (165 mg, 75%); NMR spectrum: (CDCl₃) 2.13-2.23 (m, 2H), 3.52-3.73 (m, 8H), 3.75-3.92 (m, 2H), 5.22 (s, 2H), 5.67 (t, 1H), 6.95 (d, 1H), 7.10-7.19 (m, 2H), 7.54 (d, 1H), 7.57-7.72 (m, 4H), 8.01 (d, 1H), 8.48 (s, 1H), 8.52 (d, 1H), 11.27 (bs, 1H); Mass spectrum: MH⁺ 550.

### EXAMPLE 40

### (3R)-3-[(4-{[3-Chloro-4-(pyridin-2-ylmethoxy)phenyl]amino}quinazolin-5-yl)oxy]-4-oxo-4-pyrrolidin-1-ylbutan-1-ol

The procedure described in Example 35 was repeated using 4-{[3-chloro-4-(pyridin-2-ylmethoxy)phenyl]amino}quinazolin-5-ol (obtained as described in Example 1, preparation of starting materials, 150 mg, 0.40 mmol), (S)-(−)-α-hydroxy-γ-butyrolactone (47 μl, 0.60 mmol) and pyrrolidine (164 μl, 2.0 mmol) to give the title compound as a solid (140 mg, 66%); NMR spectrum: (CDCl₃) 1.75-1.90 (m, 2H), 1.91-2.05 (m, 2H), 2.12-2.29 (m, 2H), 3.36-3.63 (m, 4H), 3.74-3.93 (m, 2H), 5.22 (s, 2H), 5.44 (dd, 1H), 6.94 (d, 1H), 6.99-7.06 (m, 2H), 7.13-7.18 (m, 1H), 7.47-7.55 (m, 2H), 7.56-7.74 (m, 3H), 8.06 (d, 1H), 8.46-8.57 (m, 2H), 11.04 (bs, 1H); Mass spectrum: MH⁺ 534.

### EXAMPLE 41

### (3R)-3-[(4-{[3-Chloro-4-(pyridin-2-ylmethoxy)phenyl]amino}quinazolin-5-yl)oxy]-4-(4-methylpiperazin-1-yl)-4-oxobutan-1-ol

The procedure described in Example 35 was repeated using 4-{[3-chloro-4-(pyridin-2-ylmethoxy)phenyl]amino}quinazolin-5-ol (obtained as described in Example 1, preparation of starting materials, 150 mg, 0.40 mmol), (S)-(−)-α-hydroxy-γ-butyrolactone (47 μl, 0.60 mmol) and 1-methylpiperazine (192 μl, 0.20 mmol) to give the title compound as a solid (201 mg, 90%); NMR spectrum: (CDCl₃) 2.13-2.24 (m, 2H), 2.28 (s, 3H), 2.35-2.52 (m, 4H), 3.56-3.73 (m, 4H), 3.77-3.90 (m, 2H), 5.22 (s, 2H), 5.57-5.64 (m, 1H), 6.91-7.00 (m, 2H), 7.13-7.18 (m, 1H), 7.42 (d, 1H), 7.47-7.55 (m, 1H), 7.57-7.73 (m, 3H), 8.06 (d, 1H), 8.52 (s, 2H), 10.83 (bs, 1H); Mass spectrum: MH⁺ 563.

### EXAMPLE 42

### 2-[(4-{[3-Chloro-4-(pyridin-2-ylmethoxy)phenyl]amino}quinazolin-5-yl)oxy]-2-methylpropanamide

To a suspension of 4-{[3-chloro-4-(pyridin-2-ylmethoxy)phenyl]amino}quinazolin-5-ol (obtained as described in Example 1, preparation of starting materials, 150 mg, 0.40 mmol) in 1,4-dioxane (25 ml) was added caesium carbonate (430 mg, 1.32 mmol) and sodium hydride (60% dispersion in mineral oil, 53 mg, 1.32 mmol). The mixture was stirred under an atmosphere of nitrogen for 30 minutes at 50° C. 2-Bromo-2,2-dimethylacetamide (219 mg, 1.32 mmol) was added and the mixture heated under an atmosphere of nitrogen to 100° C. for 16 hours. The mixture was cooled to ambient temperature and saturated ammonium chloride solution (5 ml) was added. The mixture was concentrated in vacuo and the residue shaken with a mixture of saturated sodium hydrogen carbonate solution. The resultant precipitate was removed by filtration and the aqueous layer was extracted with DCM (×6). The precipitate and DCM extracts were combined and chromatographed eluting with 0 to 4% (10:1 MeOH/conc. NH_{3(aq)}) in DCM to give a solid which was triturated with ethyl acetate to give the title compound as a white solid (70 mg, 38%); NMR spectrum: 1.75 (s, 6H), 5.32 (s, 2H), 6.89 (d, 1H), 7.28 (d, 1H), 7.37 (m, 2H), 7.48 (s, 1H), 7.59 (d, 1H), 7.53 (dd, 1H), 7.70 (t, 1H), 7.88 (td, 1H), 7.93 (s, 1H), 8.17 (d, 1H), 8.52 (s, 1H), 8.60 (d, 1H), 10.42 (s, 1H); Mass spectrum: MH⁺ 464.

### EXAMPLE 43

### 2-[(4-{[3-Chloro-4-(pyridin-2-ylmethoxy)phenyl]amino}quinazolin-5-yl)oxy]-N,2-dimethylpropanamide

To a solution of 4-[3-chloro-4-(pyridin-2-ylmethoxy)phenyl]-6,6-dimethyl-4H-[1,4]oxazepino[5,6,7-de]quinazolin-5(6H)-one (70 mg, 0.157 mmol) in THF (2 ml) was added a solution of 2M methylamine in THF (2.0 ml, 2.0 mmol). The reaction was stirred at room temperature for 1 hour and then the solvents and excess amine were removed in vacuo to give a solid which was crystallised from ethyl acetate to give the title compound as a white solid (40 mg, 53%); NMR spectrum: 1.72 (s, 6H), 2.66 (s, 3H), 5.31 (s, 2H), 6.74 (d, 1H), 7.32 (d, 1H), 7.37 (m, 2H), 7.61 (m, 2H), 7.70 (t, 1H), 7.87 (td, 1H), 8.24 (d, 1H), 8.42 (m, 1H), 8.56 (s, 1H), 8.61 (d, 1H), 10.27 (s, 1H); Mass spectrum:MH⁺ 478.

The 4-[3-chloro-4-(pyridin-2-ylmethoxy)phenyl]-6,6-dimethyl-4H-[1,4]oxazepino[5,6,7-de]quinazolin-5(6H)-one used as starting material was obtained using the general procedure as described in Reference Example 27 of WO 03/077847 as follows:

4-{[3-Chloro-4-(pyridin-2-ylmethoxy)phenyl]amino}quinazolin-5-ol (prepared as described in Example 1, preparation of starting materials, 1.50 g, 3.96 mmol) and 1,1,1-trichloro-2-methyl-2-propanol (1.66 g, 10 mmol) was suspended in acetone (100 ml) and powdered sodium hydroxide (1.44 g, 36.0 mmol) was added portionwise. The reaction was stirred for 3 hours at room temperature by which time a cream-coloured precipitate had formed. This was collected by filtration and washed with acetone. The solid was then dissolved in water and the pH of the solution was adjusted to pH=5 by the addition of saturated ammonium chloride solution which caused a light brown solid to precipitate from solution. The reaction was stirred for 2 hours then the solid was collected by filtration, washed with water and dried to give 2-[(4-{[3-chloro-4-(pyridin-2-ylmethoxy)phenyl]amino}quinazolin-5-yl)oxy]-2-methylpropanoic acid as a light brown solid (1.25 g, 68%); NMR spectrum: 1.77 (s, 6H), 5.30 (s, 2H), 7.07 (d, 1H), 7.25 (d, 1H), 7.47 (m, 2H), 7.59 (d, 1H), 7.64 (dd, 1H), 7.70 (t, 1H), 7.89 (td, 1H), 8.10 (d, 1H), 8.50 (s, 1H), 8.60 (d, 1H), 10.55 (s, 1H); Mass spectrum: MH⁺ 465.

2-[(4-{[3-Chloro-4-(pyridin-2-ylmethoxy)phenyl]amino}quinazolin-5-yl)oxy]-2-methylpropanoic acid (1.24 g, 2.67 mmol) was dissolved in DMA (30 ml) then di-iso-propylethylamine (512 μl, 2.94 mmol) and HATU (1.12 g, 2.94 mmol) were added. The mixture was stirred at room temperature until TLC analysis showed complete consumption of starting material. Solvents were removed in vacuo and the residue was partitioned between DCM and water. The DCM layer was loaded onto a silica column and eluted with 2 to 4% (10:1 MeOH/conc. NH_{3(aq)}) in DCM. Evaporation of the appropriate fractions gave 4-[3-chloro-4-(pyridin-2-ylmethoxy)phenyl]-6,6-dimethyl-4H-[1,4]oxazepino[5,6,7-de]quinazolin-5(6H)-one (1.11 g, 93% yield), which crystallised upon standing; NMR spectrum: 1.55 (s, 6H), 5.34 (s, 2H), 7.19 (dd, 1H), 7.30 (m, 2H), 7.39 (dd, 1H), 7.52 (d, 1H), 7.65 (d, 1H), 7.75 (d, 1H), 7.92 (td, 1H), 7.96 (t, 1H), 8.63 (d, 1H), 8.80 (s, 1H); Mass spectrum: MH⁺ 447.

### EXAMPLE 44

### 2-[(4-{[3-Chloro-4-(pyridin-2-ylmethoxy)phenyl]amino}quinazolin-5-yl)oxy]-N-(2-hydroxy-1,1-dimethylethyl)-2-methylpropanamide

The procedure described in Example 43 was repeated using 4-[3-chloro-4-(pyridin-2-ylmethoxy)phenyl]-6,6-dimethyl-4H-[1,4]oxazepino[5,6,7-de]quinazolin-5(6H)-one (obtained as described in Example 43, preparation of starting materials, 70 mg, 0.157 mmol) and 2-amino-2-methylpropan-1-ol (500 mg, 5.62 mmol) with the reaction refluxed for 16 hours and then chromatographed eluting with 2 to 5% (10:1 MeOH/conc. NH_{3(aq)}) in DCM to give the title compound as a solid (35 mg, 42%); NMR spectrum: 1.23 (s, 6H), 1.71 (s, 6H), 3.41 (d, 2H), 4.79 (t, 1H), 5.30 (s, 2H), 6.86 (d, 1H), 7.30 (d, 1H), 7.38 (m, 3H), 7.59 (d, 1H), 7.66 (dd, 1H), 7.71 (t, 1H), 7.88 (td, 1H), 8.23 (d, 1H), 8.55 (s, 1H), 8.60 (d, 1H), 10.36 (s, 1H); Mass spectrum: MH⁺ 536.

### EXAMPLE 45

### 2-[(4-{[3-Chloro-4-(pyridin-2-ylmethoxy)phenyl]amino}quinazolin-5-yl)oxy]-N-(2-hydroxyethyl)-2-methylpropanamide

The procedure described in Example 43 was repeated using 4-[3-chloro-4-(pyridin-2-ylmethoxy)phenyl]-6,6-dimethyl-4H-[1,4]oxazepino[5,6,7-de]quinazolin-5(6H)-one (obtained as described in Example 43, preparation of starting materials, 70 mg, 0.157 mmol) and ethanolamine (500 mg, 8.20 mmol) with the reaction refluxed for 16 hours. The resultant solid washed with iso-propanol and THF and then crystallised from ethyl acetate to give the title compound as a white solid (30 mg, 38%); NMR spectrum: 1.72 (s, 6H), 3.20 (q, 2H), 3.38 (q, 2H), 4.61 (t, 1H), 5.29 (s, 2H), 6.78 (d, 1H), 7.31 (d, 1H), 7.37 (m, 2H), 7.61 (m, 2H), 7.68 (t, 1H), 7.89 (td, 1H), 8.23 (d, 1H), 8.42 (t, 1H), 8.56 (s, 1H), 8.61 (d, 1H), 10.29 (s, 1H); Mass spectrum: MH⁺ 508.

### EXAMPLE 46

### 2-[(4-{[3-Chloro-4-(pyridin-2-ylmethoxy)phenyl]amino}quinazolin-5-yl)oxy]-N,N-bis(2-hydroxyethyl)-2-methylpropanamide

The procedure described in Example 43 was repeated using 4-[3-chloro-4-(pyridin-2-ylmethoxy)phenyl]-6,6-dimethyl-4H-[1,4]oxazepino[5,6,7-de]quinazolin-5(6H)-one (obtained as described in Example 43, preparation of starting materials, 70 mg, 0.157 mmol) and diethanolamine (500 mg, 4.76 mmol) with the reaction refluxed for 16 hours and then chromatographed eluting with 4 to 7% (10:1 MeOH/conc. NH_{3(aq)}) in DCM to give the title compound as a solid (24 mg, 28%); NMR spectrum: 1.81 (s, 6H), 3.37 (m, 2H), 3.45 (m, 2H), 3.54 (m, 2H), 3.72 (m, 2H), 4.68 (t, 1H), 4.73 (t, 1H), 5.32 (s, 2H), 6.85 (d, 1H), 7.29 (d, 1H), 7.37 (dd, 1H), 7.41 (d, 1H), 7.59 (m, 2H), 7.60 (t, 1H), 7.88 (td, 1H), 8.19 (d, 1H), 8.56 (s, 1H), 8.61 (d, 1H), 9.99 (s, 1H); Mass spectrum: MH⁺ 552.

### EXAMPLE 47

### 2-[(4-{[3-Chloro-4-(pyridin-2-ylmethoxy)phenyl]amino}quinazolin-5-yl)oxy]-N-(2-hydroxyethyl)-N,2-dimethylpropanamide

The procedure described in Example 43 was repeated using 4-[3-chloro-4-(pyridin-2-ylmethoxy)phenyl]-6,6-dimethyl-4H-[1,4]oxazepino[5,6,7-de]quinazolin-5(6H)-one (obtained as described in Example 43, preparation of starting materials, 70 mg, 0.157 mmol) and N-methylethanolamine (500 mg, 8.20 mmol) with the reaction stirred at room temperature for 16 hours. The resultant solid was crystallised from ethyl acetate to give the title compound as a white solid (39 mg, 48%); NMR spectrum: 1.82 (s, 6H), 3.13 (s, 3H), 3.54 (s, 4H), 4.34 (m, 1H), 5.28 (s, 2H), 6.86 (d, 1H), 7.27 (d, 1H), 7.33 (dd, 1H), 7.40 (d, 1H), 7.60 (m, 2H), 7.66 (t, 1H), 7.85 (td, 1H), 8.09 (d, 1H), 8.52 (s, 1H), 8.57 (d, 1H), 9.92 (s, 1H); Mass spectrum: MH⁺ 522.

### EXAMPLE 48

### (3R)-1-{2-[(4-{[3-Chloro-4-(pyridin-2-ylmethoxy)phenyl]amino}quinazolin-5-yl)oxy]-2-methylpropanoyl}pyrrolidin-3-ol

The procedure described in Example 43 was repeated using 4-[3-chloro-4-(pyridin-2-ylmethoxy)phenyl]-6,6-dimethyl-4H-[1,4]oxazepino[5,6,7-de]quinazolin-5(6H)-one (obtained as described in Example 43, preparation of starting materials, 70 mg, 0.157 mmol) and (R)-(+)-3-hydroxypyrrolidine (500 mg, 4.76 mmol) with the reaction refluxed for 16 hours and then chromatographed eluting with 3 to 6% (10:1 MeOH/conc. NH_{3(aq)}) in DCM to give the title compound as a solid (7 mg, 8%); NMR spectrum: 1.60 (m, 1H), 1.85 (s, 6H), 1.88 (m, 1H), 2.66 (m, 3H), 2.92 (dd, 1H), 5.27, (m, 1H), 5.31 (s, 2H), 6.93 (d, 1H), 7.27 (d, 1H), 7.36 (dd, 1H), 7.38 (d, 1H), 7.57 (m, 2H), 7.69 (t, 1H), 7.87 (td, 1H), 8.14 (d, 1H), 8.52 (s, 1H), 8.58 (d, 1H), 10.18 (s, 1H); Mass spectrum: MH⁺ 534.

### EXAMPLE 49

### N-(2-Hydroxyethyl)-2-methyl-2-[(4-{[3-methyl-4-(pyridin-2-ylmethoxy)phenyl]amino}quinazolin-5-yl)oxy]propanamide

The procedure described in Example 43 was repeated using 4-[3-methyl-4-(pyridin-2-ylmethoxy)phenyl]-6,6-dimethyl-4H-[1,4]oxazepino[5,6,7-de]quinazolin-5(6H)-one (30 mg, 0.070 mmol) and ethanolamine (500 μl, 8.30 mmol) with the reaction refluxed for 16 hours and then chromatographed eluting with 4 to 7% (10:1 MeOH/conc. NH_{3(aq)}) in DCM to give the title compound as a white solid (21 mg, 62%); NMR spectrum: 1.72 (s, 6H), 2.32 (s, 3H), 3.21 (q, 2H), 3.39 (q, 2H), 4.60 (t, 1H), 5.23 (s, 2H), 6.76 (d, 1H), 7.06 (d, 1H), 7.36 (m, 2H), 7.57, (d, 1H), 7.67 (m, 3H), 7.87 (td, 1H), 8.43 (t, 1H), 8.48 (s, 1H), 8.60 (d, 1H), 10.16 (s, 1H); Mass spectrum: MH⁺ 488.

The 4-[3-methyl-4-(pyridin-2-ylmethoxy)phenyl]-6,6-dimethyl-4H-[1,4]oxazepino[5,6,7-de]quinazolin-5(6H)-one used as starting material was obtained as follows:

4-Chloro-5-fluoroquinazoline (obtained as described in Example 1, preparation of starting materials, 6.76 g, 37.0 mmol) was dissolved in iso-propanol (200 ml) and 4-amino-2-methylphenol (5.00 g, 40.7 mmol) was added. The mixture was heated under reflux for 2 hours, causing a yellow solid to precipitate. The mixture was cooled to ambient temperature; the solid was collected by filtration. The solid was dissolved in a boiling mixture of methanol (500 ml) and water (100 ml) to give a brown solution. With vigorous stirring, the solution was basified with aqueous ammonia (0.880, 10 ml), causing a light brown solid to precipitate. The mixture was concentrated in vacuo to such a volume that all of the methanol had been removed, leaving the product as a suspension in aqueous solution. The suspension was cooled; the solid was collected by filtration, triturated with ethyl acetate and dried over P₂O₅ in a vacuum oven to give 2-methyl-4-[(5-fluoroquinazolin-4-yl)amino]phenol as a light brown solid (8.18 g, 82%); NMR spectrum: 3.30 (s, 3H), 6.78 (d, 1H), 7.28 (m, 2H), 7.38 (dd, 1H), 7.57 (d, 1H), 7.78 (m, 1H), 8.43 (s, 1H), 8.88 (d, 1H), 9.22 (s, 1H); Mass spectrum: MH⁺ 270.

To a suspension of 2-methyl-4-[(5-fluoroquinazolin-4-yl)amino]phenol (2.0 g, 7.43 mmol) in DMF (75 ml) was added potassium carbonate (5.13 g, 37.15 mmol) and picolyl chloride hydrochloride (1.34 g, 8.18 mmol). The reaction was sonicated for 5 minutes in an ultrasonic cleaning bath and then stirred for 3 days at room temperature. The solvent was removed in vacuo, water was then added to the residue which was then extracted with DCM (×3). The organic layer was evaporated and the residue chromatographed eluting with 0 to 4% (10:1 MeOH/conc. NH_{3(aq)}) in DCM to give 5-fluoro-N-[3-methyl-4-(pyridin-2-ylmethoxy)phenyl]quinazolin-4-amine as a white solid (1.50 g, 56%); NMR Spectrum: 2.27 (s, 3H), 5.22 (s, 2H), 7.02 (d, 1H), 7.36 (dd, 1H), 7.42 (dd, 1H), 7.48 (m, 2H), 7.58 (m, 2H), 7.85 (m, 2H), 8.51 (s, 1H), 8.61 (d, 1H), 8.98 (s, 1H); Mass spectrum: MH⁺ 360.

N-Acetylethanolamine (230 μl, 2.50 mmol) was added dropwise under nitrogen to a suspension of 60% sodium hydride dispersion (100 mg, 2.50 mmol) in anhydrous DMA (20 ml). The mixture was stirred under an atmosphere of nitrogen for 20 minutes until effervescence had ceased. 5-Fluoro-N-[3-methyl-4-(pyridin-2-ylmethoxy)phenyl]quinazolin-4-amine (360 mg, 1.00 mmol) was added, and the mixture heated under an atmosphere of nitrogen at 130° C. for 6 hours. The mixture was cooled to ambient temperature and saturated ammonium chloride solution (5 ml) was added. The mixture was concentrated in vacuo and the residue shaken with a mixture of saturated sodium hydrogen carbonate solution (100 ml). The resulting precipitate was collected by filtration and trituration of the solid with hot ethyl acetate gave 4-{[3-methyl-4-(pyridin-2-ylmethoxy)phenyl]amino}quinazolin-5-ol as a yellow solid (125 mg, 35%); NMR spectrum: 2.28 (s, 3H), 5.21 (s, 2H), 6.65 (m, 2H), 7.02 (d, 1H), 7.36 (dd, 2H), 7.52 (m, 3H), 7.56 (d, 1H), 7.87 (td, 1H), 8.36 (s, 1H), 8.59 (d, 1H); Mass spectrum: MH⁺ 359.

4-{[3-Methyl-4-(pyridin-2-ylmethoxy)phenyl]amino}quinazolin-5-ol (120 mg, 0.34 mmol) was suspended in acetone (25 ml) and 1,1,1-trichloro-2-methyl-2-propanol (166 mg, 1.00 mmol) was added followed by powdered sodium hydroxide (120 mg, 3 mmol). The reaction was stirred for 2 hours at room temperature by which time a cream-coloured precipitate had formed. This was collected by filtration and washed with acetone. The solid was then dissolved in water and the pH of the solution was adjusted to pH=5 by the addition of saturated ammonium chloride solution which caused formation of a gelatinous precipitate. The reaction was stirred for 2 hours then the solid was collected by filtration, washed with water and dried to give 2-[(4-{[3-methyl-4-(pyridin-2-ylmethoxy)phenyl]amino}quinazolin-5-yl)oxy]-2-methylpropanoic acid as a dark green solid (41 mg, 27%); NMR spectrum: 1.79 (s, 6H), 2.30 (s, 3H), 5.22 (s, 2H), 7.03 (d, 2H), 7.37 (m, 2H), 7.56 (m, 3H), 7.71 (t, 1H), 7.87 (td, 1H), 8.47 (s, 1H), 8.58 (d, 1H), 10.44 (s, 1H); Mass spectrum: MH⁺ 445.

2-[(4-{[3-Methyl-4-(pyridin-2-ylmethoxy)phenyl]amino}quinazolin-5-yl)oxy]-2-methylpropanoic acid (38 mg, 0.086 mmol) was dissolved in DMF (5 ml) then di-iso-propylethylamine (16 μl, 0.094 mmol) and HATU (36 mg, 0.094 mmol) were added. The mixture was stirred at room temperature for 1 hour. Solvents were removed in vacuo and the residue was portioned between DCM and water. The DCM layer was loaded onto a silica column; the column was eluted with 0 to 2% (10:1 MeOH/conc. NH_{3(aq)}) in DCM. Evaporation of the appropriate fractions gave 4-[3-methyl-4-(pyridin-2-ylmethoxy)phenyl]-6,6-dimethyl-4H-[1,4]oxazepino[5,6,7-de]quinazolin-5(6H)-one as a colourless gum (32 mg, 88% yield); NMR spectrum: 1.55 (s, 6H), 2.26 (s, 3H), 5.25 (s, 2H), 6.98 (dd, 1H), 7.07 (m, 2H), 7.33 (d, 1H), 7.47 (dd, 1H), 7.63 (d, 1H), 7.75 (d, 1H), 7.90 (td, 1H), 7.96 (t, 1H), 8.61 (d, 1H), 8.77 (s, 1H); Mass spectrum: MH⁺ 427.

### EXAMPLE 50

### N,2-Dimethyl-2-[(4-{[3-methyl-4-(pyridin-2-ylmethoxy)phenyl]amino}quinazolin-5-yl)oxy]propanamide

The procedure described in Example 43 was repeated using 4-[3-methyl-4-(pyridin-2-ylmethoxy)phenyl]-6,6-dimethyl-4H-[1,4]oxazepino[5,6,7-de]quinazolin-5(6H)-one (obtained as described in Example 49, preparation of starting materials, 30 mg, 0.07 mmol) and 2M methylamine in THF (5.0 ml, 5.0 mmol). The reaction was stirred at room temperature for 16 hours and then the solvents and excess amine were removed in vacuo to give a solid which was crystallised from ethyl acetate/isohexane to give the title compound as a white solid (31 mg, 97%); NMR spectrum: 1.72 (s, 6H), 2.31 (s, 3H), 2.67 (d, 3H), 5.22 (s, 2H), 6.72 (d, 1H), 7.06 (d, 1H), 7.36 (m, 2H), 7.56, (d, 1H), 7.66 (m, 3H), 7.87 (td, 1H), 8.43 (q, 1H), 8.49 (s, 1H), 8.60 (d, 1H), 10.14 (s, 1H); Mass spectrum: MH⁺ 458.

### EXAMPLE 51

### 2-{[4-({3-Methyl-4-[(6-methylpyridin-3-yl)oxy]phenyl}amino)quinazolin-5-yl]oxy}acetamide

A mixture of {[4-({3-methyl-4-[(6-methylpyridin-3-yl)oxy]phenyl}amino)quinazolin-5-yl]oxy}acetic acid (120 mg, 0.27 mmol), diisopropylethylamine (72 μl, 0.4 mmol) and HATU (155 mg, 0.41 mmol) was stirred at 50° C. for 18 hours. After cooling, gaseous ammonia was bubbled through the mixture for 15 minutes. After evaporation of the solvents in vacuo, the residue was triturated with water. The pH of the solution was adjusted to 8 by addition of 5% aqueous sodium bicarbonate solution. The resultant beige precipitate was filtered, washed with water and ether and dried over P₂O₅ under high vacuum. The precipitate was stirred in ethyl acetate for 1 hour, filtered and dried under high vacuum at 50° C. to give the title compound as a beige solid (140 mg, 78%); NMR spectrum: (400 MHz; DMSO-d6+CF₃CO₂D) 2.29 (s, 3H), 2.71 (s, 3H), 5.01 (s, 2H), 7.24 (d, 1H), 7.30 (d, 1H), 7.48 (d, 1H), 7.86 (m, 1H), 7.93 (m, 2H), 8.09 (m, 2H), 8.74 (s, 1H), 8.96 (s, 1H); Mass spectrum: MH⁺ 416.

The {[4-({3-methyl-4-[(6-methylpyridin-3-yl)oxy]phenyl}amino)quinazolin-5-yl]oxy}acetic acid used as starting material was obtained as follows:

Sodium hydride (25.6 g, 60% dispersion in oil, 0.64 mol) was added portionwise to a solution of 5-hydroxy-2-methylpyridine (70 g, 0.64 mol) in DMA (700 ml) while keeping the temperature below 40° C. At the end of the addition, the mixture was stirred at room temperature for 1 hour and 2-fluoro-5-nitrotoluene (91.3 g, 0.59 mol) in DMA (100 ml) was added slowly. The mixture was stirred at 80° C. for 3 hours, then cooled. The solvents were removed in vacuo and the residue was partitioned between ethyl acetate and water. The organic layer washed with water and brine and dried over MgSO₄. After evaporation of the solvents, the residue was purified by chromatography on silica gel eluting with 30% ethyl acetate in petroleum ether to give 2-methyl-5-(2-methyl-4-nitrophenoxy)pyridine as an oil (141 g, 98%); NMR spectrum: (400 MHz; CDCl₃) 2.43 (s, 3H), 2.59 (s, 3H), 6.74 (d, 1H), 7.21 (d, 1H), 7.27 (d, 1H), 8.00 (d, 1H), 8.17 (s, 1H), 8.32 (s, 1H).

A mixture of 2-methyl-5-(2-methyl-4-nitrophenoxy)pyridine (141 g, 0.58 mol) and 10% palladium on charcoal (13 g) in ethyl acetate (200 ml) and ethanol (700 ml) was stirred under an atmosphere of hydrogen (1.2 bar) for 5 hours. The mixture was then purged with nitrogen and the catalyst was filtered off. The filtrate was evaporated to dryness to give 3-methyl-4-[(6-methylpyridin-3-yl)oxy]aniline as a white solid (120.6 g, 98%); Mass spectrum: MH⁺ 215.

3-Methyl-4-[(6-methylpyridin-3-yl)oxy]aniline (6.42 g, 30 mmol) and 4N hydrogen chloride in dioxane (7.55 ml, 30 mmol) were added to a suspension of 4-chloro-5-fluoroquinazoline (obtained as described in Example 1, preparation of starting materials, 5 g, 27.5 mmol) in acetonitrile (100 ml). The mixture was stirred at 80° C. for 2 hours. After cooling, the precipitate washed with acetonitrile. This precipitate was partitioned between DCM and 5% aqueous sodium bicarbonate solution and the pH was adjusted to 8. The organic layer washed with brine and dried over MgSO₄. Evaporation of the solvents gave 5-fluoro-N-{3-methyl-4-[(6-methylpyridin-3-yl)oxy]phenyl}quinazolin-4-amine as a dark gum (9.3 g, 94%) which crystallised on standing; NMR spectrum: (400 MHz; CDCl₃) 2.30 (s, 3H), 2.54 (s, 3H), 6.93 (d, 1H), 7.15-7.08 (m, 2H), 7.22 (m, 1H), 7.56 (d, 1H), 7.63 (s, 1H), 7.71 (m, 2H), 8.27 (s, 1H), 8.37 (d, 1H), 8.71 (s, 1H).

A mixture of 5-fluoro-N-{3-methyl-4-[(6-methylpyridin-3-yl)oxy]phenyl}quinazolin-4-amine (10.8 g, 30 mmol) and sodium methoxide (4.86 g, 90 mmol) in methanol (250 ml) was heated at reflux for 16 hours. After cooling and evaporation of the solvents, the residue was dissolved in dichloromethane. This solution washed with water and brine and dried over MgSO₄. Evaporation of the solvents gave 5-methoxy-N-{3-methyl-4-[(6-methylpyridin-3-yl)oxy]phenyl}quinazolin-4-amine as a white solid (10.7 g, 96%); NMR spectrum: (400 MHz; CDCl₃) 2.29 (s, 3H), 2.53 (s, 3H), 4.12 (s, 3H), 6.92 (m, 2H), 7.12 (m, 2H), 7.48 (d, 1H), 7.55 (d, 1H), 7.63 (m, 2H), 8.27 (s, 1H), 8.64 (s, 1H), 9.78 (bs, 1H).

A mixture of 5-methoxy-N-{3-methyl-4-[(6-methylpyridin-3-yl)oxy]phenyl}quinazolin-4-amine (10.04 g, 27 mmol) and pyridine hydrochloride (12.42 g, 108 mmol) in pyridine (100 ml) was heated at reflux for 2 hours. After cooling and evaporation of the solvents, the residue was triturated in 5% aqueous sodium bicarbonate and the resulting mixture was stirred for 30 minutes. The yellowish precipitate was filtered, washed with water and ether, and dried over P₂O₅ under high vacuum to give 5-hydroxy-N-{3-methyl-4-[(6-methylpyridin-3-yl)oxy]phenyl}quinazolin-4-amine (9.3 g, 96%); NMR spectrum: (400 MHz) 2.20 (s, 3H), 2.44 (s, 3H), 6.71 (m, 2H), 6.96 (d, 1H), 7.23 (m, 2H), 7.47 (m, 1H), 7.60 (m, 2H), 8.18 (s, 1H), 8.36 (s, 1H).

DEAD (0.7 ml, 4.47 mmol) was added dropwise to a solution of 5-hydroxy-N-{3-methyl-4-[(6-methylpyridin-3-yl)oxy]phenyl}quinazolin-4-amine (800 mg, 2.23 mmol), methyl glycolate (0.258 ml, 3.35 mmol) and triphenylphosphine (1.17 g, 4.47 mmol) in DCM (30 ml). The mixture was stirred at room temperature for 1 hour. After evaporation of the solvents, the residue was purified by chromatography on silica gel eluting with ethyl acetate to give methyl {[4-({3-methyl-4-[(6-methylpyridin-3-yl)oxy]phenyl}amino)quinazolin-5-yl]oxy}acetate as a white solid (710 mg, 74%); Mass spectrum: MH⁺ 431.

A mixture of methyl {[4-({3-methyl-4-[(6-methylpyridin-3-yl)oxy]phenyl}amino)quinazolin-5-yl]oxy}acetate (700 mg, 1.63 mmol) and 2N aqueous sodium hydroxide (1.6 ml, 3.2 mmol) in ethanol (10 ml) and THF (10 ml) was stirred at room temperature for 18 hours. After evaporation of the solvents under vacuum, the residue was diluted in water and the pH was adjusted to 4 with diluted acetic acid. The white precipitate was filtered, washed with water and dried over P₂O₅ under high vacuum to give {[4-({3-methyl-4-[(6-methylpyridin-3-yl)oxy]phenyl}amino)quinazolin-5-yl]oxy}acetic acid as a beige solid (640 mg, 94%); NMR spectrum: (400 MHz; DMSO-d6+CF₃CO₂D) 2.29 (s, 3H), 2.69 (s, 3H), 5.16 (s, 2H), 7.24 (d, 1H), 7.44 (d, 1H), 7.48 (d, 1H), 7.85 (m, 3H), 8.06 (m, 2H), 8.71 (s, 1H), 8.98 (s, 1H).

### EXAMPLE 52

### N-(2-Hydroxyethyl)-2-{[4-({3-methyl-4-[(6-methylpyridin-3-yl)oxy]phenyl}amino)quinazolin-5-yl]oxy}acetamide

The procedure described in Example 51 was repeated with {[4-({3-methyl-4-[(6-methylpyridin-3-yl)oxy]phenyl}amino)quinazolin-5-yl]oxy}acetic acid (obtained as described in Example 51, preparation of starting materials, 140 mg, 0.32 mmol) and ethanolamine (78 μl, 1.28 mmol) and the mixture was stirred for 18 hours to give the title compound as a beige solid (115 mg, 75%); NMR spectrum: (400 MHz; DMSO-d6+CF₃CO₂D) 2.30 (s, 3H), 2.71 (s, 3H), 3.28 (t, 2H), 3.49 (t, 2H), 5.03 (s, 2H), 7.24 (d, 1H), 7.29 (d, 1H), 7.48 (d, 1H), 7.83 (m, 1H), 7.95 (m, 2H), 8.14-8.05 (m, 2H), 8.76 (s, 1H), 8.97 (s, 1H); Mass spectrum: MH⁺ 460.

### EXAMPLE 53

### N-Methyl-2-{[4-({3-methyl-4-[(6-methylpyridin-3-yl)oxy]phenyl}amino)quinazolin-5-yl]oxy}acetamide

The procedure described in Example 51 was repeated with {[4-({3-methyl-4-[(6-methylpyridin-3-yl)oxy]phenyl}amino)quinazolin-5-yl]oxy}acetic acid (obtained as described in Example 51, preparation of starting materials, 140 mg, 0.32 mmol) and methylamine to give the title compound (140 mg, 75%); NMR spectrum: (400 MHz; DMSO-d6+CF₃CO₂D) 2.30 (s, 3H), 2.70 (s, 3H), 2.73 (s, 3H), 5.02 (s, 2H), 7.25 (d, 1H), 7.30 (d, 1H), 7.48 (d, 1H), 7.86 (m, 1H), 7.94 (m, 2H), 8.13-8.05 (m, 2H), 8.75 (s, 1H), 8.97 (s, 1H); Mass spectrum: MH⁺ 430.

### EXAMPLE 54

### N-(2-Hydroxyethyl)-N-methyl-2-{[4-({3-methyl-4-[(6-methylpyridin-3-yl)oxy]phenyl}amino)quinazolin-5-yl]oxy}acetamide

The procedure described in Example 51 was repeated with {[4-({3-methyl-4-[(6-methylpyridin-3-yl)oxy]phenyl}amino)quinazolin-5-yl]oxy}acetic acid (obtained as described in Example 51, preparation of starting materials, 140 mg, 0.32 mmol) and 2-(methylamino)ethanol (105 μl, 1.28 mmol) and the mixture was stirred for 3 days to give the title compoundas a white solid (74 mg, 47%) after purification by chromatography on silica gel eluting with 0-6% methanol in DCM; NMR spectrum: (400 MHz; 100° C.) (2 rotamers) 2.21 (s, 3H), 2.44 (s, 3H), 2.97 and 3.08 (s, 3H), 3.45 (m, 2H), 3.60 (m, 2H), 4.77 and 5.01 (m, 1H), 5.15 and 5.23 (s, 2H), 6.98 (m, 1H), 7.23 (m, 3H), 7.38 (d, 1H), 7.76 (m, 1H), 7.97 (m, 1H), 8.10 (m, 1H), 8.19 (m, 1H), 8.54 (s, 1H); Mass spectrum: MH⁺ 474.

### EXAMPLE 55

### N-{3-Methyl-4-[(6-methylpyridin-3-yl)oxy]phenyl}-5-(2-oxo-2-pyrrolidin-1-ylethoxy)quinazolin-4-amine

The procedure described in Example 51 was repeated with {[4-({3-methyl-4-[(6-methylpyridin-3-yl)oxy]phenyl}amino)quinazolin-5-yl]oxy}acetic acid (obtained as described in Example 51, preparation of starting materials, 140 mg, 0.32 mmol) and pyrrolidine (108 μl, 1.28 mmol) except that the mixture was stirred at 65° C. for 4 hours to give the title compound as a beige solid (74 mg, 47%) after purification by chromatography on silica gel eluting with 0-6% methanol in DCM; NMR spectrum: (400 MHz; DMSO-d6+CF₃CO₂D) 1.84 (t, 2H), 1.97 (t, 2H), 2.30 (s, 3H), 2.70 (s, 3H), 3.44 (t, 2H), 3.50 (t, 2H), 5.23 (s, 2H), 7.24 (d, 1H), 7.47 (d, 1H), 7.51 (d, 1H), 7.85 (m, 1H), 7.91 (d, 1H), 8.00 (d, 1H), 8.09 (m, 2H), 8.75 (s, 1H), 8.96 (s, 1H); Mass spectrum: MH⁺ 470.

### EXAMPLE 56

### N-{3-Methyl-4-[(6-methylpyridin-3-yl)oxy]phenyl}-5-(2-oxo-2-piperazin-1-ylethoxy)quinazolin-4-amine

The procedure described in Example 9 was repeated with 5-hydroxy-N-{3-methyl-4-[(6-methylpyridin-3-yl)oxy]phenyl}quinazolin-4-amine (obtained as described in Example 51, preparation of starting materials, 400 mg, 1.1 mmol) and tert-butyl 4-(chloroacetyl)piperazine-1-carboxylate (prepared according to the method described by Shuttleworth S. J. et al, Bioorg. Med. Chem. Lett., 2000, 10, 2501, 306 mg, 1.2 mmol) except that at the end of the reaction after evaporation of the solvents solvents in vacuo, the residue was purified by chromatography on silica gel eluting with 0 to 4.5% methanol in DCM to give a solid (510 mg). After removal of solvents, a portion of this solid (220 mg) was stirred with TFA (5 ml) for 18 hours. After evaporation of the solvents in vacuo, the residue was diluted in water. The pH of the solution was adjusted to 11 by addition of 2N aqueous sodium hydroxide. The resulting precipitate was filtered, washed with water and ether, and dried over P₂O₅ under high vacuum to give the title compound (166 mg, 71%); NMR spectrum: (400 MHz) 2.21 (s, 3H), 2.44 (s, 3H), 2.70 (m, 2H), 2.75 (m, 2H), 3.41 (m, 2H), 3.50 (m, 2H), 5.16 (s, 2H), 6.97 (d, 1H), 7.23 (m, 3H), 7.37 (d, 1H), 7.74 (t, 1H), 7.96 (d, 1H), 8.11 (s, 1H), 8.19 (s, 1H), 8.54 (s, 1H); Mass spectrum: MH⁺ 485.

### EXAMPLE 57

### N-{3-Methyl-4-[(6-methylpyridin-3-yl)oxy]phenyl}-5-[2-(4-methylpiperazin-1-yl)-2-oxoethoxy]quinazolin-4-amine

The procedure described in Example 18 was repeated with N-{3-methyl-4-[(6-methylpyridin-3-yl)oxy]phenyl}-5-(2-oxo-2-piperazin-1-ylethoxy)quinazolin-4-amine (obtained as described in Example 56, 225 mg, 0.46 mmol). The reaction mixture was diluted with water, and the precipitate collected by filtration and then purified by chromatography on silica gel eluting with 0 to 8% methanol in DCM and trituration of the residue in ether to give the title compound as a pale solid (112 mg, 48%); NMR spectrum: (400 MHz) 2.21 (s, 6H), 2.33 (m, 2H), 2.39 (m, 2H), 2.44 (s, 3H), 3.49 (m, 2H), 3.58 (m, 2H), 5.17 (s, 2H), 6.97 (d, 1H), 7.23 (m, 3H), 7.37 (d, 1H), 7.75 (t, 1H), 7.96 (d, 1H), 8.10 (s, 1H), 8.19 (s, 1H), 8.54 (s, 1H), 11.12 (s, 1H); Mass spectrum: MH⁺ 499.

### EXAMPLE 58

### (2S)-2-{[4-({3-Methyl-4-[(6-methylpyridin-3-yl)oxy]phenyl}amino)quinazolin-5-yl]oxy}propanamide

The procedure described in Example 51 was repeated with (2S)-2-{[4-({3-methyl-4-[(6-methylpyridin-3-yl)oxy]phenyl}amino)quinazolin-5-yl]oxy}propanoic acid (150 mg, 0.34 mmol) and ammonia to give the title compound as a beige solid (115 mg, 55%); NMR spectrum: (400 MHz; DMSO-d6+CF₃CO₂D) 1.70 (d, 3H), 2.29 (s, 3H), 2.70 (s, 3H), 5.34 (q, 1H), 7.25 (d, 1H), 7.39 (d, 1H), 7.47 (d, 1H), 7.83 (m, 1H), 7.93 (m, 2H), 8.12-8.03 (m, 2H), 8.74 (s, 1H), 8.96 (s, 1H); Mass spectrum: MH⁺ 430.

The (2S)-2-{[4-({3-methyl-4-[(6-methylpyridin-3-yl)oxy]phenyl}amino)quinazolin-5-yl]oxy}propanoic acid used as starting material was obtained as follows:

The procedure described in Example 51 preparation of starting materials, was repeated with 5-hydroxy-N-{3-methyl-4-[(6-methylpyridin-3-yl)oxy]phenyl}quinazolin-4-amine (obtained as described in Example 51, preparation of starting materials, 250 mg, 0.70 mmol) and methyl (R)-lactate (0.1 ml, 1.05 mmol) to give methyl (2S)-2-{[4-({3-methyl-4-[(6-methylpyridin-3-yl)oxy]phenyl}amino)quinazolin-5-yl]oxy}propanoate (319 mg, 86%); NMR spectrum: (400 MHz; CDCl₃) 1.81 (d, 3H), 2.30 (s, 3H), 2.53 (s, 3H), 3.87 (s, 3H), 5.15 (q, 1H), 6.79 (d, 1H), 6.93 (d, 1H), 7.06-7.14 (m, 2H), 7.70-7.40(m, 3H), 7.84 (s, 1H), 8.28 (s, 1H), 8.65 (s, 1H); Mass spectrum: MH⁺ 445.

This was then treated with 2N aqueous sodium hydroxide according to the procedure described in Example 51, preparation of starting materials to give (2S)-2-{[4-({3-methyl-4-[(6-methylpyridin-3-yl)oxy]phenyl}amino)quinazolin-5-yl]oxy}propanoic acid as a solid (237 mg, 78%); NMR spectrum: (400 MHz) 1.69 (d, 3H), 2.20 (s, 3H), 2.44 (s, 3H), 5.37 (q, 1H), 6.99 (d, 1H), 7.18-7.24 (m, 3H), 7.36 (d, 1H), 7.73 (t, 1H), 7.87 (m, 2H), 8.18 (s, 1H), 8.54 (s, 1H).

### EXAMPLE 59

### (2R)-2-{[4-({3-Methyl-4-[(6-methylpyridin-3-yl)oxy]phenyl}amino)quinazolin-5-yl]oxy}propanamide

The procedure described in Example 51 was repeated with (2R)-2-{[4-({3-methyl-4-[(6-methylpyridin-3-yl)oxy]phenyl}amino)quinazolin-5-yl]oxy}propanoic acid and ammonia to give the title compound as a beige solid (155 mg, 77%); NMR spectrum: (400 MHz; DMSO-d6+CF₃CO₂D) 1.70 (d, 3H), 2.29 (s, 3H), 2.70 (s, 3H), 5.34 (q, 1H), 7.25 (d, 1H), 7.39 (d, 1H), 7.47 (d, 1H), 7.83 (m, 1H), 7.93 (m, 2H), 8.12-8.03 (m, 2H), 8.74 (s, 1H), 8.96 (s, 1H); Mass spectrum: MH⁺ 430.

The (2R)-2-{[4-({3-methyl-4-[(6-methylpyridin-3-yl)oxy]phenyl}amino)quinazolin-5-yl]oxy}propanoic acid used as starting material was obtained as follows:

The procedure described in Example 51 starting material, was repeated with 5-hydroxy-N-{3-methyl-4-[(6-methylpyridin-3-yl)oxy]phenyl}quinazolin-4-amine (obtained as described in Example 51, preparation of starting materials, 600 mg, 1.68 mmol) and methyl (S)-lactate (0.1 ml, 1.05 mmol) to give methyl (2R)-2-{[4-({3-methyl-4-[(6-methylpyridin-3-yl)oxy]phenyl}amino)quinazolin-5-yl]oxy}propanoate (623 mg, 84%); NMR spectrum: (400 MHz; CDCl₃) 1.81 (d, 3H), 2.30 (s, 3H), 2.53 (s, 3H), 3.87 (s, 3H), 5.15 (q, 1H), 6.79 (d, 1H), 6.93 (d, 1H), 7.06-7.14 (m, 2H), 7.70-7.40(m, 3H), 7.84 (s, 1H), 8.28 (s, 1H), 8.65 (s, 1H); Mass spectrum: MH⁺ 445.

This was then treated with 2N aqueous sodium hydroxide according to the procedure described in Example 51, preparation of starting materials to give (2R)-2-{[4-({3-methyl-4-[(6-methylpyridin-3-yl)oxy]phenyl}amino)quinazolin-5-yl]oxy}propanoic acid as a solid (412 mg, 83%); NMR spectrum: (400 MHz) 1.68 (d, 3H), 2.20 (s, 3H), 2.43 (s, 3H), 5.34 (q, 1H), 6.98 (d, 1H), 7.18-7.24 (m, 3H), 7.36 (d, 1H), 7.72 (t, 1H), 7.87 (m, 2H), 8.18 (s, 1H), 8.53 (s, 1H).

### EXAMPLE 60

### (2R)—N-(2-Hydroxyethyl)-N-methyl-2-{[4-({3-methyl-4-[(6-methylpyridin-3-yl)oxy]phenyl}amino)quinazolin-5-yl]oxy}propanamide

The procedure described in Example 51 was repeated with (2R)-2-{[4-({3-methyl-4-[(6-methylpyridin-3-yl)oxy]phenyl}amino)quinazolin-5-yl]oxy}propanoic acid (obtained as described in Example 59, preparation of starting materials, 200 mg, 0.46 mmol) and 2-(methylamino)ethanol (244 μl, 3.04 mmol) except that after addition of 2-(methylamino)ethanol, the mixture was stirred at 65° C. for 18 hours. Purification by chromatography on silica gel eluting with 0 to 6% methanol in DCM was followed by further purification on an HPLC column (C18, 5 microns, 19 mm diameter, 100 mm length) of a preparative HPLC-MS system eluting with a mixture of water and acetonitrile containing 2 g/l of ammonium carbonate (gradient), and trituration of the residue in ether to give the title compound as a beige solid (22 mg, 10%); NMR spectrum: (400 MHz) 1.60 (m, 3H), 2.21 (s, 3H), 2.44 (s, 3H), 2.92 and 3.18 (s, 3H), 3.7-3.3 (m, 4H), 4.73 and 5.00 (m, 1H), 5.81 and 5.90 (m, 1H), 6.98 (m, 1H), 7.36-7.24 (m, 4H), 7.71 (m, 1H), 7.90 (m, 1H), 8.02 (m, 1H), 8.19 (s, 1H), 8.52 (s, 1H), 11.02 (s, 1H); Mass spectrum: MH⁺ 488.

### EXAMPLE 61

### 2-Methyl-2-{[4-({3-methyl-4-[(6-methylpyridin-3-yl)oxy]phenyl}amino)quinazolin-5-yl]oxy}propanamide

Ammonia was bubbled through a solution of 6,6-dimethyl-4-{3-methyl-4-[(6-methylpyridin-3-yl)oxy]phenyl}-4H-[1,4]oxazepino[5,6,7-de]quinazolin-5(6H)-one (200 mg, 0.46 mmol) in DMF (3 ml) for 15 minutes. The vessel was then sealed and the mixture was stirred at room temperature for 18 hours. After evaporation of the solvents in vacuo, the residue was triturated with water. The beige precipitate was filtered, washed with water and ether, and dried over P₂O₅ under high vacuum to give the title compound as a beige solid (135 mg, 65%); NMR spectrum: (400 MHz) 1.73 (s, 6H), 2.22 (s, 3H), 2.44 (s, 3H), 6.85 (d, 1H), 7.00 (d, 1H), 7.23 (m, 2H), 7.36 (d, 1H), 7.48 (s, 1H), 7.71 (m, 2H), 7.83 (s, 1H), 7.97 (s, 1H), 8.17 (s, 1H), 8.52 (s, 1H), 10.39 (s, 1H); Mass spectrum: MH⁺ 444.

The 6,6-dimethyl-4-{3-methyl-4-[(6-methylpyridin-3-yl)oxy]phenyl}-4H-[1,4]oxazepino[5,6,7-de]quinazolin-5(6H)-one used as starting material was prepared as follows:

Sodium hydroxide (1.34 g, 33.5 mmol) was added portionwise to an ice-cooled mixture of 5-hydroxy-N-{3-methyl-4-[(6-methylpyridin-3-yl)oxy]phenyl}quinazolin-4-amine (obtained as described in Example 51, preparation of starting materials, 1.5 g, 4.19 mmol) and 2-methyl-1,1,1-trichloro-2-propanol (1.56 g, 8.38 mmol) in acetone (30 ml). The mixture was stirred at room temperature for 18 hours. The resulting precipitate was filtered and washed with acetone. The resulting solid was dissolved in water. The pH of the solution was adjusted to 4 by addition of saturated ammonium chloride solution then diluted acetic acid solution. The resulting precipitate was filtered, washed with water and ether, then dried over P₂O₅ at 50° C. to give 2-methyl-2-{[4-({3-methyl-4-[(6-methylpyridin-3-yl)oxy]phenyl}amino)quinazolin-5-yl]oxy}propanoic acid as a beige solid (1.23 g, 66%); NMR spectrum: (400 MHz) 1.81 (s, 6H), 2.21 (s, 3H), 2.44 (s, 3H), 6.97 (d, 1H), 7.02 (d, 1H), 7.23 (m, 2H), 7.38 (d, 1H), 7.70 (m, 2H), 7.77 (s, 1H), 8.18 (s, 1H), 8.50 (s, 1H); Mass spectrum: MH⁺ 445.

A mixture of 2-methyl-2-{[4-({3-methyl-4-[(6-methylpyridin-3-yl)oxy]phenyl}amino)quinazolin-5-yl]oxy}propanoic acid (700 mg, 1.6 mmol), diisopropylethylamine (279 μl, 1.6 mmol) and HATU (730 mg, 1.92 mmol) in DCM (10 ml) was stirred at room temperature for 18 hours. The mixture was diluted with DCM, washed with diluted aqueous sodium bicarbonate and brine, and dried over MgSO₄. After evaporation of the solvents, the residue was purified by chromatography on silica gel eluting with ethyl acetate to give 6,6-dimethyl-4-{3-methyl-4-[(6-methylpyridin-3-yl)oxy]phenyl}-4H-[1,4]oxazepino[5,6,7-de]quinazolin-5(6H)-one as a foam (618 mg, 92%); Mass spectrum: MH⁺ 427.

### EXAMPLE 62

### N,2-Dimethyl-2-{[4-({3-methyl-4-[(6-methylpyridin-3-yl)oxy]phenyl}amino)quinazolin-5-yl]oxy}propanamide

The procedure described in Example 61 was repeated with 6,6-dimethyl-4-{3-methyl-4-[(6-methylpyridin-3-yl)oxy]phenyl}-4H-[1,4]oxazepino[5,6,7-de]quinazolin-5(6H)-one (obtained as described in Example 61, preparation of starting materials, 200 mg, 0.46 mmol) and methylamine to give the title compound as a white solid (180 mg, 84%); NMR spectrum: (400 MHz) 1.72 (s, 6H), 2.23 (s, 3H), 2.44 (s, 3H), 2.64 (d, 3H), 6.72 (d, 1H), 7.01 (d, 1H), 7.22 (m, 2H), 7.36 (d, 1H), 7.69 (t, 1H), 7.74 (d, 1H), 7.84 (s, 1H), 8.17 (s, 1H), 8.43 (m, 1H), 8.54 (s, 1H), 10.27 (s, 1H); Mass spectrum: MH⁺ 458.

### EXAMPLE 63

### (3R)-1-{(2S)-2-[(4-{[3-chloro-4-(pyridin-2-ylmethoxy)phenyl]amino}quinazolin-5-yl)oxy]propanoyl}pyrrolidin-3-ol

Methyl (2S)-2-[(4-{[3-chloro-4-(pyridin-2-ylmethoxy)phenyl]amino}quinazolin-5-yl)oxy]propanoate (200 mg, 0.432 mmol, see Example 28 starting material) was dissolved in (S)-3 hydroxy pyrrolidine (1 ml) and the solution heated in a microwave synthesisor (CEM) at 140° C. for 20 minutes. The solution was added to water (5 ml) and extracted into dichloromethane (2×10 ml). The combined extracts were dried by passing through a phase separating column, and then loaded onto a prepacked silica column (20 g) and eluted with 1% 880 NIH3/10% methanol in DCM. The relevant fractions were combined to give the title compound as a solid (67 mg, 30%); NMR spectrum: (373K) 1.65 (d, 3H), 1.7-1.95 (bs, 1H), 1.95-2.05 (bs, 1H), 3.4-3.7 (bs, 2H), 4.35-4.45 (bs, 1H), 4.50-4.60 (bs, 1H), 5.25 (s, 2H), 5.50-5.60 (m, 1H), 7.18-7.20 (d, 1H), 7.23-7.28 (d, 1H), 7.30-7.35 (m, 1H), 7.35-7.40 (d, 1H), 7.55-7.60 (d, 1H), 7.65-7.75 (t, 1H), 7.80-7.90 (m, 2H), 8.20 (d, 1H), 8.50 (s, 1H), 8.55-8.65 (d, 1H), 10.75-10.85 (bs, 1H); Mass spectrum: MH⁺ 520.

### EXAMPLE 64

### (3S)-1-{(2S)-2-[(4-{[3-chloro-4-(pyridin-2-ylmethoxy)phenyl]amino}quinazolin-5-yl)oxy]propanoyl}pyrrolidin-3-ol

The procedure described in Example 21 was repeated using (2S)-2-[(4-{[3-chloro-4-(pyridin-2-ylmethoxy)phenyl]amino}quinazolin-5-yl)oxy]propanoic acid (200 mg, 0.44 mmol, see Example 28 starting material) and (R)-3 hydroxypyrrolidine (1 g) in THF (1 ml) to give the title compound as a solid (55 mg, 26%); NMR spectrum: (373K) 1.65 (d, 3H), 1.7-1.95 (bs, 1H), 1.95-2.05 (bs, 1H), 3.4-3.7 (bs, 2H), 4.35-4.45 (bs, 1H), 4.50-4.60 (bs, 1H), 5.25 (s, 2H), 5.50-5.60 (m, 1H), 7.18-7.20 (d, 1H), 7.23-7.28 (d, 1H), 7.30-7.35 (m, 1H), 7.35-7.40 (d, 1H), 7.55-7.60 (d, 1H), 7.65-7.75 (t, 1H), 7.80-7.90 (m, 2H), 8.20 (d, 1H), 8.50 (s, 1H), 8.55-8.65 (d, 1H), 10.75-10.85 (bs, 1H); Mass spectrum: MH⁺ 520.

### EXAMPLE 65

### (3R)-1-{(2R)-2-[(4-{[3-methyl-4-(pyridin-2-ylmethoxy)phenyl]amino}quinazolin-5-yl)oxy]propanoyl}pyrrolidin-3-ol

A solution of methyl (2R)-2-[(4-{[3-methyl-4-(pyridin-2-ylmethoxy)phenyl]amino}quinazolin-5-yl)oxy]propanoate (100 mg, 0.225 mmol) and (R)-3-hydroxypyrrolidine (500 μl, 6.03 mmol) in THF (4 ml) was heated under reflux for 16 hours. The mixture was evaporated, and the residue partitioned between DCM and water. The organic layer was concentrated in vacuo and the residue was crystallised from ethyl acetate to give the title compound as a white crystalline solid (78 mg, 69%); NMR spectrum: 1.63 (d, 3H), 1.75-2.10 (m, 2H), 2.30 (s, 1H), 3.35-3.65 (m, 3H), 3.70 (m, 1H), 4.35 (m, 1H), 4.75 (m, 1H), 5.20 (s, 1H), 5.51 (m, 1H), 7.02 (d, 1H), 7.16 (d, 1H), 7.32 (dd, 1H), 7.33 (d, 1H), 7.55 (d, 1H), 7.67 (dd, 1H), 7.73 (dd, 1H), 7.78 (d, 1H), 7.83 (ddd, 1H), 8.46 (s, 1H), 8.58 (d, 1H), 10.53 (s, 1H); Mass spectrum: MH⁺ 500.

The methyl (2R)-2-[(4-{[3-methyl-4-(pyridin-2-ylmethoxy)phenyl]amino}quinazolin-5-yl)oxy]propanoate used as starting material was obtained as follows:

To a suspension of 4-{[3-methyl-4-(pyridin-2-ylmethoxy)phenyl]amino}quinazolin-5-ol (obtained as described in Example 49, preparation of starting materials, 1253 mg, 3.50 mmol) in DCM (125 ml) was added sequentially S-methyl lactate (501 μl, 5.25 mmol), triphenylphosphine (1376 mg, 5.25 mmol) and DTAD (1208 mg, 5.25 mmol). The mixture was stirred for 3 hours; the resulting solution was loaded onto a silica column, which was eluted with ethyl acetate. Evaporation of the appropriate fractions gave methyl (2R)-2-[(4-{[3-methyl-4-(pyridin-2-ylmethoxy)phenyl]amino}quinazolin-5-yl)oxy]propanoate as a yellow foam (1360 mg, 88%); NMR spectrum: 1.71 (d, 3H), 2.30 (s, 3H), 3.79 (s, 3H), 5.22 (s, 2H), 5.50 (q, 1H), 7.04 (d, 1H), 7.14 (d, 1H), 7.35 (dd, 1H), 7.36 (d, 1H), 7.57 (d, 1H), 7.65 (dd, 1H), 7.68 (d, 1H), 7.70 (dd, 1H), 7.86 (ddd, 1H), 8.49 (s, 1H), 8.60 (dd, 1H), 10.28 (s, 1H).

### EXAMPLE 66

### (2R)—N-methyl-2-[(4-{[3-methyl-4-(pyridin-2-ylmethoxy)phenyl]amino}quinazolin-5-yl)oxy]propanamide

Methyl (2R)-2-[(4-{[3-methyl-4-(pyridin-2-ylmethoxy)phenyl]amino}quinazolin-5-yl)oxy]propanoate (obtained as described in Example 65, preparation of starting materials, 100 mg, 0.225 mmol) was treated with methylamine (2M solution in ethanol, 4 ml, 8 mmol); the mixture was irradiated in a CEM Explorer focused microwave synthesiser at 120° C. for 20 minutes. The resulting crystals were collected by filtration and washed with cold ethanol to give the title compound as a white crystalline solid (76 mg, 76%); NMR spectrum: 1.64 (d, 3H), 2.30 (s, 3H), 2.68 (d, 3H), 5.13 (q, 1H), 5.22 (s, 2H), 6.97 (d, 1H), 7.04 (d, 1H), 7.34 (d, 2H), 7.36 (dd, 2H), 7.57 (d, 1H), 7.71 (dd, 1H), 7.71 (dd, 1H), 7.74 (d, 1H), 7.87 (ddd, 1H), 8.34 (d, 1H), 8.49 (s, 1H), 8.60 (d, 1H), 10.43 (s, 1H); Mass spectrum: MH⁺ 444.

### EXAMPLE 67

### (2R)—N-(2-Hydroxyethyl)-N-methyl-2-[(4-{[3-methyl-4-(pyridin-2-ylmethoxy)phenyl]amino}quinazolin-5-yl)oxy]propanamide

A solution of methyl (2R)-2-[(4-{[3-methyl-4-(pyridin-2-ylmethoxy)phenyl]amino}quinazolin-5-yl)oxy]propanoate (obtained as described in Example 65, preparation of starting materials, 100 mg, 0.225 mmol) in N-methylethanolamine (2 ml) was heated at 75° C. for 30 minutes. The mixture was evaporated, and the residue partitioned between DCM and water. The organic layer was loaded onto a silica column, which was eluted with 0 to 4% (10:1 MeOH/conc. NH_{3(aq)}) in DCM. Evaporation of the appropriate fractions gave the title compound as a yellow foam (61 mg, 56%); NMR spectrum: 1.63 (d, 3H), 2.30 (s, 3H), 2.94 (s, 3H), 3.40-3.65 (m, 4H), 5.20 (s, 2H), 5.78 (m, 1H), 7.02 (d, 1H), 7.18 (d, 1H), 7.31 (dd, 1H), 7.32 (d, 1H), 7.55 (d, 1H), 7.66 (dd, 1H), 7.74 (dd, 1H), 7.77 (d, 1H), 7.83 (ddd, 1H), 8.45 (s, 1H), 8.58 (d, 1H), 10.69 (s, 1H); Mass spectrum: MH⁺ 488.

### EXAMPLE 68

### 5-[(1R)-1-Methyl-2-oxo-2-pyrrolidin-1-ylethoxy]-N-[3-methyl-4-(pyridin-2-ylmethoxy)phenyl]quinazolin-4-amine

A solution of methyl (2R)-2-[(4-{[3-methyl-4-(pyridin-2-ylmethoxy)phenyl]amino}quinazolin-5-yl)oxy]propanoate (obtained as described in Example 65, preparation of starting materials, 100 mg, 0.225 mmol) in pyrrolidine (3 ml) was irradiated in a CEM Explorer focused microwave synthesiser at 140° C. for 30 minutes. The mixture was evaporated, and the residue purified by flash column chromatography, eluting with 0 to 3.5% (10:1 MeOH/conc. NH_{3(aq)}) in DCM. Evaporation of the appropriate fractions and crystallisation of the residue from ethyl acetate/iso-hexane gave the title compound as a white crystalline solid (38 mg, 35%); NMR spectrum: 1.59 (d, 3H), 1.83 (m, 2H), 1.94 (m, 2H), 2.30 (s, 3H), 3.34-3.49 (m, 3H), 3.76 (m, 1H), 5.22 (s, 2H), 5.59 (q, 1H), 7.03 (d, 1H), 7.22 (d, 1H), 7.33 (d, 1H), 7.35 (dd, 1H), 7.57 (d, 1H), 7.70 (dd, 1H), 7.74 (dd, 1H), 7.82 (d, 1H), 7.87 (ddd, 1H), 8.47 (s, 1H), 8.59 (dd, 1H), 10.82 (s, 1H); Mass spectrum: MH⁺ 484.

### EXAMPLE 69

### 2-Methyl-2-[(4-{[3-methyl-4-(pyridin-2-ylmethoxy)phenyl]amino}quinazolin-5-yl)oxy]propanamide

To a suspension of 4-{[3-methyl-4-(pyridin-2-ylmethoxy)phenyl]amino}quinazolin-5-ol (obtained as described in Example 49, preparation of starting materials, 143 mg, 0.40 mmol) in 1,4-dioxane (25 ml) was added sequentially cesium carbonate (430 mg, 1.32 mmol) and sodium hydride (53 mg, 1.32 mmol). The mixture was stirred under an atmosphere of nitrogen at 50° C. for 30 minutes. 2-Bromo-2-methylpropanamide (219 mg, 1.32 mmol) was added to the resulting solution; the temperature was raised to 100° C. and the mixture stirred under an atmosphere of nitrogen for a further 16 hours. The mixture was cooled to ambient temperature, and saturated aqueous ammonium chloride solution (4 ml) was added. The mixture was evaporated, and the residue shaken with a mixture of DCM (50 ml) and saturated aqueous sodium carbonate solution. The resulting precipitate was collected by filtration, and combined with the organic layer, and concentrated in vacuo. The residue was crystallised twice from ethyl acetate to give the title compound as a white crystalline solid (39 mg, 22%); NMR spectrum: 1.72 (s, 6H), 2.42 (s, 3H), 5.22 (s, 2H), 6.84 (d, 1H), 7.04 (d, 1H), 7.33 (d, 1H), 7.36 (dd, 1H), 7.44 (s, 1H), 7.56 (d, 1H), 7.62 (dd, 1H), 7.65 (d, 1H), 7.67 (dd, 1H), 7.87 (ddd, 1H), 8.25 (s, 1H), 8.47 (s, 1H), 8.60 (dd, 1H), 10.23 (s, 1H). Mass spectrum: MH⁺ 444.

### EXAMPLE 70

### N-(2-hydroxyethyl)-2-methyl-2-{[4-({3-methyl-4-[(6-methylpyridin-3-yl)oxy]phenyl}amino)quinazolin-5-yl]oxy}propanamide

The procedure described in Example 61 was repeated using ethanolamine (4 equivalents) instead of ammonia except that the mixture was stirred at room temperature for 1 week. After evaporation of the solvents, the residue was purified by chromatography on silica gel (eluant: 0 to 6% methanol in DCM). After evaporation of the solvents, the solid was triturated in ether and dried under vacuum to give the title compound (165 mg, 72%); NMR Spectrum: (400 MHz; DMSOd₆ and CF₃CO₂D) 1.82 (s, 6H), 2.31 (s, 3H), 2.71 (s, 3H), 3.23 (m, 2H), 3.41 (m, 2H), 7.21 (d, 1H), 7.25 (d, 1H), 7.46 (d, 1H), 7.72 (m, 1H), 7.83 (m, 1H), 7.93 (d, 1H), 7.98 (t, 1H), 8.11 (d, 1H), 8.74 (s, 1H), 8.94 (s, 1H); Mass spectrum: MH⁺ 488.

### EXAMPLE 71

### N-(2-hydroxyethyl)-N,2-dimethyl-2-{[4-({3-methyl-4-[(6-methylpyridin-3-yl)oxy]phenyl}amino)quinazolin-5-yl]oxy}propanamide

The procedure described in Example 61 was repeated using 2-(methylamino)ethanol (4 equivalents) instead of ammonia except that the mixture was stirred at room temperature for 1 week. After evaporation of the solvents, the residue was purified by chromatography on silica gel (eluant: 0 to 6% methanol in DCM). After evaporation of the solvents, the solid was triturated in ether and dried under vacuum to give the title compound (55 mg, 23%); HPLC t_{R}: 2.85 min; Mass spectrum: MH⁺ 502.

### EXAMPLE 72

### (2S)—N-methyl-2-{[4-({3-methyl-4-[(6-methylpyridin-3-yl)oxy]phenyl}amino)quinazolin-5-yl]oxy}propanamide

The procedure described in Example 51 was repeated using (2S)-2-{[4-({3-methyl-4-[(6-methylpyridin-3-yl)oxy]phenyl}amino)quinazolin-5-yl]oxy}propanoic acid (150 mg, 0.34 mmol) and methylamine. After evaporation of the solvents, the residue was purified by chromatography on silica gel (eluant: 0 to 6% methanol in DCM). After evaporation of the solvents, the solid was triturated in ether and dried under vacuum to give the title compound as a solid (155 mg, 72%); NMR Spectrum: (400 MHz) 1.64 (d, 3H), 2.22 (s, 3H), 2.44 (s, 3H), 2.67 (d, 3H), 5.15 (q, 1H), 7.00 (d, 2H), 7.21 (m, 2H), 7.37 (d, 1H), 7.74 (t, 1H), 7.83 (d, 1H), 7.94 (s, 1H), 8.18 (s, 1H), 8.38 (s, 1H), 8.54 (s, 1H); Mass spectrum: MH⁺ 444.

### EXAMPLE 73

### (2S)—N-(2-hydroxyethyl)-2-{[4-({3-methyl-4-[(6-methylpyridin-3-yl)oxy]phenyl}amino)quinazolin-5-yl]oxy}propanamide

The procedure described in Example 51 was repeated using (2S)-2-{[4-({3-methyl-4-[(6-methylpyridin-3-yl)oxy]phenyl}amino)quinazolin-5-yl]oxy}propanoic acid (150 mg, 0.34 mmol) and ethanolamine (4 equivalents) except that the mixture was stirred at room temperature for 18 hours in the presence of 4 Å molecular sieves. After filtration and evaporation of the solvents, the resulting solid was triturated in DCM to give the title compound (155 mg, 67%); NMR Spectrum: (400 MHz) 1.64 (d, 3H), 2.22 (s, 3H), 2.44 (s, 3H), 3.21 (m, 2H), 3.43 (m, 2H), 4.76 (m, 1H), 5.22 (q, 1H), 7.01 (m, 2H), 7.21 (m, 2H), 7.37 (d, 1H), 7.74 (t, 1H), 7.84 (d, 1H), 7.95 (s, 1H), 8.18 (s, 1H), 8.49 (m, 1H), 8.54 (s, 1H); Mass spectrum: MH⁺ 474.

### EXAMPLE 74

### (2S)—N-(2-hydroxyethyl)-N-methyl-2-{[4-({3-methyl-4-[(6-methylpyridin-3-yl)oxy]phenyl}amino)quinazolin-5-yl]oxy}propanamide

The procedure described in Example 51 was repeated using (2S)-2-{[4-({3-methyl-4-[(6-methylpyridin-3-yl)oxy]phenyl}amino)quinazolin-5-yl]oxy}propanoic acid (150 mg, 0.34 mmol) and 2-(methylamino)ethanol (4 equivalents) except that the mixture was stirred at room temperature for 18 hours in the presence of 4 Å molecular sieves. After filtration and evaporation of the solvents, the residue was purified by chromatography on silica gel (eluant: 0 to 6% methanol in DCM) to give the title compound as a white solid (130 mg, 55%); NMR spectrum: (400 MHz) (2 rotamers) 1.60 (m, 3H), 2.21 (s, 3H), 2.44 (s, 3H), 2.92 and 3.18 (s, 3H), 3.7-3.3 (m, 4H), 4.73 and 5.00 (m, 1H), 5.81 and 5.90 (m, 1H), 6.98 (m, 1H), 7.36-7.24 (m, 4H), 7.71 (m, 1H), 7.90 (m, 1H), 8.02 (m, 1H), 8.19 (s, 1H), 8.52 (s, 1H); Mass spectrum: MH⁺ 488.

### EXAMPLE 75

### N-{3-methyl-4-[(6-methylpyridin-3-yl)oxy]phenyl}-5-[(1S)-1-methyl-2-morpholin-4-yl-2-oxoethoxy]quinazolin-4-amine

1-Hydroxybenzotriazole (23 mg, 0.17 mmol) then EDCI (32 mg, 0.17 mmol) were added to a mixture of (2S)-2-{[4-({3-methyl-4-[(6-methylpyridin-3-yl)oxy]phenyl}amino)quinazolin-5-yl]oxy}propanoic acid (60 mg, 0.14 mmol) and morpholine (18 μl, 0.21 mmol) in DMF (0.8 ml). The mixture was stirred at room temperature for 3 hours. After evaporation of the solvents under vacuum, the residue was triturated in water. The pH of the solution was adjusted to 8 by addition of 5% aqueous sodium bicarbonate. The mixture was extracted with DCM. The organic layer was washed with brine, dried over magnesium sulfate. After evaporation of the solvents, the residue was purified by chromatography on silica gel (eluant: 0 to 5% methanol in DCM) and triturated in ether-pentane to give the title compound as a white solid (31 mg, 43%); NMR Spectrum: (400 MHz) 1.57 (d, 3H), 2.21 (s, 3H), 2.44 (s, 3H), 3.8-3.4 (m, 8H), 5.87 (q, 1H), 6.98 (d, 2H), 7.21 (m, 2H), 7.29 (d, 1H), 7.35 (d, 1H), 7.74 (t, 1H), 7.90 (d, 1H), 8.03 (s, 1H), 8.18 (s, 1H), 8.54 (s, 1H); Mass spectrum: MH⁺ 500.

### EXAMPLE 76

### (3S)-1-((2S)-2-{[4-({3-methyl-4-[(6-methylpyridin-3-yl)oxy]phenyl}amino)quinazolin-5-yl]oxy}propanoyl)pyrrolidin-3-ol

The procedure described in Example 75 was repeated using (2S)-2-{[4-({3-methyl-4-[(6-methylpyridin-3-yl)oxy]phenyl}amino)quinazolin-5-yl]oxy}propanoic acid and (S)-3-pyrrolidinol, except that the mixture was directly injected on an HPLC column (C18, 5 microns, 19 mm diameter, 100 mm length) of a preparative HPLC-MS system eluting with a mixture of water and acetonitrile containing 2 g/l of ammonium carbonate (gradient). After evaporation of the solvents, the mixture was triturated in ether to give the title compound as a white foam (81 mg, 70%); NMR spectrum: (400 MHz) (2 rotamers) 1.60 (m, 3H), 2.1-1.7 (m, 2H), 2.21 (s, 3H), 2.44 (s, 3H), 3.6-3.3 (m, 3H), 3.78 (m, 1H), 4.29 and 4.38 (m, 1H), 4.98 and 5.13 (s br, 1H), 5.56 and 5.62 (m, 1H), 6.99 (d, 1H), 7.29-7.19 (m, 3H), 7.36 (m, 1H), 7.72 (m, 1H), 7.89 (m, 1H), 8.03 (s, 1H), 8.19 (s, 1H), 8.53 (s, 1H), 10.96 (s, 1H); Mass spectrum: MH⁺ 500.

### EXAMPLE 77

### (3S)-1-((2R)-2-{[4-({3-methyl-4-[(6-methylpyridin-3-yl)oxy]phenyl}amino)quinazolin-5-yl]oxy}propanoyl)pyrrolidin-3-ol

The procedure described in Example 75 was repeated using (2R)-2-{[4-({3-methyl-4-[(6-methylpyridin-3-yl)oxy]phenyl}amino)quinazolin-5-yl]oxy}propanoic acid and (S)-3-pyrrolidinol, except that the mixture was directly injected on an HPLC column (C18, 5 microns, 19 mm diameter, 100 mm length) of a preparative HPLC-MS system eluting with a mixture of water and acetonitrile containing 2 g/l of ammonium carbonate (gradient). After evaporation of the solvents, the mixture was triturated in ether to give the title compound as a white foam (170 mg, 73%); NMR spectrum: (400 MHz) (2 rotamers) 1.59 (m, 3H), 2.0-1.7 (m, 2H), 2.21 (s, 3H), 2.44 (s, 3H), 3.9-3.3 (m, 4H), 4.29 and 4.38 (m, 1H), 5.01 and 5.08 (s br, 1H), 5.62 and 5.67 (m, 1H), 6.99 (d, 1H), 7.29-7.19 (m, 3H), 7.36 (m, 1H), 7.73 (m, 1H), 7.88 (m, 1H), 8.03 (s, 1H), 8.19 (s, 1H), 8.53 (s, 1H), 11.00 (s, 1H); Mass spectrum: MH⁺ 500.

### EXAMPLE 78

### (3R)-1-((2R)-2-{[4-({3-methyl-4-[(6-methylpyridin-3-yl)oxy]phenyl}amino)quinazolin-5-yl]oxy}propanoyl)pyrrolidin-3-ol

The procedure described in Example 75 was repeated using (2R)-2-{[4-({3-methyl-4-[(6-methylpyridin-3-yl)oxy]phenyl}amino)quinazolin-5-yl]oxy}propanoic acid and (R)-3-pyrrolidinol, except that the mixture was directly injected on an HPLC column (C18, 5 microns, 19 mm diameter, 100 mm length) of a preparative HPLC-MS system eluting with a mixture of water and acetonitrile containing 2 g/l of ammonium carbonate (gradient). After evaporation of the solvents, the mixture was triturated in ether to give the title compound as a white solid (177 mg, 76%); NMR spectrum: (400 MHz) (2 rotamers) 1.60 (m, 3H), 2.1-1.7 (m, 2H), 2.21 (s, 3H), 2.44 (s, 3H), 3.6-3.3 (m, 3H), 3.78 (m, 1H), 4.29 and 4.38 (m, 1H), 4.98 and 5.13 (s br, 1H), 5.56 and 5.62 (m, 1H), 6.99 (d, 1H), 7.29-7.19 (m, 3H), 7.36 (m, 1H), 7.72 (m, 1H), 7.89 (m, 1H), 8.03 (s, 1H), 8.19 (s, 1H), 8.53 (s, 1H), 10.96 (s, 1H); Mass spectrum: MH⁺ 500.

### EXAMPLE 79

### (2R)—N-methyl-2-{[4-({3-methyl-4-[(6-methylpyridin-3-yl)oxy]phenyl}amino)quinazolin-5-yl]oxy}propanamide

The procedure described in Example 75 was repeated using (2R)-2-{[4-({3-methyl-4-[(6-methylpyridin-3-yl)oxy]phenyl}amino)quinazolin-5-yl]oxy}propanoic acid and methylamine (excess bubbled into the reaction mixture) to give the title compound as a white solid (180 mg, 87%); NMR Spectrum: (400 MHz) 1.64 (d, 3H), 2.22 (s, 3H), 2.44 (s, 3H), 2.67 (d, 3H), 5.15 (q, 1H), 7.00 (d, 2H), 7.21 (m, 2H), 7.37 (d, 1H), 7.74 (t, 1H), 7.83 (d, 1H), 7.94 (s, 1H), 8.18 (s, 1H), 8.38 (s, 1H), 8.54 (s, 1H), 10.61 (s, 1H); Mass spectrum: MH⁺ 444.

### EXAMPLE 80

### (2R)—N-(2-hydroxyethyl)-2-{[4-({3-methyl-4-[(6-methylpyridin-3-yl)oxy]phenyl}amino)quinazolin-5-yl]oxy}propanamide

The procedure described in Example 75 was repeated using (2R)-2-{[4-({3-methyl-4-[(6-methylpyridin-3-yl)oxy]phenyl}amino)quinazolin-5-yl]oxy}propanoic acid and ethanolamine to give the title compound as a white solid (188 mg, 85%); NMR Spectrum: (400 MHz) 1.64 (d, 3H), 2.22 (s, 3H), 2.44 (s, 3H), 3.21 (m, 2H), 3.43 (m, 2H), 4.76 (m, 1H), 5.22 (q, 1H), 7.01 (m, 2H), 7.21 (m, 2H), 7.37 (d, 1H), 7.74 (t, 1H), 7.84 (d, 1H), 7.95 (s, 1H), 8.18 (s, 1H), 8.49 (m, 1H), 8.54 (s, 1H); Mass spectrum: MH⁺ 474.

### EXAMPLE 81

### (2R)—N,N-dimethyl-2-{[4-({3-methyl-4-[(6-methylpyridin-3-yl)oxy]phenyl}amino)quinazolin-5-yl]oxy}propanamide

The procedure described in Example 75 was repeated using (2R)-2-{[4-({3-methyl-4-[(6-methylpyridin-3-yl)oxy]phenyl}amino)quinazolin-5-yl]oxy}propanoic acid and dimethylamine (2N solution in methanol) to give the title compound as a white solid (100 mg, 47%); NMR Spectrum: (400 MHz) 1.58 (d, 3H), 2.21 (s, 3H), 2.44 (s, 3H), 2.93 (s, 3H), 3.14 (s, 3H), 5.85 (q, 1H), 6.98 (d, 1H), 7.21 (m, 2H), 7.30 (d, 1H), 7.35 (d, 1H), 7.73 (t, 1H), 7.90 (d, 1H), 8.02 (s, 1H), 8.19 (s, 1H), 8.52 (s, 1H); Mass spectrum: MH⁺ 458.

### EXAMPLES 82 TO 116

Procedure:

1-Hydroxybenzotriazole (41 mg, 0.30 mmol) then EDCI (58 mg, 0.30 mmol) were added to a mixture of (2R)-2-{[4-({3-methyl-4-[(6-methylpyridin-3-yl)oxy]phenyl}amino)quinazolin-5-yl]oxy}propanoic acid (107 mg, 0.25 mmol) and the corresponding amine (0.37 mmol) in DMF (1 ml). The mixture was stirred at room temperature for 18 hours. The reaction mixture was directly injected on an HPLC column (C18, 5 microns, 20 mm diameter, 100 mm length) of a preparative HPLC-MS system eluting with a mixture of water and acetonitrile containing 2 g/l of ammonium carbonate (gradient). After evaporation of the solvents, the residue was dissolved in 10% methanol in DCM (0.5 ml), triturated with a mixture of ether/pentane to give the desired compound.

### EXAMPLE 82

### (2R)—N-isopropyl-2-{[4-({3-methyl-4-[(6-methylpyridin-3-yl)oxy]phenyl}amino)quinazolin-5-yl]oxy}propanamide

Starting amine: isopropylamine.

The reaction was run in a sealed vessel under irradiation in a Personal Chemistry EMRYS™ Optimizer EXP microwave synthesisor at 100° C. for 10 minutes.

Yield: 50 mg, 42%.

NMR Spectrum: (400 MHz) 1.08 (d, 6H), 1.63 (d, 3H), 2.22 (s, 3H), 2.44 (s, 3H), 3.91 (m, 1H), 5.14 (q, 1H), 7.00 (m, 2H), 7.22 (m, 2H), 7.37 (d, 1H), 7.74 (t, 1H), 7.84 (d, 1H), 7.96 (d, 1H), 8.18 (s, 1H), 8.30 (d, 1H), 8.54 (s, 1H); HPLC t_{R}: 2.95 min; Mass spectrum: MH⁺ 472.

### EXAMPLE 83

### (2R)—N-ethyl-2-{[4-({3-methyl-4-[(6-methylpyridin-3-yl)oxy]phenyl}amino)quinazolin-5-yl]oxy}propanamide

Starting amine: ethyl amine (70% aqueous solution).

The reaction was run in a sealed vessel under irradiation in a Personal Chemistry EMRYS™ Optimizer EXP microwave synthesisor at 100° C. for 10 minutes.

Yield: 59 mg, 51%.

NMR Spectrum: (400 MHz) 1.04 (t, 3H), 1.64 (d, 3H), 2.22 (s, 3H), 2.44 (s, 3H), 3.16 (m, 2H), 5.15 (q, 1H), 7.00 (m, 2H), 7.22 (m, 2H), 7.37 (d, 1H), 7.74 (t, 1H), 7.84 (dd, 1H), 7.94 (d, 1H), 8.18 (d, 1H), 8.44 (bt, 1H), 8.54 (s, 1H); HPLC t_{R}: 2.70 min; Mass spectrum: MH⁺ 458.

### EXAMPLE 84

### (2R)—N-[2-(diethylamino)ethyl]-2-{[4-({3-methyl-4-[(6-methylpyridin-3-yl)oxy]phenyl}amino)quinazolin-5-yl]oxy}propanamide

Starting amine: N,N-diethylethylenediamine.

Yield: 104 mg, 79%.

NMR Spectrum: (400 MHz; DMSOd₆ and CF₃CO₂D) 1.20 (m, 6H), 1.71 (d, 3H), 2.30 (s, 3H), 2.72 (s, 3H), 3.20 (m, 6H), 3.55 (m, 2H), 5.41 (q, 1H), 7.25 (d, 1H), 7.42 (d, 1H), 7.50 (d, 1H), 7.85 (m, 1H), 7.94 (m, 2H), 8.06 (t, 1H), 8.11 (m, 1H), 8.75 (d, 1H), 8.98 (s, 1H); HPLC t_{R}: 1.87 min; Mass spectrum: MH⁺ 529.

### EXAMPLE 85

### (2R)—N-[2-(dimethylamino)ethyl]-2-{[4-({3-methyl-4-[(6-methylpyridin-3-yl)oxy]phenyl}amino)quinazolin-5-yl]oxy}propanamide

Starting amine: N,N-dimethylethylenediamine.

Yield: 102 mg, 81%.

NMR Spectrum: (400 MHz; DMSOd₆ and CF₃CO₂D) 1.71 (d, 3H), 2.30 (s, 3H), 2.71 (s, 3H), 2.84 (s, 6H), 3.22 (m, 2H), 3.50 (m, 1H), 3.60 (m, 1H), 5.39 (q, 1H), 7.25 (d, 1H), 7.42 (d, 1H), 7.50 (d, 1H), 7.85 (m, 1H), 7.94 (m, 2H), 8.06 (t, 1H), 8.11 (m, 1H), 8.75 (d, 1H), 8.98 (s, 1H); HPLC t_{R}: 1.81 min; Mass spectrum: MH⁺ 501.

### EXAMPLE 86

### (2R)—N-cyclopropyl-2-{[4-({3-methyl-4-[(6-methylpyridin-3-yl)oxy]phenyl}amino)quinazolin-5-yl]oxy}propanamide

Starting amine: cyclopropylamine.

Yield: 67 mg, 57%.

NMR Spectrum: (400 MHz) 0.44 (m, 2H), 0.65 (m, 2H), 1.62 (d, 3H), 2.22 (s, 3H), 2.44 (s, 3H), 2.72 (m, 1H), 5.10 (q, 1H), 7.00 (m, 2H), 7.22 (m, 2H), 7.37 (d, 1H), 7.74 (t, 1H), 7.84 (dd, 1H), 7.94 (s, 1H), 8.19 (d, 1H), 8.50 (bd, 1H), 8.54 (s, 1H); HPLC t_{R}: 2.69 min; Mass spectrum: MH⁺ 470.

### EXAMPLE 87

### (2R)—N-(3-hydroxypropyl)-2-{[4-({3-methyl-4-[(6-methylpyridin-3-yl)oxy]phenyl}amino)quinazolin-5-yl]oxy}propanamide

Starting amine: 3-amino-1-propanol.

Yield: 93 mg, 76%.

NMR Spectrum: (400 MHz) 1.59 (m, 2H), 1.64 (d, 3H), 2.22 (s, 3H), 2.44 (s, 3H), 3.19 (m, 2H), 3.39 (m, 2H), 4.44 (t, 1H), 5.17 (q, 1H), 7.00 (m, 2H), 7.22 (m, 2H), 7.37 (d, 1H), 7.74 (t, 1H), 7.84 (d, 1H), 7.94 (s, 1H), 8.19 (d, 1H), 8.42 (bt, 1H), 8.54 (s, 1H); HPLC t_{R}: 2.40 min; Mass spectrum: MH⁺ 488.

### EXAMPLE 88

### (2R)—N-(2-methoxyethyl)-2-{[4-({3-methyl-4-[(6-methylpyridin-3-yl)oxy]phenyl}amino)quinazolin-5-yl]oxy}propanamide

Starting amine: 2-methoxyethylamine.

Yield: 61 mg, 50%.

NMR Spectrum: (400 MHz; DMSOd₆ and CF₃CO₂D) 1.68 (d, 3H), 2.31 (s, 3H), 2.72 (s, 3H), 3.26 (s, 3H), 3.36 (m, 2H), 3.41 (m, 2H), 5.41 (q, 1H), 7.26 (d, 1H), 7.37 (d, 1H), 7.48 (d, 1H), 7.85 (m, 1H), 7.96 (m, 2H), 8.06 (t, 1H), 8.15 (m, 1H), 8.77 (d, 1H), 8.98 (s, 1H); HPLC t_{R}: 2.57 min; Mass spectrum: MH⁺ 488.

### EXAMPLE 89

### (2R)-2-{[4-({3-methyl-4-[(6-methylpyridin-3-yl)oxy]phenyl}amino)quinazolin-5-yl]oxy}-N-(2-morpholin-4-ylethyl)propanamide

Starting amine: 4-(2-aminoethyl)morpholine.

Yield: 116 mg, 86%.

NMR Spectrum: (400 MHz) 1.65 (d, 3H), 2.22 (s, 3H), 2.35 (m, 6H), 2.44 (s, 3H), 3.28 (m, 2H), 3.49 (m, 4H), 5.19 (q, 1H), 7.00 (m, 2H), 7.22 (m, 2H), 7.37 (d, 1H), 7.74 (t, 1H), 7.84 (d, 1H), 7.94 (s, 1H), 8.19 (d, 1H), 8.42 (bt, 1H), 8.54 (s, 1H); HPLC t_{R}: 2.09 min; Mass spectrum: MH⁺ 543.

### EXAMPLE 90

### (2R)-2-{[4-({3-methyl-4-[(6-methylpyridin-3-yl)oxy]phenyl}amino)quinazolin-5-yl]oxy}-N-(2-pyrrolidin-1-ylethyl)propanamide

Starting amine: 1-(2-aminoethyl)pyrrolidine.

Yield: 84 mg, 64%.

NMR Spectrum: (400 MHz) 1.63 (m, 7H), 2.22 (s, 3H), 2.44 (s, 3H), 2.6-2.3 (m, 6H), 3.25 (m, 2H), 5.20 (q, 1H), 7.00 (m, 2H), 7.22 (m, 2H), 7.37 (d, 1H), 7.74 (t, 1H), 7.84 (d, 1H), 7.94 (s, 1H), 8.19 (d, 1H), 8.40 (bt, 1H), 8.54 (s, 1H); HPLC t_{R}: 1.90 min; Mass spectrum: MH⁺ 527.

### EXAMPLE 91

### (2R)—N-[2-(acetylamino)ethyl]-2-{[4-({3-methyl-4-[(6-methylpyridin-3-yl)oxy]phenyl}amino)quinazolin-5-yl]oxy}propanamide

Starting amine: N-acetylethylenediamine.

Yield: 44 mg, 34%.

NMR Spectrum: (400 MHz) 1.65 (d, 3H), 1.75 (s, 3H), 2.22 (s, 3H), 2.44 (s, 3H), 3.12 (m, 2H), 3.18 (m, 2H), 5.15 (q, 1H), 7.00 (m, 2H), 7.22 (m, 2H), 7.37 (d, 1H), 7.74 (t, 1H), 7.84 (d, 1H), 7.88 (bt, 1H), 7.94 (s, 1H), 8.18 (d, 1H), 8.48 (bt, 1H), 8.54 (s, 1H); HPLC t_{R}: 2.37 min; Mass spectrum: MH⁺ 515.

### EXAMPLE 92

### (2R)-2-{[4-({3-methyl-4-[(6-methylpyridin-3-yl)oxy]phenyl}amino)quinazolin-5-yl]oxy}-N-[3-(4-methylpiperazin-1-yl)propyl]propanamide

Starting amine: 1-(3-aminopropyl)-4-methylpiperazine.

Yield: 115 mg, 81%.

NMR Spectrum: (400 MHz) 1.55 (m, 2H), 1.65 (d, 3H), 2.19 (s, 3H), 2.22 (s, 3H), 2.4-2.2 (m, 10H), 2.44 (s, 3H), 3.16 (m, 2H), 5.15 (q, 1H), 7.00 (m, 2H), 7.22 (m, 2H), 7.37 (d, 1H), 7.74 (t, 1H), 7.84 (dd, 1H), 7.94 (d, 1H), 8.18 (d, 1H), 8.41 (bt, 1H), 8.54 (s, 1H); HPLC t_{R}: 1.88 min; Mass spectrum: MH⁺ 570.

### EXAMPLE 93

### (2R)-2-{[4-({3-methyl-4-[(6-methylpyridin-3-yl)oxy]phenyl}amino)quinazolin-5-yl]oxy}-N-[3-(2-oxopyrrolidin-1-yl)propyl]propanamide

Starting amine: 1-(3-aminopropyl)-2-pyrrolidinone.

Yield: 94 mg, 68%.

NMR Spectrum: (400 MHz) 1.60 (m, 2H), 1.65 (d, 3H), 1.88 (m, 2H), 2.17 (m, 2H), 2.22 (s, 3H), 2.44 (s, 3H), 3.12 (m, 4H), 3.16 (m, 2H), 5.17 (q, 1H), 7.00 (m, 2H), 7.22 (m, 2H), 7.37 (d, 1H), 7.74 (t, 1H), 7.84 (dd, 1H), 7.94 (d, 1H), 8.18 (d, 1H), 8.44 (bt, 1H), 8.54 (s, 1H); HPLC t_{R}: 2.66 min; Mass spectrum: MH⁺ 553.

### EXAMPLE 94

### (2R)-2-{[4-({3-methyl-4-[(6-methylpyridin-3-yl)oxy]phenyl}amino)quinazolin-5-yl]oxy}-N-[2-(methylthio)ethyl]propanamide

Starting amine: 2-(methylthio)ethylamine.

Yield: 103 mg, 82%.

NMR Spectrum: (400 MHz) 1.65 (d, 3H), 2.04 (s, 3H), 2.22 (s, 3H), 2.44 (s, 3H), 2.56 (m, 2H), 3.36 (m, 2H), 5.17 (q, 1H), 7.00 (m, 2H), 7.22 (m, 2H), 7.37 (d, 1H), 7.74 (t, 1H), 7.84 (dd, 1H), 7.94 (s, 1H), 8.18 (d, 1H), 8.54 (s, 1H), 8.58 (bt, 1H); HPLC t_{R}: 2.92 min; Mass spectrum: MH⁺ 504.

### EXAMPLE 95

### (2R)—N-(3-methoxypropyl)-2-{[4-({3-methyl-4-[(6-methylpyridin-3-yl)oxy]phenyl}amino)quinazolin-5-yl]oxy}propanamide

Starting amine: 3-methoxypropylamine.

Yield: 99 mg, 79%.

NMR Spectrum: (400 MHz) 1.63 (m, 2H), 1.65 (d, 3H), 2.22 (s, 3H), 2.44 (s, 3H), 3.16 (s, 3H), 3.18 (m, 2H), 3.28 (t, 2H), 5.16 (q, 1H), 7.00 (m, 2H), 7.22 (m, 2H), 7.37 (d, 1H), 7.74 (t, 1H), 7.84 (dd, 1H), 7.94 (s, 1H), 8.18 (d, 1H), 8.43 (bt, 1H), 8.54 (s, 1H); HPLC t_{R}: 2.75 min; Mass spectrum: MH⁺ 502.

### EXAMPLE 96

### (2R)—N-cyclobutyl-2-{[4-({3-methyl-4-[(6-methylpyridin-3-yl)oxy]phenyl}amino)quinazolin-5-yl]oxy}propanamide

Starting amine: cyclobutylamine.

Yield: 74 mg, 61%.

NMR Spectrum: (400 MHz; DMSOd₆ and CF₃CO₂D) 1.68 (m, 5H), 1.99 (m, 2H), 2.21 (m, 2H), 2.31 (s, 3H), 2.72 (s, 3H), 4.28 (m, 1H), 5.33 (q, 1H), 7.26 (d, 1H), 7.37 (d, 1H), 7.48 (d, 1H), 7.85 (dd, 1H), 7.96 (m, 2H), 8.06 (t, 1H), 8.15 (dd, 1H), 8.77 (d, 1H), 8.98 (s, 1H); HPLC t_{R}: 3.04 min; Mass spectrum: MH⁺ 484.

### EXAMPLE 97

### (2R)—N-[(2R)-2-hydroxypropyl]-2-{[4-({3-methyl-4-[(6-methylpyridin-3-yl)oxy]phenyl}amino)quinazolin-5-yl]oxy}propanamide

Starting amine: (R)-1-amino-2-propanol.

Yield: 43 mg, 35%.

NMR Spectrum: (400 MHz) 0.98 (d, 3H), 1.65 (d, 3H), 2.22 (s, 3H), 2.44 (s, 3H), 3.10 (t, 2H), 3.68 (m, 1H), 4.76 (bd, 1H), 5.24 (q, 1H), 7.00 (m, 2H), 7.22 (m, 2H), 7.37 (d, 1H), 7.74 (t, 1H), 7.84 (dd, 1H), 7.94 (s, 1H), 8.18 (d, 1H), 8.44 (bt, 1H), 8.54 (s, 1H); HPLC t_{R}: 2.45 min; Mass spectrum: MH⁺ 488.

### EXAMPLE 98

### (2R)—N-[(2S)-2-hydroxypropyl]-2-{[4-({3-methyl-4-[(6-methylpyridin-3-yl)oxy]phenyl}amino)quinazolin-5-yl]oxy}propanamide

Starting amine: (S)-1-amino-2-propanol.

Yield: 62 mg, 51%.

NMR Spectrum: (400 MHz) 1.00 (d, 3H), 1.65 (d, 3H), 2.22 (s, 3H), 2.44 (s, 3H), 3.10 (m, 2H), 3.66 (m, 1H), 4.75 (bd, 1H), 5.27 (q, 1H), 7.00 (m, 2H), 7.22 (m, 2H), 7.37 (d, 1H), 7.74 (t, 1H), 7.84 (dd, 1H), 7.94 (s, 1H), 8.18 (d, 1H), 8.41 (bt, 1H), 8.54 (s, 1H); HPLC t_{R}: 2.40 min; Mass spectrum: MH⁺ 488.

### EXAMPLE 99

### (2R)—N-[(2S)-2,3-dihydroxypropyl]-2-{[4-({3-methyl-4-[(6-methylpyridin-3-yl)oxy]phenyl}amino)quinazolin-5-yl]oxy}propanamide

Starting amine: (S)-3 amino-1,2-propanediol.

Yield: 95 mg, 76%.

NMR Spectrum: (400 MHz) 1.64 (d, 3H), 2.22 (s, 3H), 2.44 (s, 3H), 3.09 (m, 1H), 3.28 (m, 3H), 3.52 (m, 1H), 4.55 (bt, 1H), 4.84 (bd, 1H), 5.26 (q, 1H), 7.00 (m, 2H), 7.22 (m, 2H), 7.37 (d, 1H), 7.74 (t, 1H), 7.84 (dd, 1H), 7.94 (s, 1H), 8.18 (d, 1H), 8.42 (bt, 1H), 8.54 (s, 1H); HPLC t_{R}: 2.33 min; Mass spectrum: MH⁺ 504.

### EXAMPLE 100

### (2R)—N-[(1R)-2-hydroxy-1-methylethyl]-2-{[4-({3-methyl-4-[(6-methylpyridin-3-yl)oxy]phenyl}amino)quinazolin-5-yl]oxy}propanamide

Starting amine: (R)-2-amino-1-propanol.

Yield: 83 mg, 68%.

NMR Spectrum: (D400 MHz) 1.04 (d, 3H), 1.63 (d, 3H), 2.21 (s, 3H), 2.44 (s, 3H), 3.30 (m, 2H), 3.85 (m, 1H), 4.79 (bt, 1H), 5.22 (q, 1H), 7.00 (m, 2H), 7.22 (m, 2H), 7.37 (d, 1H), 7.74 (t, 1H), 7.84 (dd, 1H), 7.96 (s, 1H), 8.18 (d, 1H), 8.24 (bd, 1H), 8.54 (s, 1H); HPLC t_{R}: 2.41 min; Mass spectrum: MH⁺ 488.

### EXAMPLE 101

### (2R)—N-[(1S)-2-hydroxy-1-methylethyl]-2-{[4-({3-methyl-4-[(6-methylpyridin-3-yl)oxy]phenyl}amino)quinazolin-5-yl]oxy}propanamide

Starting amine: (s)-2-amino-1-propanol.

Yield: 15 mg, 12%.

NMR Spectrum: (400 MHz) 1.06 (d, 3H), 1.63 (d, 3H), 2.22 (s, 3H), 2.44 (s, 3H), 3.30 (m, 2H), 3.85 (m, 1H), 4.76 (bt, 1H), 5.19 (q, 1H), 7.00 (m, 2H), 7.22 (m, 2H), 7.37 (d, 1H), 7.74 (t, 1H), 7.84 (dd, 1H), 7.97 (s, 1H), 8.19 (d, 1H), 8.25 (bd, 1H), 8.54 (s, 1H); HPLC t_{R}: 2.44 min; Mass spectrum: MH⁺ 488.

### EXAMPLE 102

### N-{3-methyl-4-[(6-methylpyridin-3-yl)oxy]phenyl}-5-[(1R)-1-methyl-2-morpholin-4-yl-2-oxoethoxy]quinazolin-4-amine

Starting amine: morpholine.

Yield: 36 mg, 29%.

NMR Spectrum: (400 MHz) 1.57 (d, 3H), 2.21 (s, 3H), 2.44 (s, 3H), 3.8-3.3 (m, 8H), 5.88 (q, 1H), 6.98 (d, 1H), 7.20 (m, 2H), 7.29 (d, 1H), 7.36 (d, 1H), 7.74 (t, 1H), 7.90 (dd, 1H), 8.03 (s, 1H), 8.19 (d, 1H), 8.53 (s, 1H); HPLC t_{R}: 2.63 min; Mass spectrum: MH⁺ 500.

### EXAMPLE 103

### (2R)—N-[2-(dimethylamino)ethyl]-N-methyl-2-{[4-({3-methyl-4-[(6-methylpyridin-3-yl)oxy]phenyl}amino)quinazolin-5-yl]oxy}propanamide

Starting amine: N,N,N′-trimethylethylenediamine.

Yield: 38 mg, 30%.

HPLC t_{R}: 1.80 min; Mass spectrum: MH⁺ 513.

### EXAMPLE 104

### 5-[(1R)-1-methyl-2-(4-methylpiperazin-1-yl)-2-oxoethoxy]-N-{3-methyl-4-[(6-methylpyridin-3-yl)oxy]phenyl}quinazolin-4-amine

Starting amine: N-methylpiperazine.

Yield: 85 mg, 66%.

NMR Spectrum: (400 MHz) 1.56 (d, 3H), 2.20 (s, 3H), 2.21 (s, 3H), 2.4-2.2 (m, 4H), 2.44 (s, 3H), 3.7-3.4 (m, 4H), 5.88 (q, 1H), 6.98 (d, 1H), 7.20 (m, 2H), 7.29 (d, 1H), 7.36 (d, 1H), 7.74 (t, 1H), 7.89 (dd, 1H), 8.02 (s, 1H), 8.18 (d, 1H), 8.52 (s, 1H); HPLC t_{R}: 1.88 min; Mass spectrum: MH⁺ 513.

### EXAMPLE 105

### [(2R)-1-((2R)-2-{[4-({3-methyl-4-[(6-methylpyridin-3-yl)oxy]phenyl}amino)quinazolin-5-yl]oxy}propanoyl)pyrrolidin-2-yl]methanol

Starting amine: (R)-2-pyrrolidinemethanol.

Yield: 94 mg, 73%.

HPLC t_{R}: 2.56 min; Mass spectrum: MH⁺ 514.

### EXAMPLE 106

### [(2S)-1-((2R)-2-{[4-({3-methyl-4-[(6-methylpyridin-3-yl)oxy]phenyl}amino)quinazolin-5-yl]oxy}propanoyl)pyrrolidin-2-yl]methanol

Starting amine: (S)-2-pyrrolidinemethanol.

Yield: 74 mg, 58%.

HPLC t_{R}: 2.58 min; Mass spectrum: MH⁺ 514.

### EXAMPLE 107

### 1-((2R)-2-{[4-({3-methyl-4-[(6-methylpyridin-3-yl)oxy]phenyl}amino)quinazolin-5-yl]oxy}propanoyl)piperidin-4-ol

Starting amine: 4-hydroxypiperidine.

Yield: 81 mg, 63%.

NMR Spectrum: (400 MHz) 1.5-1.2 (m, 2H), 1.56 (d, 3H), 1.9-1.7 (m, 2H), 2.21 (s, 3H), 2.44 (s, 3H), 3.3-3.1 (m, 2H), 4.0-3.7 (m, 3H), 4.81 (m, 1H), 5.88 (m, 1H), 6.98 (d, 1H), 7.20 (m, 2H), 7.32 (m, 2H), 7.73 (m, 1H), 7.89 (d, 1H), 8.03 (s, 1H), 8.19 (d, 1H), 8.52 (s, 1H); HPLC t_{R}: 2.44 min; Mass spectrum: MH⁺ 514.

### EXAMPLE 108

### (2R)—N,N-bis(2-hydroxyethyl)-2-{[4-({3-methyl-4-[(6-methylpyridin-3-yl)oxy]phenyl}amino)quinazolin-5-yl]oxy}propanamide

Starting amine: diethanolamine.

Yield: 34 mg, 26%.

NMR Spectrum: (400 MHz; DMSOd₆ and CF₃CO₂D) 1.66 (d, 3H), 2.31 (s, 3H), 2.72 (s, 3H), 3.8-3.2 (m, 8H), 6.06 (q, 1H), 7.26 (d, 1H), 7.48 (d, 1H), 7.65 (d, 1H), 7.86 (dd, 1H), 7.95 (d, 1H), 8.04 (m, 2H), 8.17 (dd, 1H), 8.78 (d, 1H), 8.97 (s, 1H); HPLC t_{R}: 2.15 min; Mass spectrum: MH⁺ 516.

### EXAMPLE 109

### (2R)—N-ethyl-N-(2-hydroxyethyl)-2-{[4-({3-methyl-4-[(6-methylpyridin-3-yl)oxy]phenyl}amino)quinazolin-5-yl]oxy}propanamide

Starting amine: 2-ethylaminoethanol.

Yield: 45 mg, 36%.

HPLC t_{R}: 2.47 min; Mass spectrum: MH⁺ 502.

### EXAMPLE 110

### (2R)—N,N-bis(2-methoxyethyl)-2-{[4-({3-methyl-4-[(6-methylpyridin-3-yl)oxy]phenyl}amino)quinazolin-5-yl]oxy}propanamide

Starting amine: bis(2-methoxyethyl)amine.

Yield: 27 mg, 20%.

HPLC t_{R}: 2.97 min; Mass spectrum: MH⁺ 546.

### EXAMPLE 111

### 5-[(1R)-2-(4-ethylpiperazin-1-yl)-1-methyl-2-oxoethoxy]-N-{3-methyl-4-[(6-methylpyridin-3-yl)oxy]phenyl}quinazolin-4-amine

Starting amine: N-ethylpiperazine.

Yield: 75 mg, 57%.

NMR Spectrum: (400 MHz) 1.01 (t, 3H), 1.57 (d, 3H), 2.21 (s, 3H), 2.4-2.2 (m, 6H), 2.44 (s, 3H), 3.7-3.4 (m, 4H), 5.88 (q, 1H), 6.98 (d, 1H), 7.20 (m, 2H), 7.29 (d, 1H), 7.35 (d, 1H), 7.74 (t, 1H), 7.89 (dd, 1H), 8.02 (s, 1H), 8.18 (d, 1H), 8.52 (s, 1H); HPLC t_{R}: 1.80 min; Mass spectrum: MH⁺ 527.

### EXAMPLE 112

### (3R)-1-((2R)-2-{[4-({3-methyl-4-[(6-methylpyridin-3-yl)oxy]phenyl}amino)quinazolin-5-yl]oxy}propanoyl)piperidin-3-ol

Starting amine: (R)-3-hydroxypiperidine.

Yield: 72 mg, 56%.

HPLC t_{R}: 2.47 min; Mass spectrum: MH⁺ 514.

### EXAMPLE 113

### (3S)-1-((2R)-2-{[4-({3-methyl-4-[(6-methylpyridin-3-yl)oxy]phenyl}amino)quinazolin-5-yl]oxy}propanoyl)piperidin-3-ol

Starting amine: (S)-3-hydroxypiperidine.

Yield: 47 mg, 37%.

HPLC t_{R}: 2.45 min; Mass spectrum: MH⁺ 514.

### EXAMPLE 114

### 4-((2R)-2-{[4-({3-methyl-4-[(6-methylpyridin-3-yl)oxy]phenyl}amino)quinazolin-5-yl]oxy}propanoyl)piperazin-2-one

Starting amine: piperazin-2-one.

Yield: 91 mg, 71%.

HPLC t_{R}: 2.07 min; Mass spectrum: MH⁺ 513.

### EXAMPLE 115

### [1-((2R)-2-{[4-({3-methyl-4-[(6-methylpyridin-3-yl)oxy]phenyl}amino)quinazolin-5-yl]oxy}propanoyl)piperidin-4-yl]methanol

Starting amine: 4-(hydroxymethyl)piperidine.

Yield: 26 mg, 19%.

HPLC t_{R}: 2.36 min; Mass spectrum: MH⁺ 528.

### EXAMPLE 116

### Tert-butyl 4-((2R)-2-{[4-({3-methyl-4-[(6-methylpyridin-3-yl)oxy]phenyl}amino)quinazolin-5-yl]oxy}propanoyl)piperazine-1-carboxylate

Starting amine: 1-tert-butoxycarbonylpiperazine.

Yield: 107 mg, 71%.

HPLC t_{R}: 3.38 min; Mass spectrum: MH⁺ 599.

### EXAMPLE 117

### N-{3-methyl-4-[(6-methylpyridin-3-yl)oxy]phenyl}-5-[(1R)-1-methyl-2-oxo-2-piperazin-1-ylethoxy]quinazolin-4-amine

Hydrogen chloride (4N in dioxane, 1 ml) was added to tert-butyl 4-((2R)-2-{[4-({3-methyl-4-[(6-methylpyridin-3-yl)oxy]phenyl}amino)quinazolin-5-yl]oxy}propanoyl)piperazine-1-carboxylate (85 mg). The mixture was stirred at room temperature for 1 hour. After evaporation of the solvents, the resulting solid was dried under high vacuum to give the title compound as a hydrochloride salt (80 mg, 93%); NMR Spectrum: (400 MHz) 1.56 (d, 3H), 2.21 (s, 3H), 2.44 (s, 3H), 2.9-2.7 (m, 4H), 3.7-3.3 (m, 4H), 5.86 (q, 1H), 6.97 (d, 1H), 7.20 (m, 2H), 7.29 (d, 1H), 7.35 (d, 1H), 7.74 (t, 1H), 7.90 (dd, 1H), 8.03 (s, 1H), 8.18 (d, 1H), 8.52 (s, 1H); HPLC t_{R}: 1.51 min; Mass spectrum: MH⁺ 499.

### EXAMPLE 118

### 5-[(1R)-2-azetidin-1-yl-1-methyl-2-oxoethoxy]-N-{3-methyl-4-[(6-methylpyridin-3-yl)oxy]phenyl}quinazolin-4-amine

The procedure described in Example 75 was repeated using (2R)-2-{[4-({3-methyl-4-[(6-methylpyridin-3-yl)oxy]phenyl}amino)quinazolin-5-yl]oxy}propanoic acid (200 mg, 0.47 mmol) and azetidine to give the title compound as a white solid (160 mg, 73%); NMR Spectrum: (400 MHz) 1.59 (d, 3H), 2.22 (s, 3H), 2.27 (m, 2H), 2.44 (s, 3H), 3.98 (m, 2H), 4.24 (m, 1H), 4.42 (m, 1H), 5.40 (q, 1H), 6.99 (d, 2H), 7.21 (m, 2H), 7.37 (d, 1H), 7.74 (t, 1H), 7.83 (dd, 1H), 7.94 (d, 1H), 8.18 (d, 1H), 8.38 (s, 1H), 8.53 (s, 1H); Mass spectrum: MH⁺ 470

### EXAMPLE 119

### 1-((2R)-2-{[4-({3-methyl-4-[(6-methylpyridin-3-yl)oxy]phenyl}amino)quinazolin-5-yl]oxy}propanoyl)azetidin-3-ol

The procedure described in Example 75 was repeated using (2R)-2-{[4-({3-methyl-4-[(6-methylpyridin-3-yl)oxy]phenyl}amino)quinazolin-5-yl]oxy}propanoic acid (200 mg, 0.47 mmol) and 3-hydroxyazetidine hydrochloride [prepared from 1-tert-butoxycarbonyl-4-hydroxyazetidine (2.5 g, 14.4 mmol, Falgueyret, J. P., J. Med. Chem., 2001, 44, 94) by treatment with TFA (21 ml) in DCM (30 ml) at room temperature. After evaporation of the solvent, the mixture was diluted with water; the pH was adjusted to 11 with 2N sodium hydroxide; extraction with ether, concentration to dryness and trituration in 4N HCl in dioxane gave crude 3-hydroxyazetidine hydrochloride] to give the title compound as a white solid (40 mg, 18%) except that after 24 hours of reaction, additional 1-hydroxybenzotriazole (1.2 eq) and EDCI (1.2 eq) were added. The mixture was stirred for 18 hours more and injected on an HPLC column (C18, 5 microns, 19 mm diameter, 100 mm length) of a preparative HPLC-MS system eluting with a mixture of water and acetonitrile containing 2 g/l of ammonium carbonate (gradient); HPLC t_{R}: 2.19 min; Mass spectrum: MH⁺ 486.

### EXAMPLE 120

### (2R)—N-(2-methoxyethyl)-N-methyl-2-{[4-({3-methyl-4-[(6-methylpyridin-3-yl)oxy]phenyl}amino)quinazolin-5-yl]oxy}propanamide

The procedure described in Example 75 was repeated using (2R)-2-{[4-({3-methyl-4-[(6-methylpyridin-3-yl)oxy]phenyl}amino)quinazolin-5-yl]oxy}propanoic acid (200 mg, 0.47 mmol) and (2-methoxyethyl)methylamine to give the title compound as a white solid (155 mg, 67%); NMR Spectrum: (400 MHz; DMSOd₆ and CF₃CO₂D) (2 rotamers) 1.62 (m, 3H), 2.38 (2 singlets, 3H), 2.71 (s, 3H), 3.17 and 2.94 (s, 3H), 3.25 (2 singlets, 3H), 3.8-3.45 (m, 4H), 6.02 and 5.98 (q, 1H), 7.24 (m, 1H), 7.46 (d, 1H), 7.64 (d, 1H), 7.85 (m, 1H), 7.93 (d, 1H), 7.98 (dd, 1H), 8.05 (m, 1H), 8.13 (dd, 1H), 8.76 (s, 1H), 8.95 (s, 1H); Mass spectrum: MH⁺ 502

### EXAMPLE 121

### (2R)—N,N-diethyl-2-{[4-({3-methyl-4-[(6-methylpyridin-3-yl)oxy]phenyl}amino)quinazolin-5-yl]oxy}propanamide

The procedure described in Example 75 was repeated with (2R)-2-{[4-({3-methyl-4-[(6-methylpyridin-3-yl)oxy]phenyl}amino)quinazolin-5-yl]oxy}propanoic acid (200 mg, 0.47 mmol) and diethylamine to give the title compound as a white solid (125 mg, 55%) except that the mixture was directly injected on an HPLC column (C18, 5 microns, 19 mm diameter, 100 mm length) of a preparative HPLC-MS system eluting with a mixture of water and acetonitrile containing 2 g/l of ammonium carbonate (gradient); NMR Spectrum: (400 MHz) 1.09 (t, 3H), 1.20 (t, 3H), 1.61 (d, 3H), 2.30 (s, 3H), 2.71 (s, 3H), 3.30 (m, 1H), 3.50 (m, 3H), 5.93 (q, 1H), 7.24 (d, 1H), 7.45 (d, 1H), 7.69 (d, 1H), 7.85 (m, 1H), 7.93 (d, 1H), 7.98 (d, 1H), 8.04 (t, 1H), 8.13 (dd, 1H), 8.76 (d, 1H), 8.96 (s, 1H); Mass spectrum: MH⁺ 486.

### EXAMPLE 122

### N-{3-methyl-4-[(6-methylpyridin-3-yl)oxy]phenyl}-5-[(1R)-1-methyl-2-oxo-2-pyrrolidin-1-ylethoxy]quinazolin-4-amine

The procedure described in Example 75 was repeated using (2R)-2-{[4-({3-methyl-4-[(6-methylpyridin-3-yl)oxy]phenyl}amino)quinazolin-5-yl]oxy}propanoic acid (200 mg, 0.47 mmol) and pyrrolidine to give the title compound as a white solid (140 mg, 62%) except that the mixture was directly injected on an HPLC column (C18, 5 microns, 19 mm diameter, 100 mm length) of a preparative HPLC-MS system eluting with a mixture of water and acetonitrile containing 2 g/l of ammonium carbonate (gradient); NMR Spectrum: (400 MHz) 1.60 (d, 3H), 1.82 (m, 2H), 1.94 (m, 2H), 2.21 (s, 3H), 2.44 (s, 3H), 3.6-3.3 (m, 3H), 3.76 (m, 1H), 5.62 (q, 1H), 6.99 (d, 1H), 7.28-7.18 (m, 3H), 7.35 (d, 1H), 7.73 (t, 1H), 7.88 (dd, 1H), 8.03 (d, 1H), 8.18 (d, 1H), 8.53 (s, 1H); Mass spectrum: MH⁺ 484

### EXAMPLE 123

### (2R)—N-(3-hydroxypropyl)-N-methyl-2-{[4-({3-methyl-4-[(6-methylpyridin-3-yl)oxy]phenyl}amino)quinazolin-5-yl]oxy}propanamide

The procedure described in Example 75 was repeated using (2R)-2-{[4-({3-methyl-4-[(6-methylpyridin-3-yl)oxy]phenyl}amino)quinazolin-5-yl]oxy}propanoic acid (200 mg, 0.47 mmol) and (3-hydroxypropyl)methylamine (S. Koepke, J. Org. Chem. 1979, 44, 2718) to give the title compound as a white solid (115 mg, 50%) except that the mixture was directly injected on an HPLC column (C18, 5 microns, 19 mm diameter, 100 mm length) of a preparative HPLC-MS system eluting with a mixture of water and acetonitrile containing 2 g/l of ammonium carbonate (gradient); NMR Spectrum: (400 MHz) (2 rotamers) 1.59 (m, 3H), 1.75 (m, 2H), 2.21 (s, 3H), 2.44 (s, 3H), 3.13 and 2.90 (s, 3H), 3.6-3.3 (m, 4H), 4.70 and 4.45 (m, 1H), 5.87 and 5.81 (q, 1H), 6.99 (m, 1H), 7.30-7.20 (m, 3H), 7.35 (d, 1H), 7.73 (t, 1H), 7.90 (m, 1H), 8.03 and 7.99 (d, 1H), 8.19 (d, 1H), 8.52 (s, 1H), 11.04 and 11.02 (s, 1H); Mass spectrum: MH⁺ 502.

### EXAMPLES 124 TO 137

Procedure:

A mixture of 5-[(1R)-1-methyl-2-morpholin-4-yl-2-oxoethoxy]quinazolin-4(3H)-one (120 mg, 0.4 mmol), phosphorus oxychloride (0.04 ml, 0.48 mmol) and diisopropylethylamine (0.18 ml, 1.0 mmol) in 1,2-dicholoroethane (2 ml) was stirred at 80° C. for 3 hours. The mixture was cooled. The appropriate aniline (0.42 mmol) was added and the solvents were evaporated under vacuum. The residue was diluted with acetonitrile (2 ml). The mixture was stirred at 80° C. for 1 hour. The solvents were evaporated under vacuum. The residue was diluted in a mixture of DMF-water (3.5 ml:0.5 ml) containing 2 drops of 30% aqueous ammonia and was injected on an HPLC column (C18, 5 microns, 19 mm diameter, 100 mm length) of a preparative HPLC-MS system eluting with a mixture of water and acetonitrile containing 2 g/l of ammonium carbonate (gradient) to give the desired compound.

### EXAMPLE 124

### N-[3-fluoro-4-(pyridin-3-yloxy)phenyl]-5-[(1R)-1-methyl-2-morpholin-4-yl-2-oxoethoxy]quinazolin-4-amine

Starting aniline: 3-fluoro-4-(pyridin-3-yloxy)aniline.

Yield: 191 mg; 59% from 0.66 mmol scale, except that after evaporation of the crude mixture, the residue was diluted with 10% 7N methanolic ammonia in DCM and, after evaporation of the solvents, purified by chromatography on silica gel (eluant: 5% 7N methanolic ammonia in DCM).

NMR Spectrum: (400 MHz; CDCl₃) 1.73 (d, 3H), 3.56 (m, 2H), 3.76 (m, 6H), 5.42 (q, 1H), 6.83 (d, 1H), 7.14 (t, 1H), 7.25 (m, 2H), 7.53 (d, 1H), 7.64 (t, 1H), 7.84 (d, 1H), 8.27 (dd, 1H), 8.34 (m, 1H), 8.45 (d, 1H), 8.69 (s, 1H); Mass spectrum: MH⁺ 490

The 3-fluoro-4-(pyridin-3-yloxy)aniline used as starting material was made from 1,2-difluoro-4-nitrobenzene and 3-hydroxypyridine according to Example 51, starting material.

3-(2-fluoro-4-nitrophenoxy)pyridine: Yield: 13.2 g, 89%; Mass spectrum: MH⁺ 235.

3-fluoro-4-(pyridin-3-yloxy)aniline: Yield: 11.5 g, 100%, except that hydrogenation was performed in ethanol with platinum oxide as a catalyst; Mass spectrum: MH⁺ 205.

The 5-[(1R)-1-methyl-2-morpholin-4-yl-2-oxoethoxy]quinazolin-4(3H)-one used as starting material was made as follows:

Sodium hydride (1.24 g, 60% in oil, 31 mmol) was added portionwise to a solution of 5-methoxyquinazolin-4(3H)-one (5 g, 28.4 mmol, Int. Patent Appl. WO96/09294 pages 28 and 29) in anhydrous DMF (50 ml) while maintaining the temperature at 25° C. The mixture was stirred at room temperature for 30 minutes. Chloromethyl pivalate (4.45 ml, 31 mmol) was added at room temperature for 3 hours. Additional sodium hydride (0.12 g, 3 mmol) and chloromethyl pivalate (0.67 ml, 4.5 mmol) were added and the mixture was stirred another hour. After evaporation of the solvents under high vacuum, the mixture was diluted with water and extracted with DCM. After drying with magnesium sulfate and evaporation of the solvents, the residue was purified by chromatography on silica gel (eluant: ethyl acetate-petroleum ether, 6:4 to 8:2) to give (5-methoxy-4-oxoquinazolin-3(4H)-yl)methyl pivalate as a white solid (7.4 g, 90%); HPLC t_{R}: 2.69 min; Mass spectrum: MH⁺ 291.

Magnesium bromide (7 g, 38 mmol) was added to a solution of (5-methoxy-4-oxoquinazolin-3(4H)-yl)methyl pivalate (7.4 g, 25.5 mmol) in pyridine (25 ml). The mixture was stirred at 120° C. for one hour. After cooling, the solvents were evaporated under high vacuum. Diluted acetic acid (15 ml in 100 ml water) was added. The precipitated solid was filtered, washed with water and dried under high vacuum in the presence of P2O₅ to give (5-hydroxy-4-oxoquinazolin-3(4H)-yl)methyl pivalate as a white solid (6.33 g, 90%); NMR Spectrum: (400 MHz; CDCl₃) 1.23 (s, 9H), 5.93 (s, 2H), 6.99 (d, 1H), 7.22 (d, 1H), 7.68 (t, 1H), 8.21 (s, 1H); Mass spectrum: MH⁺ 277.

Triphenylphosphine (8.92 g, 34 mmol), 4-((S)-2-hydroxypropionyl)morpholine (3.98 g, 25 mmol; Tasaka A., Chem. Pharm. Bull. 1993, 41, 1035) and DTAD (7.83 g, 34 mmol) were added successively to a solution of (5-hydroxy-4-oxoquinazolin-3(4H)-yl)methyl pivalate (5.8 g, 21 mmol) in DCM (60 ml). The mixture was stirred at room temperature for 45 minutes. After evaporation of the solvents under vacuum, the residue was diluted with 7N methanolic ammonia (200 ml). The mixture was stirred at room temperature for 18 hours. After evaporation of the solvents, the residue was purified by chromatography on silica gel (eluant: 5 to 15% 7N methanolic ammonia in DCM) to give 5-[(1R)-1-methyl-2-morpholin-4-yl-2-oxoethoxy]quinazolin-4(3H)-one as a beige solid (4.77 g, 75%); HPLC t_{R}: 1.53 min; Mass spectrum: 304.

### EXAMPLE 125

### N-{3-chloro-4-[(6-methylpyridin-3-yl)oxy]phenyl}-5-[(1R)-1-methyl-2-morpholin-4-yl-2-oxoethoxy]quinazolin-4-amine

Starting aniline: 3-chloro-4-[(6-methylpyridin-3-yl)oxy]aniline.

Yield: 61 mg; 30%.

NMR Spectrum: (400 MHz; CDCl₃) 1.70 (d, 3H), 2.51 (s, 3H), 3.54 (m, 2H), 3.72 (m, 6H), 5.37 (q, 1H), 6.81 (d, 1H), 7.02 (d, 1H), 7.07 (d, 1H), 7.13 (dd, 1H), 7.46 (d, 1H), 7.59 (t, 1H), 7.91 (dd, 1H), 8.27 (d, 1H), 8.37 (d, 1H), 8.60 (s, 1H); Mass spectrum: MH⁺ 520.

The 3-chloro-4-[(6-methylpyridin-3-yl)oxy]aniline used as starting material was made from 2-chloro-1-fluoro-4-nitrobenzene and 2-hydroxy-5-methylpyridine according to Example 51, starting material.

5-(2-chloro-4-nitrophenoxy)-2-methylpyridine: Yield: 13.3 g, 91%; Mass spectrum: MH⁺ 265.

3-chloro-4-[(6-methylpyridin-3-yl)oxy]aniline: Yield: 11.7 g, 100%, except that hydrogenation was performed in ethanol with platinum oxide as a catalyst; NMR

Spectrum: (400 MHz; CDCl₃) 2.51 (s, 3H), 3.70 (m, 2H), 6.56 (dd, 1H), 6.78 (d, 1H), 6.88 (d, 1H), 7.05 (s, 2H), 8.20 (s, 1H).

### EXAMPLE 126

### N-[3-chloro-4-(pyridin-3-yloxy)phenyl]-5-[(1R)-1-methyl-2-morpholin-4-yl-2-oxoethoxy]quinazolin-4-amine

Starting aniline: 3-chloro-4-(pyridin-3-yloxy)aniline.

Yield: 230 mg; 46% on 0.99 mmol scale except that after evaporation of the crude mixture, the residue was diluted with 10% 7N methanolic ammonia in DCM and, after evaporation of the solvents, purified by chromatography on silica gel (eluant: 5% 7N methanolic ammonia in DCM).

NMR Spectrum: (400 MHz; CDCl₃) 1.73 (d, 3H), 3.56 (m, 2H), 3.75 (m, 6H), 5.41 (q, 1H), 6.83 (d, 1H), 7.10 (d, 1H), 7.25 (m, 2H), 7.51 (d, 1H), 7.63 (t, 1H), 7.99 (dd, 1H), 8.34 (m, 1H), 8.43 (m, 2H), 8.69 (s, 1H); Mass spectrum: MH⁺ 506.

The 3-chloro-4-(pyridin-3-yloxy)aniline used as starting material was made from 2-chloro-1-fluoro-4-nitrobenzene and 3-hydroxypyridine according to Example 51, starting material.

3-(2-chloro-4-nitrophenoxy)pyridine: Yield: 12.7 g, 96%; Mass spectrum: MH⁺ 251.

3-chloro-4-(pyridin-3-yloxy)aniline: Yield: 11.2 g, 100%, except that hydrogenation was performed in ethanol with platinum oxide as a catalyst; NMR Spectrum: (400 MHz; CDCl₃) 3.50 (m, 2H), 6.58 (dd, 1H), 6.79 (s, 1H), 6.92 (d, 1H), 7.13 (m, 1H), 7.20 (m, 1H), 8.29 (d, 1H), 8.32 (s, 1H).

### EXAMPLE 127

### 5-[(1R)-1-methyl-2-morpholin-4-yl-2-oxoethoxy]-N-{4-[(6-methylpyridin-3-yl)oxy]phenyl}quinazolin-4-amine

Starting aniline: 4-[(6-methylpyridin-3-yl)oxy]aniline.

Yield: 41 mg; 21%.

NMR Spectrum: (400 MHz; CDCl₃) 1.72 (d, 3H), 2.53 (s, 3H), 3.55 (m, 2H), 3.72 (m, 6H), 5.38 (q, 1H), 6.80 (d, 1H), 7.03 (m, 2H), 7.10 (d, 1H), 7.24 (m, 1H), 7.48 (d, 1H), 7.58 (t, 1H), 7.96 (d, 2H), 8.32 (d, 1H), 8.62 (s, 1H); Mass spectrum: MH⁺ 486

The 4-[(6-methylpyridin-3-yl)oxy]aniline used as starting material was made from 1-fluoro-4-nitrobenzene and 2-hydroxy-5-methylpyridine according to Example 51, starting material:

2-methyl-5-(4-nitrophenoxy)pyridine: Yield: 15.8 g, 95%; Mass spectrum: MH⁺ 231.

4-[(6-methylpyridin-3-yl)oxy]aniline: Yield: 13.6 g, 100%, except that hydrogenation was performed in ethanol with platinum oxide as a catalyst; Mass spectrum: MH⁺ 201.

### EXAMPLE 128

### 5-[(1R)-1-methyl-2-morpholin-4-yl-2-oxoethoxy]-N-[4-(pyridin-3-yloxy)phenyl]-quinazolin-4-amine

Starting aniline: 4-pyridin-3-yloxy)aniline.

Yield: 26 mg; 14%.

NMR Spectrum: (400 MHz; CDCl₃) 1.72 (d, 3H), 3.56 (m, 2H), 3.73 (m, 6H), 5.39 (q, 1H), 6.80 (d, 1H), 7.07 (d, 2H), 7.24 (m, 1H), 7.32 (m, 1H), 7.49 (d, 1H), 7.61 (t, 1H), 8.01 (m, 2H), 8.34 (m, 1H), 8.43 (m, 1H), 8.69 (s, 1H); Mass spectrum: MH⁺ 472.

The 4-(pyridin-3-yloxy)aniline used as starting material was made from 1-fluoro-4-nitrobenzene and 3-hydroxypyridine according to Example 51, starting material.

3-(4-nitrophenoxy)pyridine: Yield: 10.3 g, 75%; Mass spectrum: MH⁺ 217.

4-(pyridin-3-yloxy)aniline: Yield: 8.7 g, 98%, except that hydrogenation was performed in ethanol with platinum oxide as a catalyst; Mass spectrum: MH⁺ 187.

### EXAMPLE 129

### N-{3-methoxy-4-[(6-methylpyridin-3-yl)oxy]phenyl}-5-[(1R)-1-methyl-2-morpholin-4-yl-2-oxoethoxy]quinazolin-4-amine

Starting aniline: 3-methoxy-4-[(6-methylpyridin-3-yl)oxy]aniline.

Yield: 42 mg; 21%.

NMR Spectrum: (400 MHz; CDCl₃) 1.73 (d, 3H), 2.51 (s, 3H), 3.56 (m, 2H), 3.72 (m, 6H), 3.89 (s, 3H), 5.40 (q, 1H), 6.81 (d, 1H), 7.00 (d, 1H), 7.05 (d, 1H), 7.14 (dd, 1H), 7.50 (d, 1H), 7.60 (m, 2H), 7.97 (d, 1H), 8.27 (d, 1H), 8.66 (s, 1H); Mass spectrum: MH⁺ 516.

The 3-methoxy-4-[(6-methylpyridin-3-yl)oxy]aniline used as starting material was made from 2-bromo-5-nitroanisole and 2-hydroxy-5-methylpyridine according to Example 51, starting material.

5-(2-methoxy-4-nitrophenoxy)-2-methylpyridine: Yield: 14.4 g, 83%, except that the reaction was run in DMF at 110° C. for 16 hours; Mass spectrum: MH⁺ 261.

3-methoxy-4-[(6-methylpyridin-3-yl)oxy]aniline: Yield: 12.2 g, 100%, except that hydrogenation was performed in ethanol with platinum oxide as a catalyst; Mass spectrum: MH⁺ 231.

### EXAMPLE 130

### N-[3-methoxy-4-(pyridin-3-yloxy)phenyl]-5-[(1R)-1-methyl-2-morpholin-4-yl-2-oxoethoxy]quinazolin-4-amine

Starting aniline: 3-methoxy-4-(pyridin-3-yloxy)aniline.

Yield: 21 mg; 11%.

NMR Spectrum: (400 MHz; CDCl₃) 1.73 (d, 3H), 3.56 (m, 2H), 3.73 (m, 6H), 3.89 (s, 3H), 5.40 (q, 1H), 6.81 (d, 1H), 7.05 (d, 1H), 7.20 (m, 2H), 7.49 (d, 1H), 7.61 (t, 1H), 7.66 (dd, 1H), 8.01 (d, 1H), 8.27 (m, 1H), 8.38 (d, 1H), 8.66 (s, 1H); Mass spectrum: MH⁺ 502.

The 3-methoxy-4-(pyridin-3-yloxy)aniline used as starting material was made from 2-bromo-5-nitroanisole and 3-hydroxypyridine according to Example 51, starting material.

3-(2-methoxy-4-nitrophenoxy)pyridine Yield: 6.65 g, 65%, except that the reaction was run in DMF at 110° C. for 16 hours; Mass spectrum: MH⁺ 247.

3-methoxy-4-(pyridin-3-yloxy)aniline: Yield: 5.74 g, 100%, except that hydrogenation was performed in ethanol with platinum oxide as a catalyst; Mass spectrum: MH⁺ 217.

### EXAMPLE 131

### N-{3-fluoro-4-[(6-methylpyridin-3-yl)oxy]phenyl}-5-[(1R)-1-methyl-2-morpholin-4-yl-2-oxoethoxy]quinazolin-4-amine

Starting aniline: 3-fluoro-4-[(6-methylpyridin-3-yl)oxy]aniline.

Yield: 31 mg; 16%.

NMR Spectrum: (400 MHz; CDCl₃) 1.71 (d, 3H), 2.52 (s, 3H), 3.55 (m, 2H), 3.74 (m, 6H), 5.39 (q, 1H), 6.81 (d, 1H), 7.08 (m, 2H), 7.17 (dd, 1H), 7.49 (d, 1H), 7.55 (t, 1H), 7.78 (m, 1H), 8.23 (dd, 1H), 8.31 (d, 1H), 8.61 (s, 1H); Mass spectrum: MH⁺ 504.

The 3-fluoro-4-[(6-methylpyridin-3-yl)oxy]aniline used as starting material was made from 1,2-difluoro-4-nitrobenzene and 2-hydroxy-5-methylpyridine according to Example 51, starting material.

5-(2-fluoro-4-nitrophenoxy)-2-methylpyridine: Yield: 17.3 g, 96%; Mass spectrum: MH⁺ 249.

3-fluoro-4-[(6-methylpyridin-3-yl)oxy]aniline: Yield: 14.7 g, 96%, except that hydrogenation was performed in ethanol with platinum oxide as a catalyst; Mass spectrum: MH⁺ 219.

### EXAMPLE 132

### N-{3-cyano-4-[(6-methylpyridin-3-yl)oxy]phenyl}-5-[(1R)-1-methyl-2-morpholin-4-yl-2-oxoethoxy]quinazolin-4-amine

Starting aniline: 3-cyano-4-[(6-methylpyridin-3-yl)oxy]aniline.

Yield: 64 mg; 32%.

NMR Spectrum: (400 MHz; CDCl₃) 1.69 (d, 3H), 2.56 (s, 3H), 3.54 (m, 2H), 3.73 (m, 6H), 5.40 (q, 1H), 6.82 (d, 1H), 6.89 (d, 1H), 7.17 (d, 1H), 7.30 (dd, 1H), 7.47 (d, 1H), 7.61 (t, 1H), 8.24 (dd, 1H), 8.34 (d, 1H), 8.59 (d, 1H), 8.63 (s, 1H); Mass spectrum: MH⁺ 511.

The 3-cyano-4-[(6-methylpyridin-3-yl)oxy]aniline used as starting material was made from 2-fluoro-5-nitrobenzonitrile and 2-hydroxy-5-methylpyridine according to Example 51, starting material.

5-(2-cyano-4-nitrophenoxy)-2-methylpyridine: Yield: 13.7 g, 81%; Mass spectrum: MH⁺ 256.

3-cyano-4-[(6-methylpyridin-3-yl)oxy]aniline: Yield: 11.8 g, 98%, except that hydrogenation was performed in ethanol with platinum oxide as a catalyst; Mass spectrum: MH⁺ 226.

### EXAMPLE 133

### N-[3-cyano-4-(pyridin-3-yloxy)phenyl]-5-[(1R)-1-methyl-2-morpholin-4-yl-2-oxoethoxy]quinazolin-4-amine

Starting aniline: 3-cyano-4-(pyridin-3-yloxy)aniline.

Yield: 32 mg; 16%.

NMR Spectrum: (400 MHz; CDCl₃) 1.72 (d, 3H), 3.56 (m, 2H), 3.76 (m, 6H), 5.41 (q, 1H), 6.84 (d, 1H), 6.98 (d, 1H), 7.34 (m, 1H), 7.41 (m, 1H), 7.53 (d, 1H), 7.65 (t, 1H), 8.33 (dd, 1H), 8.46 (d, 1H), 8.49 (d, 1H), 8.62 (d, 1H), 8.68 (s, 1H); Mass spectrum: MH⁺ 497.

The 3-cyano-4-(pyridin-3-yloxy)aniline used as starting material was made from 2-fluoro-5-nitrobenzonitrile and 3-hydroxypyridine according to Example 51, starting material.

3-(2-cyano-4-nitrophenoxy)pyridine: Yield: 12.0 g, 95%; Mass spectrum: MH⁺ 242.

3-cyano-4-(pyridin-3-yloxy)aniline: Yield: 10.2 g, 87%, except that hydrogenation was performed in ethanol with platinum oxide as a catalyst; Mass spectrum: MH⁺ 212.

### EXAMPLE 134

### 5-[(1R)-1-methyl-2-morpholin-4-yl-2-oxoethoxy]-N-[3-methyl-4-(pyridin-2-yloxy)phenyl]quinazolin-4-amine

Starting aniline: 3-methyl-4-(pyridin-2-yloxy)aniline.

Yield: 17 mg; 9%.

NMR Spectrum: (400 MHz; CDCl₃) 1.73 (d, 3H), 2.22 (s, 3H), 3.57 (m, 2H), 3.71 (m, 6H), 5.37 (q, 1H), 6.79 (d, 1H), 6.85 (d, 1H), 6.95 (m, 1H), 7.09 (d, 1H), 7.48 (d, 1H), 7.59 (t, 1H), 7.65 (m, 1H), 7.78 (dd, 1H), 7.89 (d, 1H), 8.18 (m, 1H), 8.63 (s, 1H); Mass spectrum: MH⁺ 486.

The 3-methyl-4-(pyridin-2-yloxy)aniline used as starting material was prepared as follows:

2-fluoropyridine (16.9 g, 174 mmol) was added to a mixture of 2-methyl-4-nitrophenol (25 g, 158 mmol) and potassium carbonate (65.7 g, 475 mmol) in DMA (125 ml). The mixture was heated at 200° C. for 18 hours. After cooling, the solids were filtered off and rinsed. The resulting filtrate was evaporated under high vacuum. The residue was diluted with water and extracted with DCM. The organic layer was dried over magnesium sulfate. After evaporation of the solvents, the residue was purified by chromatography on silica gel (eluant: DCM) to give 2-(2-methyl-4-nitrophenoxy)pyridine as a yellowish solid (14.7 g, 40%); Mass spectrum: MH⁺ 231.

2-(2-Methyl-4-nitrophenoxy)pyridine (14.7, 63.8 mmol) was converted into 3-methyl-4-(pyridin-2-yloxy)aniline by hydrogenation with platinum oxide in ethanol using a procedure similar to Example 51, starting material.

3-methyl-4-(pyridin-2-yloxy)aniline: Yield: 11.6 g, 91% (white solid); Mass spectrum: MH⁺ 201.

### EXAMPLE 135

### 5-[(1R)-1-methyl-2-morpholin-4-yl-2-oxoethoxy]-N-[3-methyl-4-(pyridin-3-yloxy)phenyl]quinazolin-4-amine

Starting aniline: 3-methyl-4-(pyridin-3-yloxy)aniline.

Yield: 59 mg; 31%.

NMR Spectrum: (400 MHz; CDCl₃) 1.73 (d, 3H), 2.26 (s, 3H), 3.56 (m, 2H), 3.72 (m, 6H), 5.38 (q, 1H), 6.79 (d, 1H), 6.98 (d, 1H), 7.19 (m, 2H), 7.48 (d, 1H), 7.60 (t, 1H), 7.80 (dd, 1H), 7.95 (d, 1H), 8.28 (m, 1H), 8.38 (d, 1H), 8.64 (s, 1H); Mass spectrum: MH⁺ 486.

The 3-methyl-4-(pyridin-3-yloxy)aniline used as starting material was made from 2-fluoro-5-nitrotoluene and 3-hydroxypyridine according to Example 51, starting material.

3-(2-methyl-4-nitrophenoxy)pyridine: Yield: 13.5 g, 93%; Mass spectrum: MH⁺ 231.

3-methyl-4-(pyridin-3-yloxy)aniline: Yield: 11.5 g, 98%, except that hydrogenation was performed in ethanol with platinum oxide as a catalyst; NMR Spectrum: (400 MHz; CDCl₃) 2.10 (s, 3H), 3.5 (m, 2H), 6.53 (dd, 1H), 6.60 (d, 1H), 6.79 (d, 1H), 7.08 (m, 1H), 7.17 (m, 1H), 8.24 (d, 1H), 8.30 (s, 1H).

### EXAMPLE 136

### 5-[(1R)-1-methyl-2-morpholin-4-yl-2-oxoethoxy]-N-[3-methyl-4-(pyridin-4-yloxy)phenyl]quinazolin-4-amine

Starting aniline: 3-methyl-4-(pyridin-4-yloxy)aniline.

Yield: 60 mg; 13% on 0.99 mmol scale except that after evaporation of the crude mixture, the residue was diluted with 10% 7N methanolic ammonia in DCM and, after evaporation of the solvents, purified by chromatography on silica gel (eluant: 5% 7N methanolic ammonia in DCM).

NMR Spectrum: (400 MHz; CDCl₃) 1.73 (d, 3H), 2.19 (s, 3H), 3.56 (m, 2H), 3.73 (m, 6H), 5.39 (q, 1H), 6.80 (m, 3H), 7.03 (d, 1H), 7.47 (d, 1H), 7.60 (t, 1H), 7.87 (dd, 1H), 7.99 (d, 1H), 8.42 (d, 2H), 8.68 (s, 1H), 10.82 (s, 1H); Mass spectrum: MH⁺ 486.

The 3-methyl-4-(pyridin-4-yloxy)aniline used as starting material was prepared as follows:

A mixture of 4-amino-2-methylphenol (5.5 g, 45 mmol), 4-chloropyridine hydrochloride (7.4 g, 49.5 mmol) and potassium tert-butoxide (15 g, 135 mmol) in DMF (17 ml)-DMPU (70 ml) was heated at 100° C. for 20 hours. After cooling, the mixture was diluted with water and extracted with ether. The organic layer washed with water and brine and dried over magnesium sulfate. After evaporation of the solvents, the residue was purified by chromatography on silica gel (eluant: ethyl acetate) to give 3-methyl-4-(pyridin-4-yloxy)aniline as a light brown solid (4.3 g, 48%); Mass spectrum: MH⁺ 201.

### EXAMPLE 137

### 5-[(1R)-1-methyl-2-morpholin-4-yl-2-oxoethoxy]-N-[3-methyl-4-(pyrazin-2-yloxy)phenyl]quinazolin-4-amine

Starting aniline: 3-methyl-4-(pyrazin-2-yloxy)aniline.

Yield: 140 mg; 29% on 0.99 mmol scale except that after evaporation of the crude mixture, the residue was diluted with 10% 7N methanolic ammonia in DCM and, after evaporation of the solvents, purified by chromatography on silica gel (eluant: 5% 7N methanolic ammonia in DCM).

NMR Spectrum: (400 MHz; CDCl₃) 1.74 (d, 3H), 2.23 (s, 3H), 3.57 (m, 2H), 3.73 (m, 6H), 5.38 (q, 1H), 6.81 (d, 1H), 7.10 (d, 1H), 7.49 (d, 1H), 7.61 (t, 1H), 7.86 (dd, 1H), 7.97 (d, 1H), 8.10 (m, 1H), 8.25 (d, 1H), 8.43 (s, 1H), 8.65 (s, 1H); Mass spectrum: MH⁺ 487.

The 3-methyl-4-(pyrazin-2-yloxy)aniline used as starting material was prepared as follows:

A mixture of 2-methyl-4-nitrophenol (1.4 g, 9.2 mmol), 2-chloropyrazine (1.16 g, 10.1 mmol), cesium carbonate (6 g, 18.4 mmol) and copper(I) iodide (175 mg, 0.92 mmol) in DMA (7 ml) was irradiated in a Personal Chemistry EMRYS™ Optimizer EXP microwave synthesisor at 200° C. for 15 minutes. After cooling, the solids were filtered off and rinsed. The resulting filtrate was evaporated under high vacuum. The residue was diluted with DCM and purified by chromatography on silica gel (eluant: DCM) to give 2-(2-methyl-4-nitrophenoxy)pyrazine as a yellowish solid (2.4 g, 38%); NMR Spectrum: (400 MHz; CDCl₃) 2.31 (s, 3H), 7.22 (d, 1H), 8.10 (s, 1H), 8.14 (dd, 1H), 8.21 (s, 1H), 8.35 (s, 1H), 8.55 (s, 1H).

2-(2-Methyl-4-nitrophenoxy)pyrazine (2.38 g) was converted into 3-methyl-4-(pyrazin-2-yloxy)aniline by hydrogenation with platinum oxide in ethanol using a procedure similar to Example 51, starting material; 3-methyl-4-(pyrazin-2-yloxy)aniline (1.35 g, 65%); Mass spectrum: MH⁺ 202.

### EXAMPLES 138 TO 143

A mixture of 5-[(1R)-1-methyl-2-morpholin-4-yl-2-oxoethoxy]quinazolin-4(3H)-one (120 mg, 0.4 mmol), triphenylphosphine (312 mg, 1.19 mmol) and carbon tetrachloride (1.1 ml, 12 mmol) in 1,2-dichloroethane (3 ml) was stirred at 45° C. for 2 hours. The mixture was cooled. The corresponding aniline (0.42 mmol) was added and the solvents were evaporated under vacuum. The residue was diluted with acetonitrile (2 ml) and 4N hydrogen chloride in dioxane (2 drops) was added. The mixture was stirred at 75° C. for 4 hours. The solvents were evaporated under vacuum. The residue was diluted in DCM, washed with saturated aqueous bicarbonate. The organic layer was dried over magnesium sulfate and purified by chromatography on silica gel (eluant: 5% methanol in DCM) to give the desired compound.

### EXAMPLE 138

### 5-[(1R)-1-Methyl-2-morpholin-4-yl-2-oxoethoxy]-N-[3-methyl-4-(1,3-thiazol-2-yloxy)phenyl]quinazolin-4-amine

Starting aniline: 3-methyl-4-(1,3-thiazol-2-yloxy)aniline.

Yield: 97 mg; 50%.

NMR Spectrum: (400 MHz; CDCl₃) 1.70 (d, 3H), 2.30 (s, 3H), 3.54 (m, 2H), 3.70 (m, 6H), 5.36 (q, 1H), 6.75 (m, 2H), 7.20 (m, 2H), 7.44 (d, 1H), 7.56 (t, 1H), 7.86 (dd, 1H), 7.98 (s, 1H), 8.63 (s, 1H); Mass spectrum: MH⁺ 492.

The 3-methyl-4-(1,3-thiazol-2-yloxy)aniline used as starting material was prepared as follows:

2-Chlorothiazole (4.71 g, 39.4 mmol; Boga C., J. Organomet. Chem., 1999, 588, 155) was slowly added to a mixture of 4-amino-2-methylphenol (5 g, 39.4 mmol) and potassium hydroxide (2.21 g, 39.4 mmol) in DMA (50 ml) preheated at 60° C. The mixture was heated at 135° C. for 24 hours. After cooling, the solvent was evaporated under high vacuum. The residue was diluted with water (pH>9) and extracted with ether. The organic layer washed with brine and dried over magnesium sulfate. After evaporation of the solvents, the residue was purified by chromatography on silica gel (eluant: 50% ethyl acetate in petroleum ether) to give 3-methyl-4-(1,3-thiazol-2-yloxy)aniline as a brown oil (4.5 g, 55%); Mass spectrum: MH⁺ 207.

### EXAMPLE 139

### N-{4-[(6-Methoxypyridin-3-yl)oxy]-3-methylphenyl}-5-[(1R)-1-methyl-2-morpholin-4-yl-2-oxoethoxy]quinazolin-4-amine

Starting aniline: 4-[(6-methoxypyridin-3-yl)oxy]-3-methylaniline.

Yield: 70 mg; 29% on a 0.46 mmol scale.

NMR Spectrum: (400 MHz; CDCl₃) 1.73 (d, 3H), 2.33 (s, 3H), 3.56 (m, 2H), 3.73 (m, 6H), 3.92 (s, 3H), 5.38 (q, 1H), 6.71 (d, 1H), 6.79 (d, 1H), 6.84 (d, 1H), 7.25 (m, 1H), 7.48 (d, 1H), 7.60 (t, 1H), 7.66 (dd, 1H), 7.87 (s, 1H), 7.91 (d, 1H), 8.63 (s, 1H); Mass spectrum: MH⁺ 516.

The 4-[(6-methoxypyridin-3-yl)oxy]-3-methylaniline used as starting material was prepared from 2-fluoro-5-nitrotoluene and 5-hydroxy-2-methoxypyridine (Adams G., J. Am. Chem. Soc., 1947, 69, 1806) according to Example 51, starting material.

2-methoxy-5-(2-methyl-4-nitrophenoxy)pyridine: Yield: 0.98 g, 54%; Mass spectrum: MH⁺ 261.

4-[(6-methoxypyridin-3-yl)oxy]-3-methylaniline: Yield: 0.85 g, 98%, except that hydrogenation was performed in ethanol with platinum oxide as a catalyst; NMR Spectrum: (400 MHz; CDCl₃) 2.14 (s, 3H), 3.53 (m, 2H), 3.89 (s, 3H), 6.49 (dd, 1H), 6.57 (d, 1H), 6.66 (d, 1H), 6.71 (d, 1H), 7.15 (dd, 1H), 7.79 (d, 1H).

### EXAMPLE 140

### 5-[(1R)-1-methyl-2-morpholin-4-yl-2-oxoethoxy]-N-[3-methyl-4-(1,3-thiazol-5-yloxy)phenyl]quinazolin-4-amine

Starting aniline: 3-methyl-4-(1,3-thiazol-5-yloxy)aniline.

Yield: 4.5 mg; 5% on a 0.2 mmol scale.

NMR Spectrum: (400 MHz; CDCl₃) 1.73 (d, 3H), 2.37 (s, 3H), 3.56 (m, 2H), 3.73 (m, 6H), 5.40 (q, 1H), 6.81 (d, 1H), 7.04 (d, 1H), 7.39 (s, 1H), 7.51 (d, 1H), 7.61 (t, 1H), 7.77 (dd, 1H), 7.91 (d, 1H), 8.35 (s, 1H), 8.63 (s, 1H); Mass spectrum: MH⁺ 492.

The 3-methyl-4-(1,3-thiazol-5-yloxy)aniline used as starting material was prepared as follows:

5-Chlorothiazole (190 mg, 1.58 mmol; Reynaud P., Bull. Soc. Chem. Fr., 1962, 1735) was slowly added to a mixture of 4-amino-2-methylphenol (200 mg, 1.58 mmol) and potassium hydroxide (90 mg, 1.58 mmol) in DMA (5 ml) at room temperature. The mixture was irradiated in a Personal Chemistry EMRYS™ Optimizer EXP microwave synthesisor at 160° C. for 1 hour. After cooling, the solvent was evaporated under high vacuum. The residue was diluted with water (pH>9) and extracted with ether. The organic layer washed with brine and dried over magnesium sulfate. After evaporation of the solvents, the residue was directly injected on an HPLC column (C18, 5 microns, 19 mm diameter, 100 mm length) of a preparative HPLC-MS system eluting with a mixture of water and acetonitrile containing 2 g/l of ammonium carbonate (gradient) to give 3-methyl-4-(1,3-thiazol-5-yloxy)aniline as a brown oil (30 mg, 9%); Mass spectrum: MH⁺ 207.

### EXAMPLE 141

### 5-[(1R)-1-methyl-2-morpholin-4-yl-2-oxoethoxy]-N-[3-methyl-4-(pyrimidin-5-yloxy)phenyl]quinazolin-4-amine

Starting aniline: 3-methyl-4-(pyrimidin-5-yloxy)aniline.

Yield: 66 mg; 41% on a 0.33 mmol scale.

NMR Spectrum: (400 MHz) 1.57 (d, 3H), 2.24 (s, 3H), 3.8-3.3 (m, 8H), 5.88 (q, 1H), 7.13 (d, 1H), 7.30 (d, 1H), 7.36 (d, 1H), 7.75 (t, 1H), 7.97 (dd, 1H), 8.10 (d, 1H), 8.53 (s, 2H), 8.55 (s, 1H), 8.95 (s, 1H), 11.09 (s, 1H); Mass spectrum: MH⁺ 487.

The 3-methyl-4-(pyrimidin-5-yloxy)aniline used as starting material was prepared as follows:

A mixture of 4-amino-2-methylphenol (1.77 g, 14.4 mmol), 5-bromopyrimidine (2.29 g, 14.4 mmol), potassium carbonate (2.98 g, 21.6 mmol) in DMSO (10 ml) was irradiated in a Personal Chemistry EMRYS™ Optimizer EXP microwave synthesisor at 150° C. for 2.5 hours. Copper(I) iodide (1.37 g, 7.2 mmol) was added and the mixture was irradiated in the microwave at 150° C. for 40 minutes more. After cooling, the mixture was partitioned with water and ethyl acetate. After filtration of the insoluble, the organic layer washed with water and brine and dried over magnesium sulfate. After evaporation of the solvents, the residue was purified by chromatography on silica gel (eluant: 30% up to 60% ethyl acetate in petroleum ether) to give 3-methyl-4-(pyrimidin-5-yloxy)aniline as a brown solid (315 mg, 11%); Mass spectrum: MH⁺ 202.

### EXAMPLE 142

### 5-[2-methyl-4-({5-[(1R)-1-methyl-2-morpholin-4-yl-2-oxoethoxy]quinazolin-4-yl}amino)phenoxy]pyridine-2-carbonitrile

Starting aniline: 5-(4-amino-2-methylphenoxy)pyridine-2-carbonitrile.

Yield: 243 mg; 58% on a 0.82 mmol scale.

NMR Spectrum: (400 MHz; CDCl₃) 1.74 (d, 3H), 2.23 (s, 3H), 3.57 (m, 2H), 3.75 (m, 6H), 5.42 (q, 1H), 6.83 (d, 1H), 7.03 (d, 1H), 7.19 (m, 1H), 7.69-7.46 (m, 3H), 7.91 (dd, 1H), 8.05 (d, 1H), 8.47 (d, 1H), 8.67 (s, 1H); Mass spectrum: MH⁺ 511.

The 5-(4-amino-2-methylphenoxy)pyridine-2-carbonitrile used as starting material was prepared as follows:

A mixture of 4-amino-2-methylphenol (3 g, 23.6 mmol), 5-chloropyridine-2-carbonitrile (3.6 g, 26 mmol; PCT Int. Appl. WO2001012627, Example 1 p 21) and sodium hydride (992 mg, 24.8 mmol, 60% dispersion in oil) in DMF (30 ml) was heated at 80° C. for 1 hour. After cooling, the mixture was diluted with water and extracted with DCM. The organic layer washed with water and brine and dried over magnesium sulfate. After evaporation of the solvents, the residue was purified by chromatography on silica gel (eluant: 40% up to 50% ethyl acetate in petroleum ether) to give 5-(4-amino-2-methylphenoxy)pyridine-2-carbonitrile as a light brown oil (5.25 g, 98%) which crystallised on standing; Mass spectrum: MH⁺ 226.

### EXAMPLE 143

### 5-[(1R)-1-methyl-2-morpholin-4-yl-2-oxoethoxy]-N-[3-methyl-4-(pyridazin-3-yloxy)phenyl]quinazolin-4-amine

Starting aniline: 3-methyl-4-(pyridazin-3-yloxy)aniline.

Yield: 89 mg; 56% on a 0.33 mmol scale.

NMR Spectrum: (400 MHz) 1.58 (d, 3H), 2.13 (s, 3H), 3.8-3.3 (m, 8H), 5.88 (q, 1H), 7.17 (d, 1H), 7.30 (d, 1H), 7.36 (d, 1H), 7.47 (d, 1H), 7.75 (m, 2H), 7.90 (dd, 1H), 8.00 (d, 1H), 8.53 (s, 1H), 8.99 (m, 1H), 11.09 (s, 1H); Mass spectrum: MH⁺ 487.

The 3-methyl-4-(pyridazin-3-yloxy)aniline used as starting material was prepared as follows:

A mixture of 4-amino-2-methylphenol (550 mg, 4.47 mmol), 3-chloropyridazine (510 mg, 4.47 mmol; Libermann et al., Bull. Soc. Chem. Fr., 1962, 1735), potassium carbonate (926 mg, 6.71 mmol) in DMA (10 ml) was irradiated in a Personal Chemistry EMRYS™ Optimizer EXP microwave synthesisor at 180° C. for 50 minutes. After cooling, the mixture was partitioned with water and dichloromethane. After filtration of the insoluble, the organic layer washed with water and brine and dried over magnesium sulfate. After evaporation of the solvents, the residue was purified by chromatography on silica gel (eluant: ethyl acetate) to give 3-methyl-4-(pyridazin-3-yloxy)aniline as a brown solid (638 mg, 71%); Mass spectrum: MH⁺ 202.

### EXAMPLE 144

### (2R)—N-(2-hydroxyethyl)-2-{[4-({3-methoxy-4-[(6-methylpyridin-3-yl)oxy]phenyl}amino)quinazolin-5-yl]oxy}-N-methylpropanamide

A mixture of methyl (2R)-2-{[4-({3-methoxy-4-[(6-methylpyridin-3-yl)oxy]phenyl}amino)quinazolin-5-yl]oxy}propanoate (184 mg, 0.40 mmol), 2-(methylamino)ethanol (0.19 ml, 1.2 mmol) and 4 Å molecular sieves in methanol (5 ml) was stirred at 65° C. for 4 hours. After filtration, the mixture was evaporated under vacuum, triturated with ether. The residue was purified by chromatography on silica gel (eluant: 5% methanol in DCM) to give the title compound (90 mg, 45%); NMR Spectrum: (400 MHz) (2 rotamers) 1.60 (m, 3H), 2.42 (s, 3H), 3.18 and 2.94 (s, 3H), 3.7-3.4 (m, 4H), 3.82 and 3.80 (s, 3H), 4.99 and 4.75 (t, 3H), 5.95 and 5.85 (m, 1H), 7.19-7.13 (m, 3H), 7.4-7.3 (m, 2H), 7.75 (m, 1H), 7.91 (m, 1H), 8.06 and 8.02 (m, 1H), 8.13 (d, 1H), 8.57 (s, 1H), 11.21 and 11.17 (bs, 1H); Mass spectrum: MH⁺ 504.

The methyl (2R)-2-{[4-({3-methoxy-4-[(6-methylpyridin-3-yl)oxy]phenyl}amino)quinazolin-5-yl]oxy}propanoate used as starting material was made from 4-chloro-5-fluoroquinazoline, 3-methoxy-4-[(6-methylpyridin-3-yloxy]aniline and methyl (S)-lactate according to the procedure in Example 51, starting material.

5-fluoro-N-{3-methoxy-4-[(6-methylpyridin-3-yl)oxy]phenyl}quinazolin-4-amine: Yield: 4.4 g, 77%; Mass spectrum: MH⁺ 377.

5-methoxy-N-{3-methoxy-4-[(6-methylpyridin-3-yl)oxy]phenyl}quinazolin-4-amine: Yield: 2.5 g, 93%; Mass spectrum: MH⁺ 389.

5-hydroxy-N-{3-methoxy-4-[(6-methylpyridin-3-yl)oxy]phenyl}quinazolin-4-amine: Yield: 2.3 g, 95%; Mass spectrum: MH⁺ 375.

Methyl (2R)-2-{[4-({3-methoxy-4-[(6-methylpyridin-3-yl)oxy]phenyl}amino)quinazolin-5-yl]oxy}propanoate: Yield: 2.05 g, 72%; NMR Spectrum: (400 MHz; CDCl₃) 1.80 (d, 3H), 2.53 (s, 3H), 3.87 (s, 3H), 3.91 (s, 3H), 5.17 (q, 1H), 6.82 (d, 1H), 7.02 (d, 1H), 7.07 (d, 1H), 7.16 (m, 1H), 7.46 (dd, 1H), 7.53 (d, 1H), 7.64 (t, 1H), 7.94 (d, 1H), 8.29 (d, 1H), 8.68 (s, 1H); Mass spectrum: MH⁺ 461.

### EXAMPLE 145

### (2R)-2-{[4-({3-methoxy-4-[(6-methylpyridin-3-yl)oxy]phenyl}amino)quinazolin-5-yl]oxy}-N,N-dimethylpropanamide

The procedure described in Example 144 was repeated using saturated dimethylamine in methanol (2 ml) instead of 2-(methylamino)ethanol to give the title compound (140 mg, 74%) except that the reaction was run at room temperature; NMR Spectrum: (400 MHz; CDCl₃) 1.72 (d, 3H), 3.07 (s, 3H), 3.15 (s, 3H), 3.91 (s, 3H), 5.44 (q, 1H), 6.82 (d, 1H), 7.06-7.01 (m, 2H), 7.13 (dd, 1H), 7.47 (d, 1H), 7.61 (t, 1H), 7.70 (dd, 1H), 8.00 (s, 1H), 8.30 (d, 1H), 8.66 (s, 1H); Mass spectrum: MH⁺ 474.

### EXAMPLE 146

### (2R)—N-ethyl-2-{[4-({3-methoxy-4-[(6-methylpyridin-3-yl)oxy]phenyl}amino)quinazolin-5-yl]oxy}propanamide

The procedure described in Example 144 was repeated using 70% aqueous methylamine instead of 2-(methylamino)ethanol to give the title compound (77 mg, 50%) except that the reaction was run at room temperature; NMR Spectrum: (400 MHz) 1.05 (t, 3H), 1.64 (d, 3H), 2.42 (s, 3H), 3.18 (m, 2H), 3.80 (s, 3H), 5.18 (q, 1H), 7.04 (d, 1H), 7.19-7.13 (m, 3H), 7.39 (d, 1H), 7.75 (m, 2H), 7.98 (s, 1H), 8.13 (d, 1H), 8.46 (m, 1H), 8.58 (s, 1H); Mass spectrum: MH⁺ 474.

### EXAMPLE 147

### (2R)—N-(2-hydroxyethyl)-2-{[4-({3-methoxy-4-[(6-methylpyridin-3-yl)oxy]phenyl}amino)quinazolin-5-yl]oxy}propanamide

The procedure described in Example 144 was repeated using ethanolamine instead of 2-(methylamino)ethanol to give the title compound (140 mg, 88%); NMR Spectrum: (400 MHz) 1.63 (d, 3H), 2.42 (s, 3H), 3.24 (m, 2H), 3.44 (m, 2H), 3.80 (s, 3H), 4.79 (m, 1H), 5.26 (q, 1H), 7.05 (d, 1H), 7.19-7.11 (m, 3H), 7.38 (d, 1H), 7.75 (m, 2H), 7.98 (s, 1H), 8.13 (d, 1H), 8.53 (m, 1H), 8.58 (s, 1H); Mass spectrum: MH⁺ 490.

### EXAMPLES 148 TO 150

Procedure:

EDCI (69 mg, 0.36 mmol) was added to a solution of (2R)-2-{[4-({3-methoxy-4-[(6-methylpyridin-3-yl)oxy]phenyl}amino)quinazolin-5-yl]oxy}propanoic acid (132 mg, 0.30 mmol), the appropriate amine (0.44 mmol) and 2-hydroxypyridine-N-oxide (40 mg, 0.36 mmol) in DMF (1 ml). The mixture was stirred at room temperature for 18 hours. The reaction mixture was directly injected on an HPLC column (C18, 5 microns, 20 mm diameter, 100 mm length) of a preparative HPLC-MS system eluting with a mixture of water and acetonitrile containing 2 g/l of ammonium carbonate (gradient) to give the desired compound.

### EXAMPLE 148

### 4-((2R)-2-{[4-({3-methoxy-4-[(6-methylpyridin-3-yl)oxy]phenyl}amino)quinazolin-5-yl]oxy}propanoyl)piperazin-2-one

Starting amine: piperazin-2-one.

Yield: 110 mg, 70%.

HPLC t_{R}: 1.95 min; Mass spectrum: MH⁺ 529.

### EXAMPLE 149

### (2R)—N-(2-methoxyethyl)-2-{[4-({3-methoxy-4-[(6-methylpyridin-3-yl)oxy]phenyl}amino)quinazolin-5-yl]oxy}-N-methylpropanamide

Starting amine: (2-methoxyethyl)methylamine.

Yield: 105 mg, 69%.

NMR Spectrum: (400 MHz; CDCl₃) (2 rotamers) 1.72 (m, 3H), 2.52 (s, 3H), 3.21 and 3.05 (s, 3H), 3.33 (s, 3H), 3.8-3.4 (m, 4H), 3.92 and 3.90 (s, 3H), 5.72 and 5.45 (q, 1H), 6.95 and 6.84 (d, 1H), 7.01 (m, 1H), 7.06 (d, 1H), 7.15 (m, 1H), 7.53 (m, 1H), 7.65 (m, 2H), 7.98 (dd, 1H), 8.29 (d, 1H), 8.66 (d, 1H); Mass spectrum: MH⁺ 518.

### EXAMPLE 150

### (3R)-1-((2R)-2-{[4-({3-methoxy-4-[(6-methylpyridin-3-yl)oxy]phenyl}amino)quinazolin-5-yl]oxy}propanoyl)piperidin-3-ol

Starting amine: (R)-3-hydroxypiperidine hydrochloride (except that 1 equivalent of triethylamine was added).

Yield: 105 mg, 67%.

HPLC t_{R}: 2.10 min; Mass spectrum: MH⁺ 530.

The (2R)-2-{[4-({3-methoxy-4-[(6-methylpyridin-3-yl)oxy]phenyl}amino)quinazolin-5-yl]oxy}propanoic acid used as starting material was prepared from methyl (2R)-2-{[4-({3-methoxy-4-[(6-methylpyridin-3-yl)oxy]phenyl}amino)quinazolin-5-yl]oxy}propanoate using the procedure described in Example 51, starting material.

Yield: 1.6 g, 83%; NMR Spectrum: (400 MHz; DMSOd₆ and CF₃CO₂D) 1.76 (d, 3H), 2.72 (s, 3H), 3.85 (s, 3H), 5.56 (q, 1H), 7.43 (d, 1H), 7.54 (m, 2H), 7.67 (dd, 1H), 7.90 (m, 2H), 8.07 (m, 2H), 8.70 (d, 1H), 9.03 (s, 1H); Mass spectrum: MH⁺ 447.

### EXAMPLE 151

### N-{3-methoxy-4-[(6-methylpyridin-3-yl)oxy]phenyl}-5-[(1R)-1-methyl-2-oxo-2-piperazin-1-ylethoxy]quinazolin-4-amine

A mixture of tert-butyl 4-((2R)-2-{[4-({3-methoxy-4-[(6-methylpyridin-3-yl)oxy]phenyl}amino)quinazolin-5-yl]oxy}propanoyl)piperazine-1-carboxylate (100 mg, 0.16 mmol) in 5N HCl in propanol (1 ml) was stirred at room temperature for 1 hour. Ether was added and the precipitate was collected to give the title compound as a hydrochloride salt (82 mg, 80%); NMR Spectrum: (400 MHz; DMSOd₆ and CF₃CO₂D) 1.63 (d, 3H), 2.71 (s, 3H), 3.13 (m, 1H), 3.25 (m, 3H), 3.62 (m, 1H), 3.80 (m, 1H), 3.84 (s, 3H), 3.98 (m, 2H), 6.05 (q, 1H), 7.43 (d, 1H), 7.53 (d, 1H), 7.63 (d, 1H), 7.80 (m, 1H), 7.90 (d, 1H), 7.98 (m, 1H), 8.12-8.05 (m, 2H), 8.68 (d, 1H), 9.03 (s, 1H); Mass spectrum: MH⁺ 515.

The tert-butyl 4-((2R)-2-{[4-({3-methoxy-4-[(6-methylpyridin-3-yl)oxy]phenyl}amino)quinazolin-5-yl]oxy}propanoyl)piperazine-1-carboxylate used as starting material was made according to procedure in Example 148 using 1-tert-butoxypiperazine as the amine; Yield: 120 mg, 66%; Mass spectrum: MH⁺ 615.

### EXAMPLES 152 TO 155

The procedure described in Example 144 was repeated using methyl (2R)-2-[(4-{[3-methyl-4-(pyridin-2-yloxy)phenyl]amino}quinazolin-5-yl)oxy]propanoate and the appropriate amine to give the descried compound.

### EXAMPLE 152

### (2R)—N,N-dimethyl-2-[(4-{[3-methyl-4-(pyridin-2-yloxy)phenyl]amino}quinazolin-5-yl)oxy]propanamide

Starting amine: saturated dimethylamine in methanol, except that the reaction was run at room temperature.

Yield: 140 mg, 79%.

NMR Spectrum: (400 MHz; CDCl₃) 1.74 (d, 3H), 2.23 (s, 3H), 3.06 (s, 3H), 3.15 (s, 3H), 5.41 (q, 1H), 6.82 (d, 1H), 6.86 (d, 1H), 6.96 (m, 1H), 7.10 (d, 1H), 7.51 (d, 1H), 7.67-7.59 (m, 2H), 7.82 (dd, 1H), 7.92 (d, 1H), 8.19 (m, 1H), 8.64 (s, 1H); Mass spectrum: MH⁺ 444.

### EXAMPLE 153

### (2R)—N-ethyl-2-[(4-{[3-methyl-4-(pyridin-2-yloxy)phenyl]amino}quinazolin-5-yl)oxy]propanamide

Starting amine: saturated ethylamine in methanol, except that the reaction was run at room temperature.

Yield: 135 mg, 78%.

NMR Spectrum: (400 MHz; CDCl₃) 1.13 (t, 3H), 1.85 (d, 3H), 2.23 (s, 3H), 3.37 (m, 2H), 4.91 (q, 1H), 6.30 (m, 1H), 6.81 (d, 1H), 6.91 (d, 1H), 6.97 (m, 1H), 7.11 (d, 1H), 7.50 (d, 1H), 7.72-7.59 (m, 4H), 8.17 (m, 1H), 8.65 (s, 1H); Mass spectrum: MH⁺ 444.

### EXAMPLE 154

### (2R)—N-(2-hydroxyethyl)-2-[(4-{[3-methyl-4-(pyridin-2-yloxy)phenyl]amino}quinazolin-5-yl)oxy]propanamide

Starting amine: ethanolamine.

Yield: 105 mg, 66%.

NMR Spectrum: (400 MHz) 1.64 (d, 3H), 2.11 (s, 3H), 3.22 (m, 2H), 3.43 (m, 2H), 4.76 (m, 1H), 5.22 (q, 1H), 7.03 (d, 2H), 7.09 (m, 2H), 7.37 (d, 1H), 7.74 (t, 1H), 7.88-7.76 (d, 3H), 8.12 (m, 1H), 8.48 (bt, 1H), 8.53 (s, 1H); Mass spectrum: MH⁺ 460.

### EXAMPLE 155

### (2R)—N-(2-hydroxyethyl)-N-methyl-2-[(4-{[3-methyl-4-(pyridin-2-yloxy)phenyl]amino}quinazolin-5-yl)oxy]propanamide

Starting amine: 2-(methylamino)ethanol.

Yield: 100 mg, 61%.

HPLC t_{R}: 2.16 min; Mass spectrum: MH⁺ 474.

The methyl (2R)-2-[(4-{[3-methyl-4-(pyridin-2-yloxy)phenyl]amino}quinazolin-5-yl)oxy]propanoate used as starting material was made from 4-chloro-5-fluoroquinazoline, 3-methyl-4-(pyridin-2-yloxy)aniline and methyl (S)-lactate according to the procedure in Example 51, starting material.

5-fluoro-N-{3-methyl-4-(pyridin-2-yloxy)phenyl}quinazolin-4-amine: Yield: 5.95 g, 78%; Mass spectrum: MH⁺ 347.

5-methoxy-N-{3-methyl-4-(pyridin-2-yloxy)phenyl}quinazolin-4-amine: Yield: 3.4 g, 97%; Mass spectrum: MH⁺ 359.

5-hydroxy-N-{3-methyl-4-(pyridin-2-yloxy)phenyl}quinazolin-4-amine: Yield: 2.97 g, 97%; Mass spectrum: MH⁺ 345.

Methyl (2R)-2-[(4-{[3-methyl-4-(pyridin-2-yloxy)phenyl]amino}quinazolin-5-yl)oxy]propanoate: Yield: 2.5 g, 71%; NMR Spectrum: (400 MHz; CDCl₃) 1.80 (d, 3H), 2.24 (s, 3H), 3.86 (s, 3H), 5.14 (q, 1H), 6.78 (d, 1H), 6.88 (d, 1H), 6.97 (m, 1H), 7.11 (d, 1H), 7.49 (d, 1H), 7.74-7.59 (m, 3H), 7.83 (d, 1H), 8.19 (m, 1H), 8.65 (s, 1H); Mass spectrum: MH⁺ 431.

### EXAMPLES 156 TO 158

The procedure described in Examples 148 to 150 was repeated using (2R)-2-[(4-{[3-methyl-4-(pyridin-2-yloxy)phenyl]amino}quinazolin-5-yl)oxy]propanoic acid and the appropriate amine to give the desired compound.

### EXAMPLE 156

### 4-{(2R)-2-[(4-{[3-methyl-4-(pyridin-2-yloxy)phenyl]amino}quinazolin-5-yl)oxy]propanoyl}piperazin-2-one

Starting amine: piperazin-2-one.

Yield: 90 mg, 50%.

HPLC t_{R}: 2.11 min; Mass spectrum: MH⁺ 499.

### EXAMPLE 157

### (2R)—N-(2-methoxyethyl)-N-methyl-2-[(4-{[3-methyl-4-pyridin-2-yloxy)phenyl]amino}quinazolin-5-yl)oxy]propanamide

Starting amine: (2-methoxyethyl)methylamine.

Yield: 95 mg, 56%.

NMR Spectrum: (400 MHz; CDCl₃) (2 rotamers) 1.73 (m, 3H), 2.23 (s, 3H), 3.21 and 3.04 (s, 3H), 3.34 and 3.32 (s, 3H), 3.8-3.4 (m, 4H), 5.70 and 5.41 (q, 1H), 6.97-6.81 (m, 3H), 7.10 (d, 1H), 7.67-7.59 (m, 3H), 7.82 and 7.80 (d, 1H), 7.92 (d, 1H), 8.20 (m, 1H), 8.64 (m, 1H); Mass spectrum: MH⁺ 488.

### EXAMPLE 158

### (3R)-1-{(2R)-2-[(4-{[3-methyl-4-(pyridin-2-yloxy)phenyl]amino}quinazolin-5-yl)oxy]propanoyl}piperidin-3-ol

Starting amine: (R)-3-hydroxypiperidine hydrochloride (except that 1 equivalent of triethylamine was added).

Yield: 110 mg, 63%.

HPLC t_{R}: 2.28 min; Mass spectrum: MH⁺ 500.

The (2R)-2-[(4-{[3-methyl-4-(pyridin-2-yloxy)phenyl]amino}quinazolin-5-yl)oxy]propanoic acid used as starting material was prepared from methyl (2R)-2-[(4-{[3-methyl-4-(pyridin-2-yloxy)phenyl]amino}quinazolin-5-yl)oxy]propanoate using the procedure described in Example 51, starting material.

Yield: 1.2 g, 89%; NMR Spectrum: (400 MHz; DMSOd₆ and CF₃CO₂D) 1.74 (d, 3H), 2.15 (s, 3H), 5.54 (q, 1H), 7.15-7.09 (m, 2H), 7.22 (d, 1H), 7.51-7.46 (m, 2H), 7.73 (m, 2H), 7.88 (m, 1H), 8.04 (t, 1H), 8.14 (m, 1H), 8.97 (s, 1H); Mass spectrum: MH⁺ 417.

### EXAMPLE 159

### 5-[(1R)-1-methyl-2-oxo-2-piperazin-1-ylethoxy]-N-[3-methyl-4-(pyridin-2-yloxy)phenyl]quinazolin-4-amine

The procedure in Example 151 was repeated using tert-butyl 4-{(2R)-2-[(4-{[3-methyl-4-(pyridin-2-yloxy)phenyl]amino}quinazolin-5-yl)oxy]propanoyl}piperazine-1-carboxylate to give the title compound as a hydrochloride salt (80 mg, 78%); NMR Spectrum: (400 MHz; DMSOd₆ and CF₃CO₂D) 1.63 (d, 3H), 2.16 (s, 3H), 3.12 (m, 1H), 3.25 (m, 3H), 3.61 (m, 1H), 3.78 (m, 1H), 3.96 (m, 2H), 6.03 (q, 1H), 7.15-7.10 (m, 2H), 7.22 (d, 1H), 7.47 (d, 1H), 7.59 (d, 1H), 7.71 (m, 1H), 7.81 (d, 1H), 7.88 (m, 1H), 8.07 (m, 1H), 8.14 (m, 1H), 8.95 (s, 1H); Mass spectrum: MH⁺ 485.

The tert-butyl 4-{(2R)-2-[(4-{[3-methyl-4-(pyridin-2-yloxy)phenyl]amino}quinazolin-5-yl)oxy]propanoyl}piperazine-1-carboxylate used as starting material was made according to procedure in Example 156 using 1-tert-butoxycarbonylpiperazine as the amine; Yield: 115 mg, 56%; Mass spectrum: MH⁺ 585.

### EXAMPLE 160

### {5-[2-methyl-4-({5-[(1R)-1-methyl-2-morpholin-4-yl-2-oxoethoxy]quinazolin-4-yl}amino)phenoxy]pyridin-2-yl}methanol

The procedure described in Examples 138 to 143 was repeated using [5-(4-amino-2-methylphenoxy)pyridin-2-yl]methanol to give the title compound (86 mg; 20%); NMR Spectrum: (400 MHz; CDCl₃) 1.74 (d, 3H), 2.29 (s, 3H), 3.42 (m, 1H), 3.57 (m, 2H), 3.74 (m, 6H), 4.74 (s, 2H), 5.42 (q, 1H), 6.84 (d, 1H), 6.96 (d, 1H), 7.26-7.18 (m, 3H), 7.57 (m, 1H), 7.79 (dd, 1H), 7.94 (d, 1H), 8.32 (d, 1H), 8.64 (s, 1H); Mass spectrum: MH⁺ 516.

The [5-(4-amino-2-methylphenoxy)pyridin-2-yl]methanol used as starting material was made from 2-fluoro-5-nitrotoluene and 3-hydroxy-6-hydroxymethylpyridine (Deady L., Australian J. Chem., 1983, 2565) according to Example 51, starting material:

[5-(2-methyl-4-nitrophenoxy)pyridin-2-yl]methanol: Yield: 6.75 g, 85%; Mass spectrum: MH⁺ 261.

[5-(4-amino-2-methylphenoxy)pyridin-2-yl]methanol: Yield: 0.44 g, 100% (except that hydrogenation was performed in ethanol with platinum oxide as a catalyst); Mass spectrum: MH⁺ 231.

### EXAMPLE 161

### N-{4-[(6-fluoropyridin-3-yl)oxy]-3-methylphenyl}-5-[(1R)-1-methyl-2-morpholin-4-yl-2-oxoethoxy]quinazolin-4-amine

The procedure described in Example 138 was repeated using 4-[(6-fluoropyridin-3-yl)oxy]-3-methylaniline to give the title compound (135 mg; 29%); NMR Spectrum: (400 MHz; CDCl₃) 1.74 (d, 3H), 2.29 (s, 3H), 3.57 (m, 2H), 3.74 (m, 6H), 5.40 (q, 1H), 6.81 (d, 1H), 6.87 (m, 1H), 6.93 (d, 1H), 7.35 (m, 1H), 7.50 (d, 1H), 7.62 (t, 1H), 7.80 (dd, 1H), 7.91 (m, 1H), 7.95 (d, 1H), 8.65 (s, 1H); Mass spectrum: MH⁺ 504.

The 4-[(6-fluoropyridin-3-yl)oxy]-3-methylaniline used as starting material was made from 2-fluoro-5-nitrotoluene and 3-hydroxy-6-fluoropyridine (Ding Y. S, Nuclear Medecine and Biology, 2000, 27, 381) according to Example 51, starting material:

2-fluoro-5-(2-methyl-4-nitrophenoxy)pyridine: Yield: 2.01 g, 96%.

4-[(6-fluoropyridin-3-yl)oxy]-3-methylaniline: Yield: 1.67 g, 95% (except that hydrogenation was performed in ethanol with platinum oxide as a catalyst); Mass spectrum: MH⁺ 219.

### EXAMPLE 162

### N-(4-{[6-(fluoromethyl)pyridin-3-yl]oxy}-3-methylphenyl)-5-[(1R)-1-methyl-2-morpholin-4-yl-2-oxoethoxy]quinazolin-4-amine

The procedure described in Example 138 was repeated using 4-{[6-(fluoromethyl)pyridin-3-yl]oxy}-3-methylaniline to give the title compound (225 mg; 47%); NMR Spectrum: (400 MHz; CDCl₃) 1.74 (d, 3H), 2.27 (s, 3H), 3.56 (m, 2H), 3.74 (m, 6H), 4.74 (s, 2H), 5.40 (m, 1H), 5.45 (d, 2H), 6.81 (d, 1H), 6.98 (d, 1H), 7.23 (m, 1H), 7.37 (d, 1H), 7.51 (d, 1H), 7.62 (m, 1H), 7.82 (dd, 1H), 7.97 (d, 1H), 8.37 (d, 1H), 8.65 (s, 1H); Mass spectrum: MH⁺ 518.

The 4-{[6-(fluoromethyl)pyridin-3-yl]oxy}-3-methylaniline used as starting material was made as follows:

(Diethylamino)sulfur trifluoride (1.56 ml, 11.8 mmol) was added to a solution of [5-(2-methyl-4-nitrophenoxy)pyridin-2-yl]methanol (2.56 g, 9.8 mmol, see Example 160) in DCM (50 ml). The mixture was stirred at room temperature for 90 minutes. Saturated aqueous ammonium chloride was added. The mixture was extracted with DCM. The organic layer was dried over magnesium sulfate. After evaporation of the solvents, the residue was purified by chromatography on silica gel (eluant: 20% to 30% ethyl acetate in petroleum ether) to give 2-(fluoromethyl)-5-(2-methyl-4-nitrophenoxy)pyridine as a pale solid (2.11 g, 82%); Mass spectrum: MH 263.

The 2-(fluoromethyl)-5-(2-methyl-4-nitrophenoxy)pyridine was converted into 4-{[6-(fluoromethyl)pyridin-3-yl]oxy}-3-methylaniline as described in Example 51 starting material, except that hydrogenation was performed in ethanol with platinum oxide as a catalyst; Yield: 760 mg, 41%; Mass spectrum: MH⁺ 233.

### EXAMPLE 163

### N-{3-methyl-4-[(1-methyl-1H-pyrazol-4-yl)oxy]phenyl}-5-[(1R)-1-methyl-2-morpholin-4-yl-2-oxoethoxy]quinazolin-4-amine

The procedure described in Example 138 was repeated using 3-methyl-4-[(1-methyl-1H-pyrazol-4-yl)oxy]aniline to give the title compound (338 mg; 84%); NMR Spectrum: (400 MHz;) 1.56 (d, 3H), 2.28 (s, 3H), 3.43-3.71 (m, 8H), 3.79 (s, 3H), 5.86 (m, 1H), 6.91 (d, 1H), 7.26 (d, 1H), 7.31 (s, 1H), 7.33 (d, 1H), 7.62 (s, 1H), 7.71-7.77 (m, 2H), 7.90 (d, 1H), 8.48 (s, 1H); Mass spectrum: MH⁺ 489.

The 3-methyl-4-[(1-methyl-1H-pyrazol-4-yl)oxy]aniline used as starting material was made as follows:

A solution of lithium bis(trimethylsilyl)amide (1M in hexane, 16.6 ml) was added dropwise to a solution of 4-t-butyldimethylsilyloxypyrazole (3.0 g, 15.1 mmol, described in Crowell, T. A. et al, PCT Int. Appl., 1999, WO 9929672, preparation 3 p30) in THF (65 ml) at room temperature. After 45 minutes, iodomethane (1.13 ml, 18.2 mmol) was added and the reaction mixture was heated at 40° C. for 3 hours. The mixture was then cooled down, neutralized with saturated ammonium chloride and extracted with ethyl acetate. After evaporation, the residue was dissolved in THF, then tetrabutylammonium fluoride (1 M in THF, 18.9 ml) and acetic acid (2.16 ml) were added and the solution stirred for 1 hour. Saturated ammonium chloride was added and the mixture extracted with ethyl acetate. Evaporation of the solvent and purification of the residue on silica gel (2 to 5% methanol in a 1:1 mixture of ethyl acetate and DCM) provided 1-methyl-4-hydroxy-1H-pyrazole (1.28 g, 86%); Mass spectrum: MH⁺ 99.

Sodium hydride (60%, 428 mg, 10.7 mmol) was added portionwise to 1-methyl-4-hydroxy-1H-pyrazole (996 mg, 10.1 mmol) in DMA (10 ml). After 15 minutes, 2-fluoro-5-nitrotoluene (1.58 g, 10.2 mmol) was added and the mixture was stirred at room temperature for 2 hours. The mixture was partitioned between water and ethyl acetate and the organic phase was dried, evaporated, and the residue purified on silica gel (40 to 70% ethyl acetate in petroleum ether) to give 1-methyl-4-(2-methyl-4-nitrophenoxy)-1H-pyrazole as a solid (2.11 g, 89%); Mass spectrum: MH⁺ 234.

1-methyl-4-(2-methyl-4-nitrophenoxy)-1H-pyrazole (2.23 g) was converted into 3-methyl-4-[(1-methyl-1H-pyrazol-4-yl)oxy]aniline as described in Example 51 starting material, except that hydrogenation was performed in ethanol with platinum oxide as a catalyst; Yield: 1.62 g, 91%; NMR Spectrum: (400 MHz) 2.08 (s, 3H), 3.72 (s, 3H), 4.78 (s, 2H), 6.35 (dd, 1H), 6.43 (d, 1H), 6.66 (d, 1H), 7.12 (s, 1H), 7.36 (s, 1H).

### EXAMPLE 164

### N-{3-chloro-4-[(1-methyl-1H-pyrazol-4-yl)oxy]phenyl}-5-[(1R)-1-methyl-2-morpholin-4-yl-2-oxoethoxy]quinazolin-4-amine

The procedure described in Example 138 was repeated using 3-chloro-4-[(1-methyl-1H-pyrazol-4-yl)oxy]aniline to give the title compound (150 mg; 52%); NMR Spectrum: (400 MHz) 1.56 (d, 3H), 3.45-3.71 (m, 8H), 3.81 (s, 3H), 5.87 (m, 1H), 7.11 (d, 1H), 7.31 (d, 1H), 7.37 (d, 1H), 7.38 (s, 1H), 7.62 (m, 1H), 7.74 (s, 1H), 7.77 (d, 1H), 7.93 (dd, 1H), 8.42 (d, 1H), 8.54 (s, 1H); Mass spectrum: MH⁺ 509.

The 3-chloro-4-[(1-methyl-1H-pyrazol-4-yl)oxy]aniline used as starting material was made as follows:

Sodium hydride (60%, 50 mg, 1.26 mmol) was added portionwise to 1-methyl-4-hydroxy-1H-pyrazole (118 mg, 1.2 mmol, described in example 104) in DMA (1 ml). After 15 minutes, 3-chloro-4-fluoro-nitrobenzene (211 mg, 1.2 mmol) was added and the reaction mixture was stirred at room temperature for 1 hour. The mixture was partitioned between water and ethyl acetate and the organic phase was dried, evaporated, and the residue purified on silica gel (40 to 70% ethyl acetate in petroleum ether) to give 1-methyl-4-(2-chloro-4-nitrophenoxy)-1H-pyrazole as a solid (248 mg, 81%); Mass spectrum: MH⁺ 254.

1-methyl-4-(2-chloro-4-nitrophenoxy)-1H-pyrazole was converted into 3-chloro-4-[(1-methyl-1H-pyrazol-4-yl)oxy]aniline as described in Example 51 starting material, except that hydrogenation was performed in ethanol with platinum oxide as a catalyst; Yield: 129 mg, 63%; Mass spectrum: MH⁺ 224.

### EXAMPLE 165

### 5-[(1R)-1-methyl-2-morpholin-4-yl-2-oxoethoxy]-N-[3-methyl-4-(pyridin-2-ylmethoxy)phenyl]quinazolin-4-amine

The procedure described in Example 138 was repeated using 3-methyl-4-(pyridin-2-ylmethoxy)aniline (AstraZeneca, PCT Int. Appl. WO2003040108, example 4.4) to give the title compound (100 mg; 30%); NMR Spectrum: (400 MHz) 1.56 (d, 3H), 2.29 (s, 3H), 3.8-3.3 (m, 8H), 5.21 (s, 2H), 5.84 (q, 1H), 7.01 (d, 1H), 7.25 (d, 1H), 7.33 (m, 2H), 7.56 (d, 1H), 7.73 (m, 2H), 7.81 (d, 1H), 7.86 (m, 1H), 8.46 (s, 1H), 8.59 (d, 1H), 10.88 (s, 1H); Mass spectrum: MH⁺ 500.

### EXAMPLES 166 TO 169

The procedure described in Example 144 was repeated using methyl (2R)-2-[(4-{[3-chloro-4-(pyridin-2-yloxy)phenyl]amino}quinazolin-5-yl)oxy]propanoate and the appropriate amine to give the desired compound.

### EXAMPLE 166

### (2R)-2-[(4-{[3-chloro-4-(pyridin-2-yloxy)phenyl]amino}quinazolin-5-yl)oxy]-N,N-dimethylpropanamide

Starting amine: saturated dimethylamine in methanol (the reaction was run at room temperature).

Yield: 131 mg, 64%.

NMR Spectrum: (400 MHz; DMSOd₆ and CF₃CO₂D) 1.61 (d, 3H), 2.96 (s, 3H), 3.14 (s, 3H), 5.99 (q, 1H), 7.17 (m, 2H), 7.47 (m, 2H), 7.66 (d, 1H), 7.91 (m, 2H), 8.07 (t, 1H), 8.14 (m, 1H), 8.21 (m, 1H), 9.02 (s, 1H); Mass spectrum: MH⁺ 464.

### EXAMPLE 167

### (2R)-2-[(4-{[3-chloro-4-(pyridin-2-yloxy)phenyl]amino}quinazolin-5-yl)oxy]-N-(2-hydroxyethyl)-N-methylpropanamide

Starting amine: 2-(methylamino)ethanol.

Yield: 180 mg, 81%.

NMR Spectrum: (400 MHz; DMSOd₆ and CF₃CO₂D) (2 rotamers) 1.63 (m, 3H), 3.17 and 2.94 (s, 3H), 3.70-3.40 (m, 4H), 6.05 and 5.96 (q, 1H), 7.17 (m, 2H), 7.51-7.45 (m, 2H), 7.64 (m, 1H), 7.91 (m, 2H), 8.06 (m, 1H), 8.25-8.15 (m, 2H), 9.01 (m, 1H); Mass spectrum: MH⁺ 494.

### EXAMPLE 168

### (2R)-2-[(4-{[3-chloro-4-(pyridin-2-yloxy)phenyl]amino}quinazolin-5-yl)oxy]-N-(2-hydroxyethyl)propanamide

Starting amine: ethanolamine.

Yield: 137 mg, 65%.

NMR Spectrum: (400 MHz; DMSOd₆ and CF₃CO₂D) 1.69 (d, 3H), 3.25 (m, 2H), 3.47 (m, 2H), 5.40 (q, 1H), 7.17 (m, 2H), 7.39 (d, 1H), 7.49 (m, 2H), 7.86 (m, 1H), 7.90 (m, 1H), 8.07 (t, 1H), 8.15 (m, 1H), 8.20 (m, 1H), 9.02 (s, 1H); Mass spectrum: MH⁺ 480.

### EXAMPLE 169

### (2R)-2-[(4-{[3-chloro-4-(pyridin-2-yloxy)phenyl]amino}quinazolin-5-yl)oxy]-N-ethyl-N-(2-hydroxyethyl)propanamide

Starting amine: 2-(ethylamino)ethanol.

Yield: 115 mg, 50%.

NMR Spectrum: (400 MHz; DMSOd₆ and CF₃CO₂D) (2 rotamers) 1.20 and 1.10 (t, 3H), 1.63 (m, 3H), 3.70-3.20 (m, 6H), 6.03 and 5.94 (q, 1H), 7.17 (m, 2H), 7.50-7.45 (m, 2H), 7.71 and 7.64 (d, 1H), 7.91 (m, 2H), 8.06 (m, 1H), 8.25-8.15 (m, 2H), 9.01 (m, 1H); Mass spectrum: MH⁺ 508.

The methyl (2R)-2-[(4-{[3-chloro-4-(pyridin-2-yloxy)phenyl]amino}quinazolin-5-yl)oxy]propanoate was prepared as follows:

Sodium hydride (0.46 g, 60% dispersion in oil, 11.4 mmol) was added portionwise to a solution of 2-hydroxypyridine (1.08 g, 11.4 mmol). The reaction mixture was stirred at room temperature for 30 minutes. 2-Chloro-1-fluoro-4-nitrobenzene (2 g, 11.4 mmol) was added. The reaction mixture was then stirred at room temperature for 18 hours. The mixture was diluted with water and extracted with ether. The organic layer washed with water and brine, and dried over magnesium sulfate. After evaporation of the solvents, the residue was purified by chromatography on silica gel (eluant: 0% to 12% ethyl acetate in petroleum ether) to give 2-(2-chloro-4-nitrophenoxy)pyridine as a solid (1.23 g, 43%). NMR Spectrum: (400 MHz; CDCl₃) 7.10 (m, 2H), 7.37 (d, 1H), 7.80 (m, 1H), 8.20-8.14 (m, 2H), 8.40 (s, 1H).

The 2-(2-chloro-4-nitrophenoxy)pyridine was converted into 3-chloro-4-(pyridin-2-yloxy)aniline as described in Example 51 starting material, except that hydrogenation was performed in ethanol with platinum oxide as a catalyst; Yield: 375 mg, 85%; Mass spectrum: MH⁺ 221.

The procedure described in Example 51 starting material was repeated with 3-chloro-4-(pyridin-2-yloxy)aniline, 4-chloro-5-fluoroquinazoline and methyl (S)-lactate to give:

N-[3-chloro-4-(pyridin-2-yloxy)phenyl]-5-fluoroquinazolin-4-amine as a beige solid (4.1 g, 96%); Mass spectrum: MH⁺ 367.

N-[3-chloro-4-(pyridin-2-yloxy)phenyl]-5-methoxyquinazolin-4-amine as a beige solid (4.67 g, 100%); Mass spectrum: MH⁺ 379.

4-{[3-chloro-4-(pyridin-2-yloxy)phenyl]amino}quinazolin-5-ol as a pale yellow solid (4.73, 95%); Mass spectrum: MH⁺ 365.

methyl (2R)-2-[(4-{[3-chloro-4-(pyridin-2-yloxy)phenyl]amino}quinazolin-5-yl)oxy]propanoate (4.65 g, 80%) (except that DTAD was used instead of DEAD); Mass spectrum: MH⁺ 451.

### EXAMPLES 170 TO 173

The procedure described in Examples 148 to 150 was repeated with (2R)-2-[(4-{[3-chloro-4-(pyridin-2-yloxy)phenyl]amino}quinazolin-5-yl)oxy]propanoic acid and the appropriate amine to give the desired compound.

### EXAMPLE 170

### N-[3-chloro-4-(pyridin-2-yloxy)phenyl]-5-[(1R)-1-methyl-2-morpholin-4-yl-2-oxoethoxy]quinazolin-4-amine

Starting amine: morpholine.

Yield: 150 mg, 52%.

NMR Spectrum: (400 MHz; DMSOd₆ and CF₃CO₂D) 1.62 (d, 3H), 3.70-3.45 (m, 8H), 6.02 (q, 1H), 7.17 (m, 2H), 7.47 (m, 2H), 7.63 (d, 1H), 7.90 (m, 2H), 8.08 (t, 1H), 8.13 (m, 1H), 8.20 (m, 1H), 9.03 (s, 1H); Mass spectrum: MH⁺ 506.

### EXAMPLE 171

### 1-{(2R)-2-[(4-{[3-chloro-4-(pyridin-2-yloxy)phenyl]amino}quinazolin-5-yl)oxy]propanoyl}piperidin-3-ol

Starting amine: 3-hydroxypiperidine.

Yield: 85 mg, 35%

HPLC t_{R}: 2.94 min; Mass spectrum: MH⁺ 520.

### EXAMPLE 172

### 4-{(2R)-2-[(4-{[3-chloro-4-(pyridin-2-yloxy)phenyl]amino}quinazolin-5-yl)oxy]propanoyl}piperazin-2-one

Starting amine: piperazin-2-one.

Yield: 150 mg, 63%

HPLC t_{R}: 2.71 min; Mass spectrum: MH⁺ 519.

### EXAMPLE 173

### (2R)-2-[(4-{[3-chloro-4-(pyridin-2-yloxy)phenyl]amino}quinazolin-5-yl)oxy]-N-(2-methoxyethyl)-N-methylpropanamide

Starting amine: (2-methoxyethyl)methylamine.

Yield: 140 mg, 61%.

NMR Spectrum: (400 MHz; DMSOd₆ and CF₃CO₂D) 2 rotamers; 1.56 (m, 3H), 3.10 and 2.88 (s, 3H), 3.18 (s, 3H), 3.72-3.45 (m, 4H), 5.97-5.88 (m, 1H), 7.11 (m, 2H), 7.42 (m, 2H), 7.58 (d, 1H), 7.83 (m, 2H), 7.99 (m, 1H), 8.16-8.05 (m, 2H), 8.95 (m, 1H); Mass spectrum: MH⁺ 508.

The (2R)-2-[(4-{[3-chloro-4-(pyridin-2-yloxy)phenyl]amino}quinazolin-5-yl)oxy]propanoic acid was prepared from methyl (2R)-2-[(4-{[3-chloro-4-(pyridin-2-yloxy)phenyl]amino}quinazolin-5-yl)oxy]propanoate using the procedure described in Example 51 starting material; Yield: 2.25 g, 79% (solid); Mass spectrum: MH⁺ 437.

### EXAMPLE 174

### N-[3-chloro-4-(pyridin-2-yloxy)phenyl]-5-[(1R)-1-methyl-2-oxo-2-piperazin-1-ylethoxy]quinazolin-4-amine

The procedure described in Example 151 was repeated using tert-butyl 4-{(2R)-2-[(4-{[3-chloro-4-(pyridin-2-yloxy)phenyl]amino}quinazolin-5-yl)oxy]propanoyl}piperazine-1-carboxylate to give the title compound as a hydrochloride salt (130 mg, 71%); NMR Spectrum: (DMSOd₆ and CF₃CO₂D) 1.62 (d, 3H), 3.11 (m, 1H), 3.26 (m, 3H), 3.61 (m, 1H), 3.78 (m, 1H), 3.95 (m, 2H), 6.03 (q, 1H), 7.17 (m, 2H), 7.50 (m, 2H), 7.60 (d, 1H), 7.90 (m, 2H), 8.19-8.08 (m, 3H), 9.03 (s, 1H); Mass spectrum: MH⁺ 505.

The tert-butyl 4-{(2R)-2-[(4-{[3-chloro-4-(pyridin-2-yloxy)phenyl]amino}quinazolin-5-yl)oxy]propanoyl}piperazine-1-carboxylate used as starting material was made according to the procedure in Example 170 using 1-tert-butoxycarbonylpiperazine as the amine; Yield: 180 mg, 65%; Mass spectrum: MH⁺ 605.

### EXAMPLE 175

### N-[3-chloro-4-(1,3-thiazol-2-yloxy)phenyl]-5-[(1R)-1-methyl-2-morpholin-4-yl-2-oxoethoxy]quinazolin-4-amine

The procedure described in Examples 138 to 143 was repeated using 3-chloro-4-(1,3-thiazol-2-yloxy)aniline to give the title compound (135 mg mg; 40%); NMR Spectrum: (400 MHz) 1.57 (d, 3H), 3.80-3.50 (m, 8H), 5.89 (q, 1H), 7.25 (m, 2H), 7.35 (d, 1H), 7.40 (d, 1H), 7.58 (d, 1H), 7.79 (t, 1H), 8.12 (dd, 1H), 8.55 (d, 1H), 8.62 (s, 1H), 11.32 (s, 1H); Mass spectrum: MH⁺ 512.

The 3-chloro-4-(1,3-thiazol-2-yloxy)aniline used as starting material was prepared from 2-chlorothiazole and 4-amino-2-chlorophenol using the procedure described in Example 138 starting material; Yield: 0.52 g, 33% (brown oil); Mass spectrum: MH⁺ 227.

### EXAMPLE 176

### (2S)—N,N-dimethyl-2-[{4-({3-methyl-4-[(6-methylpyridin-3-yl)oxy]phenyl}amino)quinazolin-5-yl]oxy}propanamide

Sodium hydride (66 mg, 1.66 mmol, 60% in oil) was added portionwise to a mixture of 5-fluoro-N-{3-methyl-4-[(6-methylpyridin-3-yl)oxy]phenyl}quinazolin-4-amine (300 mg, 0.83 mmol, described in example 51, starting material) and (2S)-2-hydroxy-N,N-dimethylpropanamide (188 mg, 2.5 mmol, Larcheveque et al, Synthesis, 1986, 60) in THF (3 ml). The mixture was stirred at 70° C. for 4 hours. After cooling, the mixture was evaporated to dryness, extracted with a mixture of water and DCM. The organic layer was dried over magnesium sulfate. After evaporation of the solvents, the residue was directly injected on an HPLC column (C18, 5 microns, 19 mm diameter, 100 mm length) of a preparative HPLC-MS system eluting with a mixture of water and acetonitrile containing 2 g/l of ammonium carbonate (gradient). After evaporation of the solvents, the mixture was dissolved in dichloromethane and evaporated under vacuum to give the title compound (220 mg, 58%) as a white foam; NMR Spectrum: (400 MHz; CDCl₃) 1.73 (d, 3H), 2.28 (s, 3H), 2.52 (s, 3H), 3.07 (s, 3H), 3.15 (s, 3H), 5.42 (q, 1H), 6.80 (d, 1H), 6.93 (d, 1H), 7.11-7.05 (m, 2H), 7.46 (d, 1H), 7.60 (t, 1H), 7.79 (dd, 1H), 7.94 (d, 1H), 8.29 (d, 1H), 8.64 (s, 1H); Mass spectrum: MH⁺ 458.

### EXAMPLE 177

### (2R)-2-{[4-({3-chloro-4-[(6-methylpyridin-3-yl)oxy]phenyl}amino)quinazolin-5-yl]oxy}-N-(2-hydroxyethyl)-N-methylpropanamide

The procedure described in Example 144 was repeated using methyl (2R)-2-{[4-({3-chloro-4-[(6-methylpyridin-3-yl)oxy]phenyl}amino)quinazolin-5-yl]oxy}propanoate and 2-(methylamino)ethanol to give the title compound (160 mg, 73%) except that the mixture was heated for 18 hours and no molecular sieves were used; NMR Spectrum: (400 MHz) (2 rotamers) 1.60 (m, 3H), 2.45 (s, 3H), 3.18 and 2.94 (s, 3H), 3.7-3.4 (m, 4H), 4.98 and 4.74 (t, 1H), 5.92 and 5.82 (m, 1H), 7.26-7.23 (m, 3H), 7.40-7.35 (m, 2H), 7.75 (m, 1H), 8.04 (m, 1H), 8.23 (s, 1H), 8.54 (m, 1H), 8.60 (s, 1H), 11.24 (br s, 1H); Mass spectrum: MH⁺ 508.

The methyl (2R)-2-{[4-({3-chloro-4-[(6-methylpyridin-3-yl)oxy]phenyl}amino)quinazolin-5-yl]oxy}propanoate used as starting material was made from 4-chloro-5-fluoroquinazoline, 3-chloro-4-[(6-methylpyridin-3-yl)oxy]aniline (see Example 125, starting material) and methyl (S)-lactate according to the procedure described in Example 51, starting material.

N-{3-chloro-4-[(6-methylpyridin-3-yl)oxy]phenyl}-5-fluoroquinazolin-4-amine: Yield: 3.48 g, 83%; Mass spectrum: MH⁺ 381.

N-{3-chloro-4-[(6-methylpyridin-3-yl)oxy]phenyl}-5-methoxyquinazolin-4-amine: Yield: 2.92 g, 98%; Mass spectrum: MH⁺ 393.

N-{3-chloro-4-[(6-methylpyridin-3-yl)oxy]phenyl}-5-hydroxyquinazolin-4-amine: Yield: 2.6 g, 93%; Mass spectrum: MH⁺ 379.

Methyl (2R)-2-{[4-({3-chloro-4-[(6-methylpyridin-3-yl)oxy]phenyl}amino)quinazolin-5-yl]oxy}propanoate: Yield: 2.65 g, 86%; NMR Spectrum: (400 MHz; CDCl₃) 1.80 (d, 3H), 2.54 (s, 3H), 3.89 (s, 3H), 5.17 (q, 1H), 6.81 (d, 1H), 7.05 (d, 1H), 7.10 (d, 1H), 7.16 (m, 1H), 7.51 (d, 1H), 7.64 (t, 1H), 7.83 (m, 1H), 8.30 (m, 2H), 8.69 (s, 1H), 10.5 (br s, 1H); Mass spectrum: MH⁺ 465.

### EXAMPLE 178

### (2R)-2-{[4-({3-chloro-4-[(6-methylpyridin-3-yl)oxy]phenyl}amino)quinazolin-5-yl]oxy}-N,N-dimethylpropanamide

The procedure described in Example 144 was repeated using methyl (2R)-2-{[4-({3-chloro-4-[(6-methylpyridin-3-yl)oxy]phenyl}amino)quinazolin-5-yl]oxy}propanoate and saturated dimethylamine in methanol (2 ml) to give the title compound (110 mg, 52%) except that the reaction was run at room temperature; NMR Spectrum: (400 MHz) 1.57 (d, 3H), 2.45 (s, 3H), 2.94 (s, 3H), 3.14 (s, 3H), 5.86 (q, 1H), 7.25 (m, 3H), 7.40-7.35 (m, 2H), 7.77 (t, 1H), 8.05 (dd, 1H), 8.23 (s, 1H), 8.54 (d, 1H), 8.60 (s, 1H), 11.27 (br s, 1H); Mass spectrum: MH⁺ 478.

### EXAMPLE 179

### N-{3-chloro-4-[(6-fluoropyridin-3-yl)oxy]phenyl}-5-[(1R)-1-methyl-2-morpholin-4-yl-2-oxoethoxy]quinazolin-4-amine

The procedure described in Example 138 was repeated using 3-chloro-4-[(6-fluoropyridin-3-yl)oxy]aniline to give the title compound (350 mg; 72%); NMR Spectrum: (400 MHz; CDCl₃) 1.73 (d, 3H), 3.56 (m, 2H), 3.74 (m, 6H), 5.41 (q, 1H), 6.83 (d, 1H), 6.89 (m, 1H), 7.07 (d, 1H), 7.38 (m, 1H), 7.50 (d, 1H), 7.63 (t, 1H), 7.94 (m, 1H), 7.99 (m, 1H), 8.43 (d, 1H), 8.69 (s, 1H); Mass spectrum: MH⁺ 524.

The 3-chloro-4-[(6-fluoropyridin-3-yl)oxy]aniline used as starting material was made from 3-chloro-4-fluoro-nitrobenzene and 3-hydroxy-6-fluoropyridine (Ding Y. S, Nuclear Medecine and Biology, 2000, 27, 381) according to Example 51, starting material:

2-fluoro-5-(2-chloro-4-nitrophenoxy)pyridine: Yield: 2.31 g, 92%.

3-chloro-4-[(6-fluoropyridin-3-yl)oxy]aniline: Yield: 1.95 g, 90% (except that hydrogenation was performed in ethanol with platinum oxide as a catalyst); Mass spectrum: MH⁺ 239.

### EXAMPLE 180

### N-[3-chloro-4-(pyrazin-2-yloxy)phenyl]-5-[(1R)-1-methyl-2-morpholin-4-yl-2-oxoethoxy]quinazolin-4-amine

The procedure described in Example 138 was repeated using 3-chloro-4-(pyrazin-2-yloxy)aniline to give the title compound (86 mg; 28%); NMR Spectrum: (400 MHz) 1.57 (d, 3H), 3.8-3.3 (m, 8H), 5.89 (q, 1H), 7.35 (d, 1H), 7.40 (d, 1H), 7.44 (d, 1H), 7.79 (t, 1H), 8.06 (dd, 1H), 8.21 (m, 1H), 8.41 (d, 1H), 8.47 (d, 1H), 8.60 (s, 1H), 8.67 (d, 1H), 11.29 (br s, 1H); Mass spectrum: MH⁺ 507.

The 3-chloro-4-(pyrazin-2-yloxy)aniline used as starting material was made as follows:

Potassium hydroxide (479 mg, 8.5 mmol) was added to a solution of 4-amino-2-chlorophenol (1.22 d, 8.5 mmol) in DMA (10 ml). The mixture was heated at 60° C. for 30 minutes. 2-Chloropyrazine (0.76 ml, 8.5 mmol) was added and the mixture was heated at 135° C. for 18 hours. After cooling and evaporation of the solvents, the residue was triturated in ether. The insoluble was filtered off. The filtrate was collected and dried over magnesium sulfate. After evaporation of the solvents, the residue was purified by chromatography on silica gel (eluant: 30% ethyl acetate in petroleum ether) to give 3-chloro-4-(pyrazin-2-yloxy)aniline (1.35 g, 52%) as a light brownish oil. Mass spectrum: MH⁺ 222.

### EXAMPLE 181

### N-[3-chloro-4-(1,3-thiazol-5-yloxy)phenyl]-5-[(1R)-1-methyl-2-morpholin-4-yl-2-oxoethoxy]quinazolin-4-amine

The procedure described in Example 138 was repeated using 3-chloro-4-(1,3-thiazol-5-yloxy)aniline to give the title compound (220 mg; 43%); NMR Spectrum: (400 MHz; CDCl₃) 1.72 (d, 3H), 3.56 (m, 2H), 3.74 (m, 6H), 5.40 (q, 1H), 6.82 (d, 1H), 7.14 (d, 1H), 7.46 (s, 1H), 7.50 (d, 1H), 7.62 (t, 1H), 7.95 (dd, 1H), 8.40 (m, 2H), 8.68 (s, 1H); Mass spectrum: MH⁺ 512.

The 3-chloro-4-(1,3-thiazol-5-yloxy)aniline used as starting material was made as follows:

Sodium hydride (20.4 g, 511 mmol, 60% in oil) was added portionwise to a solution of 2-chlorophenol (64.7 g, 503 mmol) in THF (600 ml) while cooling. The mixture was stirred at for 30 minutes at room temperature, then was heated at 70° C. and 2-amino-5-bromothiazole (30 g, 168 mmol, free base) was added. The mixture was heated at 80° C. for 2 hours. After cooling, the solvents were evaporated. The residue was partitioned in a mixture of ethyl acetate and water. The organic layer was dried over magnesium sulfate. After evaporation of the solvents, the residue was purified by chromatography on silica gel (eluant: gradient of ethyl acetate in petroleum ether) to give 5-(2-chlorophenoxy)-1,3-thiazol-2-amine (11.89 g, 31%) as a light brownish solid. NMR Spectrum: (400 MHz; CDCl₃) 4.92 (m, 2H), 6.79 (s, 1H), 7.03 (m, 1H), 7.11 (d, 1H), 7.20 (m, 1H), 7.40 (dd, 1H).

An aqueous solution of sodium nitrite (5.6 g, 78.7 mmol) in water (32 ml) was added dropwise over 45 minutes to a suspension of 5-(2-chlorophenoxy)-1,3-thiazol-2-amine (11.89 g, 52.5 mmol) in 84% phosphoric acid (107 ml) and 69% nitric acid (16.8 ml) cooled at −10° C. The mixture was stirred at −10° C. for one hour. Hypophosphorous acid (32.6 ml, 50% aqueous solution, 247 mmol) was added dropwise at −10° C. The mixture was stirred at −10° C. for 2 hours and at room temperature for 18 hours. The mixture was cooled to −50° C. and a concentrated solution of aqueous sodium hydroxide was added dropwise until pH 7 while maintaining the temperature of the mixture below 0° C. The mixture was diluted with water and extracted with DCM. The organic layer was dried over magnesium sulfate. After evaporation of the solvents, the residue was purified by chromatography on silica gel (eluant: 20-30% ethyl acetate in petroleum ether) to give 5-(2-chlorophenoxy)-1,3-thiazole (4.17 g, 38%) as an orange oil; NMR Spectrum: (400 MHz; CDCl₃) 7.14-7.08 (m, 2H), 7.23 (m, 1H), 7.45 (m, 2H), 8.44 (s, 1H).

90% Nitric acid (10.57 ml, 151 mmol) was added dropwise to a solution of 5-(2-chlorophenoxy)-1,3-thiazole (4 g, 18.90 mmol) in DCM (5 ml) at 0° C. The mixture was stirred at room temperature for 17 hours. Ice was added and the pH of the solution was adjusted to 7 by addition of sodium carbonate. The mixture was extracted with ethyl acetate. The organic layer was dried over magnesium sulfate. After evaporation of the solvents, the residue was purified by chromatography on silica gel (eluant: 30-50% ethyl acetate in petroleum ether) to give 5-(2-chloro-4-nitrophenoxy)-1,3-thiazole as a pale solid (4.11 g, 85%); Mass spectrum: MH⁺ 257.

5-(2-Chloro-4-nitrophenoxy)-1,3-thiazole was converted into 3-chloro-4-(1,3-thiazol-5-yloxy)aniline by hydrogenation according to the procedure described in Example 51, starting material except that it was performed in methanol with platinum oxide as a catalyst; Yield: 0.86 g, 90%; Mass spectrum: MH⁺ 227.

### EXAMPLE 182

Pharmaceutical Compositions

The following illustrates representative pharmaceutical dosage forms of the invention as defined herein (the active ingredient being termed “Compound X”) which may be prepared, for therapeutic or prophylactic use in humans:

| (a) | Tablet I | mg/tablet |
|---|---|---|
| | Compound X | 100 |
| | Lactose Ph.Eur | 182.75 |
| | Croscarmellose sodium | 12.0 |
| | Maize starch paste (5% w/v paste) | 2.25 |
| | Magnesium stearate | 3.0 |
| (b) | Injection I | (50 mg/ml) |
| | Compound X | 5.0% w/v |
| | 1M Sodium hydroxide solution | 15.0% v/v |
| | 0.1M Hydrochloric acid (to adjust pH to 7.6) | |
| | Polyethylene glycol 400 | 4.5% w/v |
| | Water for injection to 100%. | |

The above compositions may be prepared by conventional procedures well known in the pharmaceutical art. For example, Tablet I may be prepared by blending the components together and compressing the mixture into a tablet.

1. A quinazoline derivative of the formula I: 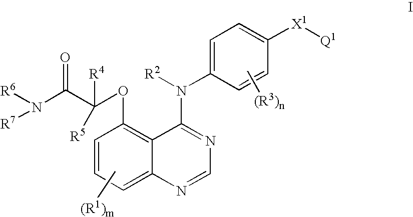
wherein:
m is 0, 1 or 2;
each R¹, which may be the same or different, is selected from hydroxy, (1-6C)alkoxy, (3-7C)cycloalkyl-oxy and (3-7C)cycloalkyl-(1-6C)alkoxy,
and wherein any CH₂ or CH₃ group within an R¹ substituent optionally bears on each said CH₂ or CH₃ group one or more substituents independently selected from halogeno, (1-6C)alkyl, hydroxy and (1-6C)alkoxy;
R² is hydrogen or (1-4C)alkyl;
n is 0, 1, 2, 3 or 4;
each R³, which may be the same or different, is selected from halogeno, cyano, (1-4C)alkyl, trifluoromethyl, (1-4C)alkoxy, (2-4C)alkenyl and (2-4C)alkynyl;
X¹ is selected from O, S, SO, SO₂, N(R¹³), CH(OR¹³), CON(R¹³), N(R¹³)CO, SO₂N(R¹³), N(R¹³)SO₂, OC(R¹³)₂, C(R¹³)₂O, SC(R¹³)₂, C(R¹³)₂S, CO, C(R¹³)₂N(R¹³) and N(R¹³)C(R¹³)₂, wherein each R¹³, which may be the same or different, is hydrogen or (1-6C)alkyl;
Q¹ is aryl or heteroaryl,
and wherein Q¹ optionally bears one or more substituents, which may be the same or different, selected from halogeno, cyano, nitro, hydroxy, amino, carboxy, carbamoyl, sulfamoyl, formyl, mercapto, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (3-6C)alkenoyl, (3-6C)alkynoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkylsulfonylamino, N-(1-6C)alkyl-(1-6C)alkylsulfonylamino, and a group of the formula:
—X²—R⁸
wherein X² is a direct bond or is selected from O, CO and N(R⁹), wherein R⁹ is hydrogen or (1-6C)alkyl, and R⁸ is selected from halogeno-(1-6C)alkyl, hydroxy-(1-6C)alkyl, carboxy-(1-6C)alkyl, (1-6C)alkoxy-(1-6C)alkyl, cyano-(1-6C)alkyl, amino-(1-6C)alkyl, N-(1-6C)alkylamino-(1-6C)alkyl, N,N-di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, (1-6C)alkoxycarbonylamino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, (1-6C)alkylthio-(1-6C)alkyl, (1-6C)alkylsulfinyl-(1-6C)alkyl, (1-6C)alkylsulfonyl-(1-6C)alkyl, sulfamoyl(1-6C)alkyl, N-(1-6C)alkylsulfamoyl(1-6C)alkyl, N,N-di-(1-6C)alkylsulfamoyl(1-6C)alkyl, (2-6C)alkanoyl-(1-6C)alkyl, (2-6C)alkanoyloxy-(1-6C)alkyl and (1-6C)alkoxycarbonyl-(1-6C)alkyl,
and wherein any CH₂ or CH₃ group within —X¹-Q¹ optionally bears on each said CH₂ or CH₃ group one or more substituents independently selected from halogeno, (1-6C)alkyl, hydroxy, cyano, amino, (1-4C)alkoxy, (1-4C)alkylamino and di-[(1-4C)alkylamino];
R⁴ and R⁵, which may be the same or different, are selected from hydrogen and (1-6C)alkyl, or
R⁴ and R⁵ together with the carbon atom to which they are attached form a (3-7C)cycloalkyl ring,
and wherein any CH₂ or CH₃ group within any of R⁴ and R⁵ optionally bears on each said CH₂ or CH₃ group one or more substituents independently selected from halogeno, hydroxy, cyano, (1-6C)alkoxy, amino, (2-6C)alkanoyl, (1-6C)alkylamino and di-[(1-6C)alkylamino];
R⁶ and R⁷, which may be the same or different, are selected from hydrogen, (1-6C)alkyl, (2-6C)alkenyl, (2-6C)alkynyl, (3-7C)cycloalkyl, (3-7C)cycloalkyl-(1-6C)alkyl, (3-7C)cycloalkenyl, (3-7C)cycloalkenyl-(1-6C)alkyl, heterocyclyl and heterocyclyl-(1-6C)alkyl, or
R⁶ and R⁷ together with the nitrogen atom to which they are attached form a saturated 4, 5, 6 or 7 membered heterocyclic ring which optionally contains one or more additional heteroatoms independently selected from oxygen, S, SO, SO₂ and NR¹⁰, wherein R¹⁰ is selected from hydrogen, (1-6C)alkyl, (2-6C)alkenyl, (2-6C)alkynyl, (1-6C)alkylsulfonyl, (1-6C)alkylcarbonyl and (1-6C)alkoxycarbonyl,
and wherein any heterocyclyl group within an R⁶ or an R⁷ substituent or any heterocyclic ring formed by R⁶, R⁷ and the nitrogen atom to which they are attached optionally bears one or more substituents, which may be the same or different, selected from halogeno, trifluoromethyl, cyano, nitro, hydroxy, amino, formyl, mercapto, (1-6C)alkyl, (2-6C)alkenyl, (2-6C)alkynyl, hydroxy-(1-6C)alkyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X³—R¹¹
wherein X³ is a direct bond or is selected from O, CO, SO₂ and N(R¹²), wherein R¹² is hydrogen or (1-4C)alkyl, and R¹¹ is selected from halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl,
and wherein any heterocyclyl group within an R⁶ or an R⁷ substituent or any heterocyclic ring formed by R⁶, R⁷ and the nitrogen atom to which they are attached optionally bears 1 or 2 oxo or thioxo substituents,
and wherein any CH₂ or CH₃ group within an R⁶ or an R⁷ substituent, other than a CH₂ group within a heterocyclyl group or heterocyclic ring, optionally bears on each said CH₂ or CH₃ group one or more substituents independently selected from halogeno, (1-6C)alkyl, hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkylsulfonylamino and N-(1-6C)alkyl-(1-6C)alkylsulfonylamino;
or a pharmaceutically acceptable salt thereof.

2. A quinazoline derivative according to claim 1, wherein:
m is 0, 1 or 2;
each R¹, which may be the same or different, is selected from hydroxy, (1-6C)alkoxy, (3-7C)cycloalkyl-oxy and (3-7C)cycloalkyl-(1-6C)alkoxy,
and wherein any CH₂ or CH₃ group within an R¹ substituent optionally bears on each said CH₂ or CH₃ group one or more substituents independently selected from halogeno, (1-6C)alkyl, hydroxy and (1-6C)alkoxy;
R² is hydrogen or (1-4C)alkyl;
n is 0, 1, 2, 3 or 4;
each R³, which may be the same or different, is selected from halogeno, (1-4C)alkyl, trifluoromethyl, (1-4C)alkoxy, (2-4C)alkenyl and (2-4C)alkynyl;
X¹ is selected from O, S, SO, SO₂, N(R¹³), CH(OR¹³), CON(R¹³), N(R¹³)CO, SO₂N(R¹³), N(R¹³)SO₂, OC(R¹³)₂, C(R¹³)₂O, SC(R¹³)₂, C(R¹³)₂S, CO, C(R¹³)₂N(R¹³) and N(R¹³)C(R¹³)₂, wherein each R¹³, which may be the same or different, is hydrogen or (1-6C)alkyl;
Q¹ is aryl or heteroaryl,
and wherein Q¹ optionally bears one or more substituents, which may be the same or different, selected from halogeno, cyano, nitro, hydroxy, amino, carboxy, carbamoyl, sulfamoyl, formyl, mercapto, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (3-6C)alkenoyl, (3-6C)alkynoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkylsulfonylamino, N-(1-6C)alkyl-(1-6C)alkylsulfonylamino, and a group of the formula:
—X²—R⁸
wherein X² is a direct bond or is selected from O, CO and N(R⁹), wherein R⁹ is hydrogen or (1-6C)alkyl, and R⁸ is selected from halogeno-(1-6C)alkyl, hydroxy-(1-6C)alkyl, carboxy-(1-6C)alkyl, (1-6C)alkoxy-(1-6C)alkyl, cyano-(1-6C)alkyl, amino-(1-6C)alkyl, N-(1-6C)alkylamino-(1-6C)alkyl, N,N-di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, (1-6C)alkoxycarbonylamino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, (1-6C)alkylthio-(1-6C)alkyl, (1-6C)alkylsulfinyl-(1-6C)alkyl, (1-6C)alkylsulfonyl-(1-6C)alkyl sulfamoyl(1-6C)alkyl, N-(1-6C)alkylsulfamoyl(1-6C)alkyl, N,N-di-(1-6C)alkylsulfamoyl(1-6C)alkyl, (2-6C)alkanoyl-(1-6C)alkyl, (2-6C)alkanoyloxy-(1-6C)alkyl or (1-6C)alkoxycarbonyl-(1-6C)alkyl,
and wherein any CH₂ or CH₃ group within —X¹-Q¹ optionally bears on each said CH₂ or CH₃ group one or more substituents independently selected from halogeno, (1-6C)alkyl, hydroxy, cyano, amino, (1-4C)alkoxy, (1-4C)alkylamino and di-[(1-4C)alkylamino];
R⁴ and R⁵, which may be the same or different, are selected from hydrogen and (1-6C)alkyl, or
R⁴ and R⁵ together with the carbon atom to which they are attached form a (3-7C)cycloalkyl ring,
and wherein any CH₂ or CH₃ group within any of R⁴ and R⁵ optionally bears on each said CH₂ or CH₃ group one or more substituents independently selected from halogeno, hydroxy, cyano, (1-6C)alkoxy, amino, (2-6C)alkanoyl, (1-6C)alkylamino and di-[(1-6C)alkylamino];
R⁶ and R⁷, which may be the same or different, are selected from hydrogen, (1-6C)alkyl, (2-6C)alkenyl, (2-6C)alkynyl, (3-7C)cycloalkyl, (3-7C)cycloalkyl-(1-6C)alkyl, (3-7C)cycloalkenyl, (3-7C)cycloalkenyl-(1-6C)alkyl, heterocyclyl and heterocyclyl-(1-6C)alkyl, or
R⁶ and R⁷ together with the nitrogen atom to which they are attached form a saturated 5 or 6 membered heterocyclic ring which optionally contains one or more additional heteroatoms independently selected from oxygen and NR¹⁰, wherein R¹⁰ is selected from hydrogen, (1-6C)alkyl, (2-6C)alkenyl, (2-6C)alkynyl, (1-6C)alkylsulfonyl and (1-6C)alkylcarbonyl;
and wherein any heterocyclyl group within an R⁶ or an R⁷ substituent or any heterocyclic ring formed by R⁶, R⁷ and the nitrogen atom to which they are attached optionally bears one or more substituents, which may be the same or different, selected from halogeno, trifluoromethyl, cyano, nitro, hydroxy, amino, formyl, mercapto, (1-6C)alkyl, (2-6C)alkenyl, (2-6C)alkynyl, hydroxy-(1-6C)alkyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X³—R¹¹
wherein X³ is a direct bond or is selected from O, CO, SO₂ and N(R¹²), wherein R¹² is hydrogen or (1-4C)alkyl, and R¹¹ is selected from halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl,
and wherein any heterocyclyl group within an R⁶ or an R⁷ substituent or any heterocyclic ring formed by R⁶, R⁷ and the nitrogen atom to which they are attached optionally bears 1 or 2 oxo or thioxo substituents;
and wherein any CH₂ or CH₃ group within an R⁶ or an R⁷ substituent, other than a CH₂ group within a heterocyclyl group or heterocyclic ring, optionally bears on each said CH₂ or CH₃ group one or more substituents independently selected from halogeno, (1-6C)alkyl, hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkylsulfonylamino and N-(1-6C)alkyl-(1-6C)alkylsulfonylamino;
or a pharmaceutically acceptable salt thereof.

3. A quinazoline derivative according to any one of the preceding claims, wherein m is 0 or 1.

4. A quinazoline derivative according to claim 3, wherein m is 0.

5. A quinazoline derivative according to any one of the preceding claims, wherein R² is hydrogen or methyl.

6. A quinazoline derivative according to claim 5, wherein R² is hydrogen.

7. A quinazoline derivative according to any one of the preceding claims, wherein n is 0 or 1.

8. A quinazoline derivative according to claim 7, wherein n is 1.

9. A quinazoline derivative according to any one of the preceding claims, wherein X¹ is selected from O, S, OC(R¹³)₂, SC(R¹³)₂, SO, SO₂, N(R¹³), CO and N(R¹³)C(R¹³)₂, wherein each R¹³, which may be the same or different, is hydrogen or (1-6C)alkyl.

10. A quinazoline derivative according to claim 9, wherein X¹ is selected from O and OC(R¹³)₂, wherein each R¹³, which may be the same or different, is hydrogen or (1-4C)alkyl.

11. A quinazoline derivative according to any one of the preceding claims, wherein Q¹ is phenyl or a 5 or 6 membered monocyclic heteroaryl ring, which ring contains 1, 2 or 3 heteroatoms independently selected from oxygen, nitrogen and sulfur, and wherein Q¹ optionally bears one or more substituents, which may be the same or different, as defined in claim 1.

12. A quinazoline derivative according to any one of the preceding claims, wherein Q¹ is selected from pyridinyl, pyrimidinyl, pyrazinyl, 1,3-thiazolyl, 1H-pyrazolyl and pyridazinyl, and wherein Q¹ optionally bears one or more substituents, which may be the same or different, as defined in claim 1.

13. A quinazoline derivative according to any one of the preceding claims, wherein Q¹ is pyridinyl, and wherein Q¹ optionally bears one or more substituents, which may be the same or different, as defined in claim 1.

14. A quinazoline derivative according to any one of the preceding claims, wherein R⁴ and R⁵, which may be the same or different, are selected from hydrogen and (1-3C)alkyl, and wherein any CH₂ or CH₃ group within any of R⁴ and R⁵ optionally bears on each said CH₂ or CH₃ group one or more substituents independently selected from halogeno, hydroxy, cyano, (1-6C)alkoxy and (2-6C)alkanoyl.

15. A quinazoline derivative according to any one of the preceding claims, wherein R⁴ is hydrogen and R⁵ is methyl.

16. A quinazoline derivative according to any one of the preceding claims, wherein R⁶ and R⁷, which may be the same or different, are selected from hydrogen, (1-6C)alkyl, (2-6C)alkenyl, (2-6C)alkynyl, (3-7C)cycloalkyl, heterocyclyl and heterocyclyl-(1-6C)alkyl, or
R⁶ and R⁷ together with the nitrogen atom to which they are attached form a saturated 4, 5 or 6 membered heterocyclic ring which optionally contains one or more additional heteroatoms independently selected from oxygen, S, SO, SO₂ and N(R¹⁰), wherein R¹⁰ is selected from hydrogen, (1-6C)alkyl, (2-6C)alkenyl, (2-6C)alkynyl, (1-6C)alkylsulfonyl, (1-6C)alkylcarbonyl and (1-6C)alkoxycarbonyl,
and wherein any heterocyclyl group within an R⁶ or an R⁷ substituent or any heterocyclic ring formed by R⁶, R⁷ and the nitrogen atom to which they are attached optionally bears one or more substituents, which may be the same or different, as defined in claim 1,
and wherein any heterocyclyl group within an R⁶ or an R⁷ substituent or any heterocyclic ring formed by R⁶, R⁷ and the nitrogen atom to which they are attached optionally bears 1 or 2 oxo or thioxo substituents,
and wherein any CH₂ or CH₃ group within an R⁶ or an R⁷ substituent, other than a CH₂ group within a heterocyclyl group or a heterocyclic ring, optionally bears on each said CH₂ or CH₃ group one or more substituents as defined in claim 1.

17. A quinazoline derivative according to any one of the preceding claims, wherein R⁶ and R⁷, which may be the same or different, are selected from hydrogen, methyl, ethyl, propyl, isopropyl, butyl, isobutyl, tert-butyl, vinyl, isopropenyl, allyl, but-2-enyl, ethynyl, 2-propynyl, butynyl, cyclopropyl, cyclobutyl, cyclopentyl, cyclohexyl, azetidinyl, pyrrolinyl, pyrrolidinyl, piperidinyl, homopiperidinyl, homopiperazinyl, dihydropyridinyl, tetrahydropyridinyl, dihydropyrimidinyl, tetrahydropyrimidinyl, tetrahydrothienyl, tetrahydrothiopyranyl, tetrahydrofuranyl, tetrahydropyranyl, cyclopropylmethyl, cyclobutylmethyl, cyclopentylmethyl, cyclohexylmethyl, 2-cyclopropylethyl, 2-cyclobutylethyl, 2-cyclopentylethyl, 2-cyclohexylethyl, azetidinylmethyl, pyrrolinylmethyl, pyrrolidinylmethyl, morpholinylmethyl, piperidinylmethyl, homopiperidinylmethyl, piperazinylmethyl, homopiperazinylmethyl, dihydropyridinylmethyl, tetrahydropyridinylmethyl, dihydropyrimidinylmethyl, tetrahydropyrimidinylmethyl, tetrahydrothienylmethyl, tetrahydrothiopyranylmethyl, thiomorpholinylmethyl, tetrahydrofuranylmethyl, tetrahydropyranylmethyl, 2-(azetidinyl)ethyl, 2-(pyrrolinyl)ethyl, 2-(pyrrolidinyl)ethyl, 2-(morpholinyl)ethyl, 2-(piperidinyl)ethyl, 2-(homopiperidinyl)ethyl, 2-(piperazinyl)ethyl, 2-(homopiperazinyl)ethyl, 2-(dihydropyridinyl)ethyl, 2-(tetrahydropyridinyl)ethyl, 2-(dihydropyrimidinyl)ethyl, 2-(tetrahydropyrimidinyl)ethyl, 2-(tetrahydrothienyl)ethyl, 2-(tetrahydrothiopyranyl)ethyl, 2-(thiomorpholinyl)ethyl, 2-(tetrahydrofuranyl)ethyl, 2-(tetrahydropyranyl)ethyl, 3-(piperazinyl)propyl and 3-(pyrrolidinyl)propyl, or
R⁶ and R⁷ together with the nitrogen atom to which they are attached form a heterocyclic ring selected from azetidin-1-yl, pyrrolidin-1-yl, pyrazolidin-1-yl, piperidin-1-yl, morpholin-4-yl and piperazin-1-yl,
and wherein when R⁶ and R⁷ together with the nitrogen atom to which they are attached form a heterocyclic ring selected from pyrazolidin-1-yl and piperazin-1-yl, any nitrogen atom apart from the NR⁶R⁷ nitrogen atom is substituted by R¹⁰, wherein R¹⁰ is selected from hydrogen, (1-4C)alkyl and (1-4C)alkoxycarbonyl,
and wherein any heterocyclyl group within an R⁶ or an R⁷ substituent or any heterocyclic ring formed by R⁶, R⁷ and the nitrogen atom to which they are attached optionally bears one or more substituents, which may be the same or different, selected from fluoro, chloro, bromo, oxo, hydroxy, hydroxymethyl, methyl, ethyl, propyl, butyl, isopropyl, isobutyl, trifluoromethyl, vinyl, isopropenyl, allyl, but-2-enyl, ethynyl, 2-propynyl, butynyl, methoxy, ethoxy, propoxy, isopropoxy, trifluoromethoxy, acetyl, propionyl, methoxymethyl, ethoxymethyl, 2-hydroxyethyl, 2-methoxyethyl, butoxycarbonyl and 2-ethoxyethyl,
and wherein any CH₂ or CH₃ group within an R⁶ or an R⁷ substituent, other than a CH₂ group within a heterocyclyl group or a heterocyclic ring, optionally bears on each said CH₂ or CH₃ group one or more substituents independently selected from fluoro, chloro, bromo, methyl, ethyl, propyl, isopropyl, hydroxy, amino, methoxy, ethoxy, methylamino, ethylamino, di-methylamino, di-ethylamino, N-methyl-N-ethylamino, acetylamino, methylsulfonyl, methylthio and ethylsulfonyl.

18. A quinazoline derivative according to any one of the preceding claims, wherein R⁶ and R⁷, which may be the same or different, are selected from hydrogen, methyl, ethyl, propyl, isopropyl, tert-butyl, allyl, 2-propynyl, cyclopropyl, cyclobutyl, piperidinyl, 2-(pyrrolidinyl)ethyl, 2-(morpholinyl)ethyl, 3-(piperazinyl)propyl and 3-(pyrrolidinyl)propyl, or
R⁶ and R⁷ together with the nitrogen atom to which they are attached form a heterocyclic ring selected from azetidin-1-yl, pyrrolidin-1-yl, piperidin-1-yl, morpholin-4-yl and piperazin-1-yl,
and wherein when R⁶ and R⁷ together with the nitrogen atom to which they are attached form a heterocyclic ring that is piperazin-1-yl, any nitrogen atom apart from the NR⁶R⁷ nitrogen atom is substituted by R¹⁰, wherein R¹⁰ is selected from hydrogen, (1-4C)alkyl and (1-4C)alkoxycarbonyl,
and wherein any heterocyclyl group within an R⁶ or an R⁷ substituent or any heterocyclic ring formed by R⁶, R⁷ and the nitrogen atom to which they are attached optionally bears one or more substituents, which may be the same or different, selected from oxo, hydroxy, hydroxymethyl, methyl, ethyl and butoxycarbonyl, and wherein any CH₂ or CH₃ group within an R⁶ or an R⁷ substituent, other than a CH₂ group within a heterocyclyl group or a heterocyclic ring, optionally bears on each said CH₂ or CH₃ group one or more substituents independently selected from hydroxy, methoxy, di-methylamino, di-ethylamino, acetylamino, methylsulfonyl and methylthio.

19. A quinazoline derivative according to any one of the preceding claims, wherein R⁶ and R⁷ are selected from (1-4C)alkyl, and wherein any CH₂ or CH₃ group within an R⁶ or an R⁷ (1-4C)alkyl substituent optionally bears on each said CH₂ or CH₃ group one or more hydroxy substituents.

20. A quinazoline derivative selected from one or more of the following:
2-[(4-{[3-chloro-4-(pyridin-2-ylmethoxy)phenyl]amino}quinazolin-5-yl)oxy]acetamide;
2-{4-[3-chloro-4-(pyridin-2-ylmethoxy)-phenylamino]-quinazolin-5-yloxy}-N-(2-methanesulfonyl-ethyl)-acetamide;
2-{4-[3-chloro-4-(pyridin-2-ylmethoxy)-phenylamino]-quinazolin-5-yloxy}-N-cyclopropyl-acetamide;
2-{4-[3-chloro-4-(pyridin-2-ylmethoxy)-phenylamino]-quinazolin-5-yloxy}-N-cyclobutyl-acetamide;
2-{4-[3-chloro-4-(pyridin-2-ylmethoxy)-phenylamino]-quinazolin-5-yloxy}-N-(2-methoxy-ethyl)-acetamide;
2-{4-[3-chloro-4-(pyridin-2-ylmethoxy)-phenylamino]-quinazolin-5-yloxy}-N-ethyl-acetamide;
N-allyl-2-{4-[3-chloro-4-(pyridin-2-ylmethoxy)-phenylamino]-quinazolin-5-yloxy}-acetamide;
2-{4-[3-chloro-4-(pyridin-2-ylmethoxy)-phenylamino]-quinazolin-5-yloxy}-N-ethyl-N-methyl-acetamide;
2-[(4-{[3-chloro-4-(pyridin-2-ylmethoxy)phenyl]amino}quinazolin-5-yl)oxy]-N-(2-morpholin-4-ylethyl)acetamide;
2-{4-[3-chloro-4-(pyridin-2-ylmethoxy)-phenylamino]-quinazolin-5-yloxy}-N-methyl-N-prop-2-ynyl-acetamide;
2-[(4-{[3-chloro-4-(pyridin-2-ylmethoxy)phenyl]amino}quinazolin-5-yl)oxy]-N-(2-hydroxyethyl)-N-methylacetamide;
2-{4-[3-chloro-4-(pyridin-2-ylethoxy)-phenylamino]-quinazolin-5-yloxy}-N-(2-methanesulfonyl-ethyl)-N-methyl-acetamide;
2-{4-[3-chloro-4-(pyridin-2-ylmethoxy)-phenylamino]-quinazolin-5-yloxy}-N-methyl-N-(1-methyl-piperidin-4-yl)-acetamide;
2-{4-[3-chloro-4-(pyridin-2-ylmethoxy)-phenylamino]-quinazolin-5-yloxy}-N-isopropyl-N-methyl-acetamide;
2-{4-[3-chloro-4-(pyridin-2-ylmethoxy)-phenylamino]-quinazolin-5-yloxy}-N-(2-dimethylamino-ethyl)-N-methyl-acetamide;
N-[3-chloro-4-(pyridin-2-ylmethoxy)phenyl]-5-(2-morpholin-4-yl-2-oxoethoxy)quinazolin-4-amine;
N-[3-chloro-4-(pyridin-2-ylmethoxy)phenyl]-5-(2-oxo-2-piperazin-1-ylethoxy)quinazolin-4-amine;
N-[3-chloro-4-(pyridin-2-ylmethoxy)phenyl]-5-[2-(4-methylpiperazin-1-yl)-2-oxoethoxy]quinazolin-4-amine;
(2R)-2-[(4-{[3-chloro-4-(pyridin-2-ylmethoxy)phenyl]amino}quinazolin-5-yl)oxy]propanamide;
(2R)-2-[(4-{[3-chloro-4-(pyridin-2-ylmethoxy)phenyl]amino}quinazolin-5-yl)oxy]-N-methylpropanamide;
(2R)-2-[(4-{[3-chloro-4-(pyridin-2-ylmethoxy)phenyl]amino}quinazolin-5-yl)oxy]-N,N-dimethylpropanamide;
(2R)-2-[(4-{[3-chloro-4-(pyridin-2-ylmethoxy)phenyl]amino}quinazolin-5-yl)oxy]-N-(2-hydroxyethyl)-N-methylpropanamide;
N-[3-chloro-4-(pyridin-2-ylmethoxy)phenyl]-5-[(1R)-1-methyl-2-oxo-2-pyrrolidin-1-ylethoxy]quinazolin-4-amine;
(3R)-1-{(2R)-2-[(4-{[3-chloro-4-(pyridin-2-ylmethoxy)phenyl]amino}quinazolin-5-yl)oxy]propanoyl}pyrrolidin-3-ol;
((2S)-1-{(2R)-2-[(4-{[3-chloro-4-(pyridin-2-ylmethoxy)phenyl]amino}quinazolin-5-yl)oxy]propanoyl}pyrrolidin-2-yl)methanol;
((2R)-1-{(2R)-2-[(4-{[3-chloro-4-(pyridin-2-ylmethoxy)phenyl]amino}quinazolin-5-yl)oxy]propanoyl}pyrrolidin-2-yl)methanol;
N-[3-chloro-4-(pyridin-2-ylmethoxy)phenyl]-5-[(1R)-1-methyl-2-morpholin-4-yl-2-oxoethoxy]quinazolin-4-amine;
(2S)-2-[(4-{[3-chloro-4-(pyridin-2-ylmethoxy)phenyl]amino}quinazolin-5-yl)oxy]-propanamide;
(2S)-2-[(4-{[3-chloro-4-(pyridin-2-ylmethoxy)phenyl]amino}quinazolin-5-yl)oxy]-N-methylpropanamide;
(2S)-2-[(4-{[3-chloro-4-(pyridin-2-ylmethoxy)phenyl]amino}quinazolin-5-yl)oxy]-N,N-dimethylpropanamide;
(2S)-2-[(4-{[3-chloro-4-(pyridin-2-ylmethoxy)phenyl]amino}quinazolin-5-yl)oxy]-N-(2-hydroxyethyl)-N-methylpropanamide;
(3R)-1-{(2S)-2-[(4-{[3-chloro-4-(pyridin-2-ylmethoxy)phenyl]amino}quinazolin-5-yl)oxy]propanoyl}pyrrolidin-3-ol;
(3S)-1-{(2S)-2-[(4-{[3-chloro-4-(pyridin-2-ylmethoxy)phenyl]amino}quinazolin-5-yl)oxy]propanoyl}pyrrolidin-3-ol;
((2S)-1-{(2S)-2-[(4-{[3-chloro-4-(pyridin-2-ylmethoxy)phenyl]amino}quinazolin-5-yl)oxy]propanoyl}pyrrolidin-2-yl)methanol;
(2R)-2-[(4-{[3-chloro-4-(pyridin-2-ylmethoxy)phenyl]amino}quinazolin-5-yl)oxy]-4-hydroxy-N-methylbutanamide;
(2R)-2-[(4-{[3-chloro-4-(pyridin-2-ylmethoxy)phenyl]amino}quinazolin-5-yl)oxy]-4-hydroxy-N-(2-hydroxy-1,1-dimethylethyl)butanamide;
(2R)-2-[(4-{[3-chloro-4-(pyridin-2-ylmethoxy)phenyl]amino}quinazolin-5-yl)oxy]-4-hydroxy-N,N-dimethylbutanamide;
(2R)-2-[(4-{[3-chloro-4-(pyridin-2-ylmethoxy)phenyl]amino}quinazolin-5-yl)oxy]-4-hydroxy-N-(2-hydroxyethyl)-N-methylbutanamide;
(3R)-3-[(4-{[3-chloro-4-(pyridin-2-ylmethoxy)phenyl]amino}quinazolin-5-yl)oxy]-4-morpholin-4-yl-4-oxobutan-1-ol;
(3R)-3-[(4-{[3-chloro-4-(pyridin-2-ylmethoxy)phenyl]amino}quinazolin-5-yl)oxy]-4-oxo-4-pyrrolidin-1-ylbutan-1-ol;
(3R)-3-[(4-{[3-chloro-4-(pyridin-2-ylmethoxy)phenyl]amino}quinazolin-5-yl)oxy]-4-(4-methylpiperazin-1-yl)-4-oxobutan-1-ol;
2-[(4-{[3-chloro-4-(pyridin-2-ylmethoxy)phenyl]amino}quinazolin-5-yl)oxy]-2-methylpropanamide;
2-[(4-{[3-chloro-4-(pyridin-2-ylmethoxy)phenyl]amino}quinazolin-5-yl)oxy]-N,2-dimethylpropanamide;
2-[(4-{[3-chloro-4-(pyridin-2-ylmethoxy)phenyl]amino}quinazolin-5-yl)oxy]-N-(2-hydroxy-1,1-dimethylethyl)-2-methylpropanamide;
2-[(4-{[3-chloro-4-(pyridin-2-ylmethoxy)phenyl]amino}quinazolin-5-yl)oxy]-N-(2-hydroxyethyl)-2-methylpropanamide;
2-[(4-{[3-chloro-4-(pyridin-2-ylmethoxy)phenyl]amino}quinazolin-5-yl)oxy]-N,N-bis(2-hydroxyethyl)-2-methylpropanamide;
2-[(4-{[3-chloro-4-(pyridin-2-ylmethoxy)phenyl]amino}quinazolin-5-yl)oxy]-N-(2-hydroxyethyl)-N,2-dimethylpropanamide;
(3R)-1-{2-[(4-{[3-chloro-4-(pyridin-2-ylmethoxy)phenyl]amino}quinazolin-5-yl)oxy]-2-methylpropanoyl}pyrrolidin-3-ol;
N-(2-hydroxyethyl)-2-methyl-2-[(4-{[3-methyl-4-(pyridin-2-ylmethoxy)phenyl]amino}quinazolin-5-yl)oxy]propanamide;
N,2-dimethyl-2-[(4-{[3-methyl-4-(pyridin-2-ylmethoxy)phenyl]amino}quinazolin-5-yl)oxy]propanamide;
2-{[4-({3-methyl-4-[(6-methylpyridin-3-yl)oxy]phenyl}amino)quinazolin-5-yl]oxy}acetamide;
N-(2-hydroxyethyl)-2-{[4-({3-methyl-4-[(6-methylpyridin-3-yl)oxy]phenyl}amino)quinazolin-5-yl]oxy}acetamide;
N-methyl-2-{[4-({3-methyl-4-[(6-methylpyridin-3-yl)oxy]phenyl}amino)quinazolin-5-yl]oxy}acetamide;
N-(2-hydroxyethyl)-N-methyl-2-{[4-({3-methyl-4-[(6-methylpyridin-3-yl)oxy]phenyl}amino)quinazolin-5-yl]oxy}acetamide;
N-{3-methyl-4-[(6-methylpyridin-3-yl)oxy]phenyl}-5-(2-oxo-2-pyrrolidin-1-ylethoxy)quinazolin-4-amine;
N-{3-methyl-4-[(6-methylpyridin-3-yl)oxy]phenyl}-5-(2-oxo-2-piperazin-1-ylethoxy)quinazolin-4-amine;
N-{3-methyl-4-[(6-methylpyridin-3-yl)oxy]phenyl}-5-[2-(4-methylpiperazin-1-yl)-2-oxoethoxy]quinazolin-4-amine;
(2S)-2-{[4-({3-methyl-4-[(6-methylpyridin-3-yl)oxy]phenyl}amino)quinazolin-5-yl]oxy}propanamide;
(2R)-2-{[4-({3-methyl-4-[(6-methylpyridin-3-yl)oxy]phenyl}amino)quinazolin-5-yl]oxy}propanamide;
(2R)—N-(2-hydroxyethyl)-N-methyl-2-{[4-({3-methyl-4-[(6-methylpyridin-3-yl)oxy]phenyl}amino)quinazolin-5-yl]oxy}propanamide;
2-methyl-2-{[4-({3-methyl-4-[(6-methylpyridin-3-yl)oxy]phenyl}amino)quinazolin-5-yl]oxy}propanamide;
N,2-dimethyl-2-{[4-({3-methyl-4-[(6-methylpyridin-3-yl)oxy]phenyl}amino)quinazolin-5-yl]oxy}propanamide;
(3R)-1-{(2S)-2-[(4-{[3-chloro-4-(pyridin-2-ylmethoxy)phenyl]amino}quinazolin-5-yl)oxy]propanoyl}pyrrolidin-3-ol;
(3S)-1-{(2S)-2-[(4-{[3-chloro-4-(pyridin-2-ylmethoxy)phenyl]amino}quinazolin-5-yl)oxy]propanoyl}pyrrolidin-3-ol;
(3R)-1-{(2R)-2-[(4-{[3-methyl-4-(pyridin-2-ylmethoxy)phenyl]amino}quinazolin-5-yl)oxy]propanoyl}pyrrolidin-3-ol;
(2R)—N-methyl-2-[(4-{[3-methyl-4-(pyridin-2-ylmethoxy)phenyl]amino}quinazolin-5-yl)oxy]propanamide;
(2R)—N-(2-hydroxyethyl)-N-methyl-2-[(4-{[3-methyl-4-(pyridin-2-ylmethoxy)phenyl]amino}quinazolin-5-yl)oxy]propanamide;
5-[(1R)-1-methyl-2-oxo-2-pyrrolidin-1-ylethoxy]-N-[3-methyl-4-(pyridin-2-ylmethoxy)phenyl]quinazolin-4-amine;
2-methyl-2-[(4-{[3-methyl-4-(pyridin-2-ylmethoxy)phenyl]amino}quinazolin-5-yl)oxy]propanamide;
N-(2-hydroxyethyl)-2-methyl-2-{[4-({3-methyl-4-[(6-methylpyridin-3-yl)oxy]phenyl}amino)quinazolin-5-yl]oxy}propanamide;
N-(2-hydroxyethyl)-N,2-dimethyl-2-{[4-({3-methyl-4-[(6-methylpyridin-3-yl)oxy]phenyl}amino)quinazolin-5-yl]oxy}propanamide;
(2S)—N-methyl-2-{[4-({3-methyl-4-[(6-methylpyridin-3-yl)oxy]phenyl}amino)quinazolin-5-yl]oxy}propanamide;
(2S)—N-(2-hydroxyethyl)-2-{[4-({3-methyl-4-[(6-methylpyridin-3-yl)oxy]phenyl}amino)quinazolin-5-yl]oxy}propanamide;
(2S)—N-(2-hydroxyethyl)-N-methyl-2-{[4-({3-methyl-4-[(6-methylpyridin-3-yl)oxy]phenyl}amino)quinazolin-5-yl]oxy}propanamide;
N-{3-methyl-4-[(6-methylpyridin-3-yl)oxy]phenyl}-5-[(1S)-1-methyl-2-morpholin-4-yl-2-oxoethoxy]quinazolin-4-amine;
(3S)-1-((2S)-2-{[4-({3-methyl-4-[(6-methylpyridin-3-yl)oxy]phenyl}amino)quinazolin-5-yl]oxy}propanoyl)pyrrolidin-3-ol;
(3S)-1-((2R)-2-{[4-({3-methyl-4-[(6-methylpyridin-3-yl)oxy]phenyl}amino)quinazolin-5-yl]oxy}propanoyl)pyrrolidin-3-ol;
(3R)-1-((2R)-2-{[4-({3-methyl-4-[(6-methylpyridin-3-yl)oxy]phenyl}amino)quinazolin-5-yl]oxy}propanoyl)pyrrolidin-3-ol;
(2R)—N-methyl-2-{[4-({3-methyl-4-[(6-methylpyridin-3-yl)oxy]phenyl}amino)quinazolin-5-yl]oxy}propanamide;
(2R)—N-(2-hydroxyethyl)-2-{[4-({3-methyl-4-[(6-methylpyridin-3-yl)oxy]phenyl}amino)quinazolin-5-yl]oxy}propanamide;
(2R)—N,N-dimethyl-2-{[4-({3-methyl-4-[(6-methylpyridin-3-yl)oxy]phenyl}amino)quinazolin-5-yl]oxy}propanamide;
(2R)—N-isopropyl-2-{[4-({3-methyl-4-[(6-methylpyridin-3-yl)oxy]phenyl}amino)quinazolin-5-yl]oxy}propanamide;
(2R)—N-ethyl-2-{[4-({3-methyl-4-[(6-methylpyridin-3-yl)oxy]phenyl}amino)quinazolin-5-yl]oxy}propanamide;
(2R)—N-[2-(diethylamino)ethyl]-2-{[4-({3-methyl-4-[(6-methylpyridin-3-yl)oxy]phenyl}amino)quinazolin-5-yl]oxy}propanamide;
(2R)—N-[2-(dimethylamino)ethyl]-2-{[4-({3-methyl-4-[(6-methylpyridin-3-yl)oxy]phenyl}amino)quinazolin-5-yl]oxy}propanamide;
(2R)—N-cyclopropyl-2-{[4-({3-methyl-4-[(6-methylpyridin-3-yl)oxy]phenyl}amino)quinazolin-5-yl]oxy}propanamide;
(2R)—N-(3-hydroxypropyl)-2-{[4-({3-methyl-4-[(6-methylpyridin-3-yl)oxy]phenyl}amino)quinazolin-5-yl]oxy}propanamide;
(2R)—N-(2-methoxyethyl)-2-{[4-({3-methyl-4-[(6-methylpyridin-3-yl)oxy]phenyl}amino)quinazolin-5-yl]oxy}propanamide;
(2R)-2-{[4-({3-methyl-4-[(6-methylpyridin-3-yl)oxy]phenyl}amino)quinazolin-5-yl]oxy}-N-(2-morpholin-4-ylethyl)propanamide;
(2R)-2-{[4-({3-methyl-4-[(6-methylpyridin-3-yl)oxy]phenyl}amino)quinazolin-5-yl]oxy}-N-(2-pyrrolidin-1-ylethyl)propanamide;
(2R)—N-[2-(acetylamino)ethyl]-2-{[4-({3-methyl-4-[(6-methylpyridin-3-yl)oxy]phenyl}amino)quinazolin-5-yl]oxy}propanamide;
(2R)-2-{[4-({3-methyl-4-[(6-methylpyridin-3-yl)oxy]phenyl}amino)quinazolin-5-yl]oxy}-N-[3-(4-methylpiperazin-1-yl)propyl]propanamide;
(2R)-2-{[4-({3-methyl-4-[(6-methylpyridin-3-yl)oxy]phenyl}amino)quinazolin-5-yl]oxy}-N-[3-(2-oxopyrrolidin-1-yl)propyl]propanamide;
(2R)-2-{[4-({3-methyl-4-[(6-methylpyridin-3-yl)oxy]phenyl}amino)quinazolin-5-yl]oxy}-N-[2-(methylthio)ethyl]propanamide;
(2R)—N-(3-methoxypropyl)-2-{[4-({3-methyl-4-[(6-methylpyridin-3-yl)oxy]phenyl}amino)quinazolin-5-yl]oxy}propanamide;
(2R)—N-cyclobutyl-2-{[4-({3-methyl-4-[(6-methylpyridin-3-yl)oxy]phenyl}amino)quinazolin-5-yl]oxy}propanamide;
(2R)—N-[(2R)-2-hydroxypropyl]-2-{[4-({3-methyl-4-[(6-methylpyridin-3-yl)oxy]phenyl}amino)quinazolin-5-yl]oxy}propanamide;
(2R)—N-[(2S)-2-hydroxypropyl]-2-{[4-({3-methyl-4-[(6-methylpyridin-3-yl)oxy]phenyl}amino)quinazolin-5-yl]oxy}propanamide;
(2R)—N-[(2S)-2,3-dihydroxypropyl]-2-{[4-({3-methyl-4-[(6-methylpyridin-3-yl)oxy]phenyl}amino)quinazolin-5-yl]oxy}propanamide;
(2R)—N-[(1R)-2-hydroxy-1-methylethyl]-2-{[4-({3-methyl-4-[(6-methylpyridin-3-yl)oxy]phenyl}amino)quinazolin-5-yl]oxy}propanamide;
(2R)—N-[(1S)-2-hydroxy-1-methylethyl]-2-{[4-({3-methyl-4-[(6-methylpyridin-3-yl)oxy]phenyl}amino)quinazolin-5-yl]oxy}propanamide;
N-{3-methyl-4-[(6-methylpyridin-3-yl)oxy]phenyl}-5-[(1R)-1-methyl-2-morpholin-4-yl-2-oxoethoxy]quinazolin-4-amine;
(2R)—N-[2-(dimethylamino)ethyl]-N-methyl-2-{[4-({3-methyl-4-[(6-methylpyridin-3-yl)oxy]phenyl}amino)quinazolin-5-yl]oxy}propanamide;
5-[(1R)-1-methyl-2-(4-methylpiperazin-1-yl)-2-oxoethoxy]-N-{3-methyl-4-[(6-methylpyridin-3-yl)oxy]phenyl}quinazolin-4-amine;
[(2R)-1-((2R)-2-{[4-({3-methyl-4-[(6-methylpyridin-3-yl)oxy]phenyl}amino)quinazolin-5-yl]oxy}propanoyl)pyrrolidin-2-yl]methanol;
[(2S)-1-((2R)-2-{[4-({3-methyl-4-[(6-methylpyridin-3-yl)oxy]phenyl}amino)quinazolin-5-yl]oxy}propanoyl)pyrrolidin-2-yl]methanol;
1-((2R)-2-{[4-({3-methyl-4-[(6-methylpyridin-3-yl)oxy]phenyl}amino)quinazolin-5-yl]oxy}propanoyl)piperidin-4-ol;
(2R)—N,N-bis(2-hydroxyethyl)-2-{[4-({3-methyl-4-[(6-methylpyridin-3-yl)oxy]phenyl}amino)quinazolin-5-yl]oxy}propanamide;
(2R)—N-ethyl-N-(2-hydroxyethyl)-2-{[4-({3-methyl-4-[(6-methylpyridin-3-yl)oxy]phenyl}amino)quinazolin-5-yl]oxy}propanamide;
(2R)—N,N-bis(2-methoxyethyl)-2-{[4-({3-methyl-4-[(6-methylpyridin-3-yl)oxy]phenyl}amino)quinazolin-5-yl]oxy}propanamide;
5-[(1R)-2-(4-ethylpiperazin-1-yl)-1-methyl-2-oxoethoxy]-N-{3-methyl-4-[(6-methylpyridin-3-yl)oxy]phenyl}quinazolin-4-amine;
(3R)-1-((2R)-2-{[4-({3-methyl-4-[(6-methylpyridin-3-yl)oxy]phenyl}amino)quinazolin-5-yl]oxy}propanoyl)piperidin-3-ol;
(3S)-1-((2R)-2-{[4-({3-methyl-4-[(6-methylpyridin-3-yl)oxy]phenyl}amino)quinazolin-5-yl]oxy}propanoyl)piperidin-3-ol;
4-((2R)-2-{[4-({3-methyl-4-[(6-methylpyridin-3-yl)oxy]phenyl}amino)quinazolin-5-yl]oxy}propanoyl)piperazin-2-one;
[1-((2R)-2-{[4-({3-methyl-4-[(6-methylpyridin-3-yl)oxy]phenyl}amino)quinazolin-5-yl]oxy}propanoyl)piperidin-4-yl]methanol;
tert-butyl 4-((2R)-2-{[4-({3-methyl-4-[(6-methylpyridin-3-yl)oxy]phenyl}amino)quinazolin-5-yl]oxy}propanoyl)piperazine-1-carboxylate;
N-{3-methyl-4-[(6-methylpyridin-3-yl)oxy]phenyl}-5-[(1R)-1-methyl-2-oxo-2-piperazin-1-ylethoxy]quinazolin-4-amine;
5-[(1R)-2-azetidin-1-yl-1-methyl-2-oxoethoxy]-N-{3-methyl-4-[(6-methylpyridin-3-yl)oxy]phenyl}quinazolin-4-amine;
1-((2R)-2-{[4-({3-methyl-4-[(6-methylpyridin-3-yl)oxy]phenyl}amino)quinazolin-5-yl]oxy}propanoyl)azetidin-3-ol;
(2R)—N-(2-methoxyethyl)-N-methyl-2-{[4-({3-methyl-4-[(6-methylpyridin-3-yl)oxy]phenyl}amino)quinazolin-5-yl]oxy}propanamide;
(2R)—N,N-diethyl-2-{[4-({3-methyl-4-[(6-methylpyridin-3-yl)oxy]phenyl}amino)quinazolin-5-yl]oxy}propanamide;
N-{3-methyl-4-[(6-methylpyridin-3-yl)oxy]phenyl}-5-[(1R)-1-methyl-2-oxo-2-pyrrolidin-1-ylethoxy]quinazolin-4-amine;
(2R)—N-(3-hydroxypropyl)-N-methyl-2-{[4-({3-methyl-4-[(6-methylpyridin-3-yl)oxy]phenyl}amino)quinazolin-5-yl]oxy}propanamide;
N-[3-fluoro-4-(pyridin-3-yloxy)phenyl]-5-[(1R)-1-methyl-2-morpholin-4-yl-2-oxoethoxy]quinazolin-4-amine;
N-{3-chloro-4-[(6-methylpyridin-3-yl)oxy]phenyl}-5-[(1R)-1-methyl-2-morpholin-4-yl-2-oxoethoxy]quinazolin-4-amine;
N-[3-chloro-4-(pyridin-3-yloxy)phenyl]-5-[(1R)-1-methyl-2-morpholin-4-yl-2-oxoethoxy]quinazolin-4-amine;
5-[(1R)-1-methyl-2-morpholin-4-yl-2-oxoethoxy]-N-{4-[(6-methylpyridin-3-yl)oxy]phenyl}quinazolin-4-amine;
5-[(1R)-1-methyl-2-morpholin-4-yl-2-oxoethoxy]-N-[4-(pyridin-3-yloxy)phenyl]-quinazolin-4-amine;
N-{3-methoxy-4-[(6-methylpyridin-3-yl)oxy]phenyl}-5-[(1R)-1-methyl-2-morpholin-4-yl-2-oxoethoxy]quinazolin-4-amine;
N-[3-methoxy-4-(pyridin-3-yloxy)phenyl]-5-[(1R)-1-methyl-2-morpholin-4-yl-2-oxoethoxy]quinazolin-4-amine;
N-{3-fluoro-4-[(6-methylpyridin-3-yl)oxy]phenyl}-5-[(1R)-1-methyl-2-morpholin-4-yl-2-oxoethoxy]quinazolin-4-amine;
N-{3-cyano-4-[(6-methylpyridin-3-yl)oxy]phenyl}-5-[(1R)-1-methyl-2-morpholin-4-yl-2-oxoethoxy]quinazolin-4-amine;
N-[3-cyano-4-(pyridin-3-yloxy)phenyl]-5-[(1R)-1-methyl-2-morpholin-4-yl-2-oxoethoxy]quinazolin-4-amine;
5-[(1R)-1-methyl-2-morpholin-4-yl-2-oxoethoxy]-N-[3-methyl-4-(pyridin-2-yloxy)phenyl]quinazolin-4-amine;
5-[(1R)-1-methyl-2-morpholin-4-yl-2-oxoethoxy]-N-[3-methyl-4-(pyridin-3-yloxy)phenyl]quinazolin-4-amine;
5-[(1R)-1-methyl-2-morpholin-4-yl-2-oxoethoxy]-N-[3-methyl-4-(pyridin-4-yloxy)phenyl]quinazolin-4-amine;
5-[(1R)-1-methyl-2-morpholin-4-yl-2-oxoethoxy]-N-[3-methyl-4-(pyrazin-2-yloxy)phenyl]quinazolin-4-amine;
5-[(1R)-1-methyl-2-morpholin-4-yl-2-oxoethoxy]-N-[3-methyl-4-(1,3-thiazol-2-yloxy)phenyl]quinazolin-4-amine;
N-{4-[(6-methoxypyridin-3-yl)oxy]-3-methylphenyl}-5-[(1R)-1-methyl-2-morpholin-4-yl-2-oxoethoxy]quinazolin-4-amine;
5-[(1R)-1-methyl-2-morpholin-4-yl-2-oxoethoxy]-N-[3-methyl-4-(1,3-thiazol-5-yloxy)phenyl]quinazolin-4-amine;
5-[(1R)-1-methyl-2-morpholin-4-yl-2-oxoethoxy]-N-[3-methyl-4-(pyrimidin-5-yloxy)phenyl]quinazolin-4-amine;
5-[2-methyl-4-({5-[(1R)-1-methyl-2-morpholin-4-yl-2-oxoethoxy]quinazolin-4-yl}amino)phenoxy]pyridine-2-carbonitrile;
5-[(1R)-1-methyl-2-morpholin-4-yl-2-oxoethoxy]-N-[3-methyl-4-(pyridazin-3-yloxy)phenyl]quinazolin-4-amine;
(2R)—N-(2-hydroxyethyl)-2-{[4-({3-methoxy-4-[(6-methylpyridin-3-yl)oxy]phenyl}amino)quinazolin-5-yl]oxy}-N-methylpropanamide;
(2R)-2-{[4-({3-methoxy-4-[(6-methylpyridin-3-yl)oxy]phenyl}amino)quinazolin-5-yl]oxy}-N,N-dimethylpropanamide;
(2R)—N-ethyl-2-{[4-({3-methoxy-4-[(6-methylpyridin-3-yl)oxy]phenyl}amino)quinazolin-5-yl]oxy}propanamide;
(2R)—N-(2-hydroxyethyl)-2-{[4-({3-methoxy-4-[(6-methylpyridin-3-yl)oxy]phenyl}amino)quinazolin-5-yl]oxy}propanamide;
4-((2R)-2-{[4-({3-methoxy-4-[(6-methylpyridin-3-yl)oxy]phenyl}amino)quinazolin-5-yl]oxy}propanoyl)piperazin-2-one;
(2R)—N-(2-methoxyethyl)-2-{[4-({3-methoxy-4-[(6-methylpyridin-3-yl)oxy]phenyl}amino)quinazolin-5-yl]oxy}-N-methylpropanamide;
(3R)-1-((2R)-2-{[4-({3-methoxy-4-[(6-methylpyridin-3-yl)oxy]phenyl}amino)quinazolin-5-yl]oxy}propanoyl)piperidin-3-ol;
N-{3-methoxy-4-[(6-methylpyridin-3-yl)oxy]phenyl}-5-[(1R)-1-methyl-2-oxo-2-piperazin-1-ylethoxy]quinazolin-4-amine;
(2R)—N,N-dimethyl-2-[(4-{[3-methyl-4-(pyridin-2-yloxy)phenyl]amino}quinazolin-5-yl)oxy]propanamide;
(2R)—N-ethyl-2-[(4-{[3-methyl-4-(pyridin-2-yloxy)phenyl]amino}quinazolin-5-yl)oxy]propanamide;
(2R)—N-(2-hydroxyethyl)-2-[(4-{[3-methyl-4-(pyridin-2-yloxy)phenyl]amino}quinazolin-5-yl)oxy]propanamide;
(2R)—N-(2-hydroxyethyl)-N-methyl-2-[(4-{[3-methyl-4-(pyridin-2-yloxy)phenyl]amino}quinazolin-5-yl)oxy]propanamide;
4-{(2R)-2-[(4-{[3-methyl-4-(pyridin-2-yloxy)phenyl]amino}quinazolin-5-yl)oxy]propanoyl}piperazin-2-one;
(2R)—N-(2-methoxyethyl)-N-methyl-2-[(4-{[3-methyl-4-(pyridin-2-yloxy)phenyl]amino}quinazolin-5-yl)oxy]propanamide;
(3R)-1-{(2R)-2-[(4-{[3-methyl-4-(pyridin-2-yloxy)phenyl]amino}quinazolin-5-yl)oxy]propanoyl}piperidin-3-ol;
5-[(1R)-1-methyl-2-oxo-2-piperazin-1-ylethoxy]-N-[3-methyl-4-(pyridin-2-yloxy)phenyl]quinazolin-4-amine;
5-[(1R)-1-methyl-2-morpholin-4-yl-2-oxoethoxy]-N-[3-methyl-4-(pyridin-2-ylmethoxy)phenyl]quinazolin-4-amine;
{5-[2-methyl-4-({5-[(1R)-1-methyl-2-morpholin-4-yl-2-oxoethoxy]quinazolin-4-yl}amino)phenoxy]pyridin-2-yl}methanol;
N-{4-[(6-fluoropyridin-3-yl)oxy]-3-methylphenyl}-5-[(1R)-1-methyl-2-morpholin-4-yl-2-oxoethoxy]quinazolin-4-amine;
N-[3-chloro-4-(pyridin-2-yloxy)phenyl]-5-[(1R)-1-methyl-2-morpholin-4-yl-2-oxoethoxy]quinazolin-4-amine;
(2R)-2-[(4-{[3-chloro-4-(pyridin-2-yloxy)phenyl]amino}quinazolin-5-yl)oxy]-N-(2-hydroxyethyl)-N-methylpropanamide;
(2R)-2-[(4-{[3-chloro-4-(pyridin-2-yloxy)phenyl]amino}quinazolin-5-yl)oxy]-N,N-dimethylpropanamide;
(2R)-2-[(4-{[3-chloro-4-(pyridin-2-yloxy)phenyl]amino}quinazolin-5-yl)oxy]-N-(2-hydroxyethyl)propanamide;
(2R)-2-[(4-{[3-chloro-4-(pyridin-2-yloxy)phenyl]amino}quinazolin-5-yl)oxy]-N-ethyl-N-(2-hydroxyethyl)propanamide;
(2R)-2-[(4-{[3-chloro-4-(pyridin-2-yloxy)phenyl]amino}quinazolin-5-yl)oxy]-N-(2-methoxyethyl)-N-methylpropanamide;
4-{(2R)-2-[(4-{[3-chloro-4-(pyridin-2-yloxy)phenyl]amino}quinazolin-5-yl)oxy]propanoyl}piperazin-2-one;
N-[3-chloro-4-(pyridin-2-yloxy)phenyl]-5-[(1R)-1-methyl-2-oxo-2-piperazin-1-ylethoxy]quinazolin-4-amine;
1-{(2R)-2-[(4-{[3-chloro-4-(pyridin-2-yloxy)phenyl]amino}quinazolin-5-yl)oxy]propanoyl}piperidin-3-ol;
N-{3-methyl-4-[(1-methyl-1H-pyrazol-4-yl)oxy]phenyl}-5-[(1R)-1-methyl-2-morpholin-4-yl-2-oxoethoxy]quinazolin-4-amine;
N-{3-chloro-4-[(1-methyl-1H-pyrazol-4-yl)oxy]phenyl}-5-[(1R)-1-methyl-2-morpholin-4-yl-2-oxoethoxy]quinazolin-4-amine;
N-(4-{[6-(fluoromethyl)pyridin-3-yl]oxy}-3-methylphenyl)-5-[(1R)-1-methyl-2-morpholin-4-yl-2-oxoethoxy]quinazolin-4-amine;
N-[3-chloro-4-(1,3-thiazol-2-yloxy)phenyl]-5-[(1R)-1-methyl-2-morpholin-4-yl-2-oxoethoxy]quinazolin-4-amine;
(2S)—N,N-dimethyl-2-{[4-({3-methyl-4-[(6-methylpyridin-3-yl)oxy]phenyl}amino)quinazolin-5-yl]oxy}propanamide;
(2R)-2-{[4-({3-chloro-4-[(6-methylpyridin-3-yl)oxy]phenyl}amino)quinazolin-5-yl]oxy}-N-(2-hydroxyethyl)-N-methylpropanamide;
(2R)-2-{[4-({3-chloro-4-[(6-methylpyridin-3-yl)oxy]phenyl}amino)quinazolin-5-yl]oxy}-N,N-dimethylpropanamide;
N-{3-chloro-4-[(6-fluoropyridin-3-yl)oxy]phenyl}-5-[(1R)-1-methyl-2-morpholin-4-yl-2-oxoethoxy]quinazolin-4-amine;
N-[3-chloro-4-(pyrazin-2-yloxy)phenyl]-5-[(1R)-1-methyl-2-morpholin-4-yl-2-oxoethoxy]quinazolin-4-amine; and
N-[3-chloro-4-(1,3-thiazol-5-yloxy)phenyl]-5-[(1R)-1-methyl-2-morpholin-4-yl-2-oxoethoxy]quinazolin-4-amine;
or a pharmaceutically acceptable salt thereof.

21. A pharmaceutical composition which comprises a quinazoline derivative of the formula I, or a pharmaceutically acceptable salt thereof, as defined in any one of claims 1 to 20 in association with a pharmaceutically-acceptable diluent or carrier.

22. A quinazoline derivative of the formula I, or a pharmaceutically acceptable salt thereof, as defined in any one of claims 1 to 20 for use as a medicament.

23. A quinazoline derivative of the formula I, or a pharmaceutically acceptable salt thereof, as defined in any one of claims 1 to 20 for use in the production of an anti-proliferative effect which effect is produced alone or in part by inhibiting erbB2 receptor tyrosine kinase in a warm-blooded animal such as man.

24. A quinazoline derivative of the formula I, or a pharmaceutically acceptable salt thereof, as defined in any one of claims 1 to 20 for use in the production of an erbB2 receptor tyrosine kinase inhibitory effect in a warm-blooded animal such as man.

25. A quinazoline derivative of the formula I, or a pharmaceutically acceptable salt thereof, as defined in any one of claims 1 to 20 for use in the production of a selective erbB2 receptor tyrosine kinase inhibitory effect in a warm-blooded animal such as man.

26. A process for the preparation of a quinazoline derivative of the formula I, or a pharmaceutically acceptable salt thereof, as defined in claim 1 which comprises:
Process (a) the reaction of a quinazoline of the formula II: 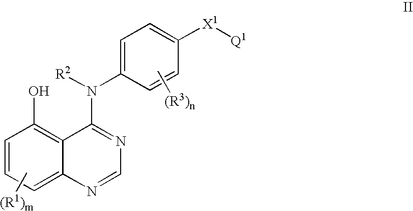
wherein R¹, R², R³, X¹, Q¹, m and n have any of the meanings defined in claim 1 except that any functional group is protected if necessary, with an amide of the formula III: 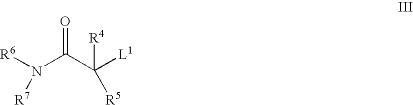
wherein R⁴, R⁵, R⁶ and R⁷ have any of the meanings defined in claim 1 except that any functional group is protected if necessary and L¹ is a suitable displaceable group; or
Process (b) the coupling of a quinazoline of the formula IV: 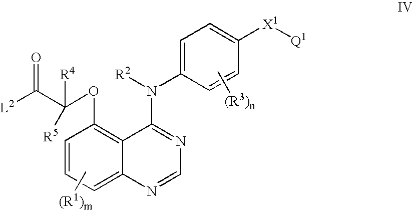
wherein R¹, R², R³, R⁴, R⁵, X¹, Q¹, m and n have any of the meanings defined in claim 1 except that any functional group is protected if necessary, and L² is a suitable displaceable group, or L² is hydroxy which is conveniently combined with a suitable coupling agent to produce a displaceable group, with an amine of the formula V: 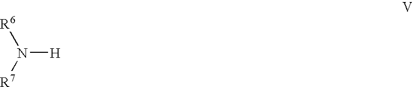
wherein R⁶ and R⁷ have any of the meanings defined in claim 1 except that any functional group is protected if necessary; or
Process (c) for quinazoline derivatives of the formula I wherein at least one of R⁴ and R⁵ is 2-hydroxyethyl, the reaction of a quinazoline of the formula VI: 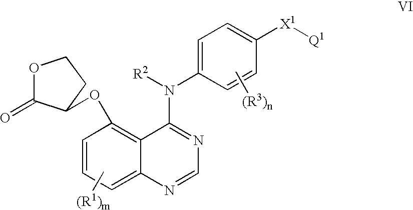
wherein R¹, R², R³, X¹, Q¹, m and n have any of the meanings defined in claim 1 except that any functional group is protected if necessary, with an amine of the formula V as defined above; or
Process (d) the reaction of a quinazoline of the formula VII: 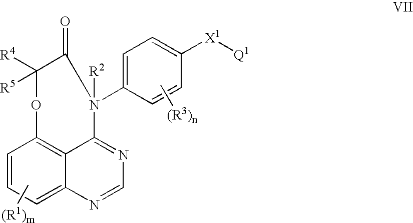
wherein R¹, R², R³, R⁴, R⁵, X¹, Q¹, m and n have any of the meanings defined in claim 1 except that any functional group is protected if necessary, with an amine of the formula V as defined above; or
Process (e) the reaction of a quinazolone of the formula VIII: 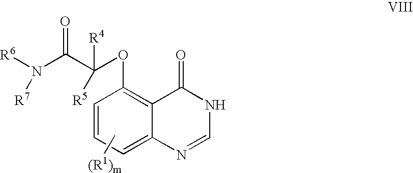
wherein R¹, R⁴, R⁵, R⁶, R⁷ and m have any of the meanings defined in claim 1 except that any functional group is protected if necessary, with a suitable activating group and an amine of the formula IX: 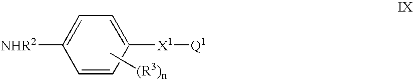
wherein R², R³, X¹, Q¹ and n have any of the meanings defined in claim 1 except that any functional group is protected if necessary; or
Process (f) when X¹ is O, S, OC(R¹³)₂ or SC(R¹³)₂, the reaction of a quinazoline of the formula X: 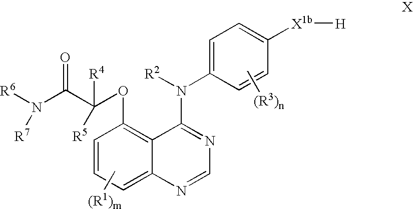
wherein R¹, R², R³, R⁴, R⁵, R⁶, R⁷, n and m have any of the meanings defined in claim 1 except that any functional group is protected if necessary and X^{1b} is O or S, with a compound of the formula Q¹-[C(R¹³)²]ᵣ-L³ wherein r is 0 or 1, L³ is a suitable displaceable group and R¹³ and Q¹ have any of the meanings defined in claim 1 except that any functional group is protected if necessary; or
Process (g) the reaction of a quinazoline of the formula XI: 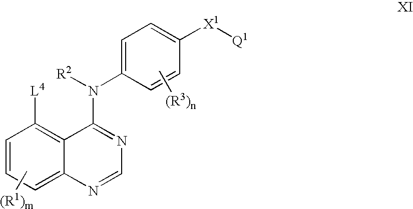
wherein L⁴ is a suitable displaceable group and R¹, R², R³, X¹, Q¹, n and m have any of the meanings defined in claim 1 except that any functional group is protected if necessary with a compound of the formula XII: 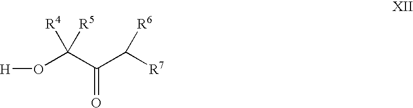
wherein R⁴, R⁵, R⁶ and R⁷ have any of the meanings defined in claim 1 except that any functional group is protected if necessary;
and thereafter, if necessary:
(i) converting a quinazoline derivative of the formula I into another quinazoline derivative of the formula I;
(ii) removing any protecting group;
(iii) forming a pharmaceutically acceptable salt.
